(19) United States
(12) Patent Application Publication
(10) Pub. No.: US 2024/0238458 A1
(43) Pub. Date: Jul. 18, 2024

(54) COMPOUNDS TARGETING FIBROBLAST-ACTIVATION PROTEIN AND METHODS OF USE THEREOF

(71) Applicant: Purdue Research Foundation, West Lafayette, IN (US)

(72) Inventors: Philip Stewart LOW, West Lafayette, IN (US); Ramesh MUKKAMALA, West Lafayette, IN (US); Madduri SRINIVASARAO, West Lafayette, IN (US); Spencer D. LINDEMAN, West Lafayette, IN (US)

(21) Appl. No.: 18/550,964

(22) PCT Filed: Mar. 12, 2022
(86) PCT No.: PCT/US2022/020084

§ 371 Date: Sep. 16, 2023

Related U.S. Application Data
(60) Provisional application No. 63/165,583, filed on Mar. 24, 2021.
(60) Provisional application No. 63/165,563, filed on Mar. 24, 2021.
(60) Provisional application No. 63/165,550, filed on Mar. 24, 2021.
(60) Provisional application No. 63/161,835, filed on Mar. 16, 2021.
(60) Provisional application No. 63/200,592, filed on Mar. 16, 2021.
(60) Provisional application No. 63/200,593, filed on Mar. 16, 2021.

(51) Int. Cl. A61K 51/08; A61K 45/06; A61P 35/00

(52) U.S. Cl. CPC A61K 51/088; A61K 45/06; A61P 35/00; A61K 2121/00; A61K 2123/00

(57) ABSTRACT

Fibroblast activation protein (FAP)-targeting compounds; methods for imaging cancer and fibrosis; and methods for treating fibrosis, an inflammatory disease/disorder, and cancer.

### PRIORITY

This patent application is related to and claims the priority benefit of: (a) U.S. Provisional Patent Application No. 63/200,593 filed Mar. 16, 2021; (b) U.S. Provisional Patent Application No. 63/165,563 filed Mar. 24, 2021; (c) U.S. Provisional Patent Application No. 63/200,592 filed Mar. 16, 2021; (d) U.S. Provisional Patent Application No. 63/165,550 filed Mar. 24, 2021; (e) U.S. Provisional Patent Application No. 63/161,835 filed Mar. 16, 2021; and (f) U.S. Provisional Patent Application No. 63/165,583 filed Mar. 24, 2021. The contents of each of the aforementioned applications are hereby incorporated by reference in their entireties herein.

### BACKGROUND

The survival and proliferation of tumors can be dependent on the tumor microenvironment (TME) including, without limitation, the percentage of tumor stroma (TSP). A high TSP can be associated with poorer long-term patient survival compared to low TSP (>50% vs. ≤50% respectively). The TSP can also be a significant prognostic factor for tumor relapse, growth, and metastasis.

Aside from cancer cells, tumors (and TSP) comprise infiltrating immune and inflammatory cells such as tumor associated fibroblasts (TAFs) or cancer-associated fibroblasts (CAFs), extracellular matrix (ECM) proteins, T cells, tumor-associated macrophages (TAMs), myeloid-suppressor cells, blood and lymphatic vasculature, etc. They aid in the growth and development of the tumor by growth factor secretion, immunosuppression, metastasis, resistance, etc.

TAFs or CAFs are one of the major types of cells present in the tumor stroma and perform several critical roles to promote tumor growth. These functions include ECM production, remodeling, and cytokine secretion. These lead to angiogenesis to promote tumor growth, signaling factor secretion to increase chemoresistance, denser tumor stroma to provide a physical blockade against immune cells, and enhanced cell motility to direct metastasis. In some instances, such processes parallel the behavior of pathogenic fibroblasts in fibrotic diseases.

In some instances, a prevalent marker of CAFs is fibroblast activation protein alpha (FAPα). FAPα is a serine protease (primarily) found on the cell surface of activated fibroblasts in diseased cells and tissue, such as in fibrotic disease, inflammatory disease, and/or cancer (e.g., fibrosis, rheumatoid arthritis, wound healing, and cancer). More than 90% of epithelial carcinomas show FAPα expression in immunohistochemical (IHC) staining. Additional FAPα expression has been found in a subset of primary glioma cell cultures and TAMs. Recently, FAPα expression has been detected in at least 28 different types of human cancers. However, FAPα expression is very low or nonexistent in the majority of adult tissues. Therefore, because the expression is restricted to the surfaces of diseased cells, such as carcinomas, FAPα is uniquely qualified as a receptor for selectively delivering pharmacotherapeutics to tumors via ligand-targeting.

Radio- and chemo-therapies and other therapies for killing tumor cells can be considered for treatment of various cancers, fibrotic disorders, and inflammatory diseases. Often however, such therapies are not used as first line therapies because of adverse (e.g., systemic) effects that can result. As a result, there is a need for targeted therapies, such as therapies employing targeted radio- and/or chem-therapeutic agents, which can target diseased cells and tissues and can treat disease (e.g., cancer, fibrotic disease, and/or inflammatory disease) with minimal or reduced off-target or systemic effects.

### SUMMARY

Provided are compounds (e.g., conjugates) of formula X:

Aₘₓ-L-B′ (X)

wherein
A is a radical of a fibroblast activation protein alpha (FAPα) ligand (i.e., a targeting moiety) (e.g., with a molecular weight below 10,000);
L is a (e.g., bi-functionalized or trifunctionalized) linker connecting one or more A groups to B′ (e.g., through a first covalent bond linking L to A and a second covalent bond linking L to B′);
B′ is (e.g., a radical of) a photodynamic therapeutic agent, a radio-imaging agent, a radiotherapeutic agent, a chemotherapeutic agent, an antifibrotic agent, or an anticancer agent (e.g., an anticancer agent that is effective against cancer cells or cancer-associated fibroblasts, myofibroblasts or other tumor microenvironment factors); and
mx is 1-6.

Also provided are compounds (e.g., conjugates) of formula I:

A-L-B′ (I)

wherein:
A comprises (e.g., a radical of) a FAPα ligand (i.e., a targeting moiety);
L comprises a (e.g., bi-functionalized or tri-functionalized) linker connecting one or more A groups to B′; and
B′ comprises (e.g., a radical of) a photodynamic therapeutic agent, a radio-imaging agent, a radiotherapeutic agent, a chemotherapeutic agent, an antifibrotic agent, or an anticancer agent (e.g., an anticancer agent that is effective against cancer cells or cancer-associated fibroblasts, myofibroblasts or other tumor microenvironment factors).

Provided are compounds (e.g., conjugates) of formula I′:

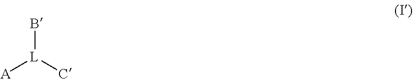

wherein:
A is a radical of a FAPα ligand (i.e., a targeting moiety) (e.g., with a molecular weight below 10,000);
L is a trivalent linker;
B′ is a radical of a phosphoinositide 3-kinase (PI3K) inhibitor, a chelating group optionally bound to an isotope, or a group covalently bound to an isotope, said isotope (or metal) being suitable for radio-imaging, radiotherapy or magnetic resonance imaging; and
C′ is a radical of an albumin binding ligand, a polyethylene glycol. (PEG)ₙ, wherein n is an integer from 0 to 32, a peptide, a peptidoglycan or a saccharide.

Also provided are compounds (e.g., conjugates) of formula (II):

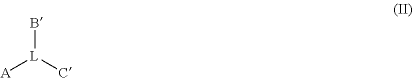

wherein
A comprises a radical of a FAPα ligand (i.e., a targeting moiety) (e.g., with a molecular weight below 10,000);
L comprises a trivalent linker; and
B′ comprises a radical of a PI3K inhibitor, a chelating group optionally bound to an isotope, or a group covalently bound to an isotope, said isotope (or metal) suitable for radio-imaging, radiotherapy or magnetic resonance imaging; and
C′ comprises a radical of an albumin binding ligand, a (PEG)ₙ wherein n is an integer from 0 to 32, a peptide, a peptidoglycan or a saccharide. Also provided is a compound represented by the structure of formula (V):

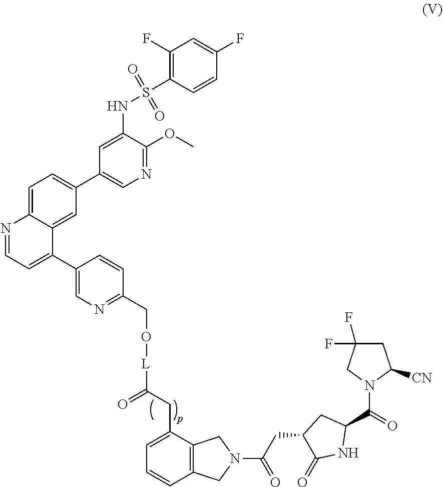

wherein:
L is a linker comprising at least one carbon atom; and
p is 0, 1, 2, or 3.

A compound is also provided that is represented by the structure of formula (X):

Aₘ-L-B′ (X)

wherein:
A is a radical of a FAPα ligand of the formula X-B:

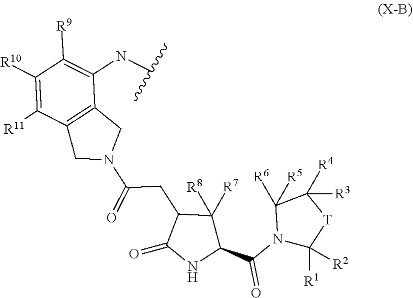

wherein:
T is substituted or unsubstituted methylene (—CH₂—), substituted or unsubstituted amino (—NH—), —O—, or —S— (e.g., wherein the substitution of T is C₁-C₃ alkyl, haloalkyl, or halo);
J is C(R^{J})₀₋₃, wherein each R^{J} is independently H or alkyl, or two or more R^{J} are taken together to form oxo;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are independently selected from group consisting of H, alkyl, and halo; and
R⁹, R¹⁰, and R¹¹ are independently selected from group consisting of H, —C₁₋₆alkyl, —C₁₋₆haloalkyl, —O—C₁₋₆alkyl, —S—C₁₋₆ alkyl, F, Cl, Br and I;
L is a linker linking A to B′;
B′ is a radical of a therapeutic agent, a radio-imaging agent, a radiotherapeutic agent, a magnetic resonance imaging agent, a chemotherapeutic agent, an antifibrotic agent, or an anticancer agent; and
m=1-6.

Such compounds can, in certain embodiments, further comprise C′, wherein: L connects C′ to the one or more A groups and B′; and C′ is a radical of an albumin binding ligand, (PEG)ₙ wherein n is an integer from 0 to 32, a peptide, a peptidoglycan or a saccharide.

In some embodiments, B′ is a radical of a PI3K inhibitor, a chelating group optionally bound to an isotope, or a group covalently bound to an isotope, said isotope (or metal) being suitable for radio-imaging, radiotherapy or magnetic resonance imaging.

Compounds are also provided that are represented by the structure of formula (I′):

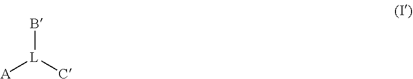

wherein:
A is a radical of a FAP ligand (i.e., a targeting moiety, for example and without limitation, a FAPα ligand) of the formula X-B:

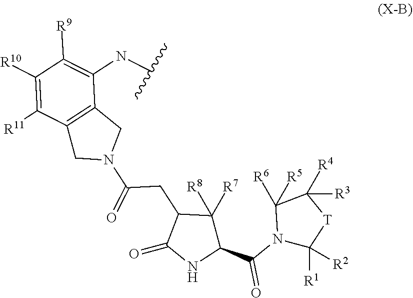

wherein:
T is substituted or unsubstituted methylene (—CH₂—), substituted or unsubstituted amino (—NH—), —O—, or —S— (e.g., wherein the substitution of T is C₁-C₃ alkyl, haloalkyl, or halo);
J is C(R^{J})₀₋₃, wherein each R^{J} is independently H or alkyl, or two or more R^{J} are taken together to form oxo;
R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are each independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl, and halo; and
R⁹, R¹⁰, and R¹¹ are each independently selected from group consisting of H, —C₁₋₆alkyl, —C₁₋₆haloalkyl, —O—C₁₋₆alkyl, —S—C₁₋₆ alkyl, F, Cl, Br and I;
L is a trivalent linker;
B′ is a radical of a chelating group optionally bound to an isotope, said isotope (or metal) being suitable for radio-imaging, radiotherapy or magnetic resonance imaging; and
C′ is a radical of an albumin binding ligand, (PEG)₁ wherein n is an integer from 0 to 32, a peptide, a peptidoglycan or a saccharide.

In certain embodiments of the compounds, A can be

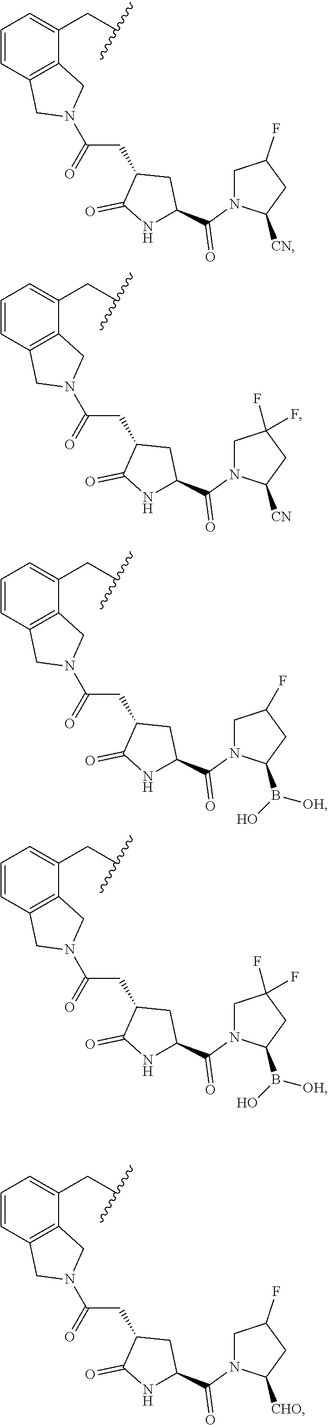 [SMILES: *Cc1cccc2c1CN(C(=O)C[C@@H]1C[C@@H](C(=O)N3CC(F)C[C@H]3C#N)NC1=O)C2 ; *Cc1cccc2c1CN(C(=O)C[C@@H]1C[C@@H](C(=O)N3CC(F)(F)C[C@H]3C#N)NC1=O)C2 ; *Cc1cccc2c1CN(C(=O)C[C@@H]1C[C@@H](C(=O)N3CC(F)C[C@H]3B(O)O)NC1=O)C2 ; *Cc1cccc2c1CN(C(=O)C[C@@H]1C[C@@H](C(=O)N3CC(F)(F)C[C@H]3B(O)O)NC1=O)C2 ; *Cc1cccc2c1CN(C(=O)C[C@@H]1C[C@@H](C(=O)N3CC(F)C[C@H]3C=O)NC1=O)C2]

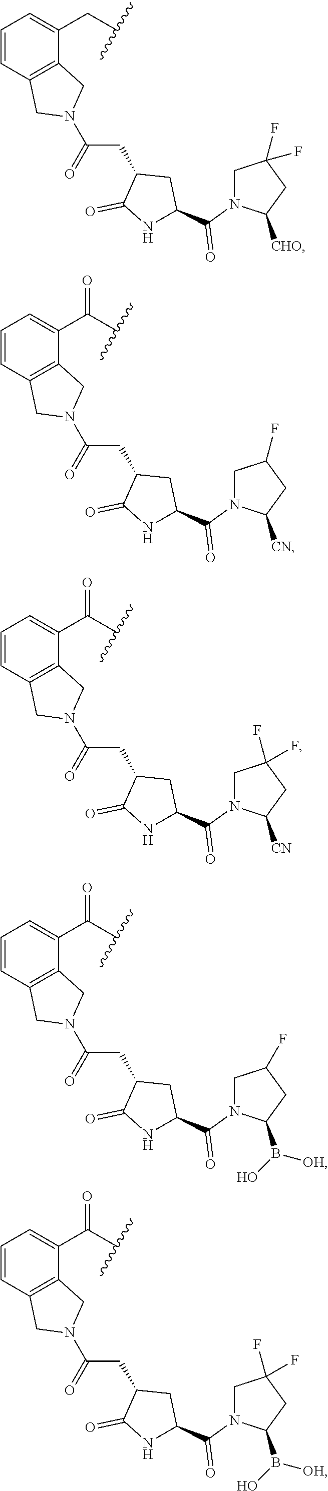 [SMILES: *Cc1cccc2c1CN(C(=O)C[C@@H]1C[C@@H](C(=O)N3CC(F)(F)C[C@H]3C=O)NC1=O)C2 ; *C(=O)c1cccc2c1CN(C(=O)C[C@@H]1C[C@@H](C(=O)N3CC(F)C[C@H]3C#N)NC1=O)C2 ; *C(=O)c1cccc2c1CN(C(=O)C[C@@H]1C[C@@H](C(=O)N3CC(F)(F)C[C@H]3C#N)NC1=O)C2 ; *C(=O)c1cccc2c1CN(C(=O)C[C@@H]1C[C@@H](C(=O)N3CC(F)C[C@H]3B(O)O)NC1=O)C2 ; *C(=O)c1cccc2c1CN(C(=O)C[C@@H]1C[C@@H](C(=O)N3CC(F)(F)C[C@H]3B(O)O)NC1=O)C2]

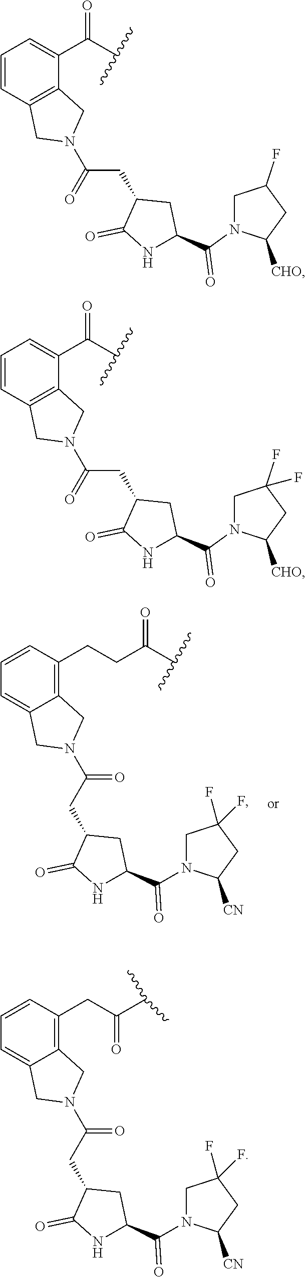 [SMILES: *C(=O)c1cccc2c1CN(C(=O)C[C@@H]1C[C@@H](C(=O)N3CC(F)C[C@H]3C=O)NC1=O)C2 ; *C(=O)c1cccc2c1CN(C(=O)C[C@@H]1C[C@@H](C(=O)N3CC(F)(F)C[C@H]3C=O)NC1=O)C2 ; *C(=O)CCc1cccc2c1CN(C(=O)C[C@@H]1C[C@@H](C(=O)N3CC(F)(F)C[C@H]3C#N)NC1=O)C2 ; *C(=O)Cc1cccc2c1CN(C(=O)C[C@@H]1C[C@@H](C(=O)N3CC(F)(F)C[C@H]3C#N)NC1=O)C2]

In certain embodiments of the compounds, A can be:

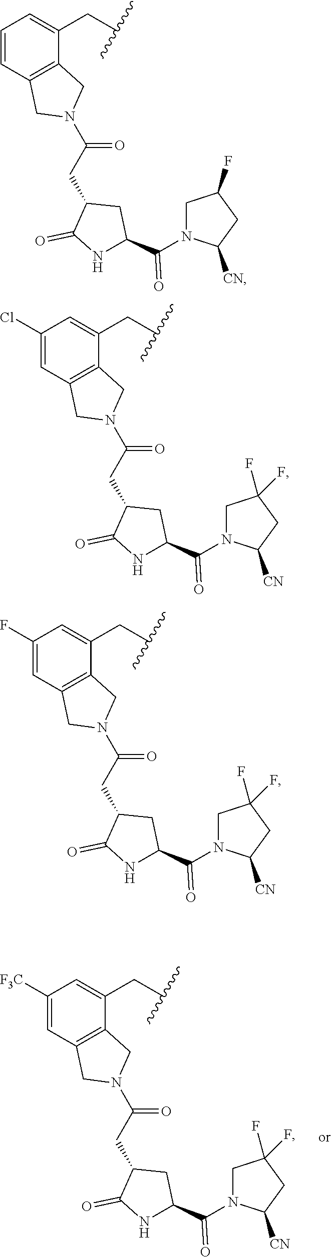

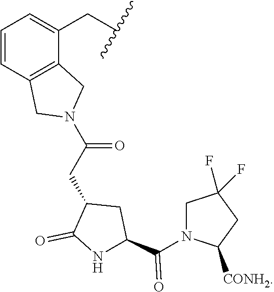 [SMILES: *Cc1cccc2c1CN(C(=O)C[C@@H]1C[C@@H](C(=O)N3CC(F)(F)C[C@H]3C(N)=O)NC1=O)C2]

In at least one embodiment of the compound, A comprises:

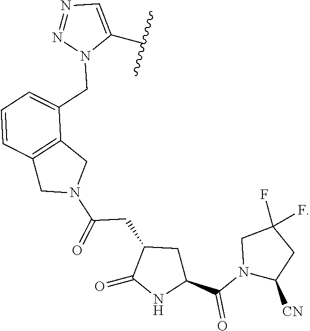 [SMILES: *c1cnnn1Cc1cccc2c1CN(C(=O)C[C@@H]1C[C@@H](C(=O)N3CC(F)(F)C[C@H]3C#N)NC1=O)C2]

Alternatively, A can comprise

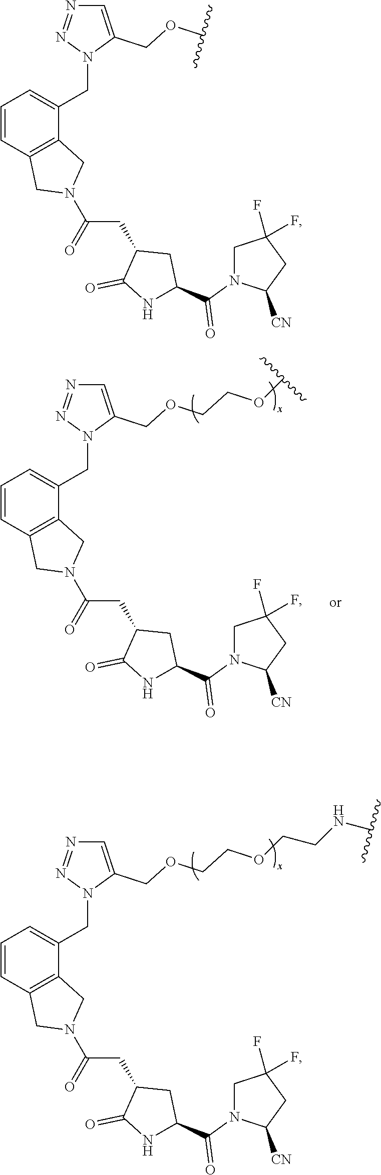

wherein x is 1-20.

In certain embodiments, A comprises:

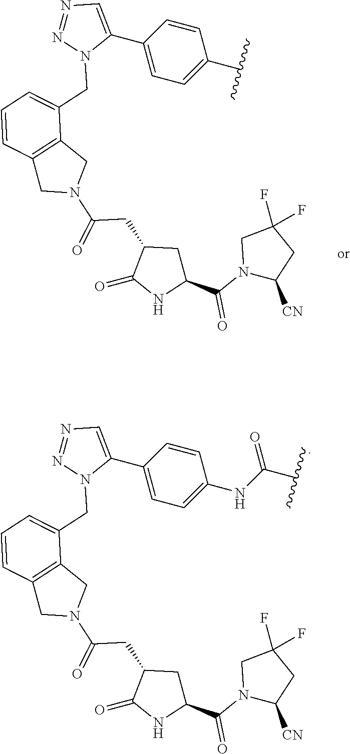 [SMILES: *c1ccc(-c2cnnn2Cc2cccc3c2CN(C(=O)C[C@@H]2C[C@@H](C(=O)N4CC(F)(F)C[C@H]4C#N)NC2=O)C3)cc1 ; *C(=O)Nc1ccc(-c2cnnn2Cc2cccc3c2CN(C(=O)C[C@@H]2C[C@@H](C(=O)N4CC(F)(F)C[C@H]4C#N)NC2=O)C3)cc1]

B′ can be a radical of a chelating group optionally bound to an isotope (or metal) suitable for radio-imaging, radiotherapy or magnetic resonance imaging.

B′ can be selected from

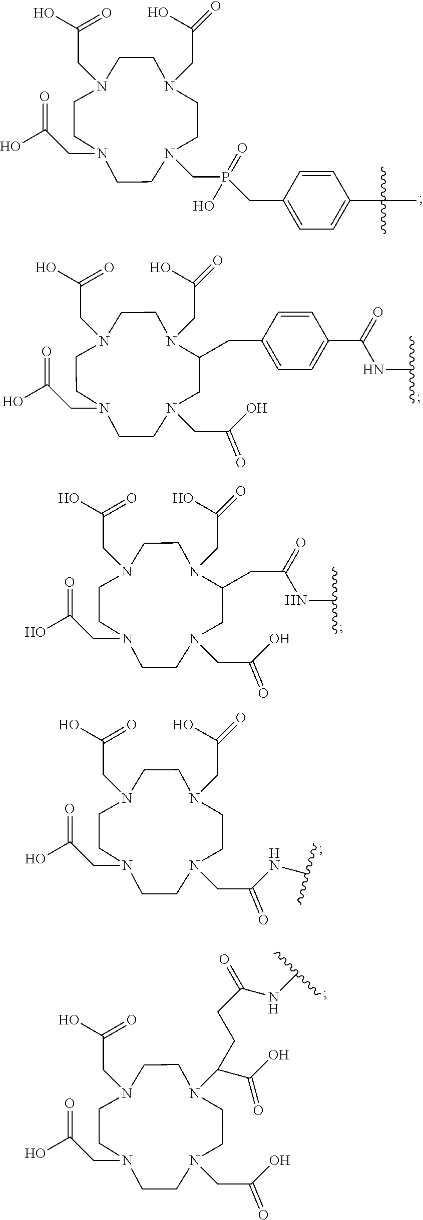

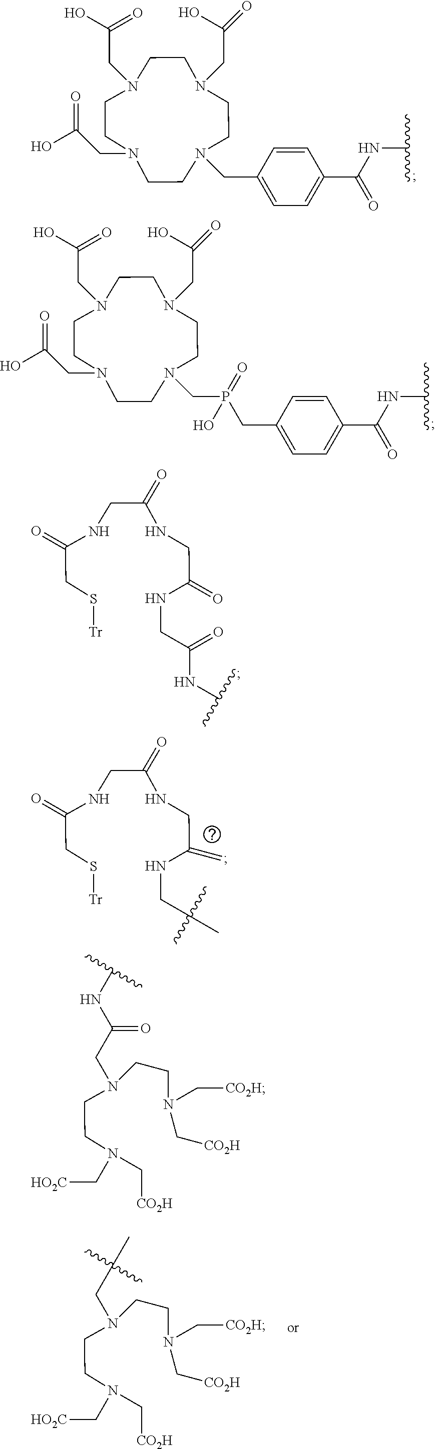

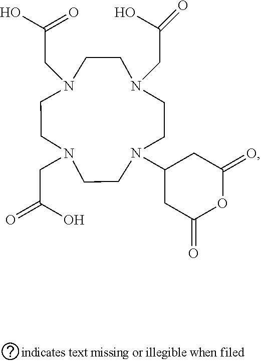 [SMILES: O=C(O)CN1CCN(CC(=O)O)CCN(C2CC(=O)OC(=O)C2)CCN(CC(=O)O)CC1]

each optionally bound to an isotope (or metal) suitable for radio-imaging, radiotherapy or magnetic resonance imaging.

In certain embodiments, B′ is selected from

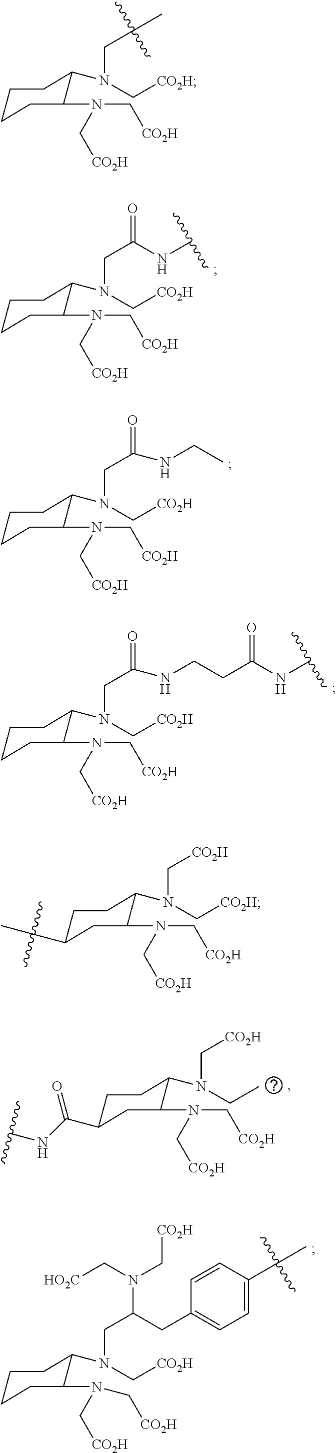

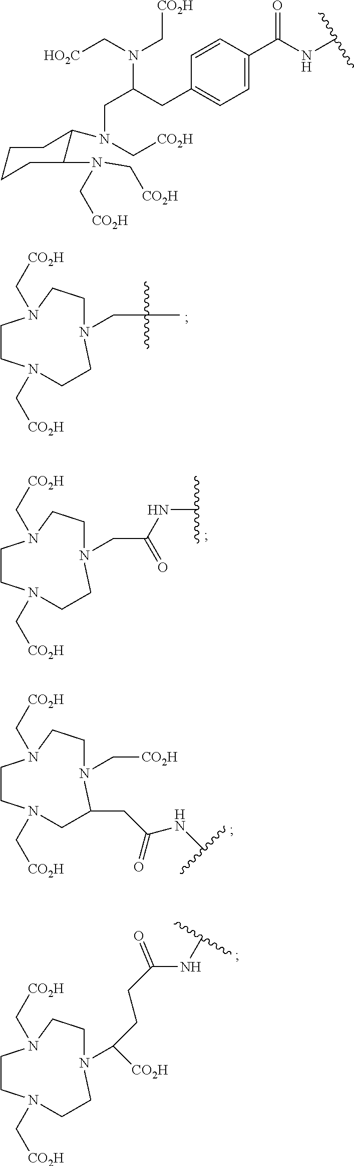 [SMILES: *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *NC(=O)CN1CCN(CC(=O)O)CCN(CC(=O)O)CC1 ; *NC(=O)CC1CN(CC(=O)O)CCN(CC(=O)O)CCN1CC(=O)O ; *NC(=O)CCC(C(=O)O)N1CCN(CC(=O)O)CCN(CC(=O)O)CC1]

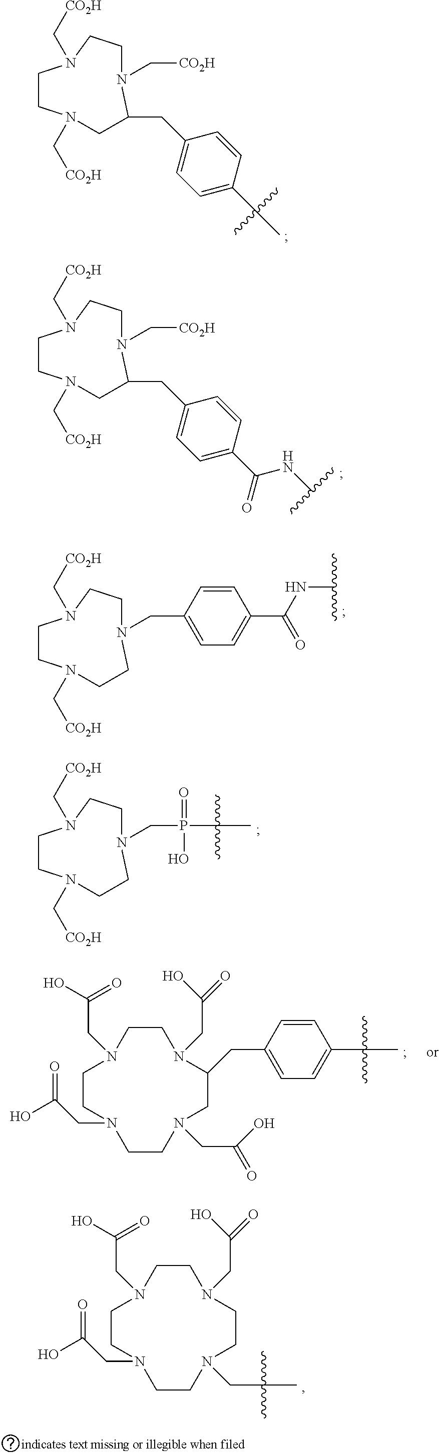

each optionally bound to an isotope (or metal) suitable for radio-imaging, radiotherapy or magnetic resonance imaging.

B′ can, for example, be a radical of a chelating group bound to an isotope suitable for PET imaging, SPECT imaging, other radio-imaging techniques, magnetic resonance imaging, or radiotherapy. B′ can, in certain embodiments, comprise a radical of DOTA. B′ can comprise a radical of an isotope chelated (or metal-chelated) DOTA. B′ can comprise a radical of 1,4,7,10-tetraazacyclododecane-1,4,7,10-tetraacetic acid. B′ can be a radical of a group covalently bound to an isotope (or metal) suitable for radio-imaging, radiotherapy or magnetic resonance imaging. B′ can comprise a group selected from

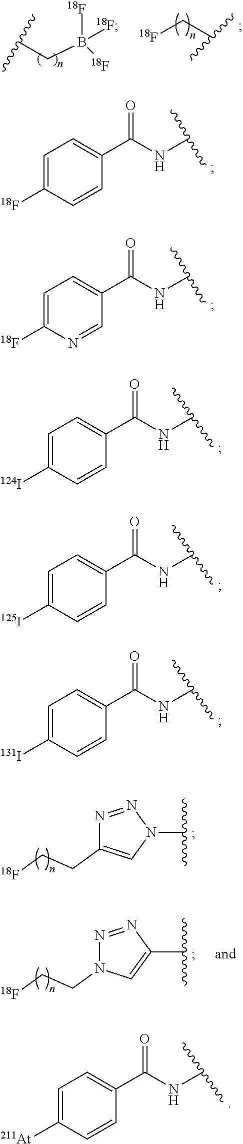

B′ can comprise

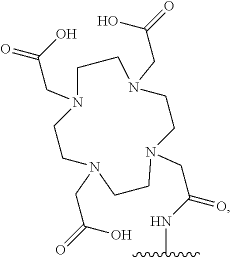 [SMILES: *NC(=O)CN1CCN(CC(=O)O)CCN(CC(=O)O)CCN(CC(=O)O)CC1]

optionally bound to an isotope (or metal) suitable for radio-imaging, radiotherapy or magnetic resonance imaging. B′ can comprise a radical of a radio-imaging, radiotherapeutic, or magnetic resonance isotope. In certain embodiments, B′ comprises a chelating group, and a radio-imaging, radiotherapeutic, or magnetic resonance isotope (or metal) which is bound to the chelating group.

In certain embodiments, B′ comprises a radio-imaging, radiotherapeutic, or magnetic resonance isotope or a chelating group and a radical of a radio-imaging, radiotherapeutic, or magnetic resonance isotope that is an isotope (e.g., a metal) bound to the chelating group, wherein the isotope is selected from ¹⁸F, ³²P, ⁴⁴Sc, ⁴⁷Sc, ⁵²Mn, ⁵⁵Co, ⁶⁴Cu, ⁶⁷Cu, ⁶⁷Ga, ⁶⁸Ga, ⁸⁶Y, ⁸⁹Sr, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁶⁹Er, ¹⁷⁷Lu, ¹⁸⁶Re, ¹⁸⁸Re, ²¹¹At, ²¹²Pb, ²¹²Bi, ²¹³Bi, ²²³Ra, ²²⁴Ra, ²²⁵Ab, ²²⁵Ac, or ²²⁷Th. The isotope of B′ can be ¹¹¹In. The isotope of B′ can be ¹⁷⁷Lu. In certain embodiments, the isotope is selected from the group consisting of: ¹¹C, ¹³C, ¹³N, ¹⁵O, ⁶⁰Co, and ¹²³I.

A of the compounds can have a binding affinity to FAPα from about 1 nM to about 25 nM. In certain embodiments, A has a binding affinity to FAP from about 1 nM to about 25 nM.

L of the compound can comprise a non-releasable linker. L can comprise a releasable linker. In certain embodiments, L comprises a PEGₙ, and n=0-36. L can comprise a peptide. L can comprise a peptidoglycan. In certain embodiments, L comprises:

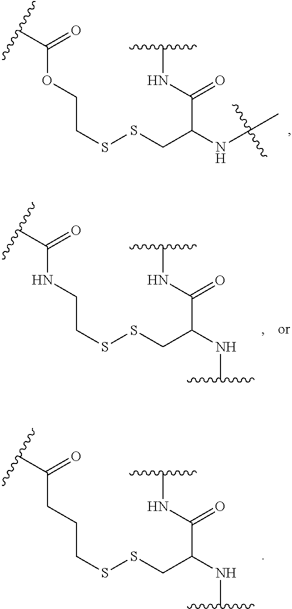 [SMILES: *NC(=O)C(CSSCCOC(*)=O)N* ; *NC(=O)C(CSSCCNC(*)=O)N* ; *NC(=O)C(CSSCCCC(*)=O)N*]

L can comprise:

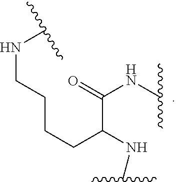 [SMILES: *NCCCCC(N*)C(=O)N*]

C′ can be a radical of an albumin binding ligand and have the following structure:

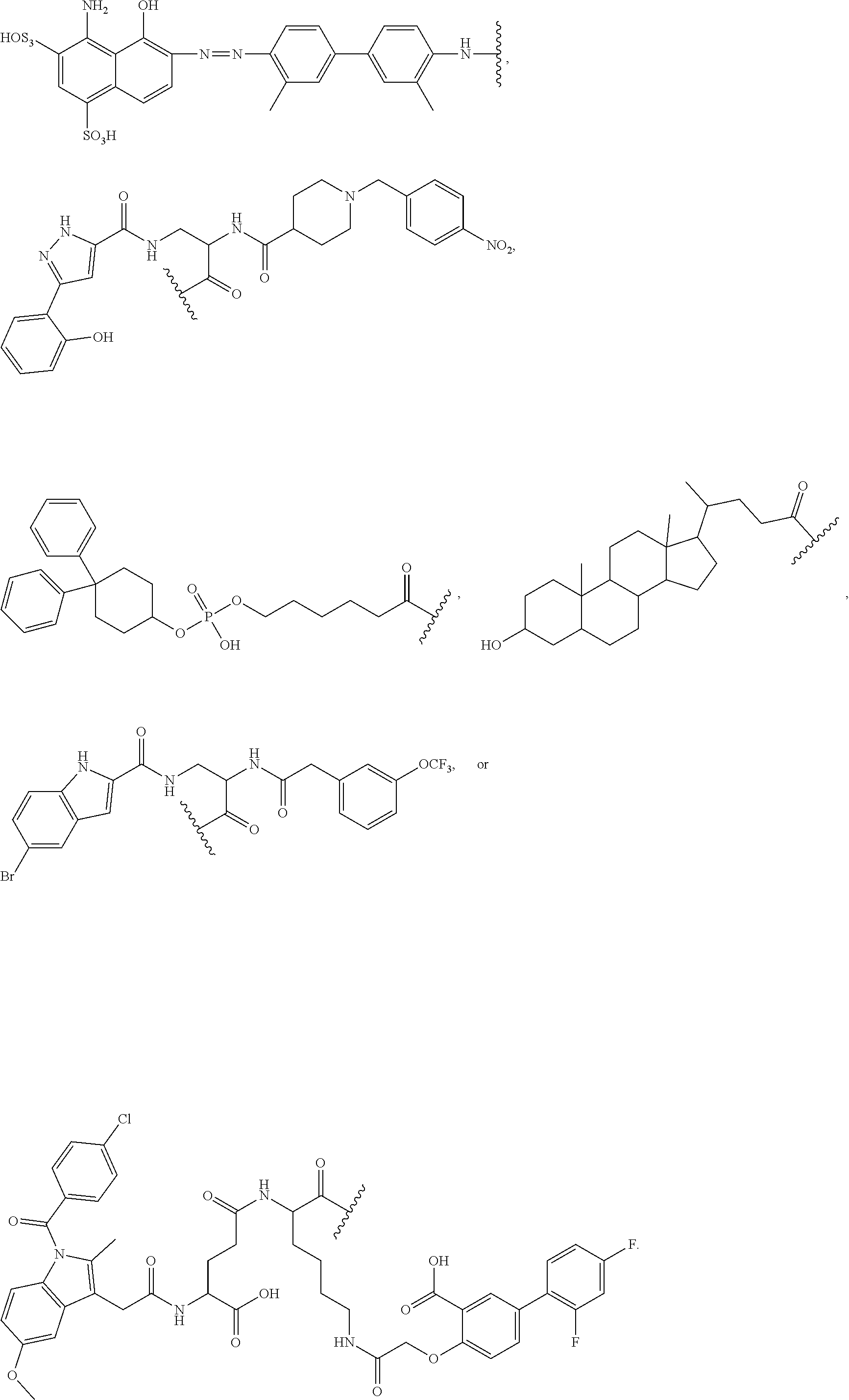

In certain embodiments, C′ is a radical of an albumin binding ligand and has the following structure:

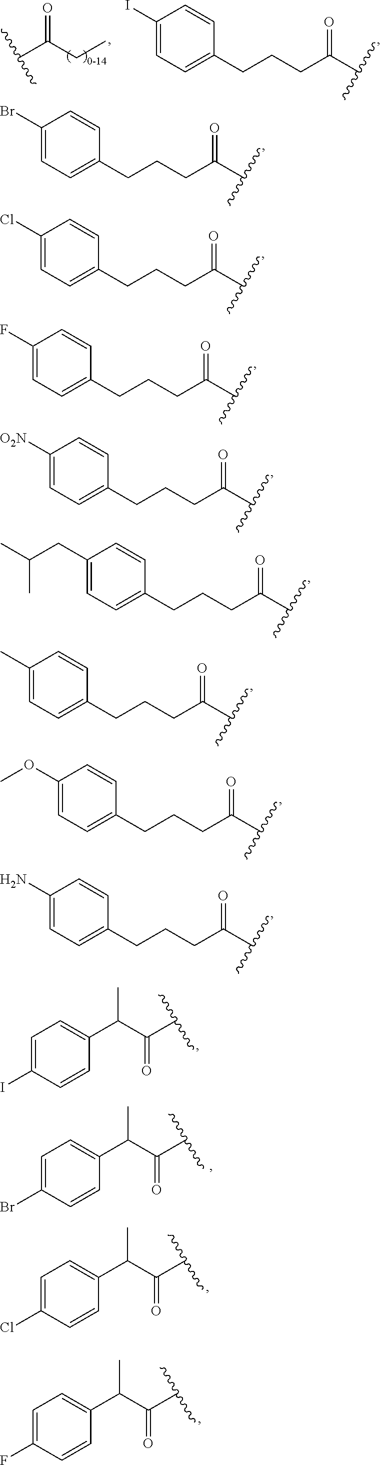

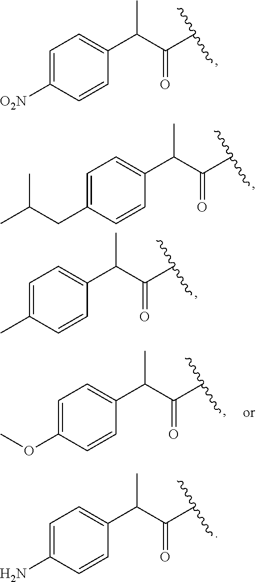

C′ can be, for example, a radical of one of the following:

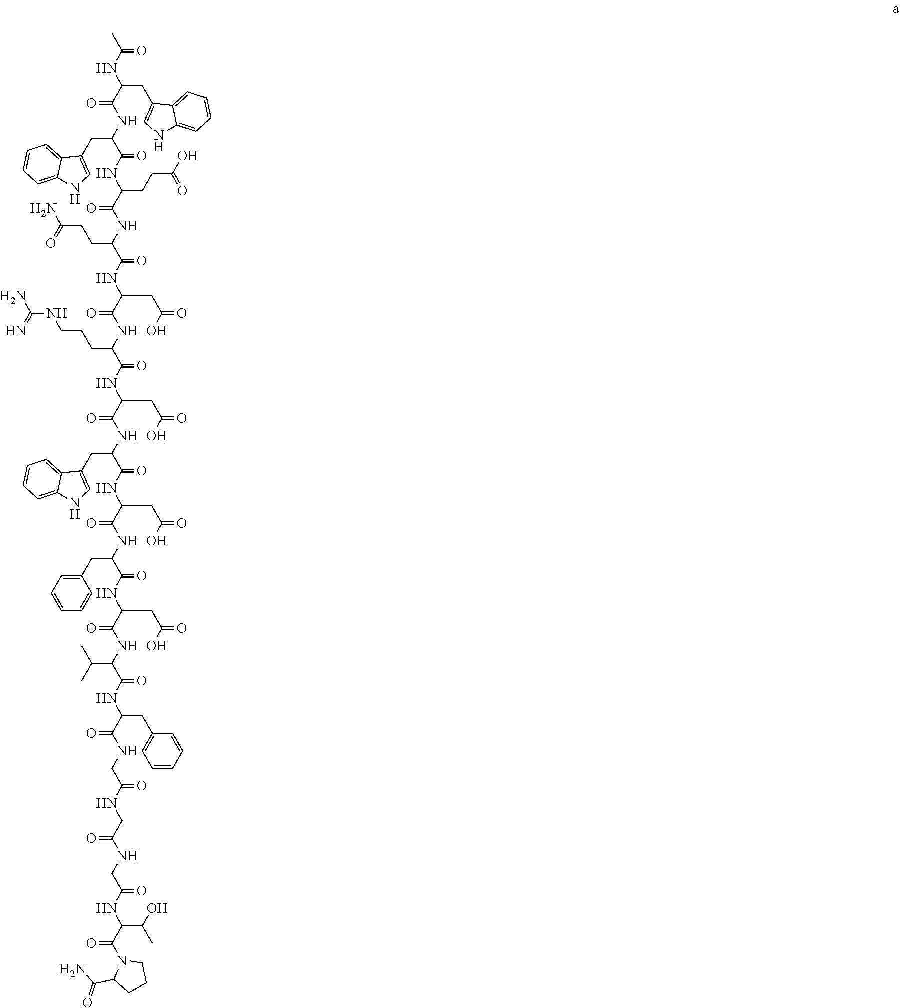

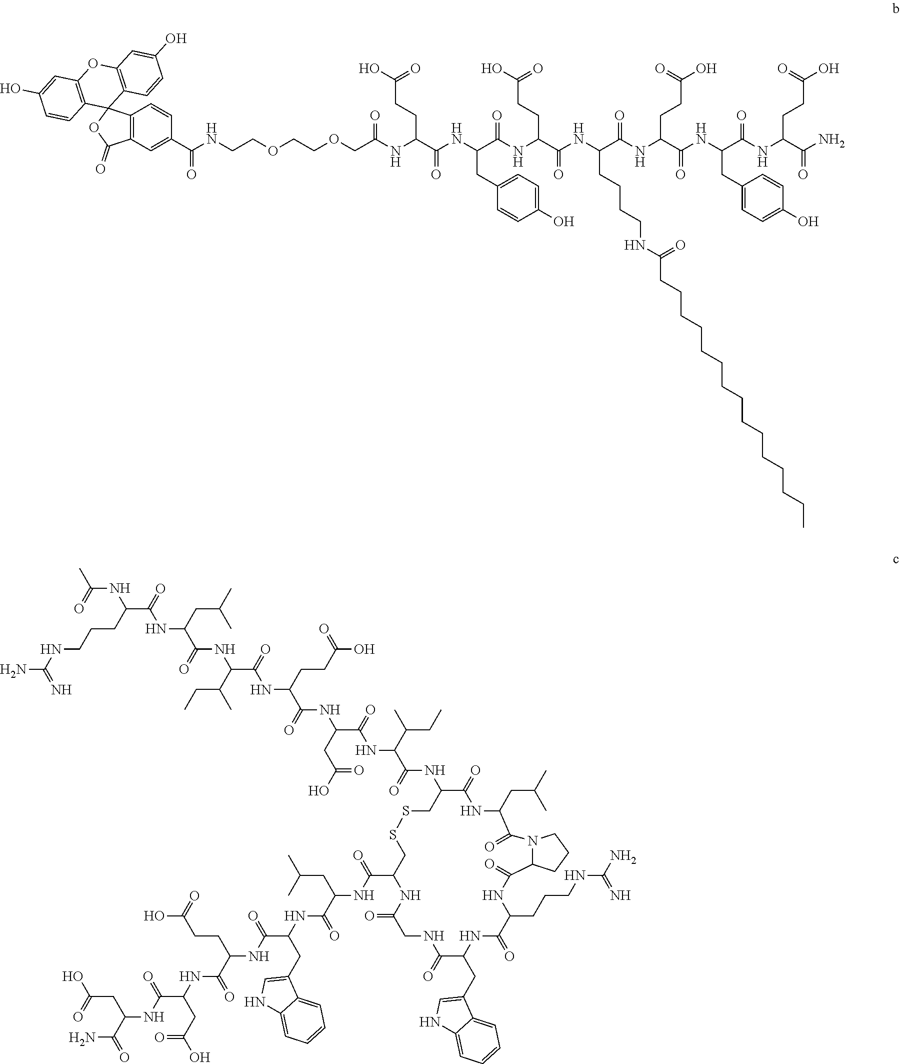

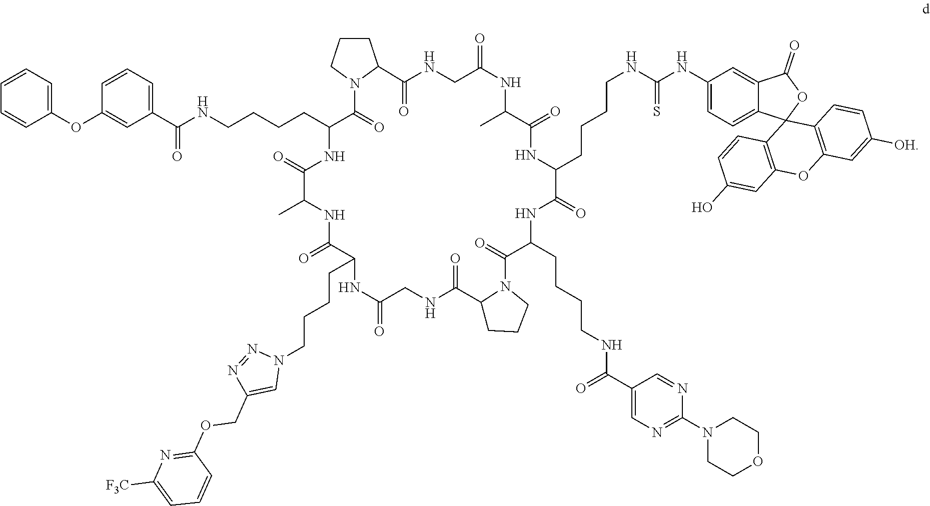

C′ can be, in some embodiments, a radical of an albumin-binding small protein scaffold comprising ABD035, ABDCon, DARPins, dsFv CA645, nanobody, and VNAR (E06). C′ can be PEGₙ, where n is an integer from 0 to 32. C′ can be a peptide. C′ can be a peptidoglycan. C′ can be a saccharide.

In certain embodiments, C′ is

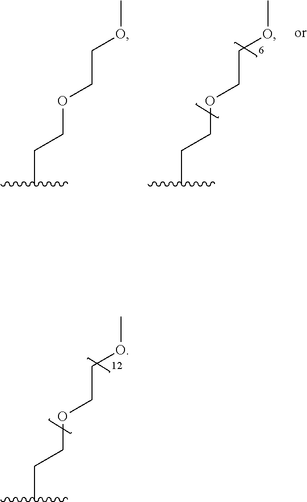

C′ can be

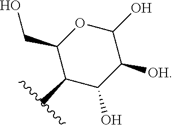 [SMILES: *[C@@H]1[C@@H](O)[C@H](O)C(O)O[C@@H]1CO]

In certain embodiments, the compounds can be represented by the structure of formula (V):

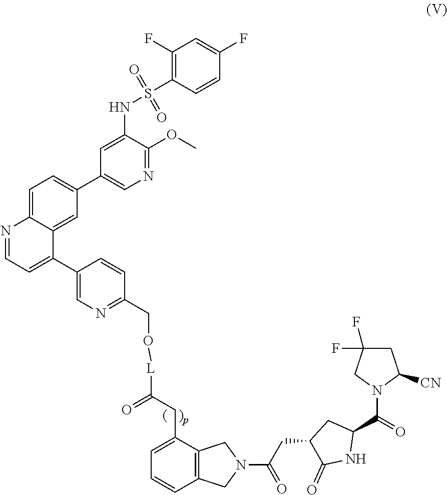

wherein:
L is a linker comprising at least one carbon atom; and
p is 0, 1, 2, or 3.

However, in some embodiments, the compound is not

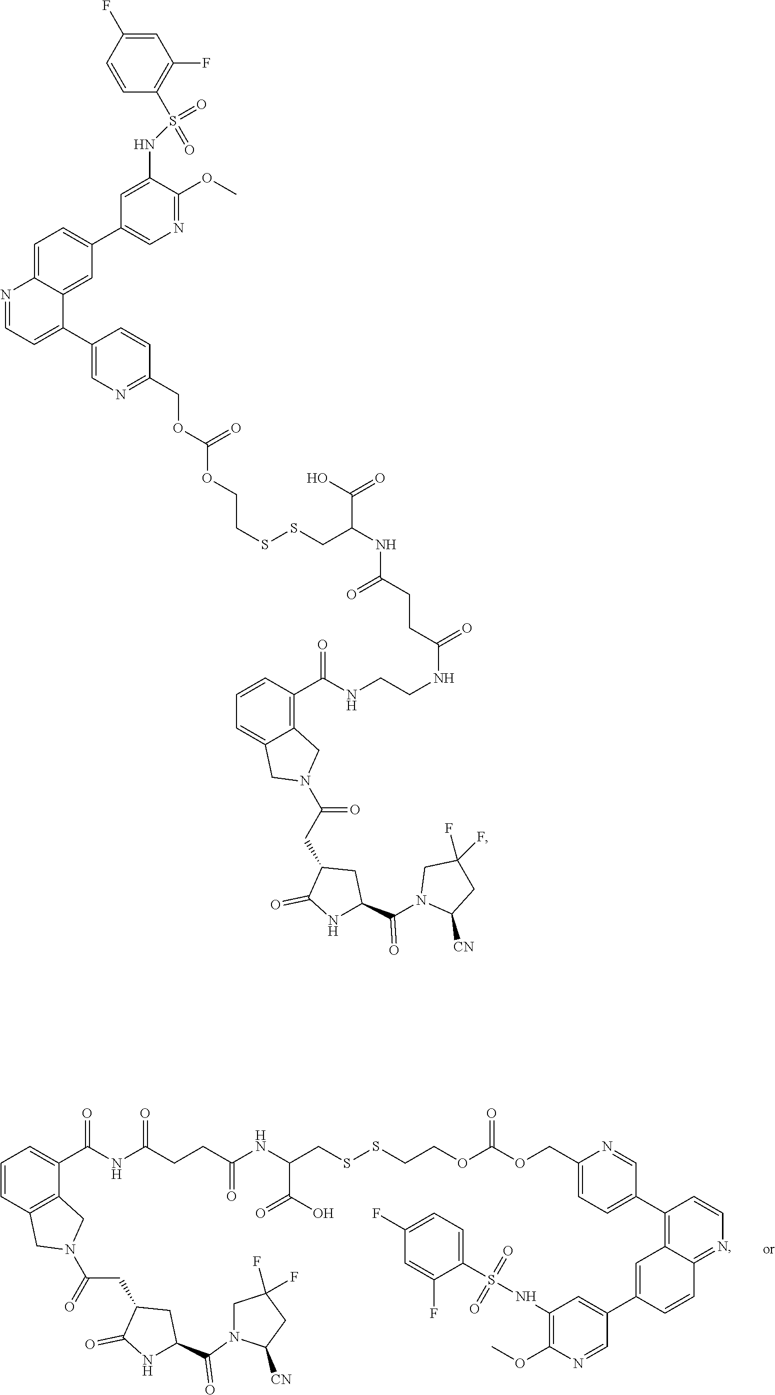 [SMILES: COc1ncc(-c2ccc3nccc(-c4ccc(COC(=O)OCCSSCC(NC(=O)CCC(=O)NCCNC(=O)c5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F ; COc1ncc(-c2ccc3nccc(-c4ccc(COC(=O)OCCSSCC(NC(=O)CCC(=O)NC(=O)c5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F]

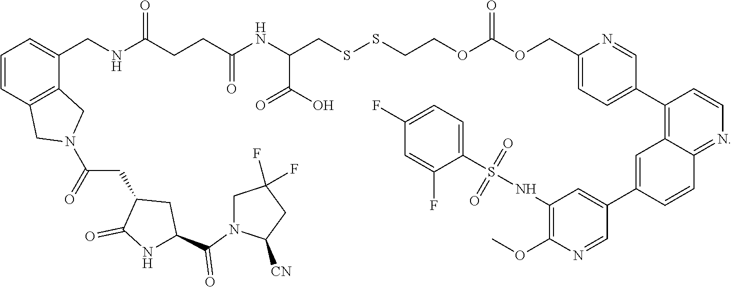 [SMILES: COc1ncc(-c2ccc3nccc(-c4ccc(COC(=O)OCCSSCC(NC(=O)CCC(=O)NCc5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F]

p can be 2. p can be 0.

Additionally, L can comprise:

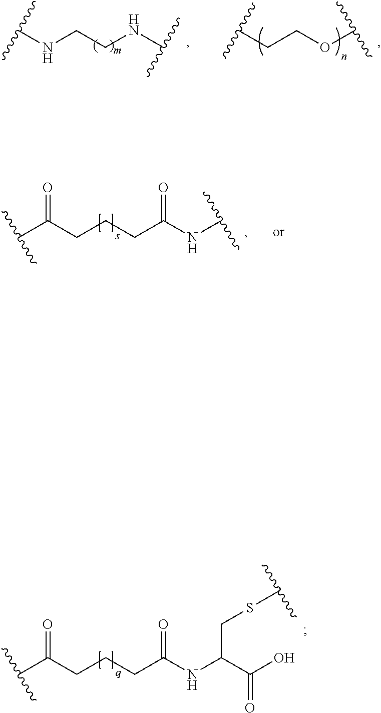

wherein
m is an integer from 1 to 9;
n is an integer from 1 to 32;
q is an integer from 0 to 4; and
s is an integer from 0 to 4.

In certain embodiments, the compound is:

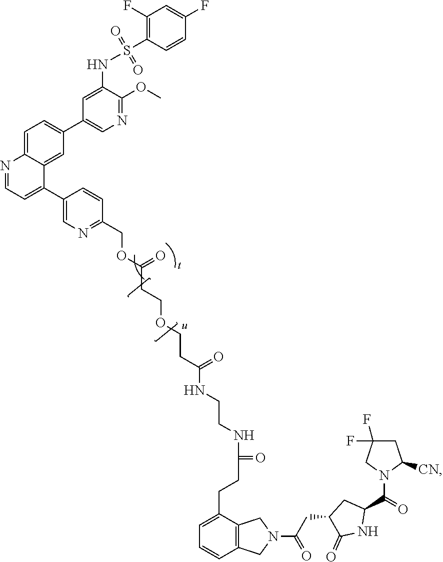

wherein t is 0 or 1 and u is an integer from 2-12.

In some embodiments, the compound is:

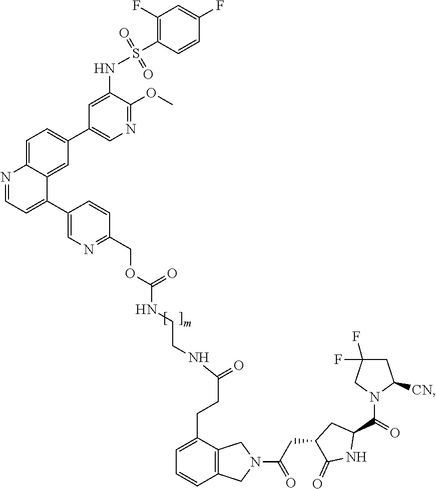

wherein m is an integer from 1 to 4.

In some embodiments, the compound is of the formula:

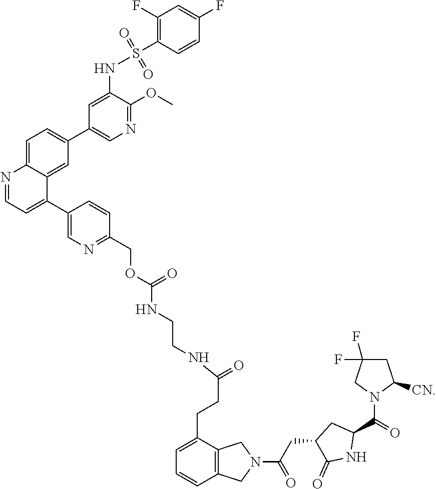 [SMILES: COc1ncc(-c2ccc3nccc(-c4ccc(COC(=O)NCCNC(=O)CCc5cccc6c5CN(C(=O)C[C@@H]5C[C@@H](C(=O)N7CC(F)(F)C[C@H]7C#N)NC5=O)C6)nc4)c3c2)cc1NS(=O)(=O)c1ccc(F)cc1F]

In some embodiments, the compound is of the formula:

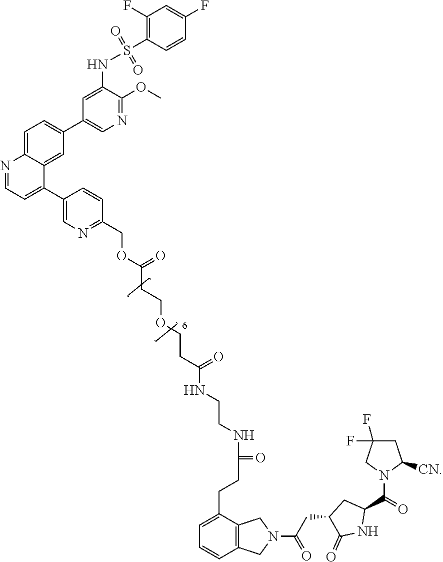

In some embodiments, the compound is of the formula:

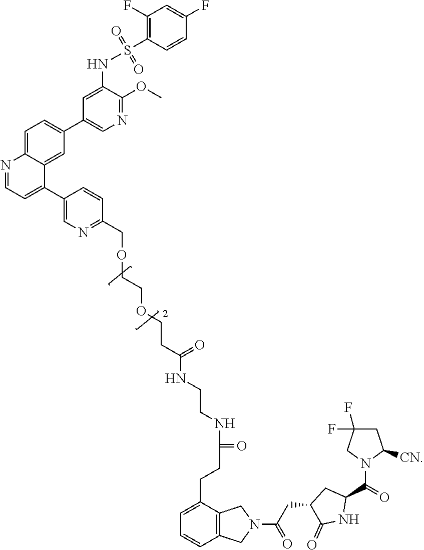

In some embodiments, the compound is of the formula:

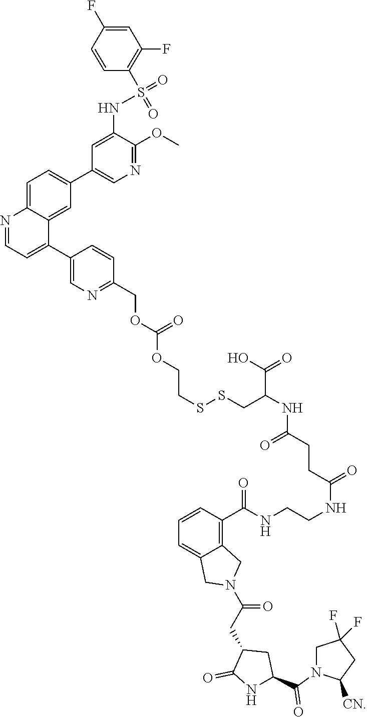 [SMILES: COc1ncc(-c2ccc3nccc(-c4ccc(COC(=O)OCCSSCC(NC(=O)CCC(=O)NCCNC(=O)c5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F]

In some embodiments, the compound is of the formula:

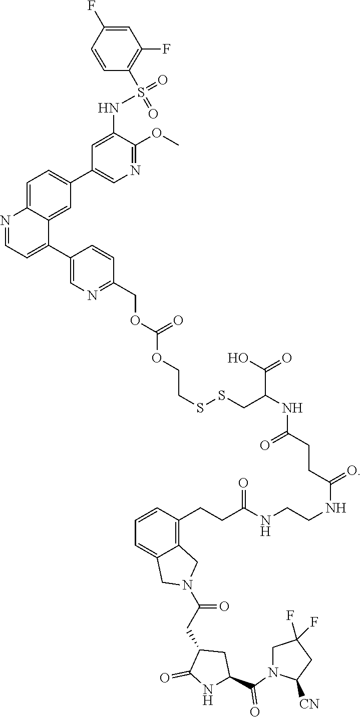 [SMILES: COc1ncc(-c2ccc3nccc(-c4ccc(COC(=O)OCCSSCC(NC(=O)CCC(=O)NCCNC(=O)CCc5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F]

In some embodiments, the compound is of the following structure:

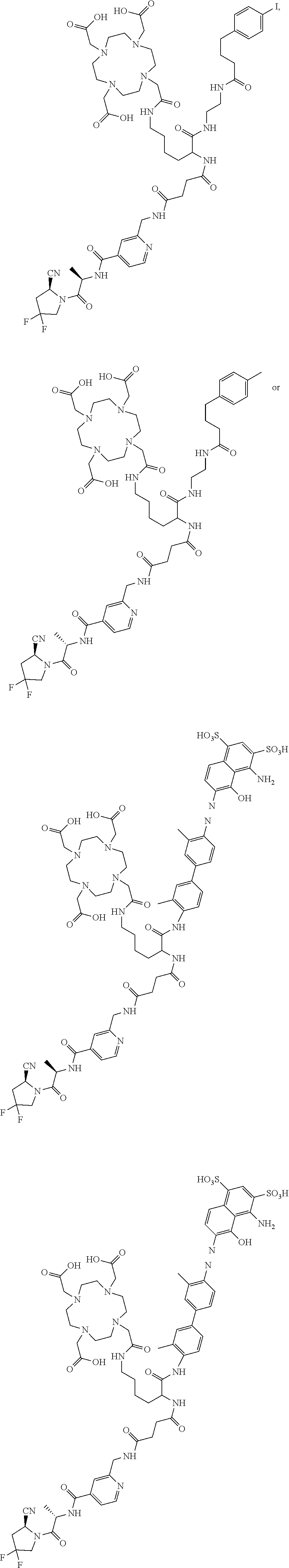

each optionally bound to an isotope (or metal) suitable for radio-imaging, radiotherapy or magnetic resonance imaging.

In some embodiments, the compound is of the following structure:

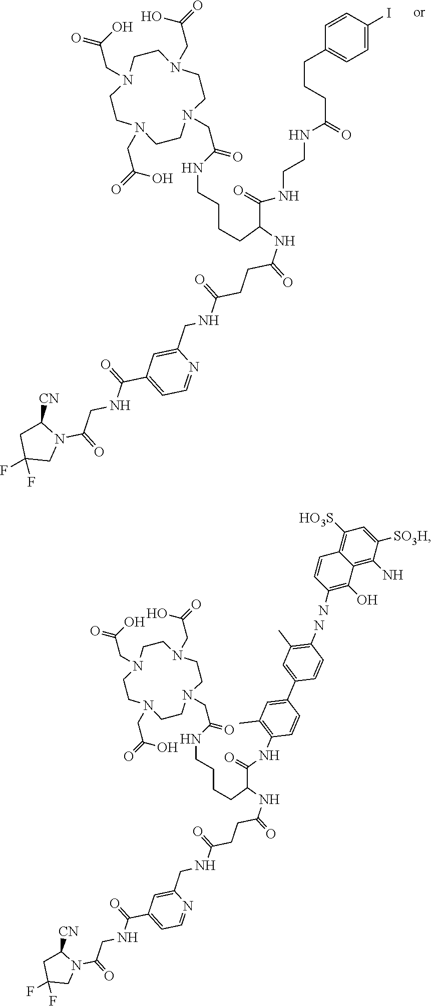

each optionally bound to an isotope (or metal) suitable for radio-imaging, radiotherapy or magnetic resonance imaging.

In some embodiments, the compound is of the following structure:

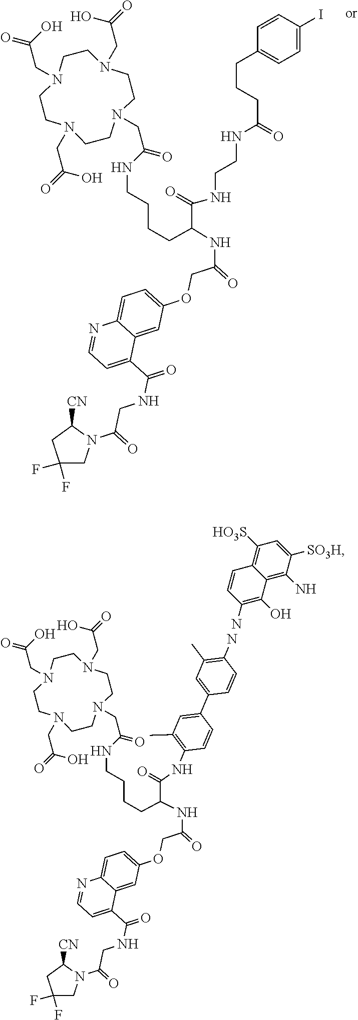

each optionally bound to an isotope (or metal) suitable for radio-imaging, radiotherapy, or magnetic resonance imaging.

In some embodiments, the compound is of the following structure:

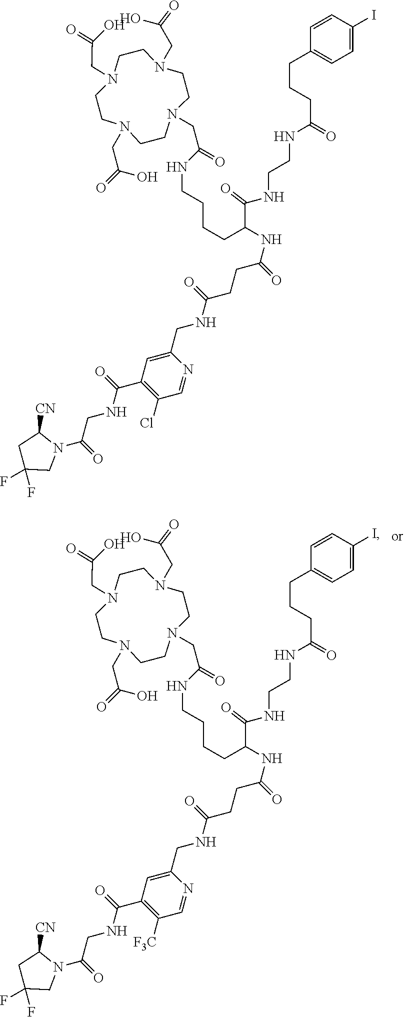 [SMILES: N#C[C@@H]1CC(F)(F)CN1C(=O)CNC(=O)c1cc(CNC(=O)CCC(=O)NC(CCCCNC(=O)CN2CCN(CC(=O)O)CCN(CC(=O)O)CCN(CC(=O)O)CC2)C(=O)NCCNC(=O)CCCc2ccc(I)cc2)ncc1Cl ; N#C[C@@H]1CC(F)(F)CN1C(=O)CNC(=O)c1cc(CNC(=O)CCC(=O)NC(CCCCNC(=O)CN2CCN(CC(=O)O)CCN(CC(=O)O)CCN(CC(=O)O)CC2)C(=O)NCCNC(=O)CCCc2ccc(I)cc2)ncc1C(F)(F)F]

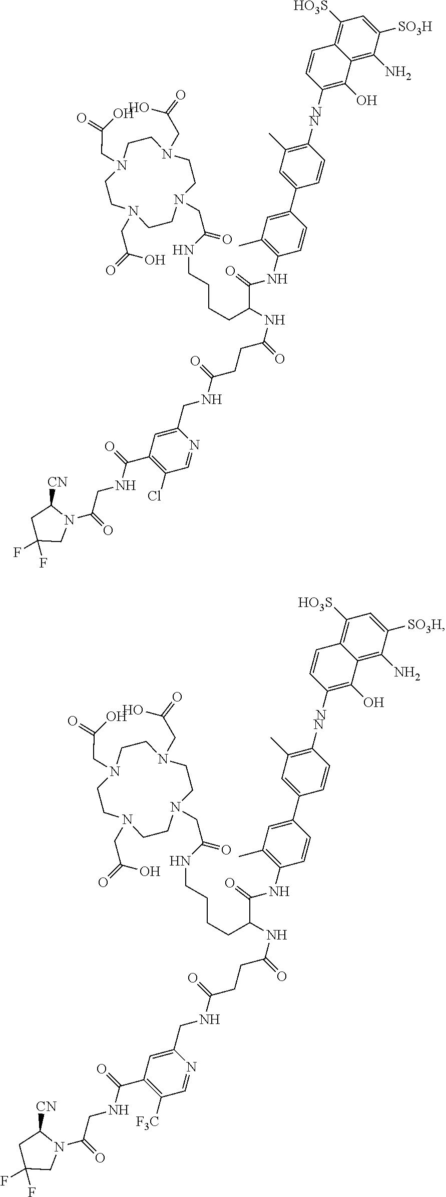

each optionally bound to an isotope (or metal) suitable for radio-imaging, radiotherapy, or magnetic resonance imaging.

In some embodiments, the compound is of the following structure:

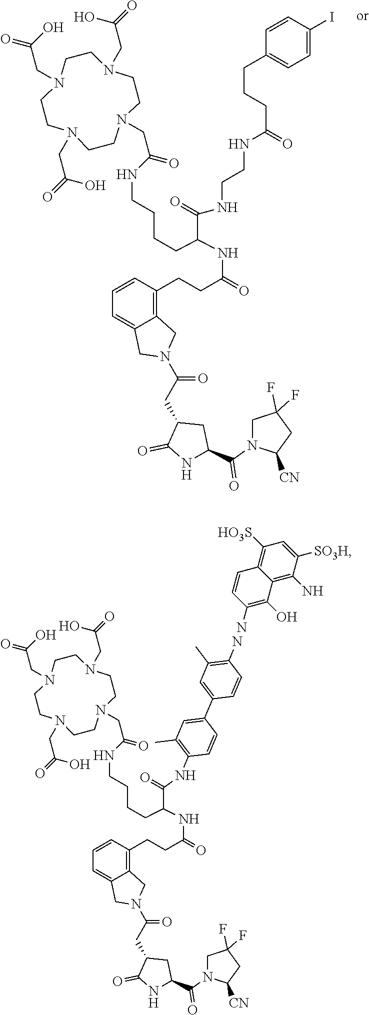

each optionally bound to an isotope (or metal) suitable for radio-imaging, radiotherapy, or magnetic resonance imaging.

In certain embodiments, the compound is of the following structure:

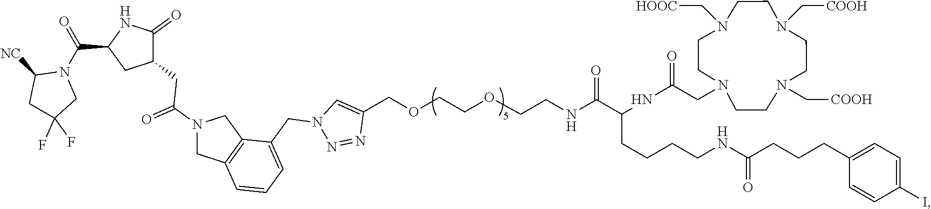

optionally bound to an isotope (or metal) suitable for radio-imaging, radiotherapy or magnetic resonance imaging.

The compound can, in certain embodiments, have the following structure:

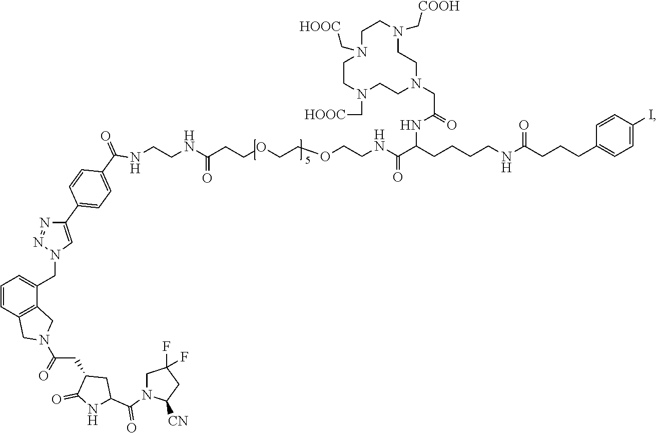

optionally bound to an isotope (or metal) suitable for radio-imaging, radiotherapy or magnetic resonance imaging.

In certain embodiments, the compound can have the following structure:

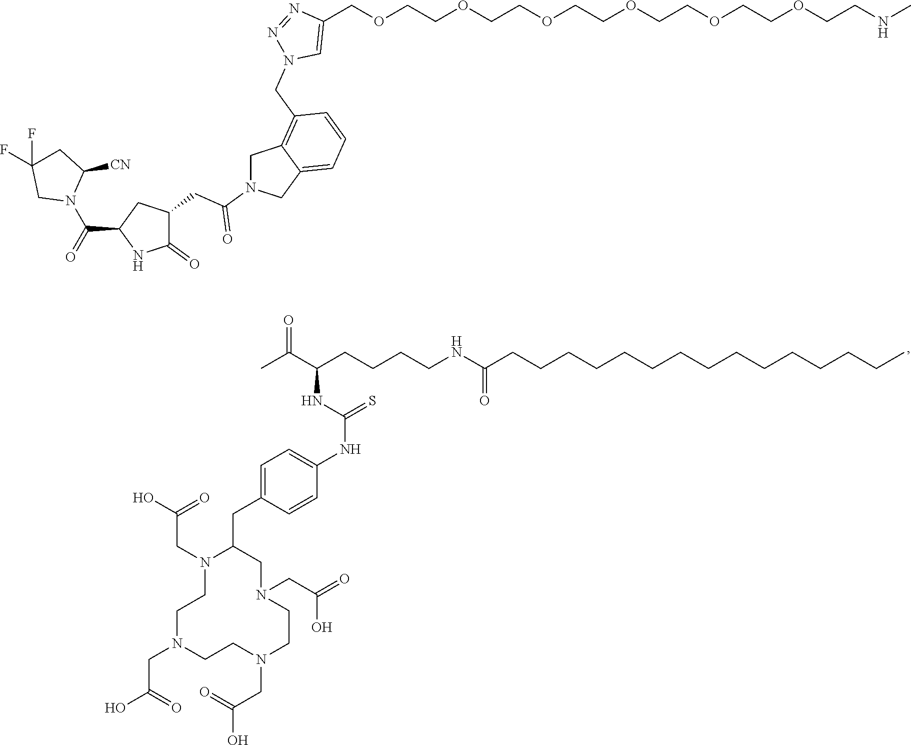 [SMILES: CNCCOCCOCCOCCOCCOCCOCc1cn(Cc2cccc3c2CN(C(=O)C[C@H]2C[C@H](C(=O)N4CC(F)(F)C[C@H]4C#N)NC2=O)C3)nn1 ; CCCCCCCCCCCCCCCC(=O)NCCCC[C@@H](NC(=S)Nc1ccc(CC2CN(CC(=O)O)CCN(CC(=O)O)CCN(CC(=O)O)CCN2CC(=O)O)cc1)C(C)=O]

optionally bound to an isotope (or metal) suitable for radio-imaging, radiotherapy or magnetic resonance imaging.

In certain embodiments, the compound can have the following structure:

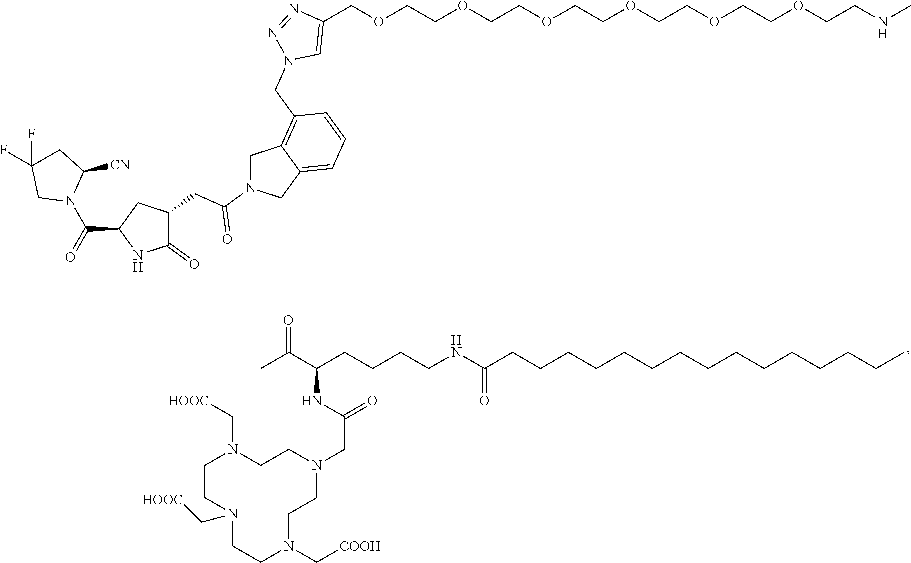 [SMILES: CNCCOCCOCCOCCOCCOCCOCc1cn(Cc2cccc3c2CN(C(=O)C[C@H]2C[C@H](C(=O)N4CC(F)(F)C[C@H]4C#N)NC2=O)C3)nn1 ; CCCCCCCCCCCCCCCC(=O)NCCCC[C@@H](NC(=O)CN1CCN(CC(=O)O)CCN(CC(=O)O)CCN(CC(=O)O)CC1)C(C)=O]

optionally bound to an isotope suitable (or metal) for radio-imaging, radiotherapy or magnetic resonance imaging.

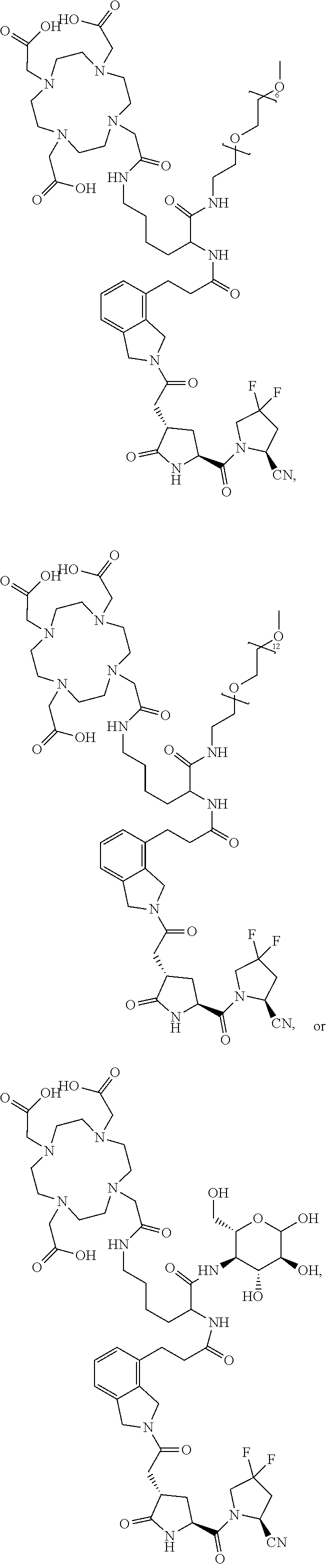

The compounds hereof can have the following structure:

each optionally bound to an isotope (or metal) suitable for radio-imaging, radiotherapy or magnetic resonance imaging.

In certain embodiments, the compounds hereof can have the following structure:

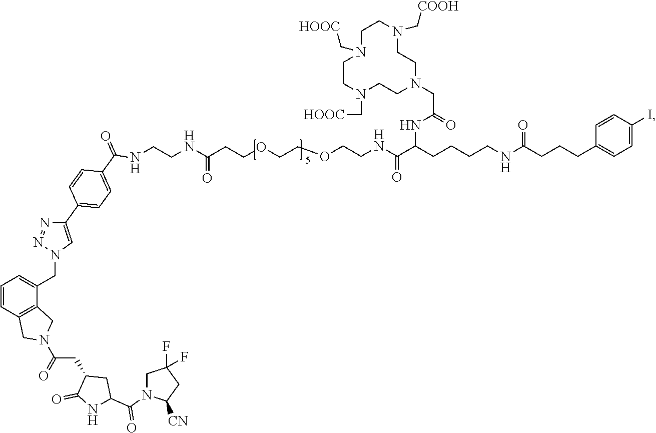

optionally bound to an isotope (or metal) suitable for radio-imaging, radiotherapy or magnetic resonance imaging.

The compounds hereof can have the following structure:

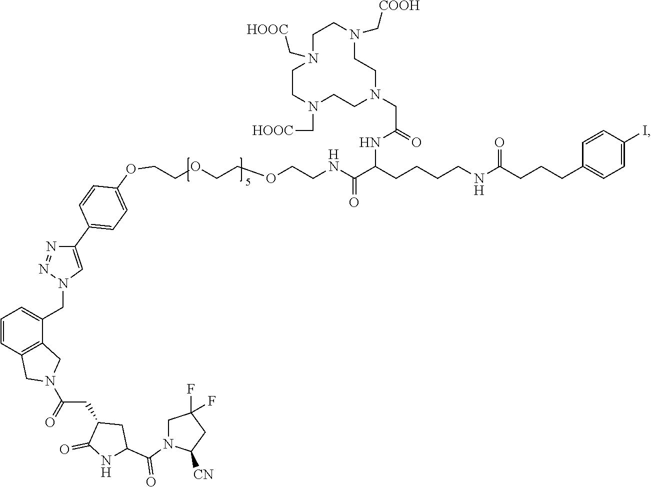

optionally bound to an isotope (or metal) suitable for radio-imaging, radiotherapy or magnetic resonance imaging

The compounds hereof can have the following structure:

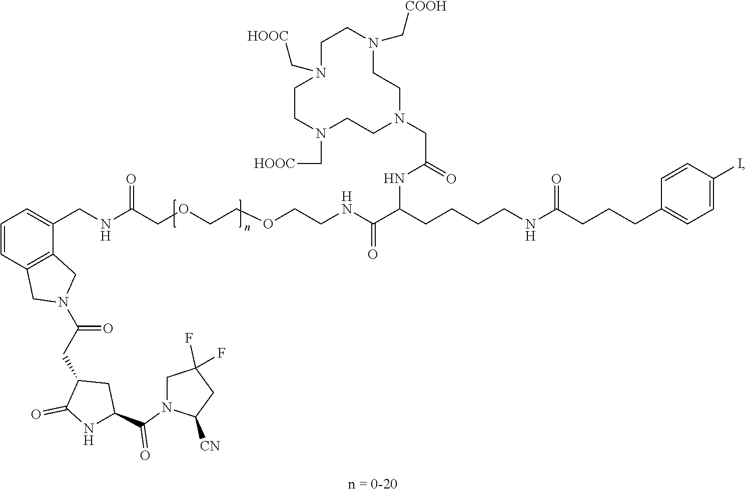

optionally bound to an isotope (or metal) suitable for radio-imaging, radiotherapy or magnetic resonance imaging.

The compound can have the following structure:

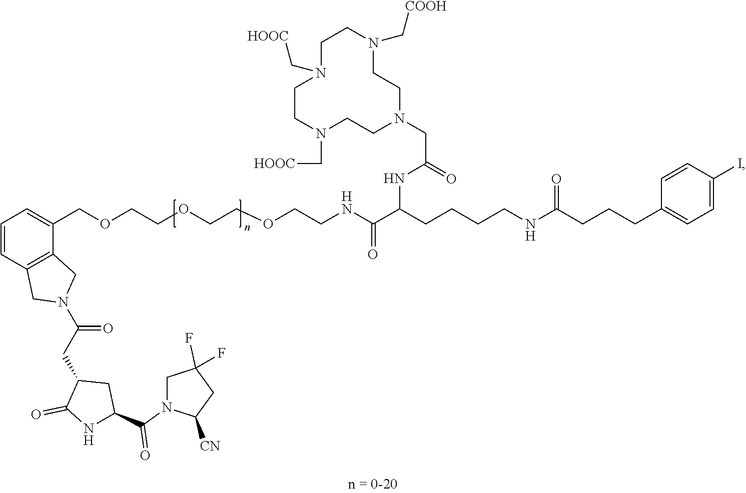

optionally bound to an isotope (or metal) suitable for radio-imaging, radiotherapy or magnetic resonance imaging

The compound can have the following structure:

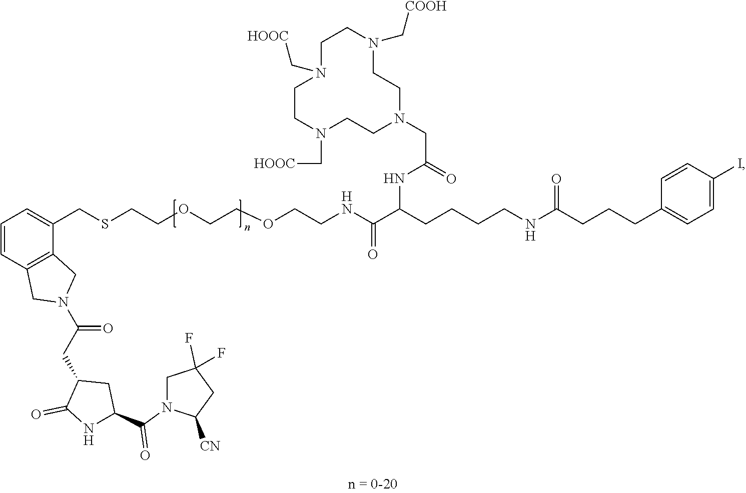

optionally bound to an isotope (or metal) suitable for radio-imaging, radiotherapy or magnetic resonance imaging.

The compound can have the following structure:

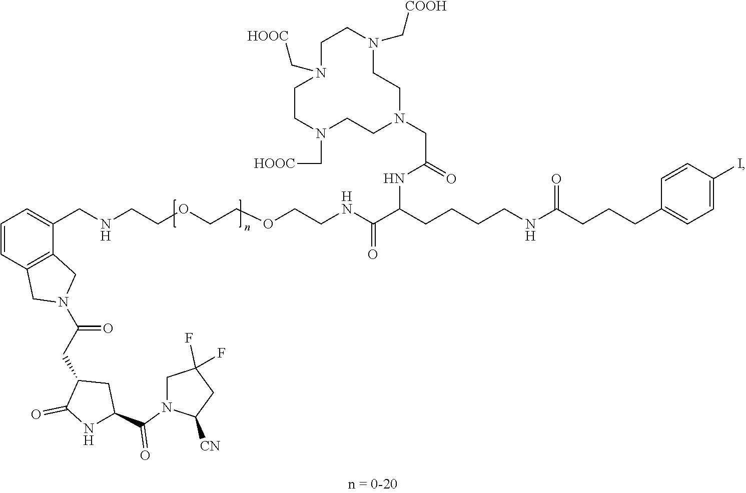

optionally bound to an isotope (or metal) suitable for radio-imaging, radiotherapy or magnetic resonance imaging.

The compound can have the following structure:

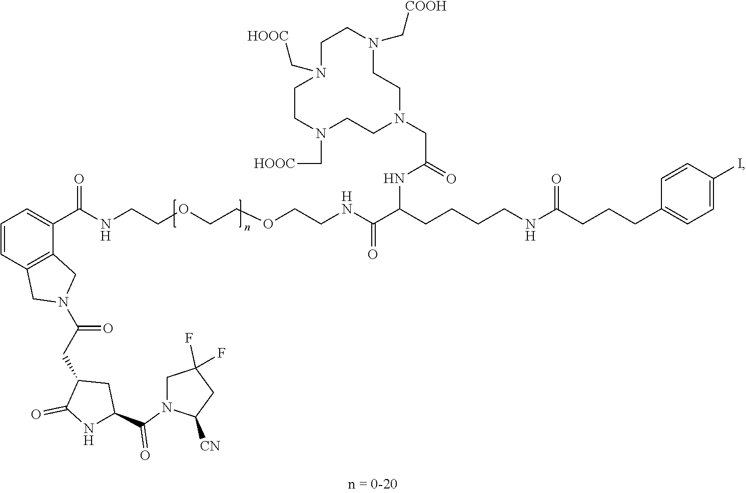

optionally bound to an isotope (or metal) suitable for radio-imaging, radiotherapy or magnetic resonance imaging.

The compound can have the following structure:

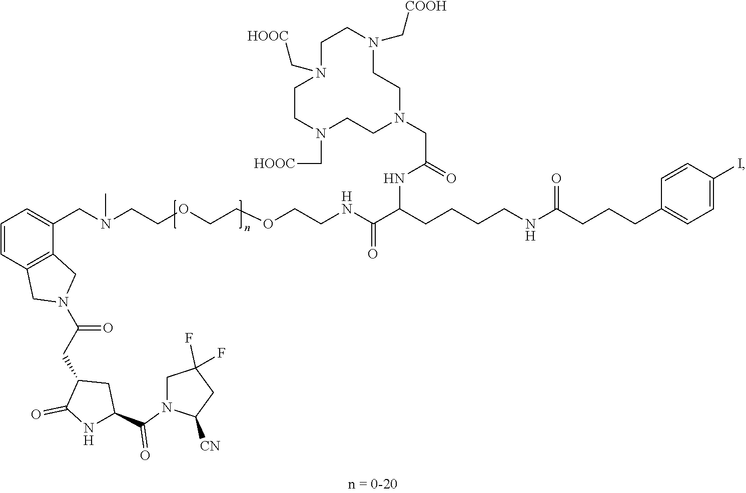

optionally bound to an isotope (or metal) suitable for radio-imaging, radiotherapy or magnetic resonance imaging.

The compound can have the following structure:

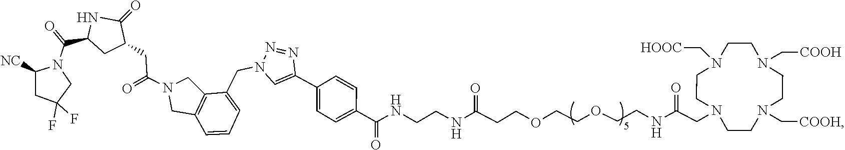

optionally bound to an isotope (or metal) suitable for radio-imaging, radiotherapy or magnetic resonance imaging.

The compound can have the following structure:

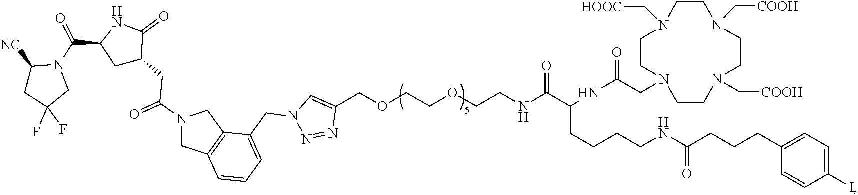

optionally bound to an isotope (or metal) suitable for radio-imaging, radiotherapy or magnetic resonance imaging.

The compound can have the following structure:

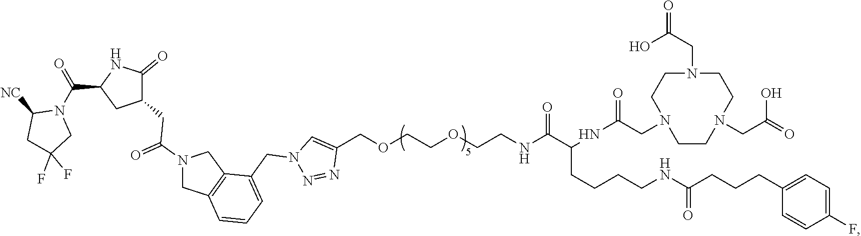

optionally bound to an isotope (or metal) suitable for radio-imaging, radiotherapy or magnetic resonance imaging.

The compound can have the following structure:

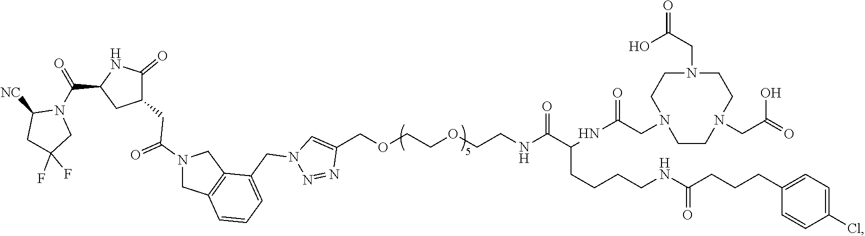

optionally bound to an isotope (or metal) suitable for radio-imaging, radiotherapy or magnetic resonance imaging.

The compound can have the following structure:

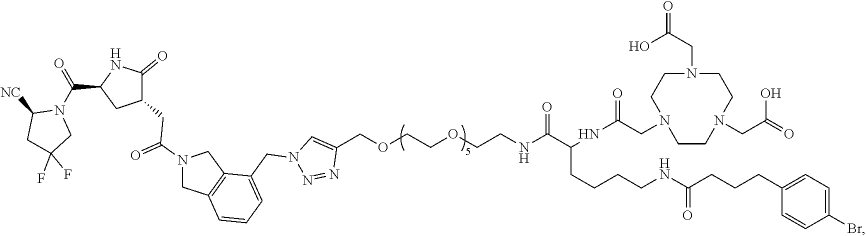

optionally bound to an isotope (or metal) suitable for radio-imaging, radiotherapy or magnetic resonance imaging.

The compound can have the following structure:

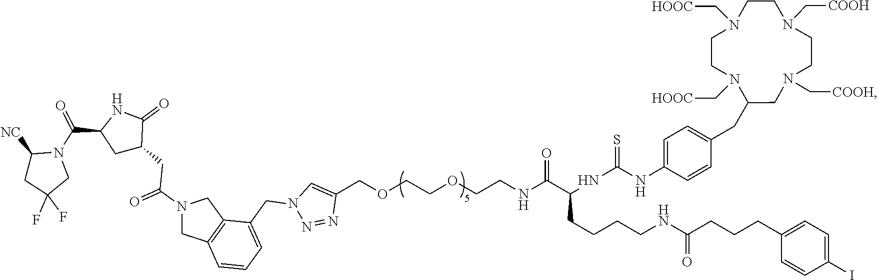

optionally bound to an isotope (or metal) suitable for radio-imaging, radiotherapy or magnetic resonance imaging.

The compound can have the following structure:

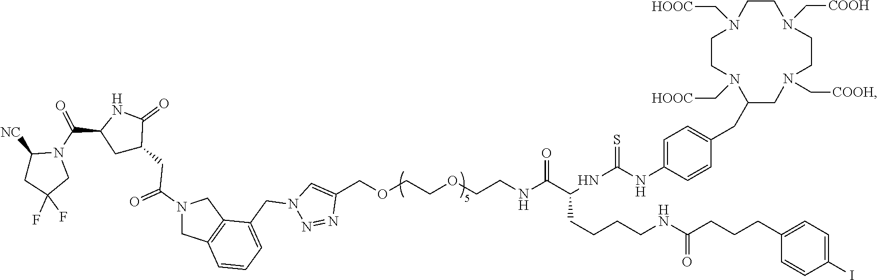

optionally bound to an isotope (or metal) suitable for radio-imaging, radiotherapy or magnetic resonance imaging.

The compound can have the following structure:

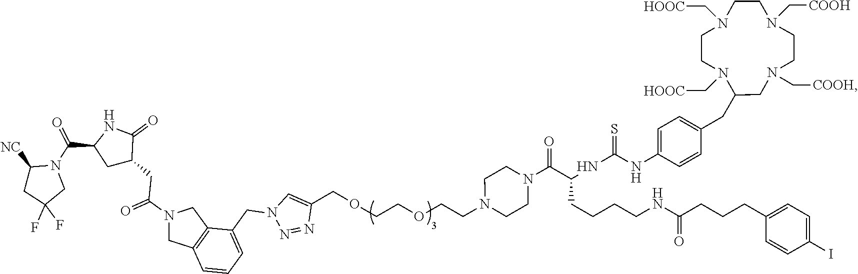

optionally bound to an isotope (or metal) suitable for radio-imaging, radiotherapy or magnetic resonance imaging.

The compound can have the following structure:

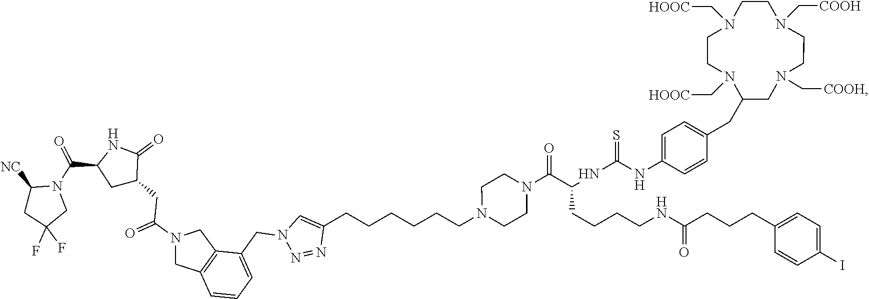 [SMILES: N#C[C@@H]1CC(F)(F)CN1C(=O)[C@@H]1C[C@@H](CC(=O)N2Cc3cccc(Cn4cc(CCCCCCN5CCN(C(=O)[C@@H](CCCCNC(=O)CCCc6ccc(I)cc6)NC(=S)Nc6ccc(CC7CN(CC(=O)O)CCN(CC(=O)O)CCN(CC(=O)O)CCN7CC(=O)O)cc6)CC5)nn4)c3C2)C(=O)N1]

optionally bound to an isotope (or metal) suitable for radio-imaging, radiotherapy or magnetic resonance imaging.

The compound can have the following structure:

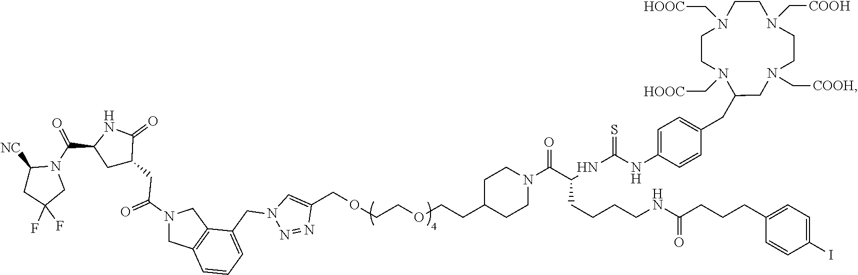

optionally bound to an isotope (or metal) suitable for radio-imaging, radiotherapy or magnetic resonance imaging.

The compound can have the following structure:

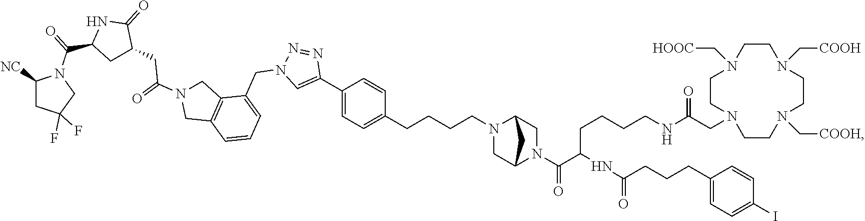 [SMILES: N#C[C@@H]1CC(F)(F)CN1C(=O)[C@@H]1C[C@@H](CC(=O)N2Cc3cccc(Cn4cc(-c5ccc(CCCCN6C[C@@H]7C[C@H]6CN7C(=O)C(CCCCNC(=O)CN6CCN(CC(=O)O)CCN(CC(=O)O)CCN(CC(=O)O)CC6)NC(=O)CCCc6ccc(I)cc6)cc5)nn4)c3C2)C(=O)N1]

optionally bound to an isotope (or metal) suitable for radio-imaging, radiotherapy or magnetic resonance imaging.

The compound can have the following structure:

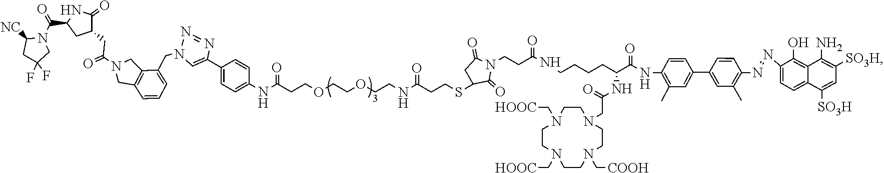

optionally bound to an isotope (or metal) suitable for radio-imaging, radiotherapy or magnetic resonance imaging.

The compound can have the following structure:

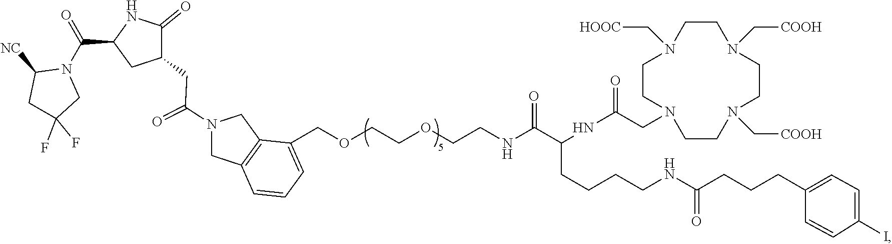

optionally bound to an isotope (or metal) suitable for radio-imaging, radiotherapy or magnetic resonance imaging.

The compound can have the following structure:

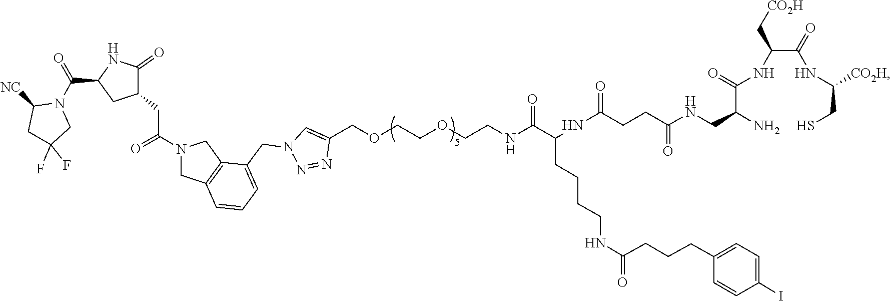

optionally bound to an isotope (or metal) suitable for radio-imaging, radiotherapy or magnetic resonance imaging.

The compound can have the following structure:

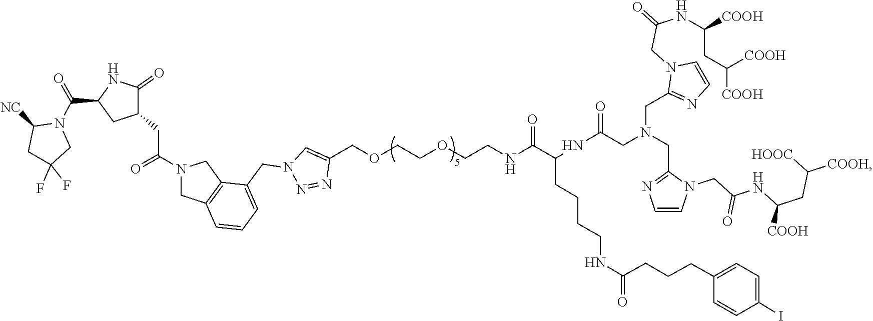

optionally bound to an isotope (or metal) suitable for radio-imaging, radiotherapy or magnetic resonance imaging.

The compound can have the following structure:

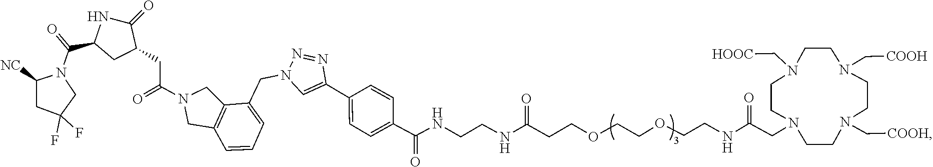

optionally bound to an isotope (or metal) suitable for radio-imaging, radiotherapy or magnetic resonance imaging.

The compound can have the following structure:

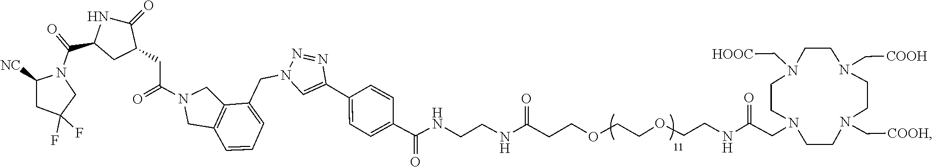

optionally bound to an isotope (or metal) suitable for radio-imaging, radiotherapy or magnetic resonance imaging.

The compound can have the following structure:

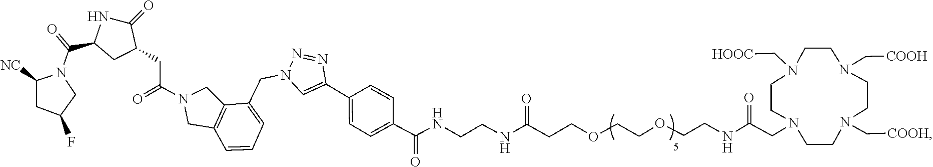

optionally bound to an isotope (or metal) suitable for radio-imaging, radiotherapy or magnetic resonance imaging.

The compound can have the following structure:

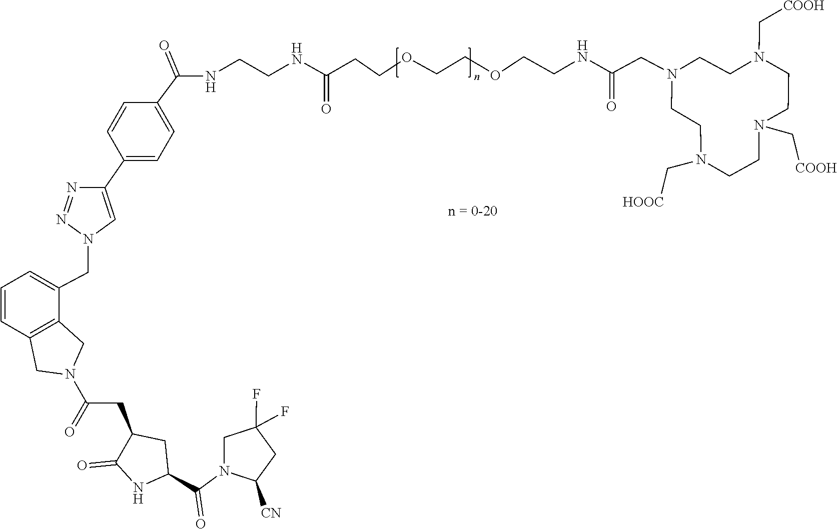

optionally bound to an isotope (or metal) suitable for radio-imaging, radiotherapy or magnetic resonance imaging.

The compound can have the following structure:

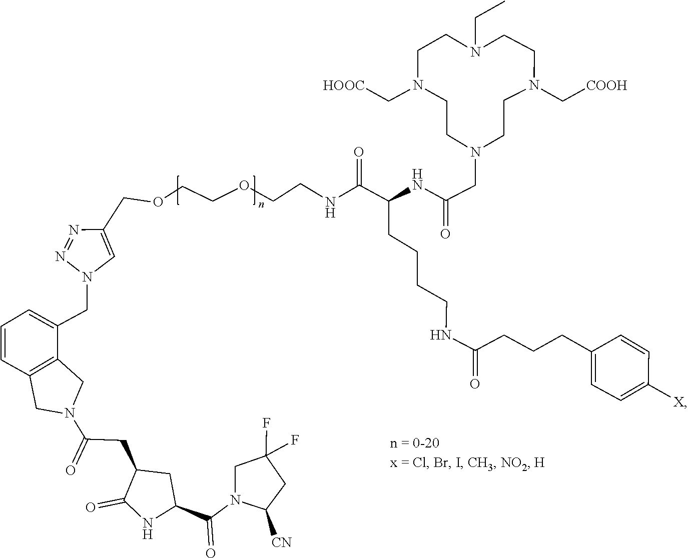

optionally bound to an isotope (or metal) suitable for radio-imaging, radiotherapy or magnetic resonance imaging.

The compound can have the following structure:

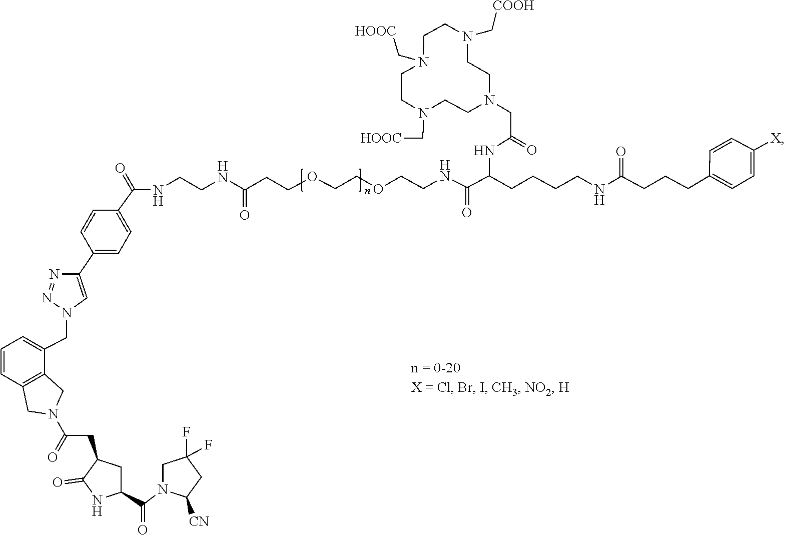

optionally bound to an isotope (or metal) suitable for radio-imaging, radiotherapy or magnetic resonance imaging.

The compound can have the following structure:

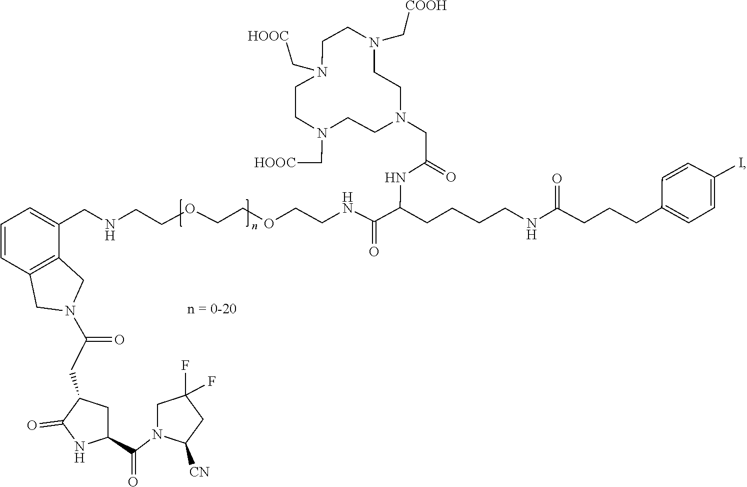

optionally bound to an isotope (or metal) suitable for radio-imaging, radiotherapy or magnetic resonance imaging.

The compound can have the following structure:

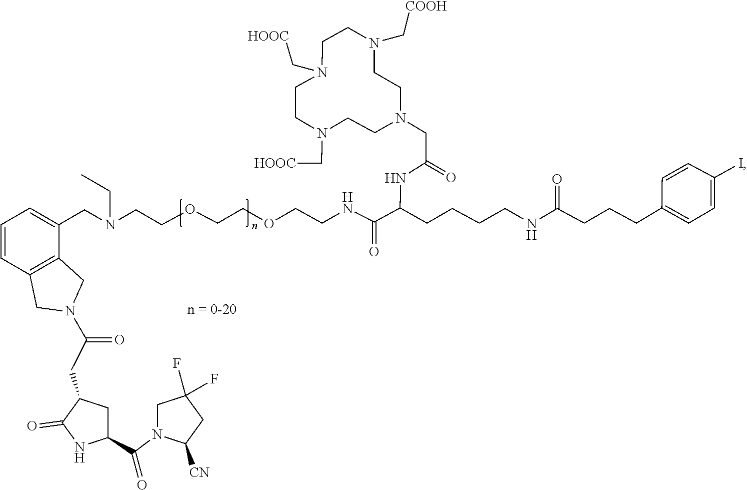

optionally bound to an isotope (or metal) suitable for radio-imaging, radiotherapy or magnetic resonance imaging.

The compound can have the following structure:

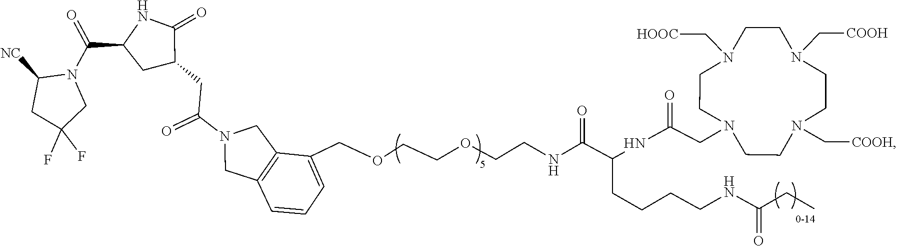

optionally bound to an isotope (or metal) suitable for radio-imaging, radiotherapy or magnetic resonance imaging.

The compound can have the following structure:

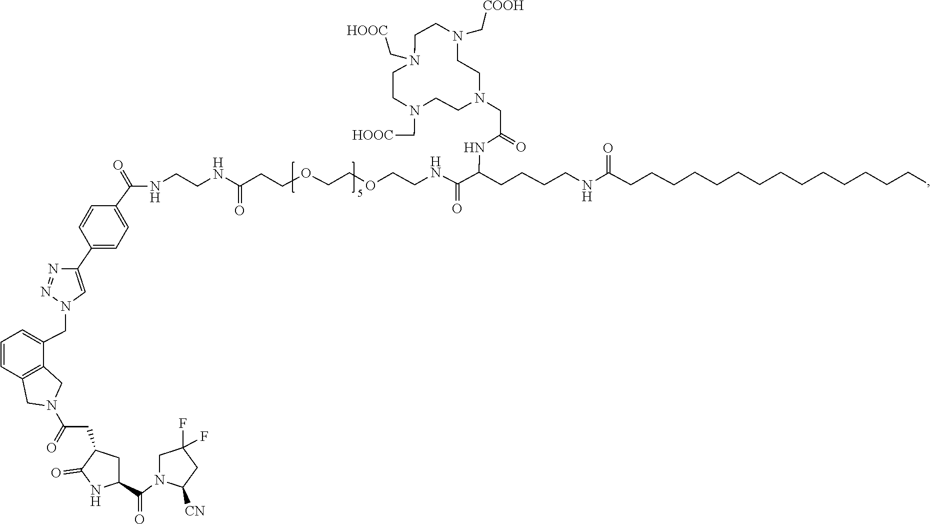

optionally bound to an isotope (or metal) suitable for radio-imaging, radiotherapy or magnetic resonance imaging.

The compound can have the following structure:

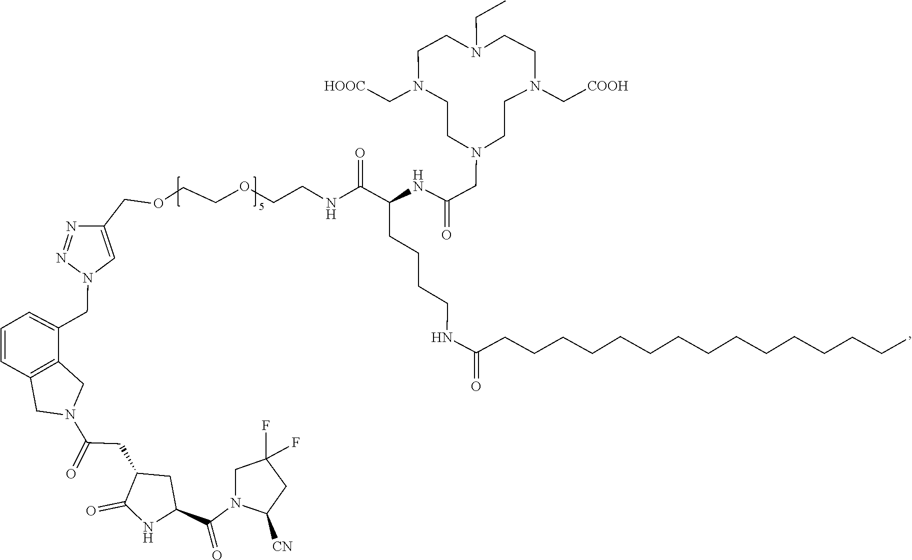

optionally bound to an isotope (or metal) suitable for radio-imaging, radiotherapy or magnetic resonance imaging.

Certain compounds hereof are represented by the structure of formula (X):

Aₘ-L-B′ (X)

wherein:
A is represented by the structure of formula X-X:

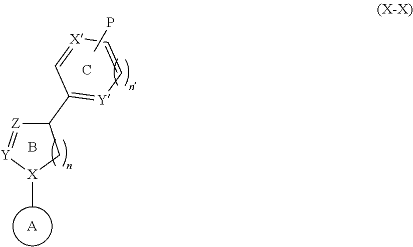

in which

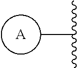

has a structure selected from the group consisting of:

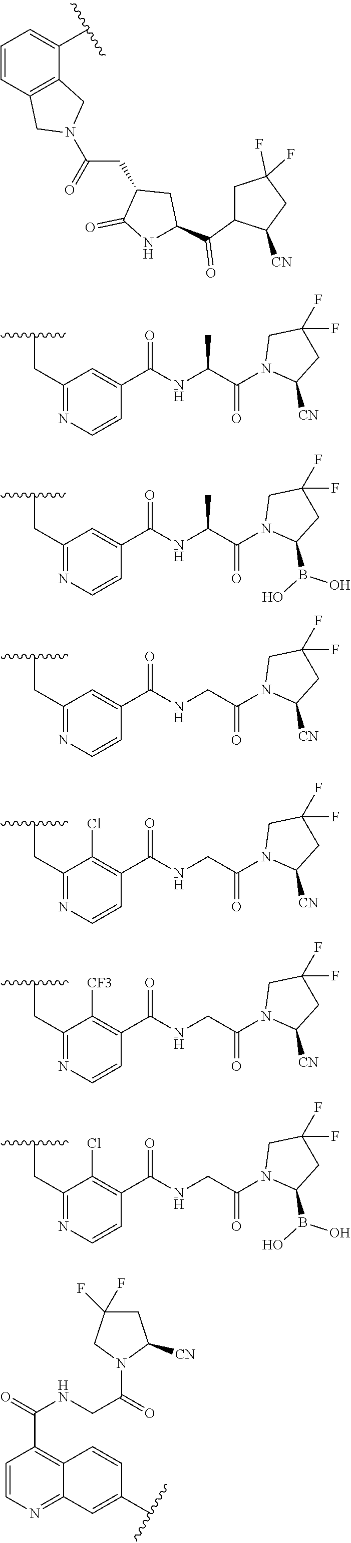

n=1-5,
n′=1-5,
ring C is optional,
X, Y and Z in ring B are independently selected from O, N and S, with the proviso that at least one of X and Y is N or Z is N,
X′ and Y′ in ring C are independent selected from O, N and S, with the proviso that at least one of X′ and Y′ is N,
P is a point of attachment of ring C, if present, to L or B′ of formula (X) and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl,
L, when present, is a linker,
B′ is a radical of an imaging agent or a radical of a therapeutic agent, and
m=1-6.

The compound can further comprise C′, wherein L connects C′ to one or more of the A groups and B′, and C′ is a radical of an albumin binding ligand, a (PEG)ₙ wherein n is an integer from 0 to 32, a peptide, a peptidoglycan or a saccharide.

In certain embodiments, B′ is a radical of a radio-imaging agent, a radiotherapeutic agent, or a magnetic resonance imaging agent.

B′ can be aromatic. In certain embodiments, n=2, n′=1, and A is represented by formula (X-Z):

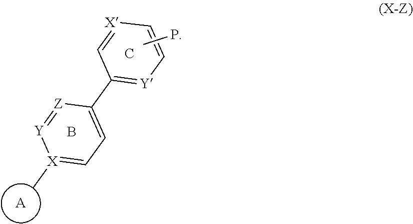

In certain embodiments, n=0-4 and A is represented by the structure of formula X-Y:

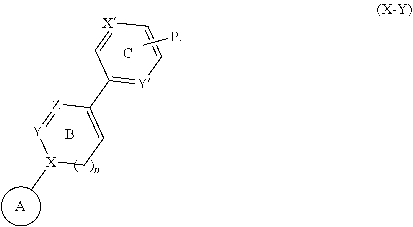

In certain embodiments, the compound has the structure:

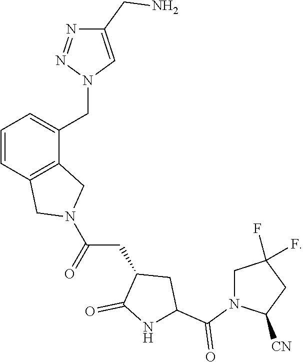 [SMILES: N#C[C@@H]1CC(F)(F)CN1C(=O)C1C[C@@H](CC(=O)N2Cc3cccc(Cn4cc(CN)nn4)c3C2)C(=O)N1]

In certain embodiments, the compound has the structure:

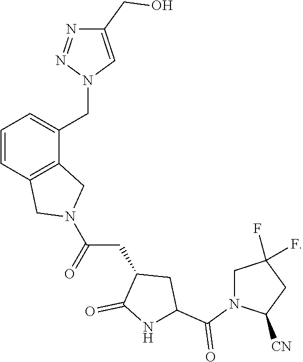 [SMILES: N#C[C@@H]1CC(F)(F)CN1C(=O)C1C[C@@H](CC(=O)N2Cc3cccc(Cn4cc(CO)nn4)c3C2)C(=O)N1]

In certain embodiments, the compound has the structure:

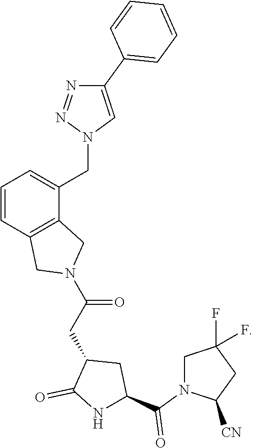 [SMILES: N#C[C@@H]1CC(F)(F)CN1C(=O)[C@@H]1C[C@@H](CC(=O)N2Cc3cccc(Cn4cc(-c5ccccc5)nn4)c3C2)C(=O)N1]

In certain embodiments, the compound has the structure:

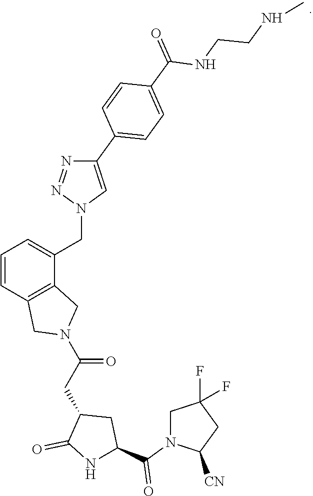 [SMILES: CNCCNC(=O)c1ccc(-c2cn(Cc3cccc4c3CN(C(=O)C[C@@H]3C[C@@H](C(=O)N5CC(F)(F)C[C@H]5C#N)NC3=O)C4)nn2)cc1]

L of the compound can comprise one or more linker groups, wherein each linker group is independently selected from the group consisting of alkyl(ene), heteroalkyl(ene), heterocycloalkyl(ene), heteroaryl, aryl, alkoxy, thioether, disulfide, carboxylic acid, anhydride, carbonate, carbamate, thioether, sugar, peptide, and peptidoglycan. L can be a single linker. L can comprise two or more linker groups. In certain embodiments, each linker group can be PEG.

In certain embodiments, L of the compound is (L¹)ₚ-W-(L²)_{q}, in which L¹ is a first linker, L² is a second linker, W is a third linker, p=1-5, and q=1-5. Each L¹ and each L² can independently comprise one or more linker groups, wherein each linker group is independently selected from the group consisting of alkyl(ene), heteroalkyl(ene), heterocycloalkyl(ene), heteroaryl, aryl, alkoxy, thioether, disulfide, carboxylic acid, anhydride, carbonate, carbamate, thioether, sugar, peptide, and peptidoglycan. In certain embodiments, each L¹ and each L² can independently comprise one or more linker groups, where each linker group independently selected from the group consisting of PEG, alkyl(ene), amide, phenyl, and triazole. In certain embodiments, W has an amine core, an aromatic core, or an alkylene core.

L, L¹, L², or L¹ and L² of the compound can have a length from 5 Angstroms to 200 Angstroms. In some embodiments, L, L¹, L², or L¹ and L² can comprise at least one linker group having the structure:

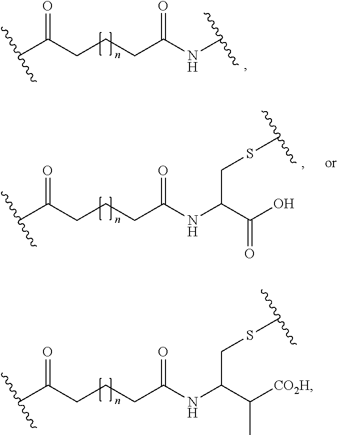

wherein n is 0 to 10.

L, L¹, L², or L¹ and L² can comprise at least one linker group having the structure:

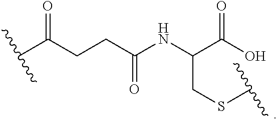 [SMILES: *SCC(NC(=O)CCC(*)=O)C(=O)O]

In other embodiments, L, L¹, L², or L¹ and L² can comprise at least one linker group having the structure:

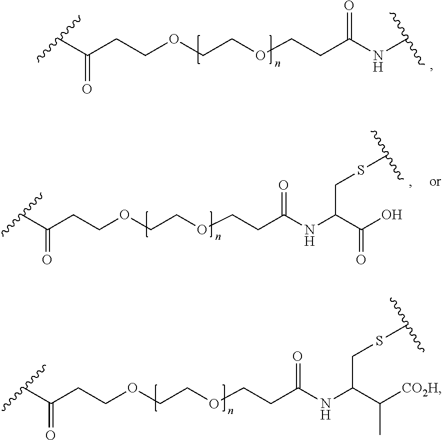

wherein n=1-32.

In certain embodiments (e.g., comprising a triazole), B′ is a radical of a dye. The dye can be, for example, fluorescent. B′ can be a radical of an anti-cancer agent. B′ can be a radical of an anti-fibrotic agent. B′ can be a radical of a PI3K inhibitor. B′ can be a radical of a radio-imaging agent comprising a chelated radioisotope. In certain embodiments, the radioisotope is selected from the group consisting of ^{99m}Tc ¹¹¹In, ¹⁸F, ⁶⁸Ga, ¹²⁴I, ¹²⁵I, and ¹³¹I. In certain embodiments, the radioisotope is selected from the group consisting of ³²P, ⁸⁹Sr, ⁹⁰Y, ¹⁵³Sm, ¹⁶⁹Er, ¹⁷⁷Lu, ¹⁸⁶Re, ¹⁸⁸Re, ¹⁴⁹Tb, ²¹¹At, ²¹²Bi, and ²²⁵Ac. B′ can be a radical of a dye. B′ can be a radical of a fluorescent dye. B′ can be a radical of an anti-cancer agent. B′ can be a radical of an anti-fibrotic agent.

Ligands are also provided. In certain embodiments, the ligand is for FAP and comprises an isoindoline scaffold into which a triazole moiety has been introduced and which has a Schrodinger molecular docking score of at least about −8.0 kcal/nol.

In certain embodiments, A of the compound is represented by the structure of formula X-X′:

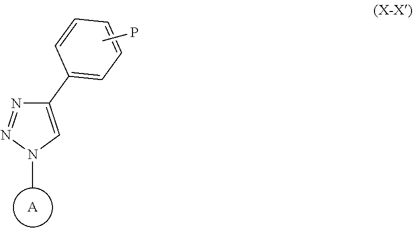

in which

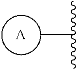

has the formula X-B, and
P is a point of attachment to L or B′ of formula (X) and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

In certain embodiments, A is represented by the structure of formula X-Y:

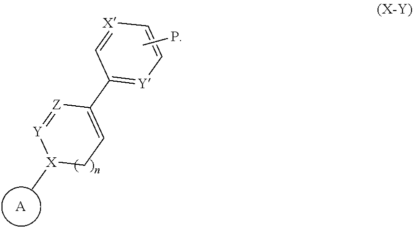

in which

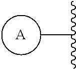

has the formula X-B,
n=0-4,
X, Y and Z are each independently selected from O, N and S, with the proviso that at least one of X and Y is N or Z is N,
X′ and Y′ are each independently selected from O, N, and S, with the proviso that at least one of X′ and Y′ is N or Z is N, and
P is a point of attachment to L or B′ of formula (X) and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

A can further be represented by the structure of formula X-Z:

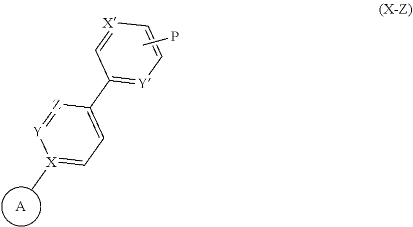

in which

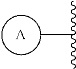

has the formula X-B,
X, Y and Z are each independently selected from O, N and S, with the proviso that at least one of X and Y is N or Z is N,
X′ and Y′ are each independently selected from O, N, and S, with the proviso that at least one of X′ and Y′ is N or Z is N, and
P is a point of attachment to L or B′ of formula (X) and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

Pharmaceutical compositions comprising any of the compounds described herein are provided. Such pharmaceutical compositions can comprise any one of the compounds hereof and a pharmaceutically acceptable carrier, for example.

Further provided is a method for imaging cancer or fibrosis (e.g., pulmonary fibrosis, renal fibrosis, or hepatic fibrosis) in a subject with the cancer or the fibrosis. The method comprises administering an effective amount of a compound or a pharmaceutical composition comprising the compound to a subject in need thereof. The method can further comprise imaging the subject. In certain embodiments, the method further comprises generating an image of the cancer or the fibrosis in the subject.

Further provided is a method for treating fibrosis in a subject. The method comprises administering an effective amount of a compound or a pharmaceutical composition comprising the compound to a subject in need thereof. The fibrosis can be, for example, selected from pulmonary fibrosis, renal fibrosis, and hepatic fibrosis.

Still further provided is a method for treating an inflammatory disease or disorder. The method comprises administering a therapeutically effective amount of a compound or a pharmaceutical composition comprising the compound (e.g., as described herein) to a subject in need thereof.

Methods of treating cancer are also provided. In certain embodiments, the method for treating cancer comprises administering a therapeutically effective amount of a compound hereof or a pharmaceutical composition comprising the compound to a subject in need thereof. The cancer can be selected from the group consisting of lung cancer, breast cancer, colorectal cancer, cervical cancer, and brain cancer (e.g., glioblastoma). In certain embodiments, the method further comprises administering chemotherapy or radiotherapy to the subject (or, for example, administering an additional treatment for the cancer to the subject).

Still further, methods of improving the affinity of a ligand, which comprises an isoindoline scaffold, for FAP are provided. In certain embodiments, the method comprises introducing a triazole moiety into the isoindoline scaffold of the ligand by molecular modeling to achieve a higher Schrodinger molecular docking score.

### BRIEF DESCRIPTION OF THE DRAWINGS

FIG. 1 shows both exemplified structures and a sampling of prophetic structures of various fibroblast activation protein (FAP). Structures FAP-3000-3017 are synthesized examples, FAP-3018-FAP-3029 are prophetic.

FIG. 2A depicts the displacement of the FAP ligand conjugated to a rhodamine fluorescent dye from HEK-FAP cells with the FAP ligand conjugated to DOTA without albumin binder at a range of concentrations (FAP-3000). FIG. 2B depicts the same displacement assay with the FAP ligand conjugated to DOTA with an iodobenzene albumin binder (FAP-3001). FIG. 2C depicts the same displacement assay with the FAP ligand conjugated to NOTA with a fluorobenzene albumin-binder (FAP-3002). FIG. 2D depicts the same displacement assay with the FAP ligand conjugated to NOTA with a chlorobenzene albumin-binder (FAP-3003). FIG. 2E depicts the same displacement assay with the FAP ligand conjugated to DOTA with a shorter PEG(4) spacer (FAP-3015). FIG. 2F depicts the same displacement assay with the FAP ligand conjugated to a longer PEG(12) spacer (FAP-3016). FIG. 2G depicts the same displacement assay with a monofluoro analog of the FAP ligand (FAP-3017).

FIG. 3A shows FAP-3000 indium-111 radiolabeling data. FIG. 3B shows FAP-3000 lutetium-177 radiolabeling data. FIG. 3C FAP-3001 indium-111 radiolabeling data. FIG. 3D shows FAP-3001 lutetium-177 radiolabeling data.

FIG. 4A depicts a binding curve of ¹¹¹In-FAP-3000 to the cancer-associated fibroblast cell line Hs894 over a range of concentrations, in the absence or presence of a 100× excess of FAP ligand as a competition blockade. FIG. 4B depicts the same binding curve assay with ¹¹¹In-FAP-3001.

FIG. 5A depicts a dosing study of ¹¹¹In-FAP-3000 using different amounts of the molecule but the same amount of radioactivity in HT29 tumor-bearing nude mice using single-photon emission computed tomography/computed tomography (SPECT/CT). FIG. 5B depicts a dosing study of ¹¹¹In-FAP-3001 using different amounts of molecule but the same amount of radioactivity in HT29 tumor-bearing nude mice using SPECT/CT.

FIG. 6A depicts a dosing study of ¹¹¹In-FAP-3000 using different amounts of the molecule but the same amount of radioactivity in 4T1 tumor-bearing nude mice using single-photon emission computed tomography/computed tomography (SPECT/CT). FIG. 6B depicts a dosing study of ¹¹¹In-FAP-3001 using different amounts of molecule but the same amount of radioactivity in 4T1 tumor-bearing nude mice using SPECT/CT. Highest intensity of radioactivity is identified by white circle(s) in each image.

FIG. 7A depicts a retention study of ¹¹¹In-FAP-3001 in 4T1 tumor-bearing Balb/c mice using SPECT/CT. FIG. 7B depicts a retention study of ¹¹¹In-FAP-3001 in KB tumor bearing mice using SPECT/CT. Highest intensity of radioactivity is identified by white circle(s) in each image.

FIG. 8A depicts a biodistribution study of ¹⁷⁷Lu-FAP-3000 in 4T1 tumor-bearing Balb/c mice at various time points. FIG. 8B depicts a biodistribution study of ¹¹¹In-FAP-3001 in 4T1 tumor-bearing Balb/c mice at various time points. SEM bar shown in both FIGS. 8A and 8B.

FIG. 9A depicts a radiotherapy study with ¹⁷⁷In-FAP-3001 radiolabeled with two different doses of Lu-177 (0.25 mCi vs. 0.5 mCi), using 4T1 tumor growth chart in Balb/c mice injected on day 0. FIG. 9B tracks the relative body weight of the mice. FIG. 9C shows a SPECT/CT scan of one of the treated mice 24 hours post-injection, with the highest intensity of radioactivity is identified by white circle(s) in each image. SEM bar shown in both FIGS. 9A and 9B.

FIG. 10A depicts a radiotherapy study with ¹⁷⁷In-FAP-3000 radiolabeled with 0.5 mCi, using KB tumor growth chart in nude mice injected on day 0. FIG. 10B depicts the survival curve of the same study. FIG. 10C tracks the relative body weight of the mice of the same study. SEM bar shown in both FIGS. 10A and 10C.

FIG. 11A depicts a radiotherapy study with ¹⁷⁷In-FAP-3001 radiolabeled 0.5 mCi using KB tumor growth chart in nude mice injected on day 0. FIG. 11B depicts the survival curve of the same study. FIG. 11C tracks the relative body weight of the mice of the same study. SEM bar shown in both FIGS. 11A and 11C.

FIG. 12A depicts a radiotherapy study with ¹⁷⁷In-FAP-3001 radiolabeled with 0.25 mCi, using HT29 tumor growth chart in nude mice injected on day 0. FIG. 12B depicts the survival curves of the same study. FIG. 12C tracks the relative body weight of the mice of the same study. FIG. 12D shows pictures of the collapsed tumors post euthanasia. SEM bar shown in both FIGS. 12A and 12C.

FIG. 13A depicts a radiotherapy study with ¹⁷⁷In-FAP-3001 radiolabeled with two different doses of Lu-177 (0.25 mCi vs. 0.5 mCi) injected on day 0, using U87MG tumor growth chart in nude mice. FIG. 13B depicts the survival curve of the same study. FIG. 13C tracks the relative body weight of the mice of the same study. SEM bar shown in both FIGS. 13A and 13C.

FIG. 14 shows images of tissue sections harvested from various organs of interest in control and treated (0.5 mCi) groups that were H&E stained for evaluation.

FIG. 15A depicts a radiotherapy study with ¹⁷⁷In-FAP-3001 (1.5 mCi injected on day 0 or two different doses—1.5 mCi+0.60 mCi injected on days 0 and 3, respectively). FIG. 15B depicts the survival curve of the same study. FIG. 15C tracks the relative body weight of the mice of the same study. FIG. 15D shows a SPECT/CT scan of one of the treated mice at various time points post-injection. SEM bar shown in both FIGS. 15A and 15C.

FIG. 16A depicts a radiotherapy study with ¹⁷⁷In-FAP-3001 radiolabeled with 1.5 mCi and injected on day 0 then measured by 2 researchers in blind fashion. FIG. 16B depicts the survival curve of the same study. FIG. 16C tracks the relative body weight of the mice of the same study. SEM bar shown in both FIGS. 16A and 16C.

FIG. 17A depicts a radiotherapy study with ¹⁷⁷In-FAP-3001 vs. ¹⁷⁷In-FAP-3005 radiolabeled with 1.5 mCi and injected on day 0. FIG. 17B depicts the survival curve of the same study. FIG. 17C tracks the relative body weight of the mice of the same study. SEM bar shown in both FIGS. 17A and 17C.

FIG. 18A shows a summary of histopathology of necropsy tissues from ¹⁷⁷Lu-FAP-3001 radiotherapy treatment at different doses and different time points post-injection.

FIG. 18B shows images of tissue sections harvested from various organs of interest in control and treated (1.5 mCi) groups at different time points point injection that were H&E stained for evaluation.

FIG. 19 depicts a SPECT/CT scan of ¹¹¹In-FAP-3001 in a pulmonary fibrosis model 24 hours post-injection. Top arrows depict lungs. Bottom arrows depict kidneys.

FIGS. 20A and 20B show the structures of various fibroblast activation protein (FAP)-phosphoinositide 3-kinase (PI3K) (FAP5-PI3K) inhibitors.

FIG. 21 shows the results of the FAP5-PI3K inhibitors after 24 hours.

FIG. 22 shows the results of the FAP5-PI3K inhibitors after 48 hours.

FIGS. 23A and 23B show graphical data related to the inhibition of phosphorylation of Akt by the FAP5-PI3K inhibitors after 24 hours of incubation (FIG. 5A) and after 48 hours of incubation (FIG. 5B).

FIG. 24 and FIG. 25 each depict mass spectrometry data confirming the desired compound at issue was produced.

FIG. 26 depicts a molecular model of a known FAP inhibitor (FAP inhibitor 1) and a molecular model of FAP-4001, and illustrates the increased interactions between FAP and FAP-4001 when docked.

FIG. 27 depicts molecular models of various compounds and the interactions with FAP when docked.

FIG. 28 shows exemplary structures of various FAP triazole scaffolds.

FIG. 29 shows exemplary structures of various compounds that do not include a linker.

FIG. 30 shows exemplary structures of various compounds that comprise a PEG linker.

FIG. 31 shows exemplary structures of various compounds that comprise an alkyl linker.

FIGS. 32A and 32B depict fluorescence images (panels I and iv) and white light images (panels ii and v), which are merged in panels ii and vi, taken from studies of the internalization of FAP-4004 (FIG. 32A) and FAP-4003 (FIG. 32B) into HT1080-FAP cells.

FIGS. 32C and 32D show graphical data related to the binding affinities and specificities of FAP-4004 (FIG. 32C) and FAP-4002 (FIG. 32D) hereof.

FIGS. 33A-33C depicts graphical data related to the analysis of the ability of a FAP-targeted ligand FAP-4002 to inhibit the closely related dipeptidyl peptidases FAP (FIG. 33A), PREP (FIG. 33B), and DPP-IV (FIG. 33C).

FIGS. 34A-34C depict data relating to confirmation of the production of certain compounds hereof, with FIGS. 34A and 34B showing ¹H nuclear magnetic resonance spectroscopy (¹H NMR) data for compound 16, and FIG. 34C showing ¹H NMR (D₂O) spectra data for FAP-4003.

FIG. 35 depicts excitation (Ex) and emission (Em) spectra of a 1 μM solution in PBS, pH 7.4, of FAP-4003.

### DETAILED DESCRIPTION

The present disclosure relates to the preparation and use of compounds and compositions that reduce the propensity for off-target toxicity following administration of therapeutic and/or imaging agents (e.g., a radio-imaging agent, a radiotherapeutic agent, a chemotherapeutic agent, an antifibrotic agent, or an anticancer agent). The term “off-target toxicity” means organ or tissue damage or a reduction in the subject's weight that is not desirable to the physician treating the subject, or any other effect on the subject that is potentially an adverse indicator to the treating physician (e.g., B cell aplasia, a fever, a drop in blood pressure, or pulmonary edema). The terms “treat,” “treating,” “treated,” or “treatment” (with respect to a disease or condition) are used regarding an approach for obtaining beneficial or desired results, such as clinical results. Such results include, but are not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic (e.g., preventative) treatment. In reference to cancer, in particular, the terms “treat,” “treating,” “treated,” or “treatment” can additionally mean reducing the size of a tumor, completely or partially removing the tumor (e.g., a complete or partial response), stabilizing disease, preventing progression of the cancer (e.g., progression free survival), or any other effect on the cancer that would be considered by a physician to be a therapeutic or prophylactic treatment of the cancer.

The compounds hereof can comprise a fibroblast activation protein (FAP)-targeted ligand (or a radical thereof) attached to a linker comprising one or more linker groups (e.g., three linker groups), wherein the linker is further attached to a therapeutic agent or imaging agent. FAP is a type II membrane bound serine protease that cleaves proline-amino acid peptide bonds and can be expressed on cancer associated fibroblasts (CAFs) and on myofibroblasts that produce collagen. In certain embodiments, the compounds can target therapeutic compounds to a FAP expressing cancer or fibrotic or inflammatory disease. In certain embodiments, this improved FAP-targeting ligand scaffold can additionally be used with albumin-binding moieties to achieve the targeted delivery of radiolabeled and other functional groups. In certain embodiments, the FAP-targeting ligand is a high affinity FAP ligand that comprises a triazole moiety (or a derivative thereof) introduced into a scaffold of the ligand. In certain embodiments, the FAP-targeting ligand is a high affinity FAP ligand that comprises a triazole moiety (or a derivative thereof) and a phenyl ring introduced into a scaffold of the ligand (e.g., an isoindoline ring scaffold). Unless otherwise specified, “high affinity” or “higher affinity” with respect to a ligand's affinity for a target means a ligand that has a Schrodinger molecular docking score of at least about −8.0 kcal/mol. In certain embodiments, the high affinity FAP ligand has an improved affinity for FAP as compared to a ligand without a triazole moiety introduced therein.

The compounds, compositions, and methods will now be described in detail. For the purposes of promoting an understanding of the principles presented herein, reference is made to the embodiments illustrated in the drawings and specific language will be used to describe the same. It will nevertheless be understood that no limitation of scope is intended by the description of these embodiments. On the contrary, this disclosure is intended to cover alternatives, modifications, and equivalents as may be included within the spirit and scope of this application as defined by the appended claims. As previously noted, while this technology may be illustrated and described in one or more preferred embodiments, the compositions, compounds and methods hereof may comprise many different configurations, forms, materials, and accessories.

### Compounds

Provided are compounds (i.e., conjugates) of formula X:

AₘₓL-B′ (X)

wherein
A is a radical of a fibroblast activation protein alpha (FAPα) ligand (i.e., a targeting moiety) (e.g., with a molecular weight below 10,000);
L is a (e.g., bi-functionalized or trifunctionalized) linker connecting one or more A groups to B′ (e.g., through a first covalent bond linking L to A and a second covalent bond linking L to B′);
B′ is an (e.g., a radical of) a photodynamic therapeutic agent, a radio-imaging agent, a radiotherapeutic agent, a chemotherapeutic agent, an antifibrotic agent, or an anticancer agent (e.g., an anticancer agent that is effective against cancer cells or cancer-associated fibroblasts, myofibroblasts or other tumor microenvironment factors); and
mx is 1-6.

In certain embodiments, mx is 1. In certain embodiments, mx is 2. In certain embodiments, mx is 3. In certain embodiments, mx is 4. In certain embodiments, mx is 5. In certain embodiments, mx is 6. mx can be 1 to 3, 2 to 4, or 1 to 5.

The disclosure also relates to compounds (i.e., conjugates) of formula I:

A-L-B′ (I)

wherein:
A comprises (e.g., a radical of) a FAPα ligand (i.e., a targeting moiety);
L comprises a (e.g., bi-functionalized or trifunctionalized) linker connecting one or more A groups to B; and
B′ comprises (e.g., a radical of) a photodynamic therapeutic agent, a radio-imaging agent, a radiotherapeutic agent, a chemotherapeutic agent, an antifibrotic agent, or an anticancer agent (e.g., an anticancer agent that is effective against cancer cells or cancer-associated fibroblasts, myofibroblasts or other tumor microenvironment factors).

Also provided are compounds (e.g., conjugates) of formula I:

A-L-B′ (I)

wherein:
A comprises (e.g., a radical of) a FAPα ligand (i.e., a targeting moiety);
L comprises a (e.g., bi-functionalized or tri-functionalized) linker connecting one or more A groups to B′; and
B′ comprises (e.g., a radical of) a radiolabeled functional group suitable for PET imaging, SPECT imaging, other radioimaging techniques, or radiotherapy.

Also provided is a compound represented by the structure of formula (X):

A-L-B′ (X)

wherein:
A is a radical of a FAPα ligand of the formula X-B:

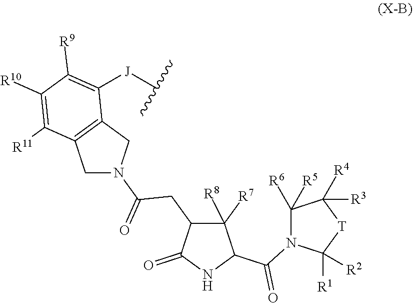

wherein:
T is substituted or unsubstituted methylene (—CH₂—), substituted or unsubstituted amino (—NH—), —O—, or —S— (e.g., wherein the substitution of T is C₁-C₃ alkyl, haloalkyl, or halo); J is C(R^{J})₀₋₃, wherein each R¹ is independently H or alkyl, or two or more R^{J} are taken together to form oxo;
R¹ and R² are independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are each independently selected from group consisting of H, alkyl, and halo; and
R⁹, R¹⁰, and R¹¹ are independently selected from group consisting of H, —C₁₋₆alkyl, —C₁₋₆haloalkyl, —O—C₁₋₆alkyl, —S—C₁₋₆ alkyl, F, Cl, Br and I;
L is a linker connecting A to B′; and
B′ is a therapeutic agent, a radio-imaging agent, a radiotherapeutic agent, a chemotherapeutic agent, an antifibrotic agent, or an anticancer agent.

In some embodiments, A of formula X is represented by the structure of formula X-X′:

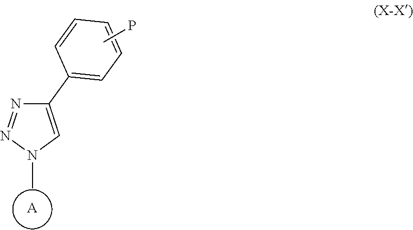

in which

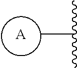

has the formula X-B, and
P is a point of attachment to L or B′ of formula (X) and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

In some embodiments, A of formula X is represented by the structure of formula X-X:

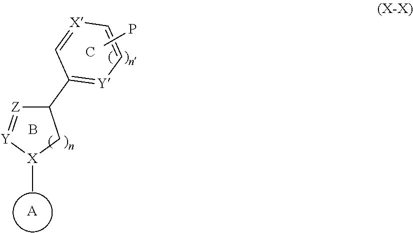

in which

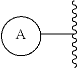

has the formula X-B
n=1-5,
n′=1-5,
ring C is optional,
X, Y and Z in ring B are independently selected from O, N and S, with the proviso that at least one of X and Y is N or Z is N,
X′ and Y′ in ring C are independent selected from O, N and S, with the proviso that at least one of X′ and Y′ is N, and
P is a point of attachment of ring C, if present, to L or B′ of formula (X) and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

In some embodiments,

[symbol]

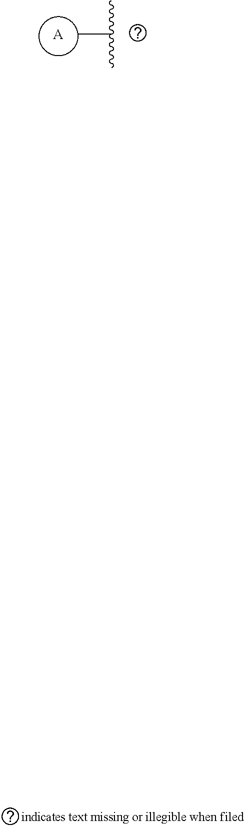

has a structure selected from the group consisting of

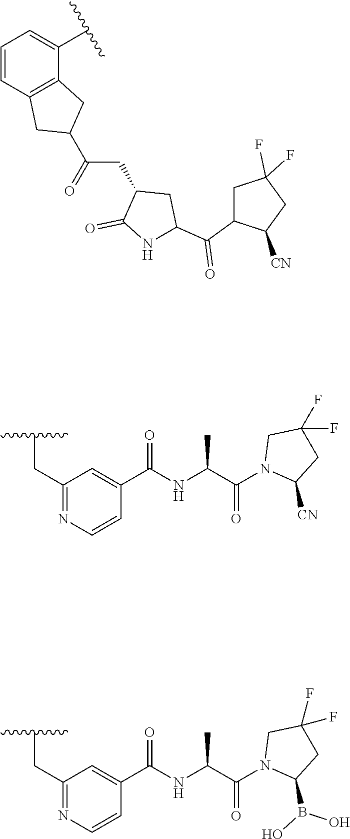 [SMILES: *c1cccc2c1CC(C(=O)C[C@@H]1CC(C(=O)C3CC(F)(F)C[C@H]3C#N)NC1=O)C2 ; *Cc1cc(C(=O)N[C@@H](C)C(=O)N2CC(F)(F)C[C@H]2C#N)ccn1 ; *Cc1cc(C(=O)N[C@@H](C)C(=O)N2CC(F)(F)C[C@H]2B(O)O)ccn1]

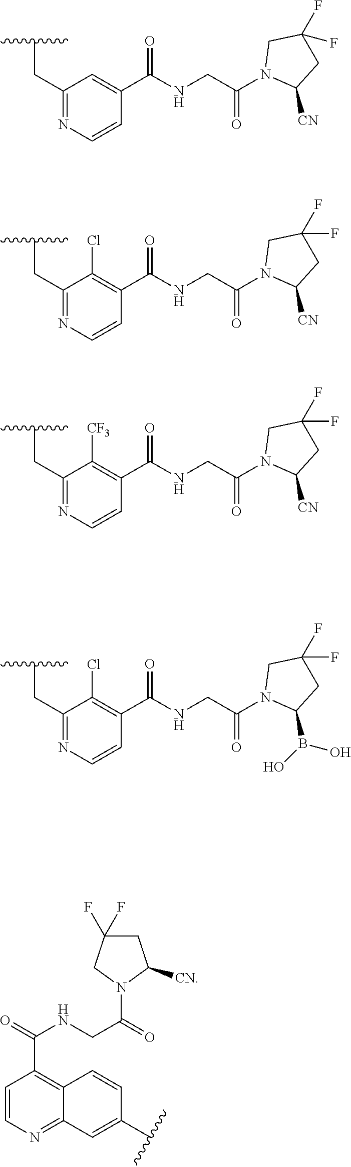 [SMILES: *Cc1cc(C(=O)NCC(=O)N2CC(F)(F)C[C@H]2C#N)ccn1 ; *Cc1nccc(C(=O)NCC(=O)N2CC(F)(F)C[C@H]2C#N)c1Cl ; *Cc1nccc(C(=O)NCC(=O)N2CC(F)(F)C[C@H]2C#N)c1C(F)(F)F ; *Cc1nccc(C(=O)NCC(=O)N2CC(F)(F)C[C@H]2B(O)O)c1Cl ; *c1ccc2c(C(=O)NCC(=O)N3CC(F)(F)C[C@H]3C#N)ccnc2c1]

In some embodiments, the compound further comprises C′, wherein:
L links C′ to the one or more A groups and B′; and
   C′ is a radical of an albumin binding ligand, a polyethylene glycolₙ (PEG)ₙ wherein n is an integer from 0 to 32, a peptide, a peptidoglycan or a saccharide.

Also provided is compound represented by a structure of formula (I′):

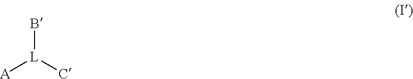

wherein:
A is a radical of a FAPα ligand (targeting moiety) of the formula X-B:

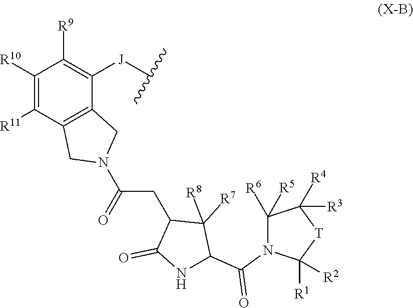

wherein:
T is substituted or unsubstituted methylene (—CH₂—), substituted or unsubstituted amino (—NH—), —O—, or —S— (e.g., wherein the substitution of T is C₁-C₃ alkyl, haloalkyl, or halo);
J is C(R^{J})₀₋₃, wherein each R^{J} is independently H or alkyl, or two or more R^{J} are taken together to form oxo;
R¹ and R² are independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are independently selected from group consisting of H, alkyl, and halo; R⁹, R¹⁰, and R¹¹ are each independently selected from group consisting of H, —C₁₋₆alkyl, —C₁₋₆haloalkyl, —O—C₁₋₆alkyl, —S—C₁₋₆ alkyl, F, Cl, Br and I; L is a trivalent linker;
B′ is a radical of a chelating group optionally bound to an isotope, said isotope (or metal) being suitable for radio-imaging, radiotherapy or magnetic resonance imaging; and
C′ is a radical of an albumin binding ligand, (PEG)ₙ wherein n is an integer from 0 to 32, a peptide, a peptidoglycan or a saccharide.

In some embodiments, B′ is a radical of a therapeutic agent or an imaging agent such as, for example, a phosphoinositide 3-kinase (PI3K) inhibitor, a chelating group optionally bound to an isotope (or metal), or a group covalently bound to an isotope (or metal), said isotope (or metal) suitable for radio-imaging, radiotherapy or magnetic resonance imaging.

In some embodiments, B′ is a radical of a chelating group optionally bound to a metal, or a group covalently bound to an isotope, said metal or isotope suitable for radio-imaging, radiotherapy or magnetic resonance imaging.

Compounds (e.g., conjugates) of formula I′ are also provided:

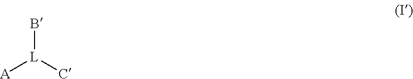

wherein
A is a radical of a FAPα ligand (i.e., a targeting moiety) (e.g., with a molecular weight below 10,000);
L is a trivalent linker;
B′ is a radical of a therapeutic agent or an imaging agent such as, for example, a PI3K inhibitor, a chelating group optionally bound to an isotope (or metal), or a group covalently bound to an isotope (or metal), said metal or isotope (or metal) suitable for radio-imaging, radiotherapy or magnetic resonance imaging; and
C′ is a radical of an albumin binding ligand, (PEG)ₙ wherein n is an integer from 0-32, a peptide, a peptidoglycan, or a saccharide.

The disclosure also relates to compounds (e.g., conjugates) of formula (II):

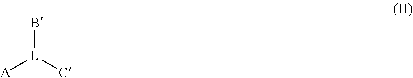

wherein:
A comprises a radical of a FAPα ligand (i.e., a targeting moiety) (e.g., with a molecular weight below 10,000);
L comprises a trivalent linker; and
B′ comprises a radical of a PI3K inhibitor, a chelating group optionally bound to a metal, or a group covalently bound to an isotope, said metal or isotope suitable for radio-imaging, radiotherapy or magnetic resonance imaging; and
C′ comprises a radical of an albumin binding ligand, (PEG)ₙ wherein n is an integer from 0-32, a peptide, a peptidoglycan, or a saccharide.

The disclosure also relates to compounds (i.e., conjugates) of formula (II):

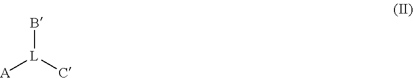

wherein:
A comprises a radical of a FAPα ligand (i.e., a targeting moiety) (e.g., with a molecular weight below 10,000);
L comprises a trivalent linker; and
B′ comprises a radical of a PI3K inhibitor, a chelating group optionally bound to an isotope (or metal), or a group covalently bound to an isotope (or metal), said isotope (or metal) suitable for radio-imaging, radiotherapy or magnetic resonance imaging; and
C′ comprises a radical of an albumin binding ligand.

In some embodiments, the compound is represented by a structure of formula (I′):

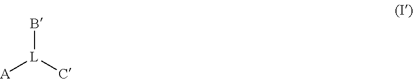

wherein:
A is a radical of a FAPα ligand (i.e., a targeting moiety) (e.g., with a molecular weight below 10,000) and comprises

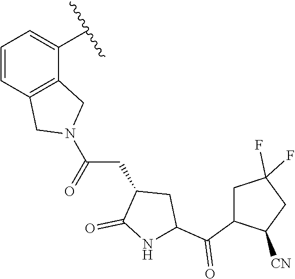

L is a trivalent linker;
B′ is a radical of a PI3K inhibitor, a chelating group optionally bound to an isotope (or metal), or a group covalently bound to an isotope (or metal), said isotope (or metal) suitable for radio-imaging, radiotherapy or magnetic resonance imaging; and
C′ is a radical of an albumin binding ligand, (PEG)ₙ wherein n is an integer from 0 to 32, a peptide, a peptidoglycan or a saccharide.

In some embodiments, the compound is represented by a structure of formula (I′):

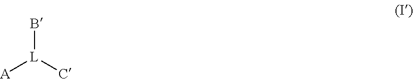

wherein:
A is a radical of a FAPα ligand (i.e., a targeting moiety) (e.g., with a molecular weight below 10,000) and comprises

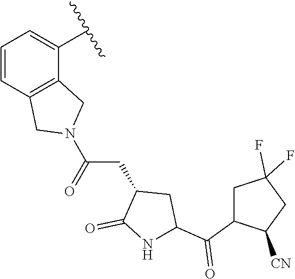

L is a trivalent linker;
B″ is a radical of a chelating group optionally bound to an isotope (or metal), said isotope (or metal) suitable for radio-imaging, radiotherapy or magnetic resonance imaging; and
C′ is a radical of an albumin binding ligand.

In some embodiments, the compound is represented by the structure of formula (X):

Aₘ-L-B′ (X)

wherein:
A is represented by the structure of formula X-X:

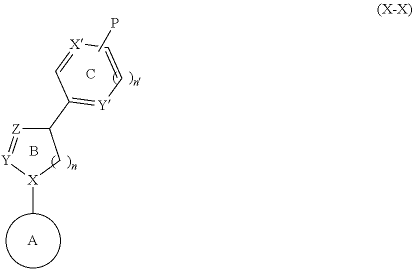

in which

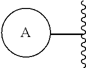

has a structure selected from the group consisting of

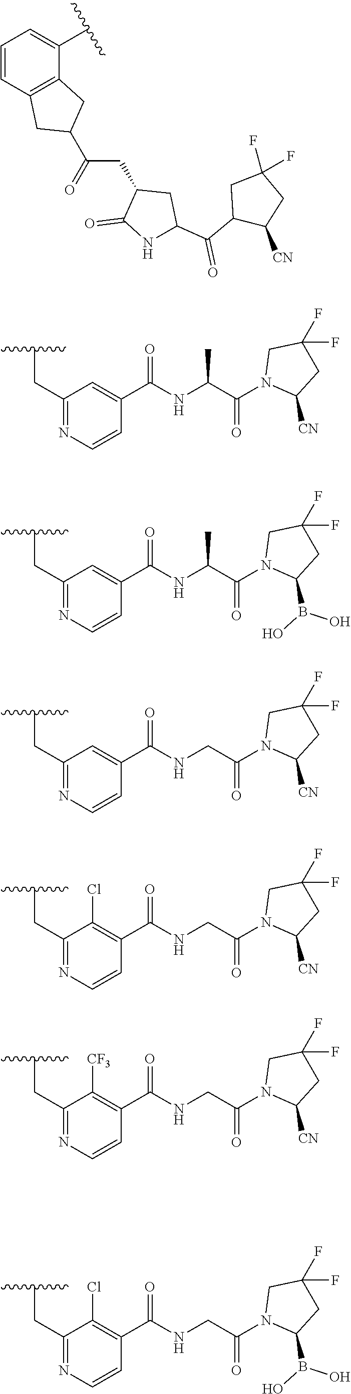

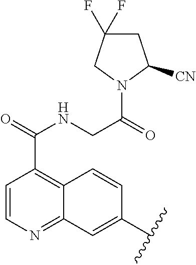 [SMILES: *c1ccc2c(C(=O)NCC(=O)N3CC(F)(F)C[C@H]3C#N)ccnc2c1]

and any other FAP ligand structure described herein;
n=1-5,
n′=1-5,
ring C is optional,
X, Y and Z in ring B are each independently selected from O, N and S, with the proviso that at least one of X and Y is N or Z is N,
X′ and Y′ in ring C are each independent selected from O, N and S, with the proviso that at least one of X′ and Y′ is N,
P is a point of attachment of ring C, if present, to L or B′ of formula (X) and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl,
L, when present, is a linker,
B′ is a radical of an imaging agent or a radical of a therapeutic agent, and
m=1-6.

The compounds can contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds are contemplated. When the compounds contain alkene double bonds, and unless specified otherwise, it is intended that this includes both E and Z geometric isomers (e.g., cis or trans). Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para- isomers around a benzene ring. One of ordinary skill in the art will further appreciate that the compounds can be “deuterated,” meaning one or more hydrogen atoms can be replaced with deuterium.

A compound can be a monovalent conjugate (e.g., a compound comprising one binding ligand (as described elsewhere herein, e.g., one FAP-binding ligand)). A compound can be a bivalent conjugate (e.g., a compound comprising one or more binding ligands (as described elsewhere herein, e.g., one or more FAP-binding ligands) conjugated to a therapeutic agent or an imaging agent (e.g., through a linker) (as described elsewhere herein)). A compound can be a multivalent conjugate (e.g., a compound comprising two or more binding ligands (as described elsewhere herein, e.g., two or more FAP-binding ligands) conjugated to a multipoint linker).

### Binding Ligands/Targeting Moieties (A)

In certain embodiments, A of formulae (X), (I), (I′) or (II) is a targeting moiety/binding ligand. The binding ligand (also referred to herein as the targeting ligand or the targeting moiety) can be a compound (or radical thereof) that binds to a biological molecule (e.g., a polypeptide (e.g., an enzyme)) localized to a particular cell, tissue, organ, or the like. The binding ligand can be a FAP ligand (or a radical thereof). The binding ligand can be a fibroblast activation protein alpha (FAPα) ligand (or a radical thereof). As used herein, a “ligand” is a molecule, ion, or atom that is attached to the central atom or ion (e.g., a drug) of a compound. “Ligand” also encompasses a binding agent that is not an agonist or antagonist and has no agonist or antagonist properties.

In certain embodiments, the targeting moiety binds to an activated fibroblast expressing FAPα and such activated fibroblast is involved in cancer or inflammatory diseases.

The targeting moiety can be, for example, a radical of FAPα ligand with a molecular weight less than about 10,000, less than 7,500, less than 5,000, less than 2,500, less than 1,000, less than 760, less than 500; from about 500 to about 10,000 g/mol, about 1,000 to about 7,500 g/mol, about 750 g/mol to about 1,500 g/mol, about 1,000, to about 5,000 g/mol or about 500 to about 2,500 g/mol.

The targeting moiety can bind to an activated fibroblast expressing FAPα where such activated fibroblast is involved in cancer or inflammatory diseases.

In certain embodiments, A has a structure represented by the formula (I-A1) or (I-A2):

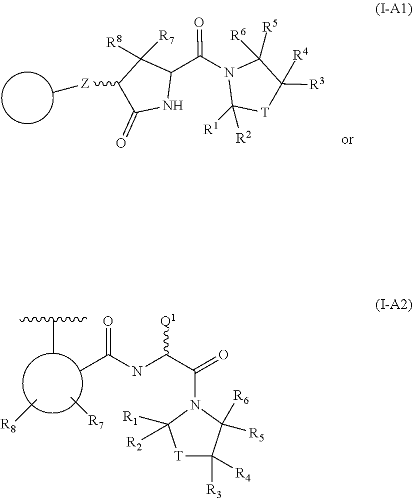

wherein:

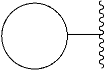

is a functionalized 5- to 10-membered N-containing aromatic or non-aromatic, mono- or bicyclic heterocycle, said heterocycle optionally further comprising 1 to 3 heteroatoms selected from oxygen, nitrogen, and sulfur;
Z, in formula (I-A1), is a bond, substituted or unsubstituted alkylene (e.g., —CH₂—), substituted or unsubstituted amino (e.g., —NH—), —O—, or —S—;
T is substituted or unsubstituted methylene (—CH₂—), substituted or unsubstituted amino (—NH—), —O—, or —S—;
R¹ and R² are independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F and 5-tetrazolyl;
R³ and R⁴ are independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are independently selected from group consisting of H, alkyl and halo; Q¹, in formula (I-A2) is selected from the group consisting of —H, —CH₃, —CH₂OH, and —CH(CH₃)₂;
and

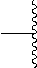

is a point of attachment of the FAPα binding ligand (e.g., through the linker, L, or an imaging/therapeutic agent moiety B′), wherein the point of attachment can be through any of the carbon atoms of the 5- to 10-membered N-containing aromatic or non-aromatic, mono- or bicyclic heterocycle, 1° or 2° amines, or a functionalized alkyl or cycloalkyl motif, as well as stereoisomers and pharmaceutically acceptable salts thereof.

A can have a structure represented by the formula I-B:

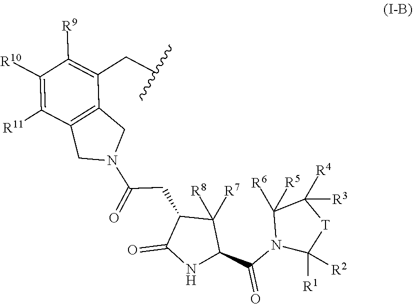

wherein:
T is substituted or unsubstituted methylene (—CH₂—), substituted or unsubstituted amino (—NH—), —O—, or —S—;
R¹ and R² are independently selected from the group consisting of —H, —CN, —CHO, —B(OH)₂, —C(O)alkyl, —C(O)aryl-, —C═C—C(O)aryl, —C═C—S(O)₂ aryl, —CO₂H,
—SO₃H, —SO₂NH₂, —PO₃H₂, —SO₂F and 5-tetrazolyl; R³ and R⁴ are independently selected from the group consisting of —H, —OH, F, Cl, Br, I, —C₁₋₆alkyl, —O—C₁₋₆alkyl, and —S—C₁₋₆ alkyl;
R⁵, R⁶, R⁷, and R⁸ are independently selected from group consisting of H, alkyl and halo; and
R⁹, R¹⁰, and R¹¹ are independently selected from group consisting of H, —C₁₋₆ alkyl, —O—C₁₋₆ alkyl, —S—C₁₋₆ alkyl, F, Cl, Br and I.

In certain embodiments, A has a structure represented by the formula I-C:

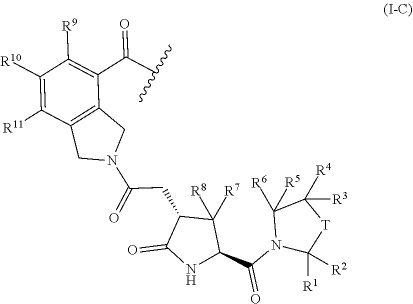

wherein:
T is substituted or unsubstituted methylene (—CH₂—), substituted or unsubstituted amino (—NH—), —O—, or —S—;
R¹ and R² are independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are independently selected from the group consisting of —H, —OH, F, Cl, Br, I, —C₁₋₆ alkyl, —O—C₁₋₆ alkyl, and —S—C₁₋₆ alkyl; R⁵, R⁶, R⁷, and R⁸ are independently selected from group consisting of H, alkyl and halo; and
R⁹, R¹⁰, and R¹¹ are independently selected from group consisting of H, —C₁₋₆ alkyl, —O—C₁₋₆ alkyl, —S—C₁₋₆ alkyl, F, Cl, Br and I.

A can have a structure represented by the following formulae:

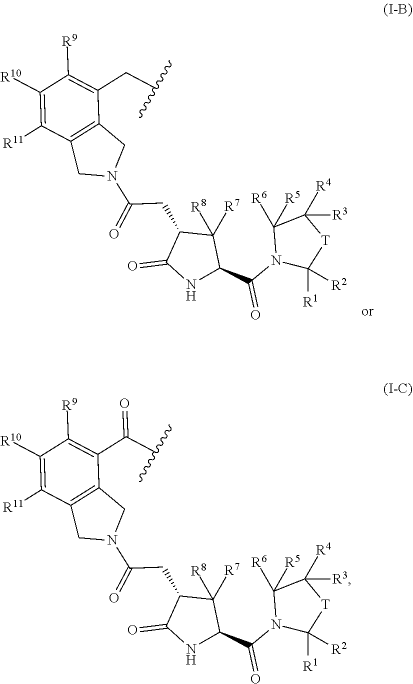

wherein T is substituted or unsubstituted methylene (—CH₂—), substituted or unsubstituted amino (—NH—), —O—, or —S—;
R¹ and R² are independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, and 5-tetrazolyl;
R³ and R⁴ are independently selected from the group consisting of —H, —OH, F, Cl, Br, I, —C₁₋₆ alkyl, —O—C₁₋₆ alkyl, and —S—C₁₋₆ alkyl;
R⁵, R⁶, R⁷, and R⁸ are independently selected from group consisting of H, alkyl and halo; and
R⁹, R¹⁰, and R¹¹ are each independently selected from group consisting of H, —C₁₋₆ alkyl, —O—C₁₋₆ alkyl, —S—C₁₋₆ alkyl, F, Cl, Br and I.

In certain embodiments, A has a structure represented by the formula X-A:

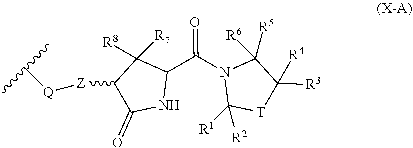

wherein:
Q is an aryl, a heteroaryl, or a heterocyclyl (e.g., comprising aryl and non-aryl ring structures) (e.g., 5- to 10-membered N-containing aromatic or non-aromatic mono- or bicyclic heterocycle, said heterocycle optionally further comprising 1 to 3 heteroatoms selected from O, N, and S);
Z is a bond, substituted or unsubstituted C₁-C₃ alkylene (e.g., —CH₂—), substituted or unsubstituted heteroalkyl (e.g., 1-3 atoms in length), amino (e.g., NH), —O—, or —S—;
T is substituted or unsubstituted methylene (—CH₂—), substituted or unsubstituted amino (—NH—), —O—, or —S— (e.g., wherein the substitution of T is C₁-C₃ alkyl, haloalkyl, or halo);
R¹ and R² are independently selected from the group consisting of —H, —CN, —CHO, —B(OH)₂, —C(O)alkyl, —C(O)aryl-, —C═C—C(O)aryl, —C═C—S(O)₂ aryl, —CO₂H, —SO₃H, —SO₂NH₂, —PO₃H₂, —SO₂F, —CONH₂, and 5-tetrazolyl;
R³ and R⁴ are independently selected from the group consisting of —H, —OH, F, Cl, Br, I, —C₁₋₆ alkyl, —O—C₁₋₆ alkyl, and —S—C₁₋₆ alkyl; and
R⁵, R⁶, R⁷, and R⁸ are independently selected from group consisting of H, alkyl, and halo.

In certain embodiments of formula (X-A), Q is attached to L (e.g., L or L₁ (see Linker section below)). Q can be aryl, heteroaryl, or heterocyclyl. The heterocyclyl can comprise aryl and non-aryl ring structures. Q can be attached to L at a heteroalkyl, an alkyl, or an aryl position of Q. Q can be attached to L at an aryl position of Q. Q can be attached to L via a nitrogen atom (e.g., of L). Q can be attached to L via a triazolyl or an amide (e.g., of L). The heteroaryl can comprise aryl and non-aryl ring structures. The heteroaryl or the heterocyclyl can comprise 1 to 3 heteroatoms selected from O, N, and S. The heterocyclyl can comprise 1 to 3 heteroatoms selected from O, N, and S. Q can be a 5- to 10-membered N-containing aromatic or non-aromatic mono- or bicyclic heterocycle (e.g., optionally comprising aryl and non-aryl ring structures). Q can be a N-attached heterocyclyl (e.g., optionally comprising aryl and non-aryl ring structures). Q can be a C₆-C₉—N-attached heterocyclyl (e.g., optionally comprising aryl and non-aryl ring structures). The N-attached heterocyclyl is attached to Z via a N-heterocycloalkyl. Q can be (e.g., an N-attached) isoindolinyl (e.g., wherein the N is attached to Z of formula (X-A)).

As noted above, in certain embodiments of formula (X-A), Z is a bond, substituted or unsubstituted C₁-C₃ alkylene, substituted or unsubstituted heteroalkylene (e.g., 1-3 atoms in length), amino (e.g., NH), —O—, or —S—. Z can be a bond. Z can be substituted methylene. Z can be —CH₂—. Z can be substituted ethylene. Z can be ethylene substituted with oxo. Z can be —C(CO)CH₂—. Z can be —CH₂CH₂—. Z can be a C₁-C₃ heteroalkylene.

In certain embodiments, A is or comprises a structure represented by the formula X-B:

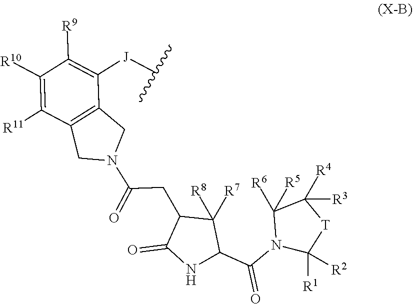

wherein:
T is substituted or unsubstituted methylene (—CH₂—), substituted or unsubstituted amino (—NH—), —O—, or —S— (e.g., wherein the substitution of T is C₁-C₃ alkyl, haloalkyl, or halo);
J is C(R^{J})₀₋₃, wherein each R^{J} is independently H or alkyl, or two or more R^{J} are taken together to form oxo;
R¹ and R² are independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are independently selected from group consisting of H, alkyl, and halo; and
R⁹, R¹⁰, and R¹¹ are independently selected from group consisting of H, —C₁₋₆alkyl, —C₁₋₆haloalkyl, —O—C₁₋₆alkyl, —S—C₁₋₆ alkyl, F, Cl, Br and I.

In certain embodiments, A is or comprises a structure represented by the formula X-C:

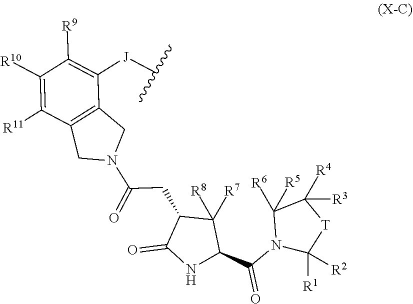

wherein:
T is substituted or unsubstituted methylene (—CH₂—), substituted or unsubstituted amino (—NH—), —O—, or —S— (e.g., wherein the substitution of T is C₁-C₃ alkyl, haloalkyl, or halo);
J is C(R^{J})₀₋₃, wherein R^{J} is independently H or alkyl, or two or more R^{J} are taken together to form oxo;
R¹ and R² are independently selected from the group consisting of —H, —CN, —CHO, —B(OH)₂, —C(O)alkyl, —C(O)aryl-, —C═C—C(O)aryl, —C═C—S(O)₂ aryl, —CO₂H, —SO₃H, —SO₂NH₂, —PO₃H₂, —SO₂F, —CONH₂, and 5-tetrazolyl;
R³ and R⁴ are independently selected from the group consisting of —H, —OH, F, Cl, Br, I, —C₁₋₆ alkyl, —O—C₁₋₆ alkyl, and —S—C₁₋₆ alkyl;
R⁵, R⁶, R⁷, and R⁸ are independently selected from group consisting of H, alkyl, and halo; and
R⁹, R¹⁰, and R¹¹ are independently selected from group consisting of H, —C₁₋₆alkyl, —C₁₋₆haloalkyl, —O—C₁₋₆alkyl, —S—C₁₋₆ alkyl, F, Cl, Br and I.

In some embodiments, J is C(R^{J})₁₋₃.

J of formula (X-B), (X-C), or (X-D) can be attached to L (e.g., L or L₁ (see Linker section below)) of a compound. J can be attached to L via a nitrogen atom. J can be attached to L via a triazolyl or an amide (e.g., of L). J can be C(R^{J})₂, wherein each R^{J} is independently H or alkyl, or both R^{J} are taken together to form oxo. J can be C₁-C₃alkyl. J can be —CH₂—. J can be —CH₂CH₂—. J can be C═O. J can be a bond.

T of formula (X-B) or (X-C) can be substituted or unsubstituted methylene (e.g., —CH₂—), substituted or unsubstituted amino (e.g., —NH—), —O—, or —S—. The substitution of T can be C₁-C₃ alkyl, C₁-C₃ haloalkyl, or (for the methylene) halo. T can be (—CH₂—). The substitution of T can be C₁-C₃ alkyl, haloalkyl, or halo. T can be unsubstituted.

In certain embodiments of formula (X-B) or (X-C), R¹ and R² are each independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl. R¹ and R² can each be independently selected from the group consisting of H, —CN, —CHO, and —B(OH)₂. R¹ and R² can each be independently selected from the group consisting of H, —CN, —CHO, and —CONH₂. R¹ can be H. R² can be —CN, —CHO, —B(OH)₂, or —CONH₂. R¹ can be H and R² can be —CN, —CHO, —B(OH)₂, or —CONH₂. R¹ can be H and R² can be —CN. R¹ can be H and R² can be —CHO. R¹ can be H and R² can be —B(OH)₂. R¹ can be H and R² can be —CONH₂.

R³ and R⁴ of formula (X-B) or (X-C) can each be independently selected from the group consisting of —H, —OH, F, Cl, Br, I, —C₁₋₆alkyl, —O—C₁₋₆alkyl, and —S—C₁₋₆alkyl. R³ and R⁴ can each be independently —H or —F. R³ can be H and R⁴ can be —F. R³ can be F and R⁴ can be —F.

In certain embodiments, R¹ is H, R² is —CN, R³ is H and R⁴ is —F. In certain embodiments, R¹ is H, R² is —CN, R³ is F, and R⁴ is —F. Additionally, R¹ can be H, R² can be —CHO, R³ can be H and R⁴ can be —F. R¹ can be H, R² can be —CHO, R³ can be F and R⁴ can be —F. R¹ can be H, R² can be —B(OH)₂, R³ can be H and R⁴ can be —F. R¹ can be H, R² can be —B(OH)₂, R³ can be F and R⁴ can be —F. R¹ can be H, R² can be —CONH₂, R³ can be H and R⁴ can be —F. R¹ can be H, R² can be —CONH₂, R³ can be F and R⁴ can be —F.

R⁵, R⁶, R⁷, and R⁸ can each be independently selected from group consisting of H, alkyl, and halo. R⁵, R⁶, R⁷, and R⁸ can each be H.

R⁹, R¹⁰, and R¹¹ can each be independently selected from group consisting of H, —C₁₋₆ alkyl, —C₁₋₆haloalkyl, —O—C₁₋₆alkyl, —S—C₁₋₆ alkyl, F, Cl, Br and I. R⁹, R¹⁰, and R¹¹ can each be independently selected from group consisting of H, —C₁₋₆haloalkyl, F, and Cl. R⁹ and R¹¹ can be H and R¹⁰ can be H, —C₁₋₆haloalkyl, F, or Cl. R⁹ and R¹¹ can be H and R¹⁰ can be H, —CF₃, F, or Cl. R⁹ and R¹¹ can be H and R¹⁰ can be —CF₃. R⁹ and R¹¹ can be H and R¹⁰ can be F. R⁹ and R¹¹ can be H and R¹⁰ can be Cl. R⁹, R¹⁰, and R¹¹ can be H.

In certain embodiments, A is or comprises a structure represented by the formula (X-D):

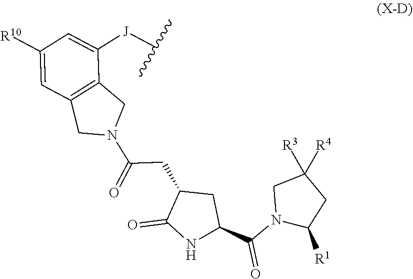

wherein:
J is C(R^{J}₂)₀₋₃, wherein each R^{J}₂ is H, or two or more R^{J}₂ are taken together to form oxo;
R¹ is selected from the group consisting of —CN, —CHO, and —B(OH)₂;
R³ and R⁴ are each independently selected from the group consisting of —H and F;
R¹⁰ is selected from the group consisting of H, —CF₃, F, Cl, Br and I.

A can be attached to L of the compounds hereof via a nitrogen atom (e.g., of L). A can be attached to L via a triazolyl or an amide (e.g., of L).

In certain embodiments, A is

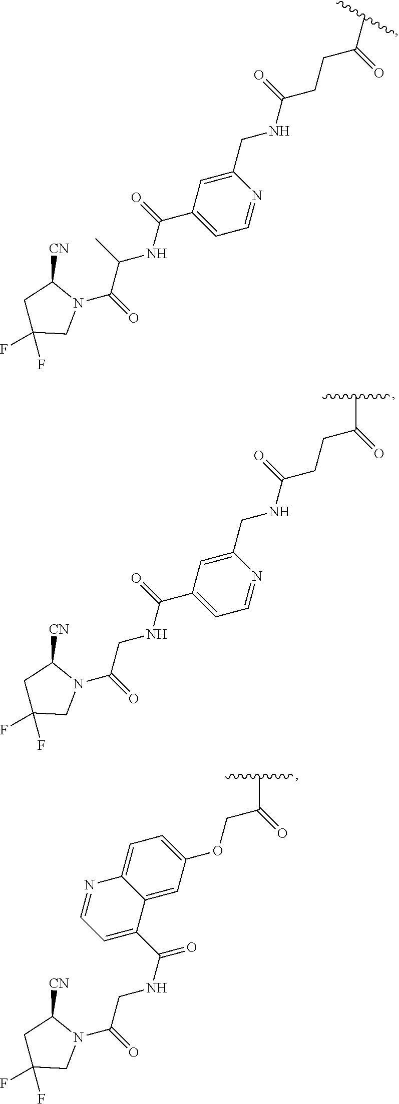 [SMILES: *C(=O)CCC(=O)NCc1cc(C(=O)NC(C)C(=O)N2CC(F)(F)C[C@H]2C#N)ccn1 ; *C(=O)CCC(=O)NCc1cc(C(=O)NCC(=O)N2CC(F)(F)C[C@H]2C#N)ccn1 ; *C(=O)COc1ccc2nccc(C(=O)NCC(=O)N3CC(F)(F)C[C@H]3C#N)c2c1]

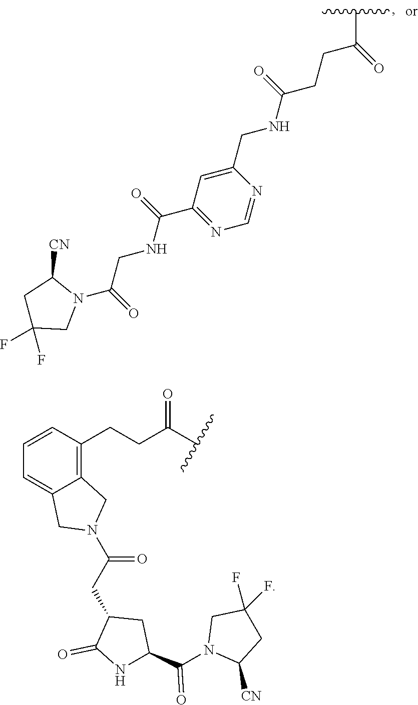 [SMILES: *C(=O)CCC(=O)NCc1cc(C(=O)NCC(=O)N2CC(F)(F)C[C@H]2C#N)ncn1 ; *C(=O)CCc1cccc2c1CN(C(=O)C[C@@H]1C[C@@H](C(=O)N3CC(F)(F)C[C@H]3C#N)NC1=O)C2]

A can be selected from the group consisting of:

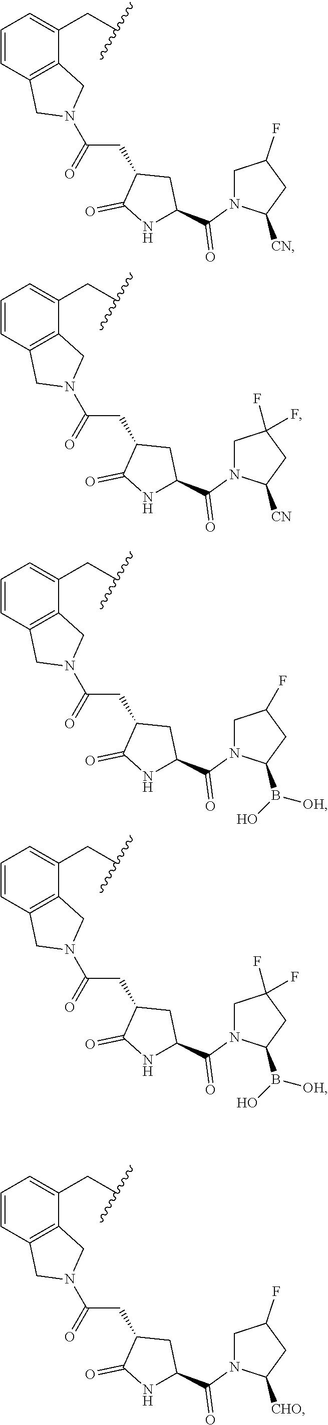 [SMILES: *Cc1cccc2c1CN(C(=O)C[C@@H]1C[C@@H](C(=O)N3CC(F)C[C@H]3C#N)NC1=O)C2 ; *Cc1cccc2c1CN(C(=O)C[C@@H]1C[C@@H](C(=O)N3CC(F)(F)C[C@H]3C#N)NC1=O)C2 ; *Cc1cccc2c1CN(C(=O)C[C@@H]1C[C@@H](C(=O)N3CC(F)C[C@H]3B(O)O)NC1=O)C2 ; *Cc1cccc2c1CN(C(=O)C[C@@H]1C[C@@H](C(=O)N3CC(F)(F)C[C@H]3B(O)O)NC1=O)C2 ; *Cc1cccc2c1CN(C(=O)C[C@@H]1C[C@@H](C(=O)N3CC(F)C[C@H]3C=O)NC1=O)C2]

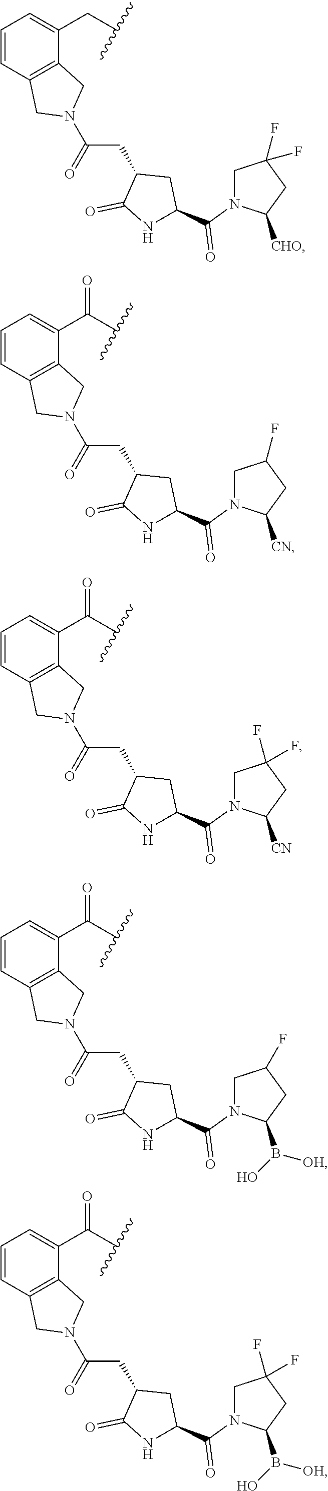 [SMILES: *Cc1cccc2c1CN(C(=O)C[C@@H]1C[C@@H](C(=O)N3CC(F)(F)C[C@H]3C=O)NC1=O)C2 ; *C(=O)c1cccc2c1CN(C(=O)C[C@@H]1C[C@@H](C(=O)N3CC(F)C[C@H]3C#N)NC1=O)C2 ; *C(=O)c1cccc2c1CN(C(=O)C[C@@H]1C[C@@H](C(=O)N3CC(F)(F)C[C@H]3C#N)NC1=O)C2 ; *C(=O)c1cccc2c1CN(C(=O)C[C@@H]1C[C@@H](C(=O)N3CC(F)C[C@H]3B(O)O)NC1=O)C2 ; *C(=O)c1cccc2c1CN(C(=O)C[C@@H]1C[C@@H](C(=O)N3CC(F)(F)C[C@H]3B(O)O)NC1=O)C2]

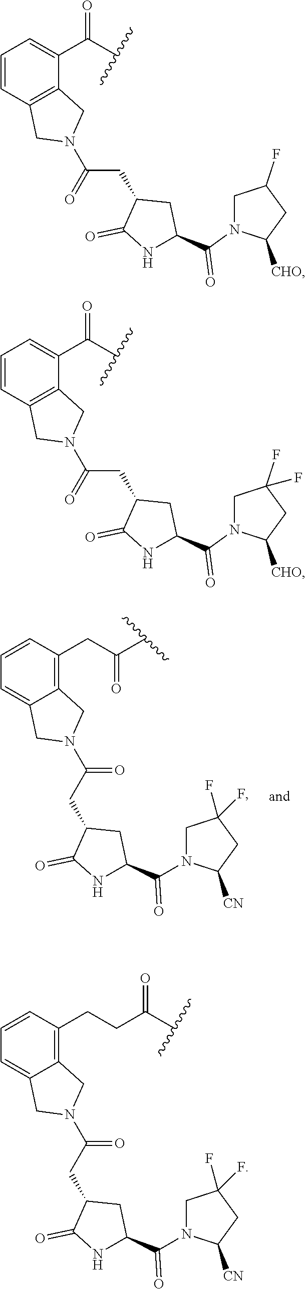 [SMILES: *C(=O)c1cccc2c1CN(C(=O)C[C@@H]1C[C@@H](C(=O)N3CC(F)C[C@H]3C=O)NC1=O)C2 ; *C(=O)c1cccc2c1CN(C(=O)C[C@@H]1C[C@@H](C(=O)N3CC(F)(F)C[C@H]3C=O)NC1=O)C2 ; *C(=O)Cc1cccc2c1CN(C(=O)C[C@@H]1C[C@@H](C(=O)N3CC(F)(F)C[C@H]3C#N)NC1=O)C2 ; *C(=O)CCc1cccc2c1CN(C(=O)C[C@@H]1C[C@@H](C(=O)N3CC(F)(F)C[C@H]3C#N)NC1=O)C2]

A of the compounds can also be selected from the group consisting of

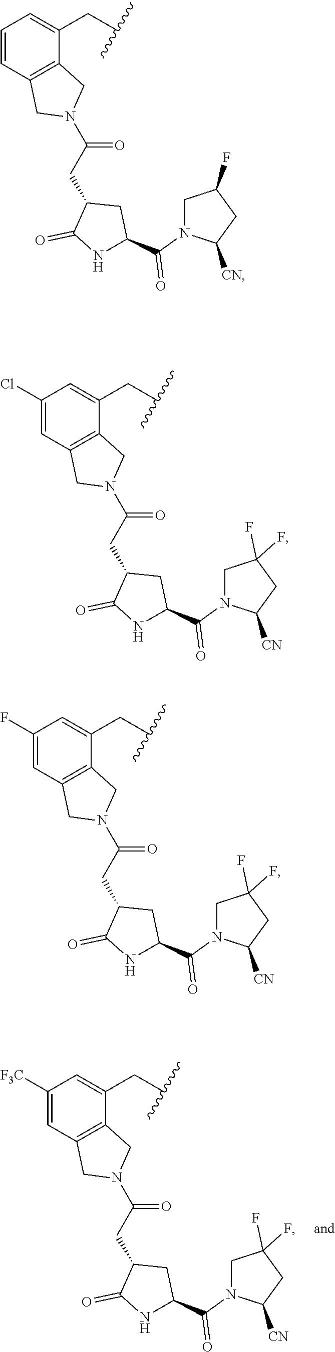 [SMILES: *Cc1cccc2c1CN(C(=O)C[C@@H]1C[C@@H](C(=O)N3C[C@@H](F)C[C@H]3C#N)NC1=O)C2 ; *Cc1cc(Cl)cc2c1CN(C(=O)C[C@@H]1C[C@@H](C(=O)N3CC(F)(F)C[C@H]3C#N)NC1=O)C2 ; *Cc1cc(F)cc2c1CN(C(=O)C[C@@H]1C[C@@H](C(=O)N3CC(F)(F)C[C@H]3C#N)NC1=O)C2 ; *Cc1cc(C(F)(F)F)cc2c1CN(C(=O)C[C@@H]1C[C@@H](C(=O)N3CC(F)(F)C[C@H]3C#N)NC1=O)C2]

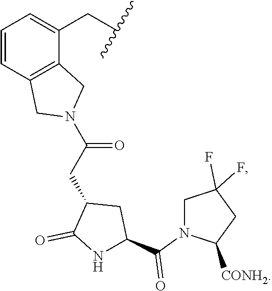 [SMILES: *Cc1cccc2c1CN(C(=O)C[C@@H]1C[C@@H](C(=O)N3CC(F)(F)C[C@H]3C(N)=O)NC1=O)C2]

A can be:

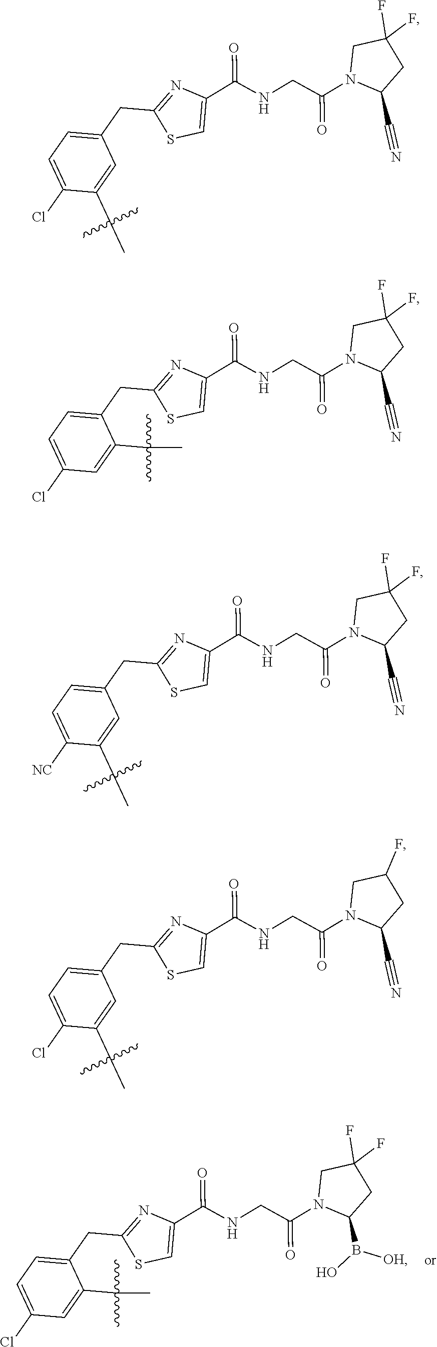 [SMILES: *c1cc(Cc2nc(C(=O)NCC(=O)N3CC(F)(F)C[C@H]3C#N)cs2)ccc1Cl ; *c1cc(Cl)ccc1Cc1nc(C(=O)NCC(=O)N2CC(F)(F)C[C@H]2C#N)cs1 ; *c1cc(Cc2nc(C(=O)NCC(=O)N3CC(F)(F)C[C@H]3C#N)cs2)ccc1C#N ; *c1cc(Cc2nc(C(=O)NCC(=O)N3CC(F)C[C@H]3C#N)cs2)ccc1Cl ; *c1cc(Cl)ccc1Cc1nc(C(=O)NCC(=O)N2CC(F)(F)C[C@H]2B(O)O)cs1]

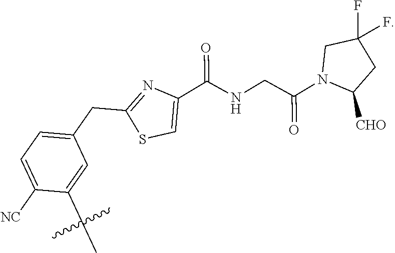 [SMILES: *c1cc(Cc2nc(C(=O)NCC(=O)N3CC(F)(F)C[C@H]3C=O)cs2)ccc1C#N]

A can be:

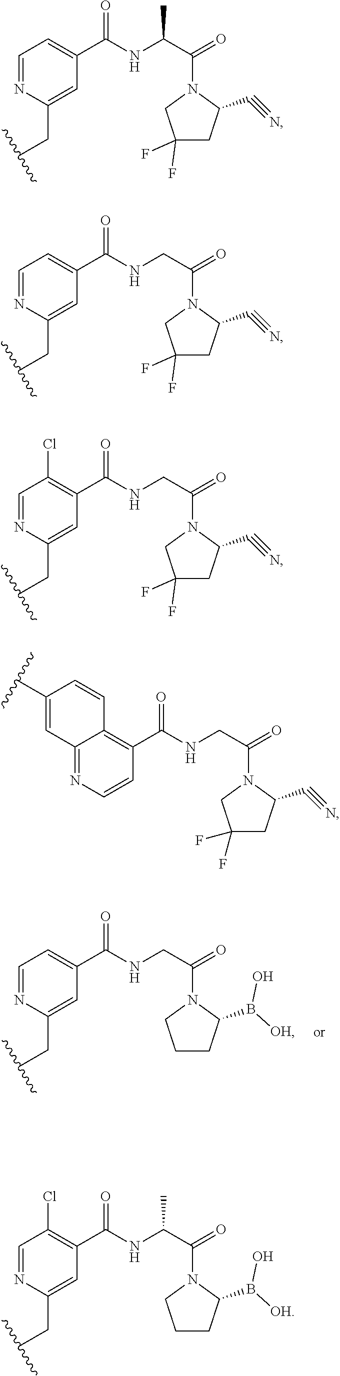 [SMILES: *Cc1cc(C(=O)N[C@@H](C)C(=O)N2CC(F)(F)C[C@H]2C#N)ccn1 ; *Cc1cc(C(=O)NCC(=O)N2CC(F)(F)C[C@H]2C#N)ccn1 ; *Cc1cc(C(=O)NCC(=O)N2CC(F)(F)C[C@H]2C#N)c(Cl)cn1 ; *c1ccc2c(C(=O)NCC(=O)N3CC(F)(F)C[C@H]3C#N)ccnc2c1 ; *Cc1cc(C(=O)NCC(=O)N2CCC[C@H]2B(O)O)ccn1 ; *Cc1cc(C(=O)N[C@H](C)C(=O)N2CCC[C@H]2B(O)O)c(Cl)cn1]

In some embodiments A is or comprises

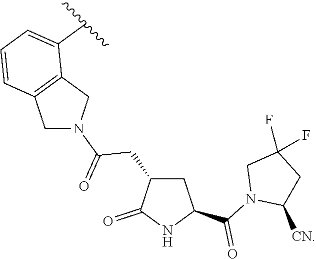 [SMILES: *c1cccc2c1CN(C(=O)C[C@@H]1C[C@@H](C(=O)N3CC(F)(F)C[C@H]3C#N)NC1=O)C2]

In some embodiments A is or comprises

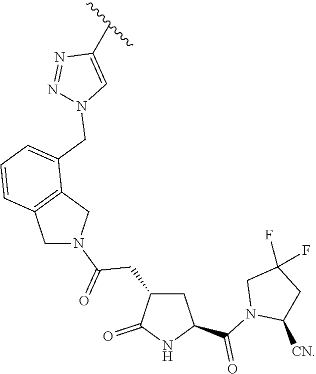 [SMILES: *c1cn(Cc2cccc3c2CN(C(=O)C[C@@H]2C[C@@H](C(=O)N4CC(F)(F)C[C@H]4C#N)NC2=O)C3)nn1]

In some embodiments A is or comprises

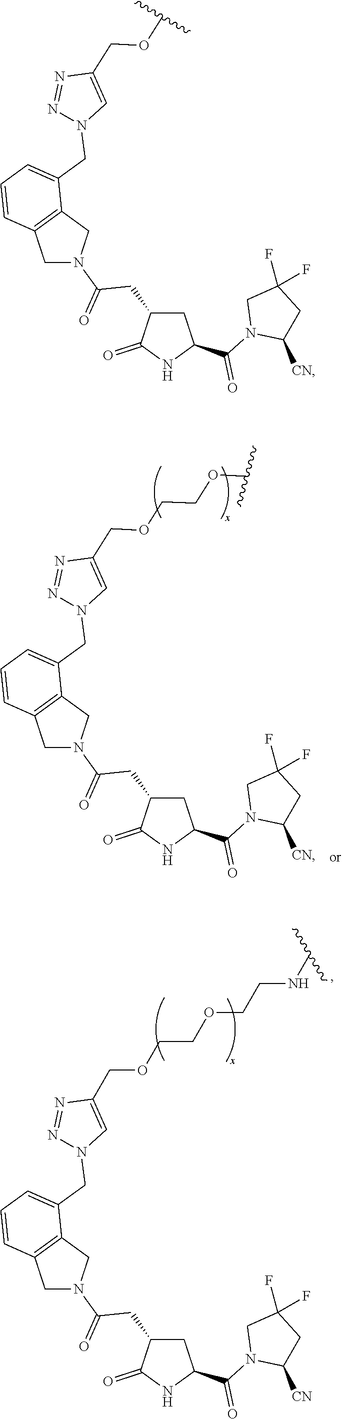

wherein x is 1-20.

In some embodiments, A is or comprises

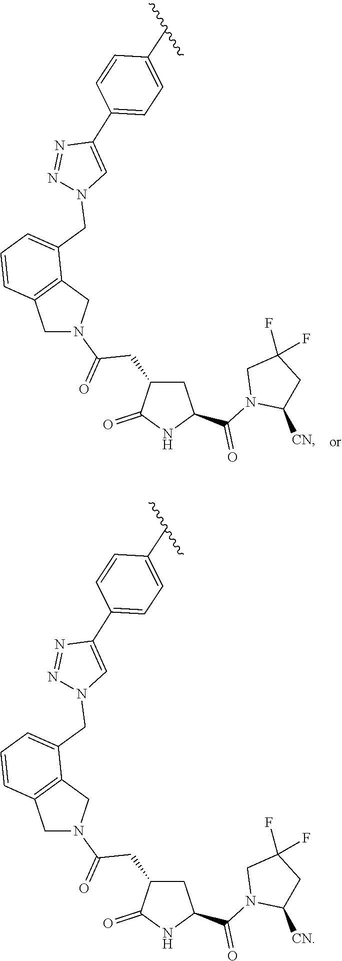 [SMILES: *c1ccc(-c2cn(Cc3cccc4c3CN(C(=O)C[C@@H]3C[C@@H](C(=O)N5CC(F)(F)C[C@H]5C#N)NC3=O)C4)nn2)cc1 ; *c1ccc(-c2cn(Cc3cccc4c3CN(C(=O)C[C@@H]3C[C@@H](C(=O)N5CC(F)(F)C[C@H]5C#N)NC3=O)C4)nn2)cc1]

In certain embodiments, A of the compounds hereof (e.g., a FAPα binding ligand) can have a binding affinity to a FAP (e.g., FAPα) in the range between about 1 nM to about 25 nM, such as 1 nM to about 25 nM or about 1 nM to 25 nM.

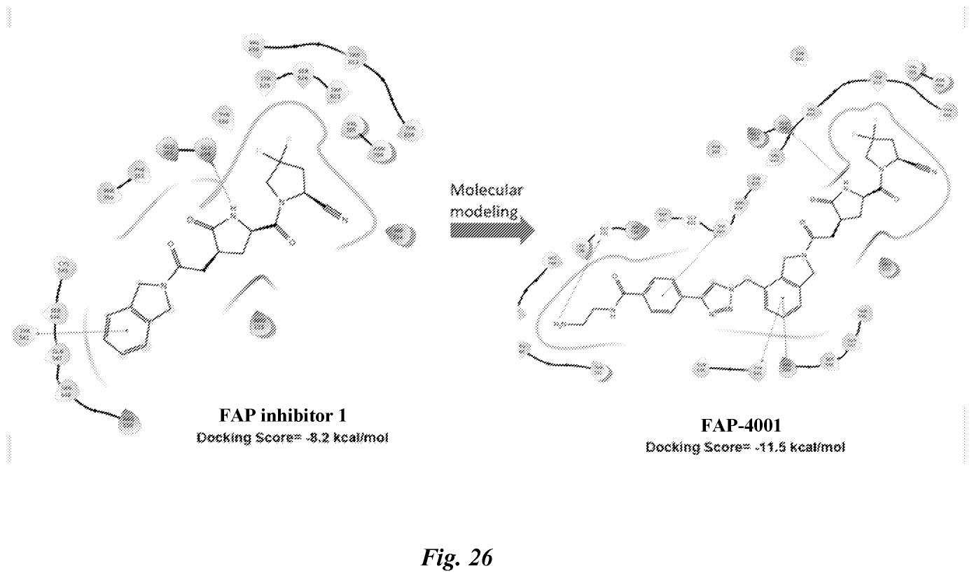

In certain embodiments, A of the compounds comprises a high affinity FAP binding ligand. Such a high affinity FAP ligand can, in certain embodiments, comprise a triazole moiety or one or more derivatives thereof. In certain embodiments, A is a FAP ligand comprising a triazole moiety within an isoindoline scaffold. In certain embodiments, A is a FAP ligand comprising a triazole moiety and a phenyl ring. In certain embodiments, A is a FAP ligand comprising an ethyldiamino aryl triazole moiety. In certain embodiments, A is a FAP ligand comprising an ethyldiamino aryl triazole moiety and a phenyl ring. In certain embodiments, the triazole moiety can additionally comprise primary or secondary amines or a functionalized alkyl or cycloalkyl motif.

Where the FAP ligand comprises a triazole moiety (or derivative thereof) (for example, introduced into an isoindoline scaffold), additional interactions with the FAP target can be provided, which can lead to a higher docking score in Schrodinger molecular docking calculations (see, e.g., FIG. 26) (as compared to a FAP ligand without a triazole moiety and/or phenyl ring attached thereto).

In certain embodiments, a ligand for FAP is provided comprising an isoindoline scaffold into which a triazole moiety has been introduced and which has a Schrodinger molecular docking score of at least about −8.2 kcal/mol.

In certain embodiments, A is

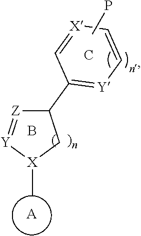

wherein:
n=1-5;
n′=1-5;
ring C is optional;
X, Y and Z in ring B are independently selected from O, N and S, with the proviso that at least one of X and Y is N or Z is N;
X′ and Y′ in ring C are independently selected from O, N and S, with the proviso that at least one of X′ and Y′ is N; and
P is a point of attachment of ring C, if present, to a linker (L) or B′ of formula (X), and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

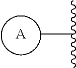

can be any FAP ligand structure provided herein. In certain embodiments,

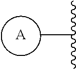

is a structure

[symbol]

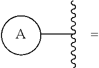

selected from the group consisting of

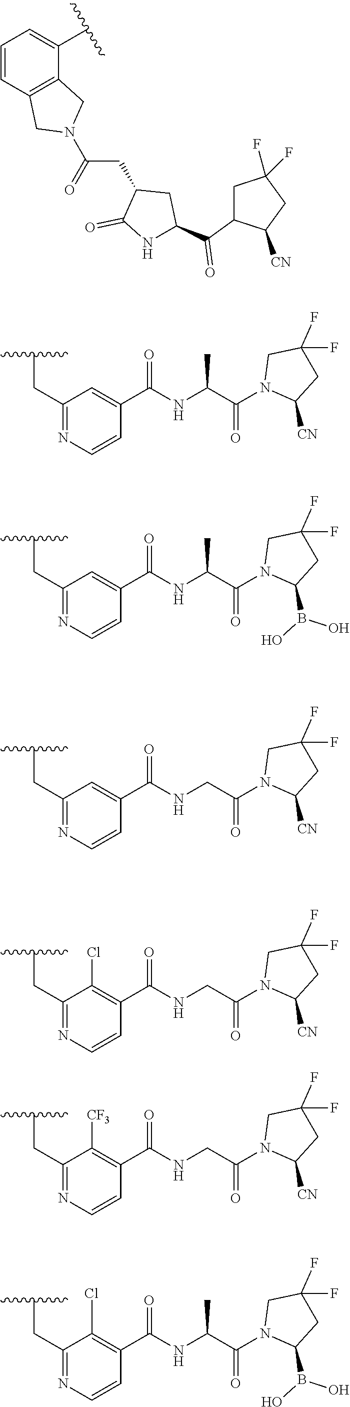

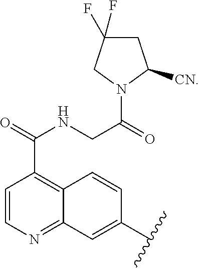 [SMILES: *c1ccc2c(C(=O)NCC(=O)N3CC(F)(F)C[C@H]3C#N)ccnc2c1]

In certain embodiments, the B ring and the C ring can each be a functionalized 5- to 10-membered N-containing aromatic or non-aromatic mono- or bicyclic heterocycle, wherein the heterocycle can optionally further comprise 1-3 heteroatoms selected from O, N, and S. In certain embodiments where ring C is not present, the point of attachment to L or B′ of formula (X) can be through any of the carbon atoms of the 5- to 10-membered N-containing B ring. Additionally, the point of attachment to L or B′ of formula (X) (e.g., P) can be with a functionalized alkyl or cycloalkyl motif or a primary or secondary amine of the B ring or the C ring.

In certain embodiments, the high affinity FAP binding ligand and further comprise C′. C′ can be linked to one or more of the A groups and B′ by L. C′ can be, for example, a radical of an albumin binding ligand, a (PEG)ₙ wherein n is an integer from 0 to 32, a peptide, a peptidoglycan or a saccharide.

In certain embodiments, ring B is non-aromatic. In certain embodiments, ring B is aromatic.

In certain embodiments, A is represented by formula (X-Z):

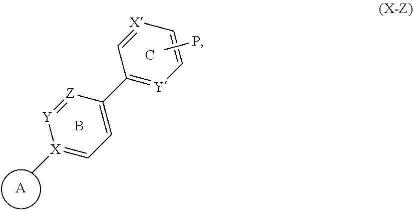

wherein
ring C is optional;
X, Y and Z in ring B are each independently selected from O, N and S, with the proviso that at least one of X and Y is N or Z is N;
X′ and Y′ in ring C are each independent selected from O, N and S, with the proviso that at least one of X′ and Y′ is N; and
P is a point of attachment of ring C, if present, to a linker (L) or B′ of formula (X), and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

In certain embodiments, A is represented by formula X-Y:

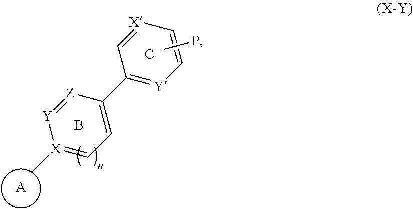

wherein
ring C is optional;
n=0-4;
X, Y and Z in ring B are independently selected from O, N and S, with the proviso that at least one of X and Y is N or Z is N;
X′ and Y′ in ring C are independently selected from O, N and S, with the proviso that at least one of X′ and Y′ is N; and
P is a point of attachment of ring C, if present, to a linker (L) or B′ of formula (X), and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

In some embodiments, A is represented by the structure of formula X-Z:

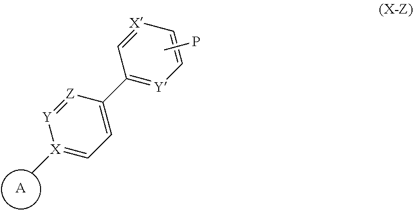

in which

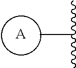

has a formula X-B (or any other FAP ligand structure described herein),
X, Y and Z are independently selected from O, N and S, with the proviso that at least one of X and Y is N or Z is N,
X′ and Y′ are independently selected from O, N, and S, with the proviso that at least one of X′ and Y′ is N or Z is N, and
P is a point of attachment to L or B′ of formula (X) and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

In some embodiments, A of the compounds hereof (e.g., a high affinity FAP ligand) can have a Schrodinger molecular docking score of at least about −8.2 kcal/mol. In certain embodiments, A of the compounds hereof can have a Schrodinger molecular docking score of at least about −11.5 kcal/mol.

Therapeutic and/or Imaging Agents (B′)

As noted above, the compounds hereof further comprise a group B′. In some embodiments, B′ is or comprises a therapeutic agent or an imaging agent (or a radical of either of the foregoing). In some embodiments, B′ is or comprises a radical of an anti-cancer agent. In some embodiments, B′ is or comprises a radical of a dye (e.g., a fluorescent dye). In some embodiments, B′ is or comprises a radical of an anti-fibrotic agent. In some embodiments, B′ is or comprises a radical of a PI3K inhibitor. In some embodiments, B′ is or comprises a radical of a radio-imaging agent comprising a chelated radioisotope (e.g., ^{99m}Tc, ¹¹¹In, ¹⁸F, ⁶⁸Ga, ¹²⁴I, ¹²⁵I, ¹³¹I, ³²P, ⁸⁹Sr, ⁹⁰Y, ¹⁵³Sm, ¹⁶⁹Er, ¹⁷⁷Lu, ¹⁸⁶Re, ¹⁸⁸Re, ¹⁴⁹Tb, ²¹¹At, ²¹²Bi, ²¹³Bi or ²²⁵Ac). B′ can be or comprise a radical of a radioisotope selected from the group consisting of ^{99m}Tc, ¹¹¹In, ¹⁸F, ⁶⁸Ga, ¹²⁴I, ¹²⁵I, and ¹³¹I. In certain embodiments, B′ is or comprises a radical of a radioisotope selected from the group consisting of ³²P, ³⁹Sr, ⁹⁰Y, ¹⁵³Sm, ¹⁶⁹Er, ¹⁷⁷Lu, ¹⁸⁶Re, ¹⁸⁸Re, ¹⁴⁹Tb, ²¹¹At, ²¹²Bi, ²¹³Bi, and ²²⁵Ac.

In certain embodiments, B′ is or comprises a radical of a PI3K inhibitor; a chelating group optionally bound to an isotope (or metal), or a group covalently bound to an isotope (or metal), said isotope or metal being suitable for radio-imaging, radiotherapy or magnetic resonance imaging; an anti-cancer agent; an anti-fibrotic agent; and/or a dye (e.g., a fluorescent dye).

In some embodiments, B′ is a radical of a chelating group optionally bound to an isotope (or metal), or a group covalently bound to an isotope (or metal), said isotope (or metal) suitable for radio-imaging, radiotherapy or magnetic resonance imaging.

In some embodiments, B′ is or comprises a radical of a radio-imaging agent, radiotherapeutic, or magnetic resonance isotope, or a chelating group and a radical of a radio-imaging agent, radiotherapeutic, or magnetic resonance isotope that is bound to the chelating group, wherein the isotope is selected from the group consisting of ³²P, ⁸⁹Sr, ⁹⁰Y, ¹⁵³Sm, ¹⁶⁹Er, ¹⁷⁷Lu, ¹⁸⁶Re, ¹⁸⁸Re, ¹⁴⁹Tb, ²¹¹At, ²¹²Bi, ²¹²Bi, and ²²⁵Ac. In some embodiments, B′ is or comprises a radical of a radio-imaging agent, radiotherapeutic, or magnetic resonance isotope, or a chelating group and a radical of a radio-imaging agent, radiotherapeutic, or magnetic resonance isotope that is bound to the chelating group, wherein the isotope is selected from ¹⁸F, ³²P, ⁴⁴Sc, ⁴⁷Sc, ⁵²Mn, ⁵⁵Co, ⁶⁴Cu, ⁶⁷Cu, ⁶⁷Ga, ⁶⁸Ga, ⁸⁶Y, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁶⁹Er, ¹⁷⁷Lu, ¹⁸⁶Re, ¹⁸⁸Re, ²¹¹At, ²¹²Pb, ²¹²Bi, ²¹³Bi, ²²³Ra, ²²⁴Ra, ²²⁵Ab, ²²⁵Ac, or ²²⁷Th. In some embodiments, the isotope is ¹¹¹In. In some embodiments, the isotope is ¹⁷⁷Lu. In some, embodiments, the isotope is wherein the isotope is selected from the group consisting of ¹¹C, ¹³C, ¹³N, ¹⁵O, ⁶⁰Co, and ¹²³I.

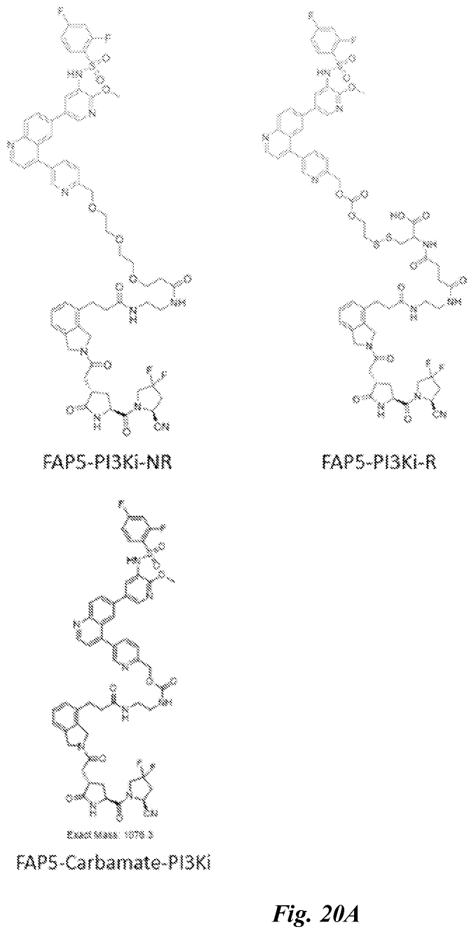

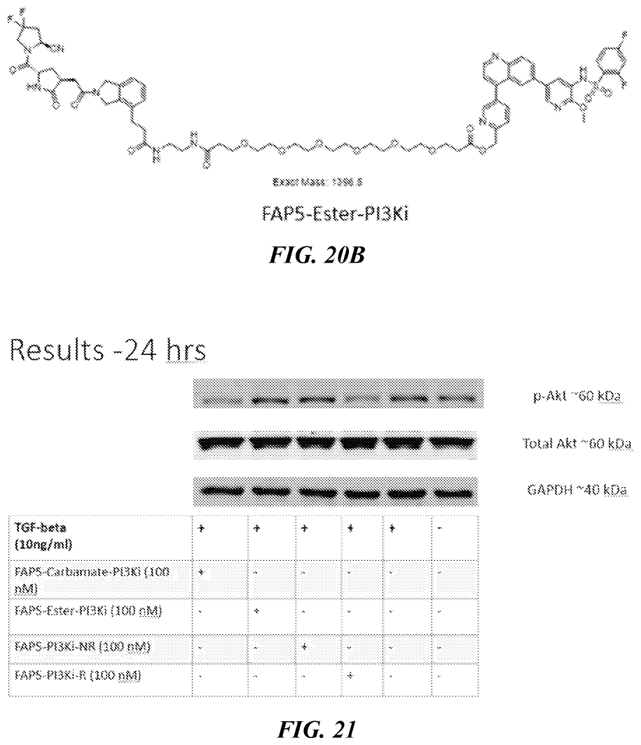

The therapeutic agent (or a radical thereof) can be any entity that can produce a desirable physiological response. The therapeutic agent (or a radical of) can be an antifibrotic agent, an anticancer agent, a chemotherapeutic agent, a photodynamic therapeutic agent, a radiotherapeutic agent, or the like. A therapeutic agent can be a compound (e.g., or a radical thereof) that is effective against (e.g., effective at eliminating, destroying, reducing (e.g., reducing the amount of), or lessening the effects of) cancer cells or pro-fibrotic cells (e.g., cancer-associated fibroblasts, myofibroblasts, or the like (e.g., other tumor microenvironment factors)). Examples of a therapeutic agent (or a radical thereof) include, but are not limited to, a photodynamic therapeutic agent, a radiotherapeutic agent, a chemotherapeutic agent, an antifibrotic agent, and an anti-cancer agent. The therapeutic agent provided herein can be a PI3K inhibitor (or a radical thereof). FIG. 20A shows the structure of various exemplary FAP5-PI3K inhibitors.

The therapeutic agent can be an anti-cancer agent (or a radical thereof). The therapeutic agent can be an anti-fibrotic agent (or a radical thereof). The therapeutic agent can be a compound (or a radical thereof) selected from a tumor growth factor (TGF) β/Smad inhibitor, a Wnt/β-catenin inhibitor, a kinase inhibitor (e.g., a kinase inhibitor for Vascular Endothelial Growth Factor Receptor (VEGFR), a kinase inhibitor for Fibroblast Growth Factor Receptors (FGFR), a kinase inhibitor for platelet-derived growth factor receptor (PDGFR), a kinase inhibitor for focal adhesion kinase (FAK), or a kinase inhibitor for Rho-associated protein kinase (ROCK)), a toll-like receptor agonist (TLR), a nuclear factor kappa-light-chain-enhancer of activated B cells (NF-κB) inhibitor, an inhibitor of collagen synthesis, and a PI3K inhibitor. In certain embodiments, B′ is a phosphoinositide-3-kinase (PI3K) inhibitor (or a radical thereof).

In certain embodiments, B′ is selected from the group consisting of:

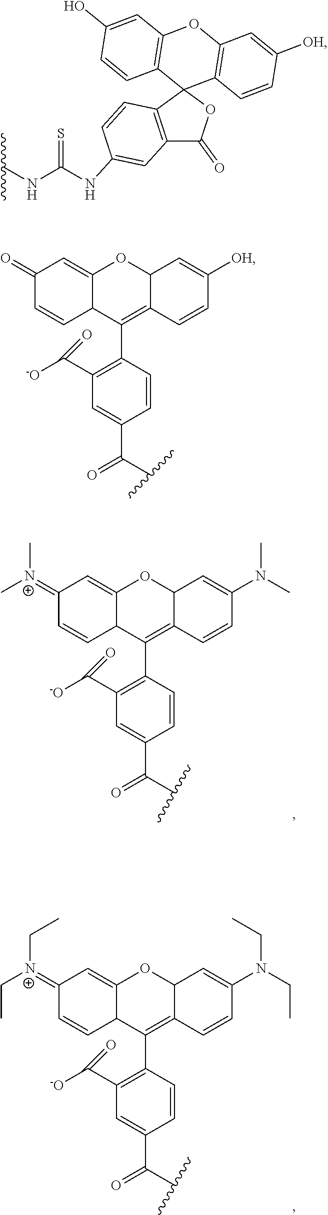

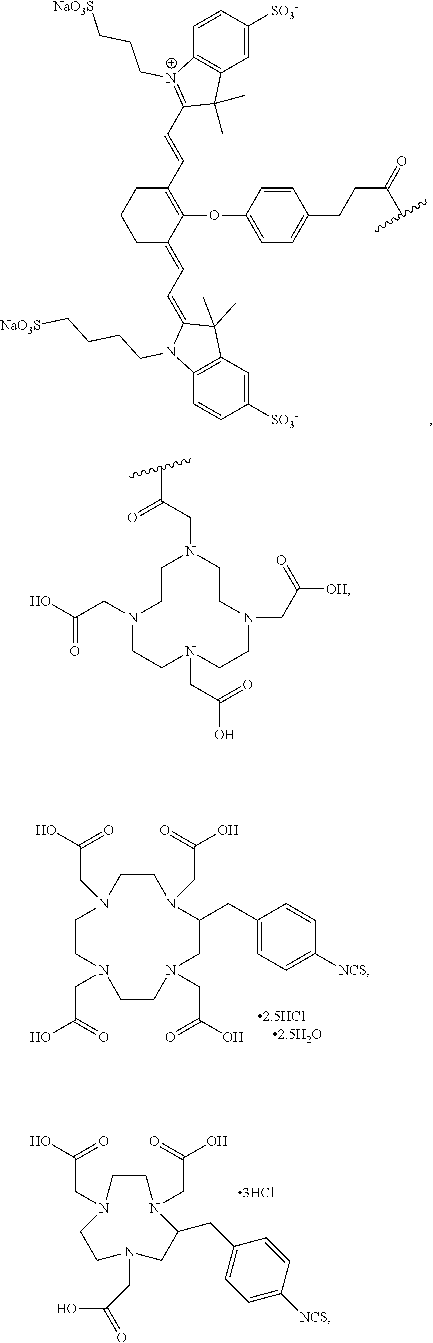

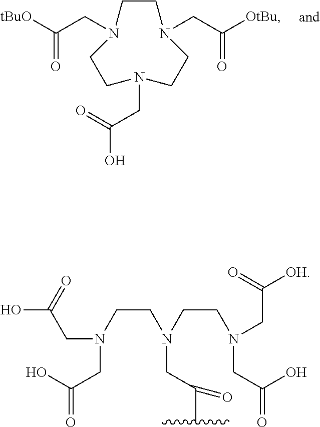 [SMILES: CC(C)(C)OC(=O)CN1CCN(CC(=O)O)CCN(CC(=O)OC(C)(C)C)CC1 ; *C(=O)CN(CCN(CC(=O)O)CC(=O)O)CCN(CC(=O)O)CC(=O)O]

In certain embodiments, B′ is an imaging agent. The imaging agent can be any compound (or a radical thereof) that emits a detectable signal (e.g., an electromagnetic signal (e.g., a radio signal, a fluorescent signal, gamma rays) or a mass). Examples of an imaging agent include, but are not limited to, a radio-imaging agent (e.g., a PET imaging agent or a SPECT imaging agent), a fluorescent imaging agent (e.g., a fluorescent dye), or the like. The imaging agent can be a magnetic resonance (MR) agent. In some embodiments, B′ comprises (e.g., a radical of) a radiolabeled functional group suitable for PET imaging, SPECT imaging, other radio-imaging techniques, magnetic resonance imaging, or radiotherapy. B′ can comprise a radical of a radio-imaging, radiotherapeutic, or magnetic resonance isotope.

B′ can comprise (e.g., a radical of) an imaging agent, a radio-imaging agent, a photodynamic therapeutic agent, a chemotherapeutic agent, an antifibrotic agent and/or a radiotherapeutic agent, wherein B′ is an anticancer agent that is effective against cancer cells or cancer-associated fibroblasts, myofibroblasts, or other tumor microenvironment factors.

B′ can be a radical of a PI3K inhibitor. B′ can be a radical of a chelating group optionally bound to an isotope (or metal). B′ can be a chelating group covalently bound to an isotope (or metal) suitable for radio-imaging, radiotherapy or magnetic resonance imaging.

B′ can be a chelating group (e.g., a chelating agent (or a radical thereof)). Representative chelating groups include, but are not limited to (including free bases thereof, such as wherein a proton (H+) of one or more CO₂H (COOH) is removed to form COO—):

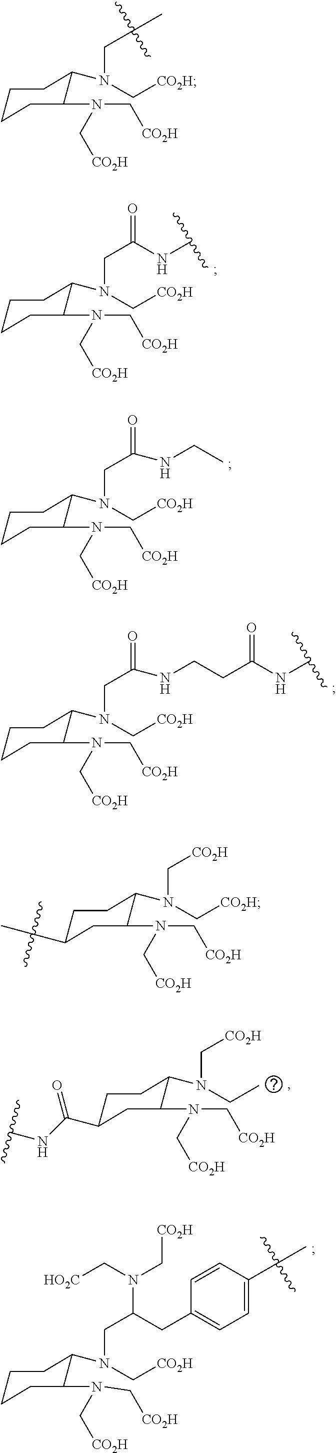

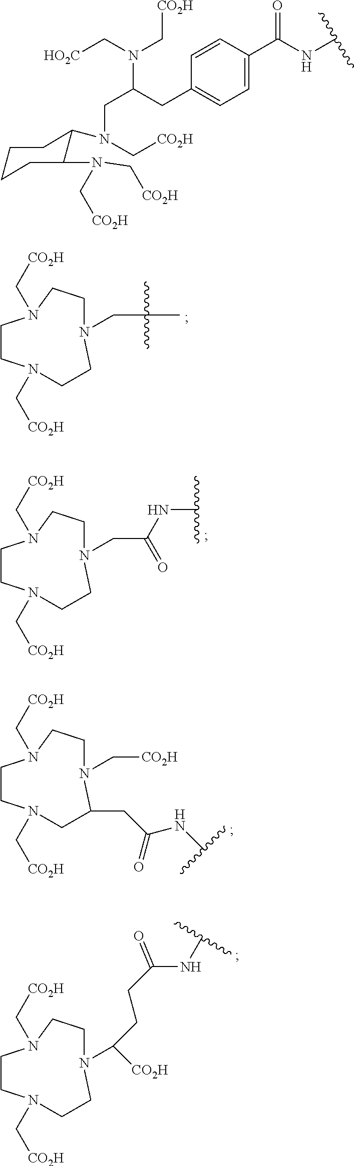 [SMILES: *NC(=O)c1ccc(CC(CN(CC(=O)O)C2CCCCC2N(CC(=O)O)CC(=O)O)N(CC(=O)O)CC(=O)O)cc1 ; *CN1CCN(CC(=O)O)CCN(CC(=O)O)CC1 ; *NC(=O)CN1CCN(CC(=O)O)CCN(CC(=O)O)CC1 ; *NC(=O)CC1CN(CC(=O)O)CCN(CC(=O)O)CCN1CC(=O)O ; *NC(=O)CCC(C(=O)O)N1CCN(CC(=O)O)CCN(CC(=O)O)CC1]

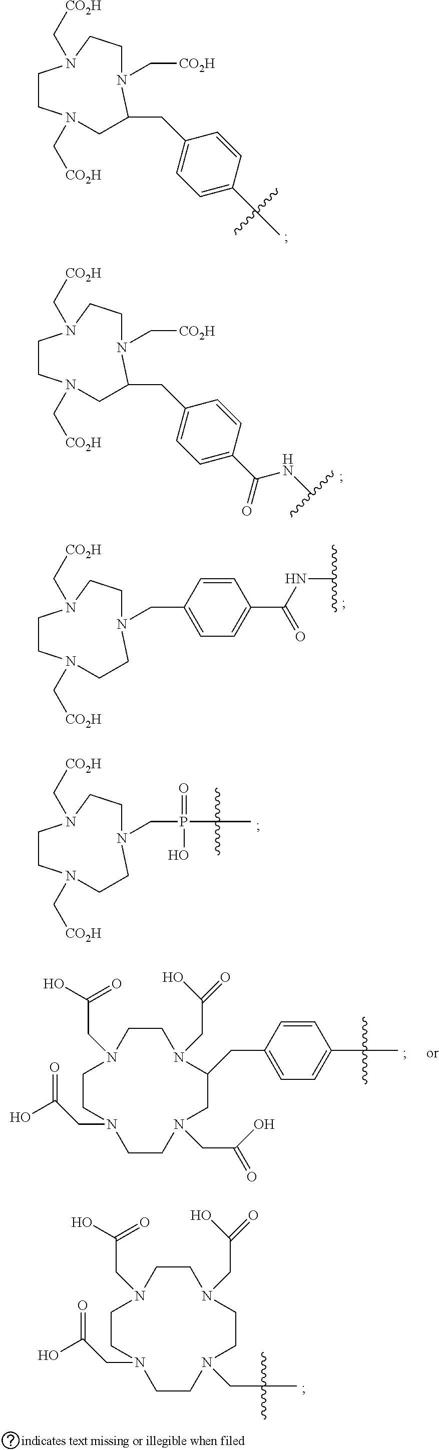

each optionally bound to an isotope (or metal) suitable for radio-imaging, radiotherapy or magnetic resonance imaging.

B′ can comprise a chelating group, which includes, but is not limited to: DOTA (1,4,7,10-tetraazacyclododecane-1,4,7,10-tetraacetic acid) or a derivative thereof; TETA (1,4,8,11-tetraazacyclotetradecane-1,4,8,11-tetraacetic acid) or a derivative thereof; SarAr (1-N-(4-Aminobenzyl)-3,6,10,13,16,19-hexaazabicyclo[6.6.6]-eicosane-1,8-diamine or a derivative thereof; NOTA (1,4,7-triazacyclononane-1,4,7-triacetic acid) or a derivative thereof, NETA (4-[2-(bis-carboxymethylamino)-ethyl]-7-carboxymethyl-[1,4,7]triazonan-1-yl) acetic acid or a derivative thereof. TRAP (1,4,7-triazacyclononane-1,4,7-tris[methyl(2-carboxyethyl)phosphinic acid) or a derivative thereof, HBED (N,N0-bis(2-hydroxybenzyl)-ethylenediamine-N,N0-diacetic acid) or a derivative thereof, 2,3-HOPO (3-hydroxypyridin-2-one) or a derivative thereof; PCTA (3,6,9,15-tetraazabicyclo[9.3.1]-pentadeca-1(15),11,13-triene-3,6,9-triacetic acid) or a derivative thereof, DFO (desferrioxamine) or a derivative thereof; DTPA (diethylenetriaminepentaacetic acid) or a derivative thereof, OCTAPA (N,N0-bis(6-carboxy-2-pyridylmethyl)-ethylenediamine-N,N0-diacetic acid) or a derivative thereof, or H2-MACROPA (N,N′-bis[(6-carboxy-2-pyridipmethyl]-4,13-diaza-18-crown-6) or a derivative thereof, H2dedpa (1,2-[[carboxy)-pyridin-2-yl]-methylamino]ethane or a derivative thereof, and EC20-head comprising β-1-diaminopropionic acid, aspartic acid, and cysteine.

B′ can comprise a radical of DOTA. In certain embodiments, B′ is an isotope- (or metal-) chelated-DOTA (1,4,7,10-tetraazacyclododecane-1,4,7,10-tetraacetic acid).

B′ can be or comprise a radical of a group covalently bound to an isotope (or metal) suitable for radio-imaging, radiotherapy or magnetic resonance imaging.

B′ can be or comprise a chelating group bound to an isotope (or metal) suitable for PET imaging, SPECT imaging, other radioimaging techniques, or radiotherapy.

B′ can be

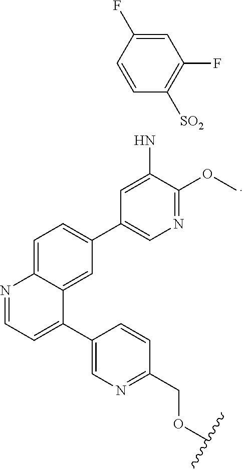

B′ can be

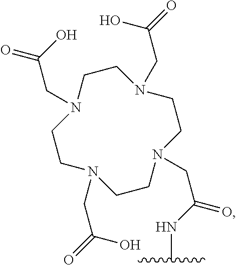 [SMILES: *NC(=O)CN1CCN(CC(=O)O)CCN(CC(=O)O)CCN(CC(=O)O)CC1]

optionally bound to an isotope (or metal) suitable for radio-imaging, radiotherapy or magnetic resonance imaging.

B′ can be one of the following chelating groups

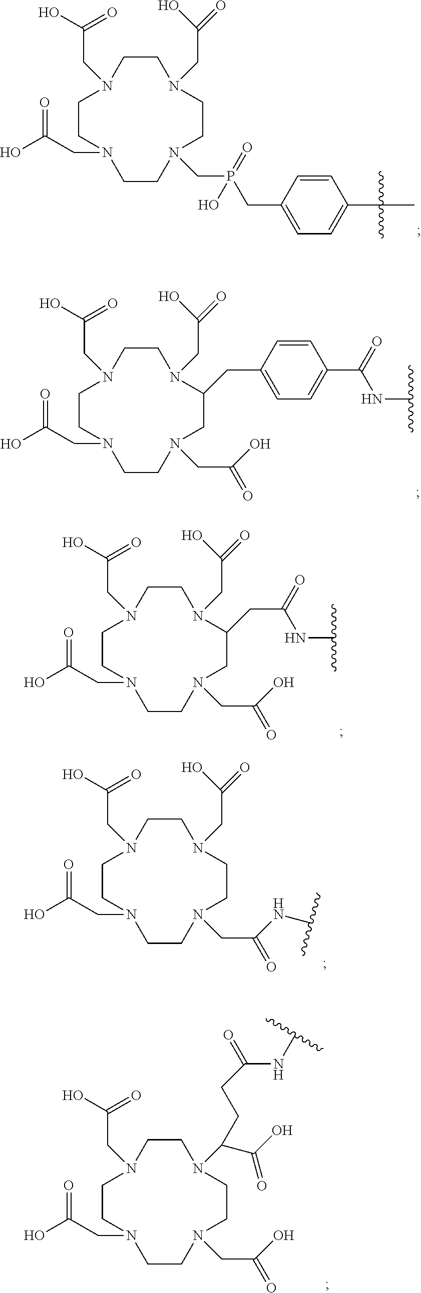

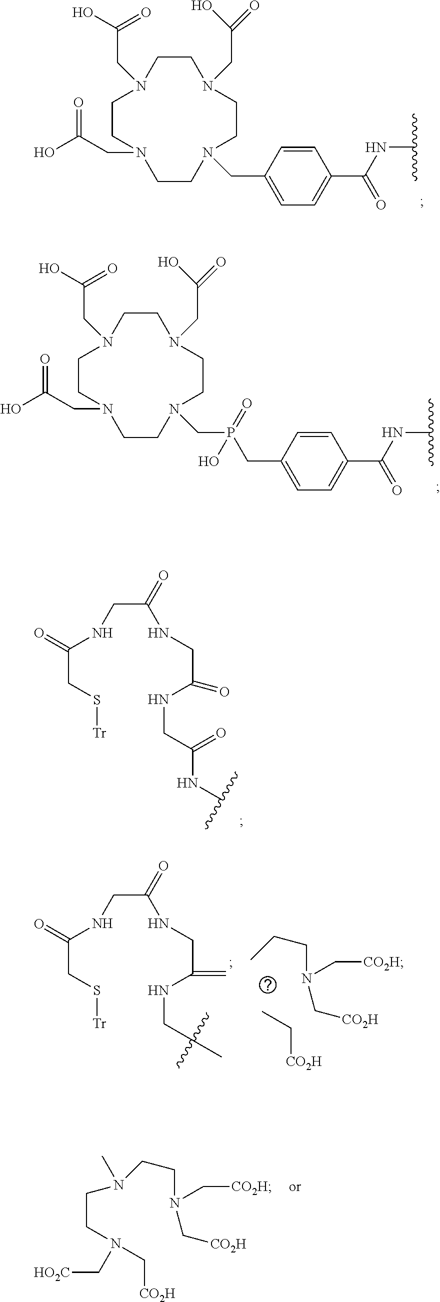

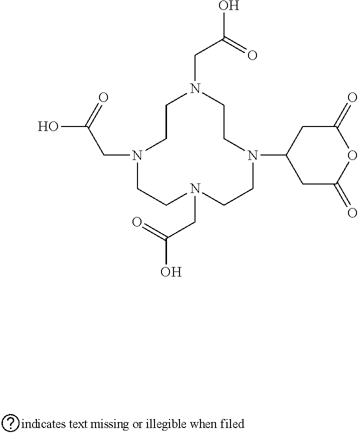 [SMILES: O=C(O)CN1CCN(CC(=O)O)CCN(C2CC(=O)OC(=O)C2)CCN(CC(=O)O)CC1]

each optionally bound to an isotope (or metal) suitable for radio-imaging, radiotherapy or magnetic resonance imaging.

B′ can comprise a magnetic resonance, radio-imaging or radiotherapeutic isotope. In some embodiments B′ comprises a chelating group, and a radio-imaging, radiotherapeutic, or magnetic resonance isotope which is a metal (e.g., a metal suitable for radio-imaging, radiotherapy or magnetic resonance imaging) bound to the chelating group. In some embodiments the isotope is the metal atom bound to the chelating group of B′. In some embodiments, the radio-imaging, radiotherapeutic or magnetic resonance isotope (or metal suitable for radio-imaging, radiotherapy or magnetic resonance imaging) is ¹⁸F, ³²P, ⁴⁴Sc, ⁴⁷Sc, ⁵²Mn, ⁵⁵Co, ⁶⁴Cu, ⁶⁷Cu, ⁶⁷Ga, ⁶⁸Ga, ⁸⁶Y, ⁸⁹Sr, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁶⁹Er, ¹⁷⁷Lu, ¹⁸⁶Re, ¹⁸⁸Re, ²¹¹At, ²¹²Pb, ²¹²Bi, ²¹³Bi, ²²³Ra, ²²⁴Ra, ²²⁵Ab, ²²⁵Ac, or ²²⁷Th. In some embodiments, the radio-imaging, radiotherapeutic or magnetic resonance isotope is ¹¹C, ¹³C, ¹³N, ¹⁵O, ⁶⁰Co, or ¹²³I. In some embodiments the radio-imaging, radiotherapeutic or magnetic resonance isotope is ²²⁵Ac, ³²P, ⁸⁹Sr, ^{117m}Sn, ¹⁵³Sm, ¹⁶⁹Er, ¹⁸⁶Re, ¹⁸⁸Re, ¹⁴⁹Tb, ²¹²Bi, or ²¹³Bi. In some embodiments, the isotope is ¹¹¹In. In some embodiments, the isotope is ¹⁷⁷Lu.

B′ can comprise a radio-imaging nuclide. The radio-imaging nuclide can be any suitable radio-imaging nuclide. The radio-imaging nuclide can be selected from the group consisting of ^{99m}Tc ¹¹¹In, ¹⁸F, ⁶⁸Ga, ¹²⁴I, ¹²⁵I, and ¹³¹I.

B′ can comprise a radiotherapeutic nuclide. The radiotherapeutic nuclide can be selected from the group consisting of ¹⁷⁷Lu, ⁹⁰Y, and ²¹¹At.

B′ can comprise a radiolabelled prosthetic group (or a radical thereof). The radiolabelled prosthetic group can comprise a radioisotope selected from the group consisting of ¹⁸F, ¹²⁴I, ¹²⁵I, ¹³¹I, and ²¹¹At.

In certain embodiments, B′ (e.g., the radiolabelled prosthetic group (or a radical thereof)) has the following structure:

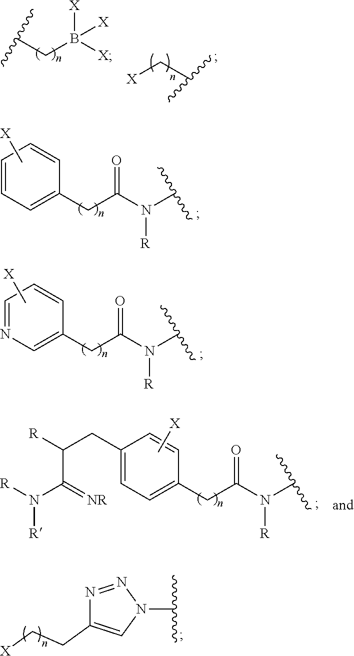

wherein:
each X is independently a radioisotope selected from the group consisting of ¹⁸F, ¹²⁴I, ¹²⁵I, ¹³¹I, and ²¹¹At;
each R or R¹ is independently H, alkyl, substituted alkyl, cycloalkyl, substituted cycloalkyl, heterocycloalkyl, aryl, substituted aryl, heteroaryl, or substituted heteroaryl; and
each n is independently an integer selected from the group consisting of 0, 1, 2, 3, 4, 5, 6, 7, 8, 9, 10, 11, 12, 13, 14, 15, 16, 17, 18, 19, and 20.

Representative radiolabelled prosthetic groups (e.g., B′) include, but are not limited to:

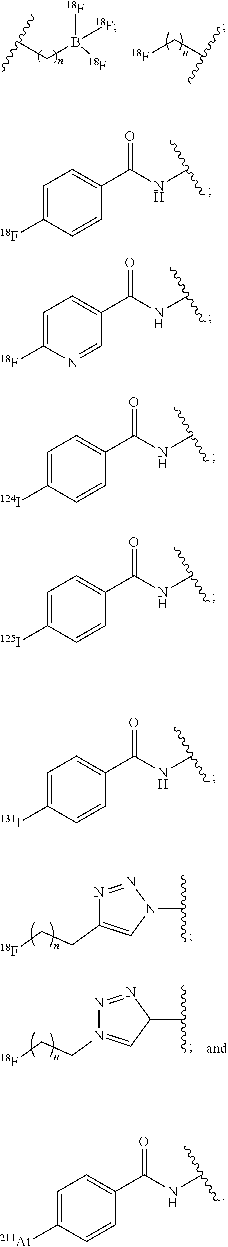

Where B′ is a chelator, in the case of radiotherapeutic nuclides, in certain embodiments B′ can chelate the nuclide.

B′ can be a PI3K inhibitor or a radical thereof.

A PI3K inhibitor (or a radical thereof) (e.g., a compound or a conjugate comprising a PI3K inhibitor (or a radical thereof)) can have the structure of formula III:

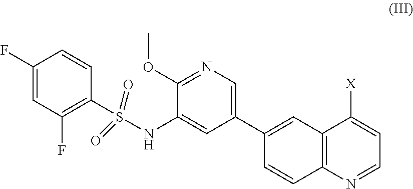

wherein:
X is selected from the group consisting of:

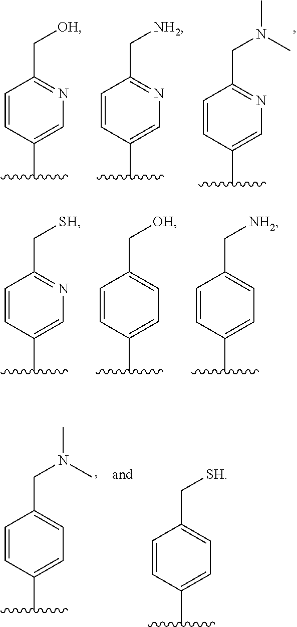

X of formula (III) can be the radical of B′ (e.g., wherein the radical is on a heteroatom (e.g., S, N, or O of X)). B′ can be attached to L of the compound via X (e.g., a hydroxyl radical of X).

A PI3K inhibitor (or a radical thereof) of B′ of the compound (e.g., a compound or a conjugate comprising a PI-3 Kinase inhibitor (or a radical thereof)) can have the structure of

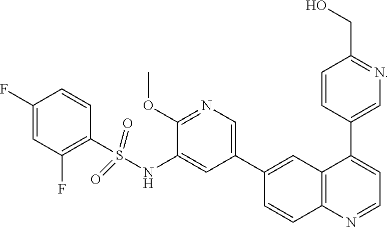 [SMILES: COc1ncc(-c2ccc3nccc(-c4ccc(CO)nc4)c3c2)cc1NS(=O)(=O)c1ccc(F)cc1F]

L can be

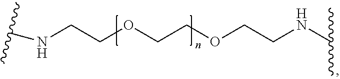

wherein n is an integer from 1 to 32. L can be

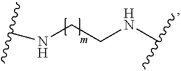

wherein m is an integer from 1 to 9. L can be

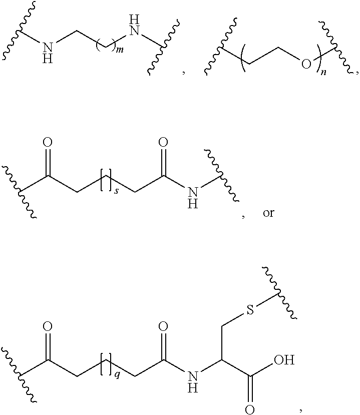

wherein m is an integer from 1 to 9, n is an integer from 1 to 32, q is an integer from 0 to 4; and s is an integer from 0 to 4.

m can be 1. m can be 2. m can be 3. m can be 4. m can be 5. m can be 6. m can be 7. m can be 8. m can be 9.

n can be 1 to 12. n can be 1. n can be 2. n can be 3. n can be 4. n can be 5. n can be 6. n can be 7. n can be 8. n can be 9. n can be 10. n can be 11. n can be 12. n can be 13. n can be 14. n can be 15. n can be 16. n can be 17. n can be 18. n can be 19. n can be 20. n can be 21. n can be 22. n can be 23. n can be 24. n can be 25. n can be 26. n can be 27. n can be 28. n can be 29. n can be 30. n can be 31. n can be 32.

q can be 0. q can be 1. q can be 2. q can be 3. q can be 4.

s can be 0. s can be 1. s can be 2. s can be 3. s can be 4.

### Linker (L)

L of the compound is a linker, such as any suitable linker. As used herein, the term “linker” generally refers to a portion of a compound that forms a chemical bond with an A (e.g., a binding ligand) and/or B′ (e.g., a therapeutic agent or a imaging agent) and/or C′ (e.g., an albumin binding ligand, a PEG, a peptide, a peptidoglycan or a saccharide). In particular, a “linker” can link two or more functional parts of a molecule to form a compound provided herein. Illustratively, the linker may comprise atoms selected from C, N, O, S, Si, and P; C, N, O, S, and P; or C, N, O, and S. The linker can link different functional capabilities of the compound, such as the FAP ligand and the DOTA chelator group. The linker can comprise a several linker groups, such as, for example, in the range from about 2 to about 100 atoms in the contiguous backbone.

The linker can be a releasable linker. The linker can be a non-releasable linker. L can be a trivalent linker. L can be a biofunctionalized linker. For example, in at least formulae (X) and (I), the (e.g., bifunctionalized) linker can form a chemical bond with A and B′. L can be a (e.g., bifunctionalized) linker linking one or more A groups to B′ (e.g., through a first covalent bond connecting L to A and a second covalent bond linking L to B′).

L can comprise one or more linker groups, each linker group independently selected from the group consisting of alkyl(ene), heteroalkyl(ene), heterocycloalkyl(ene), heteroaryl, aryl, alkoxy, thioether, disulfide, carboxylic acid, anhydride, carbonate, carbamate, thioether, sugar, peptide, and peptidoglycan. L can comprise one or more linker groups, each linker group independently selected from the group consisting of PEG, alkyl(ene), disulfide, amide, carboxylic acid, anhydride, carbonate, ester, carbamate, thioether, triazole, sugar, and peptide. L can comprise one or more linker group, each linker group independently selected from the group consisting of PEG, alkyl(ene), disulfide, amide, carboxylic acid, carbonate, ester, phenyl, triazole, and carbamate. L can comprise one or more linker groups, each linker group independently selected from the group consisting of PEG, alkyl(ene), disulfide, amide, carboxylic acid, phenyl, triazole, ester, and carbonate. L can comprise one or more linker groups, each linker group independently selected from the group consisting of PEG, alkyl(ene), disulfide, amide, carboxylic acid, ester, and carbonate. L can comprise one or more linker groups, each linker group independently selected from the group consisting of PEG, alkyl(ene), disulfide, and amide. L can comprise one or more linker groups, each linker group independently selected from the group consisting of alkyl(ene), disulfide, and amide. L can comprise one or more linker groups, each linker group independently selected from the group consisting of amide, alkyl(ene), PEG, phenyl, and triazole. L can comprise one or more linker groups, each linker group independently selected from the group consisting of PEG, alkyl(ene), and amide. L can comprise one or more linker groups, each linker group independently selected from the group consisting of alkyl(ene) and amide. L can comprise one or more linker groups, each linker group independently selected from the group consisting of PEG and amide. The linker can comprise one or more triazole linker groups. The linker can comprise one or more disulfide linker groups. The linker can comprise one or more amide linker groups. The linker can comprise one or more PEG linker groups.

L can be a non-releasable linker (e.g., bivalently (e.g., covalently) attached to B′ and A). L can be a releasable linker (e.g., bivalently (e.g., covalently) attached to B′ and A).

In certain embodiments, for example and without limitation in formulae (I′) and (II), L comprises three linker groups, each linker group independently selected from the group consisting of alkyl(ene), heteroalkyl(ene), heterocycloalkyl(ene), heteroaryl, aryl, alkoxy, thioether, disulfide, carboxylic acid, anhydride, carbonate, carbamate, thioether, sugar, and peptide. In certain embodiments, L comprises three linker groups, each linker group independently selected from the group consisting of PEG, alkyl(ene), disulfide, amide, carboxylic acid, anhydride, carbonate, ester, carbamate, thioether, triazole, sugar, and peptide. L can also comprise three linker groups, each linker group independently selected from the group consisting of PEG, alkyl(ene), disulfide, amide, carboxylic acid, carbonate, ester, phenyl, triazole, and carbamate. In certain embodiments, L comprises three linker groups, each linker group independently selected from the group consisting of PEG, alkyl(ene), disulfide, amide, carboxylic acid, phenyl, triazole, ester, and carbonate. L can comprise three linker groups, each linker group independently selected from the group consisting of PEG, alkyl(ene), disulfide, amide, carboxylic acid, ester, and carbonate. L can comprise three linker groups, each linker group independently selected from the group consisting of PEG, alkyl(ene), disulfide, and amide. L can comprise three linker groups, each linker group independently selected from the group consisting of alkyl(ene), disulfide, and amide. L can comprise three linker groups, each linker group independently selected from the group consisting of amide, alkyl(ene), PEG, phenyl, and triazole. L can comprise three linker groups, each linker group independently selected from the group consisting of PEG, alkyl(ene), and amide. L can comprise three linker groups, each linker group independently selected from the group consisting of alkyl(ene) and amide. L comprises three linker groups, each linker group independently selected from the group consisting of PEG and amide.

L can comprise one or more releasable groups.

L can be attached (e.g., covalently) to A via an amide linker group. L can be attached (e.g., covalently) to B′ via an amide linker group. L can be attached (e.g., covalently) to C′ via an amide linker group. L can be independently (such as covalently) attached to A, B′, and (in formulae (I′) and (II)) C′ via amide linker groups.

L can be attached (e.g., covalently) to A via a triazole linker group. L can be attached (e.g., covalently) to B′ via a triazole linker group. L can be independently (such as covalently) attached to A and B′ via triazole linker groups.

L can be attached (e.g., covalently) to A via a triazole linker group. L can be attached to A via a triazole linker group and attached to B′ via an amide linker group.

L can be attached (e.g., covalently) to B′ via a carbamate linker group. L can be attached to A via an amide linker group and attached to B′ via a carbamate linker group.

The linker can be a releasable linker. The linker can be a non-releasable linker.

The linker can be a reductively cleavable linker (e.g., disulfide). The linker can be an oxidatively cleavable linkers (e.g., amino-aromatic groups). The linker can be or comprise an oxime ester. The linker can be or comprise a hydrazone. The linker can be or comprise a PEG, (n=0 to 36). The linker can be or comprise a peptide. The linker can be or comprise a peptidoglycan.

L can be (L¹)-(L²) or (L¹)-(L²)-(L³), wherein:
L¹ is a first linker;
L² is a second linker; and
(where applicable) L³ is a third linker.

L¹, L² and L³ can be the same. Two of L¹, L² and L³ can be the same. L¹, L² and L³ can be different. L¹ can be connected to the A group (and the L² group). L² can be connected to the B′ group (and the L² and L³ groups). L³ can be connected to the C′ group (and the L² group).

Each L¹, L² and L³ can be independently a length from 15 to 200 angstroms (Å).

L can be (L¹)ₚ-W-(L²)_{q} in which:
L¹ is a first linker;
L² is a second linker;
p=1-5; and
q=1-5.

In certain embodiments, each L¹ and each L² independently comprises one or more linker groups, each linker group independently selected from the group consisting of PEG, alkyl(ene), amide, phenyl, and triazole.

W (where present) can be an amine core, an aromatic core, or an alkylene core.

Each L¹ and L² can be independently a length from 5 to 200 Å.

L can have the following structure:

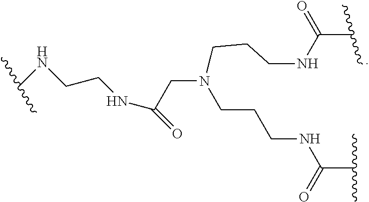 [SMILES: *NCCNC(=O)CN(CCCNC(*)=O)CCCNC(*)=O]

L can be:

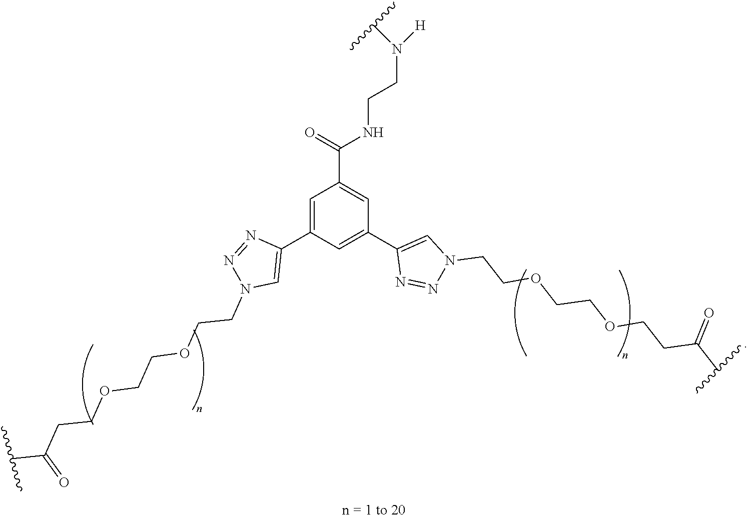

L can be:

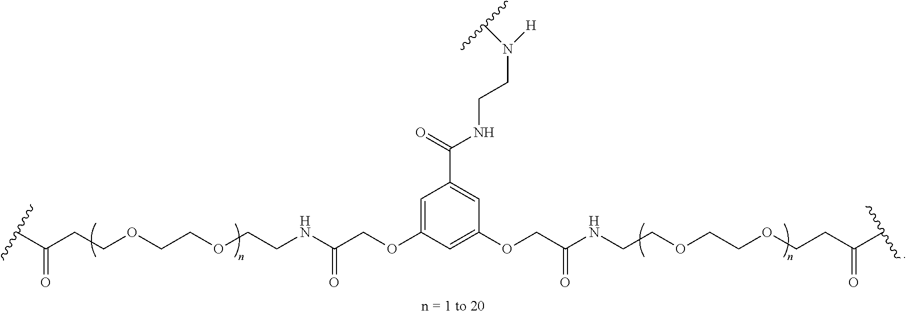

L can comprise at least one linker group, each linker group selected from the group consisting of PEG, alkyl, sugar, and peptide. The linker can be a PEG- (e.g., pegylated-), alkyl-, sugar-, and peptide-based dual linker.

L can be a non-releasable linker (e.g., bivalently (e.g., covalently) attached to B′ and A). L can be a releasable linker (e.g., bivalently (e.g., covalently) attached to B′ and A).

L can comprise one or more linker groups having the following structure:

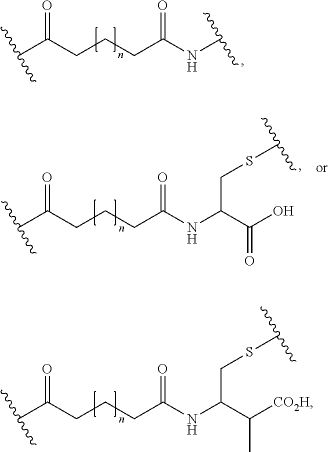

wherein n is 0 to 10.

L can comprise one or more linker groups (e.g., L¹, L² and/or L³ (if applicable)) having the following structure:

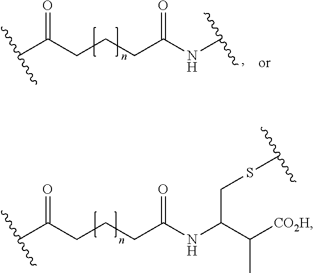

wherein n is 0 to 10.

L can comprise one or more linker groups having the following structure:

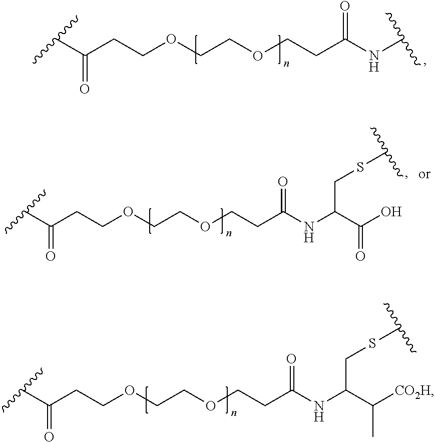

wherein n is 1 to 32.

L can comprise one or more linker groups having the following structure:

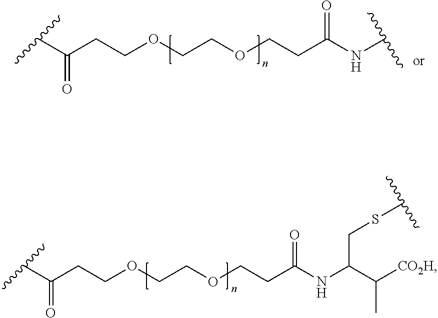

wherein n is 1 to 32.

L can comprise one or more linker groups having the following structure:

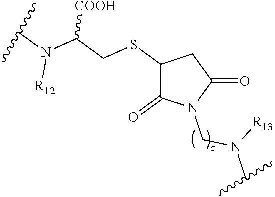

wherein:
R₁₂ and R₁₃ can each be independently H or C₁-C₆ alkyl; and
z is an integer from 1 to 8.

L can comprise one or more linker groups having the following structure:

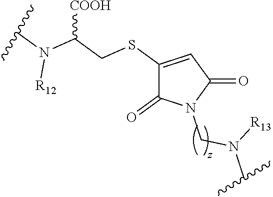

wherein:
R₁₂ and R₁₃ can each be independently H or C₁-C₆ alkyl; and
z is an integer from 1 to 8.

L can comprise one or more linker groups (e.g., L¹ and each L²) having the following structure:

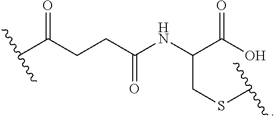 [SMILES: *SCC(NC(=O)CCC(*)=O)C(=O)O]

L can comprise one or more linker groups having the following structure:

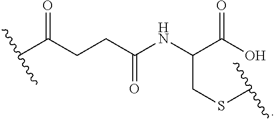 [SMILES: *SCC(NC(=O)CCC(*)=O)C(=O)O]

L can comprise one or more linker groups having the following structure:

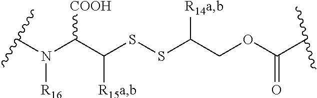

wherein:
R₁₆ is H or C₁-C₆ alkyl; and
R₁₄ₐ, R_{14b}, R₁₅ₐ, and R_{15b} can each be independently H or C₁-C₆ alkyl.

L can comprise the following structure:

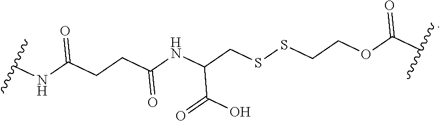 [SMILES: *NC(=O)CCC(=O)NC(CSSCCOC(*)=O)C(=O)O]

L can comprise one or more linker groups having the following structure:

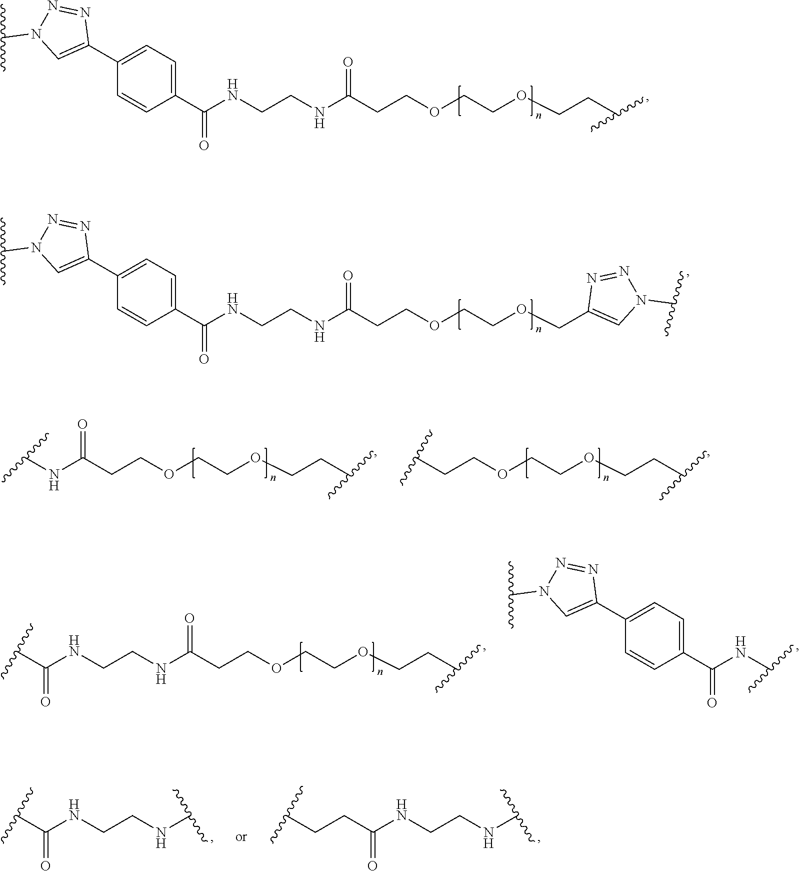

wherein n is 0 to 15.

L can comprise a reductively cleavable linker. L can comprise an oxidatively cleavable linker. L can comprise an oxime ester. L can comprise a hydrazone. L can comprise a PEGn, and n=0-36. L can comprise a peptide. L can comprise a peptidoglycan.

L can be

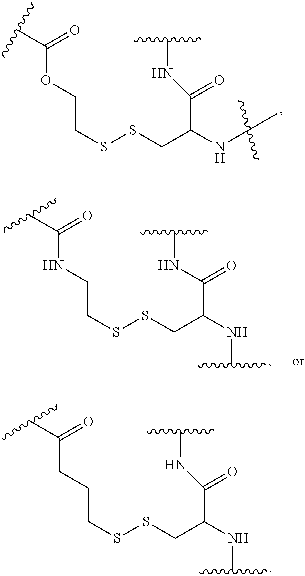 [SMILES: *NC(=O)C(CSSCCOC(*)=O)N* ; *NC(=O)C(CSSCCNC(*)=O)N* ; *NC(=O)C(CSSCCCC(*)=O)N*]

L can be

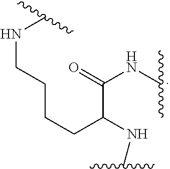 [SMILES: *NCCCCC(N*)C(=O)N*]

### C′

In certain embodiments of the compounds hereof (e.g., a compound having the structure of formula (I′) or (II)), the compound further comprises C′ coupled with a linker of the compound.

C′ can be any pharmacokinetic extender. In certain embodiments, C′ is an albumin binding ligand. C′ can be albumin-binding small protein scaffold comprising albumin binding domain 035 (ABD035), albumin binding domain Con, which is a peptide of a three-helix bundle 45 amino acids in length (ABDCon), designed ankyrin repeat proteins (DARPins), disulfide-stabilized Fv fragment (dsFv) CA645 (an anti-albumin antibody), any nanobody that complexes with human serum albumin (nanobody), and variable new antigen receptor E06 (VNAR (E06)).

In certain embodiments, C′ is or comprises:

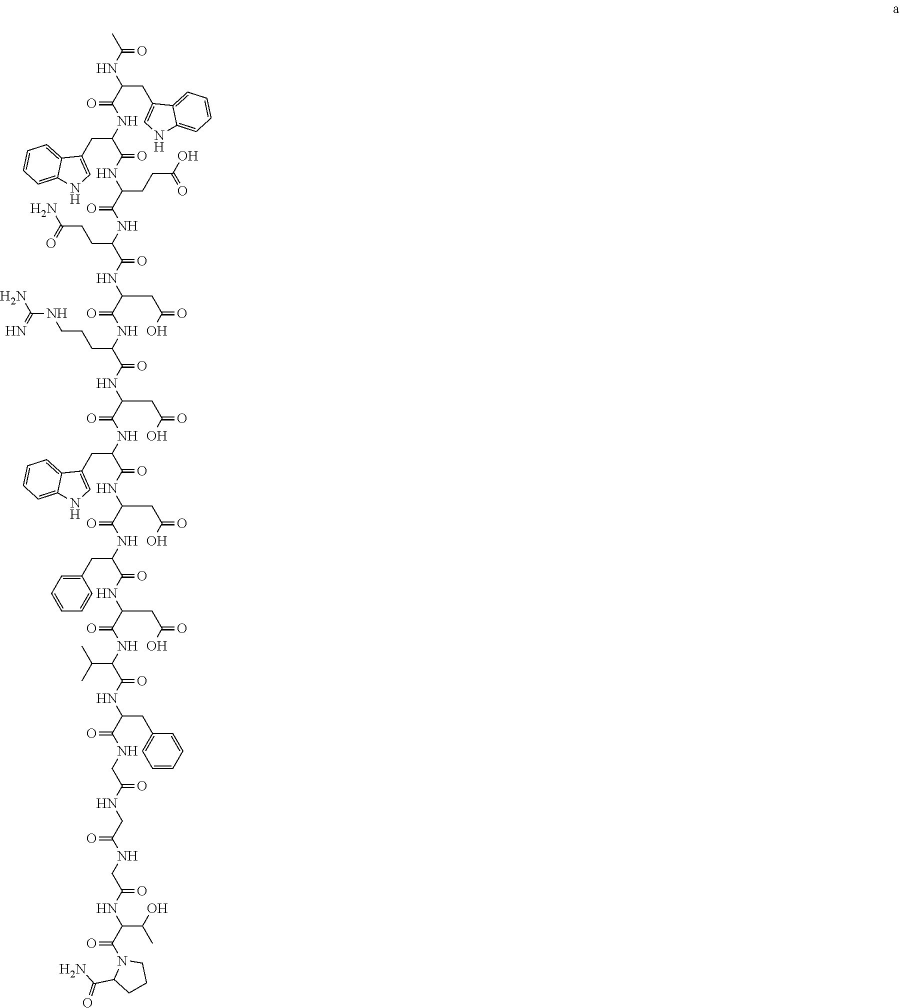 [SMILES: CC(=O)NC(Cc1c[nH]c2ccccc12)C(=O)NC(Cc1c[nH]c2ccccc12)C(=O)NC(CCC(=O)O)C(=O)NC(CCC(N)=O)C(=O)NC(CC(=O)O)C(=O)NC(CCCNC(=N)N)C(=O)NC(CC(=O)O)C(=O)NC(Cc1c[nH]c2ccccc12)C(=O)NC(CC(=O)O)C(=O)NC(Cc1ccccc1)C(=O)NC(CC(=O)O)C(=O)NC(C(=O)NC(Cc1ccccc1)C(=O)NCC(=O)NCC(=O)NCC(=O)NC(C(=O)N1CCCC1C(N)=O)C(C)O)C(C)C]

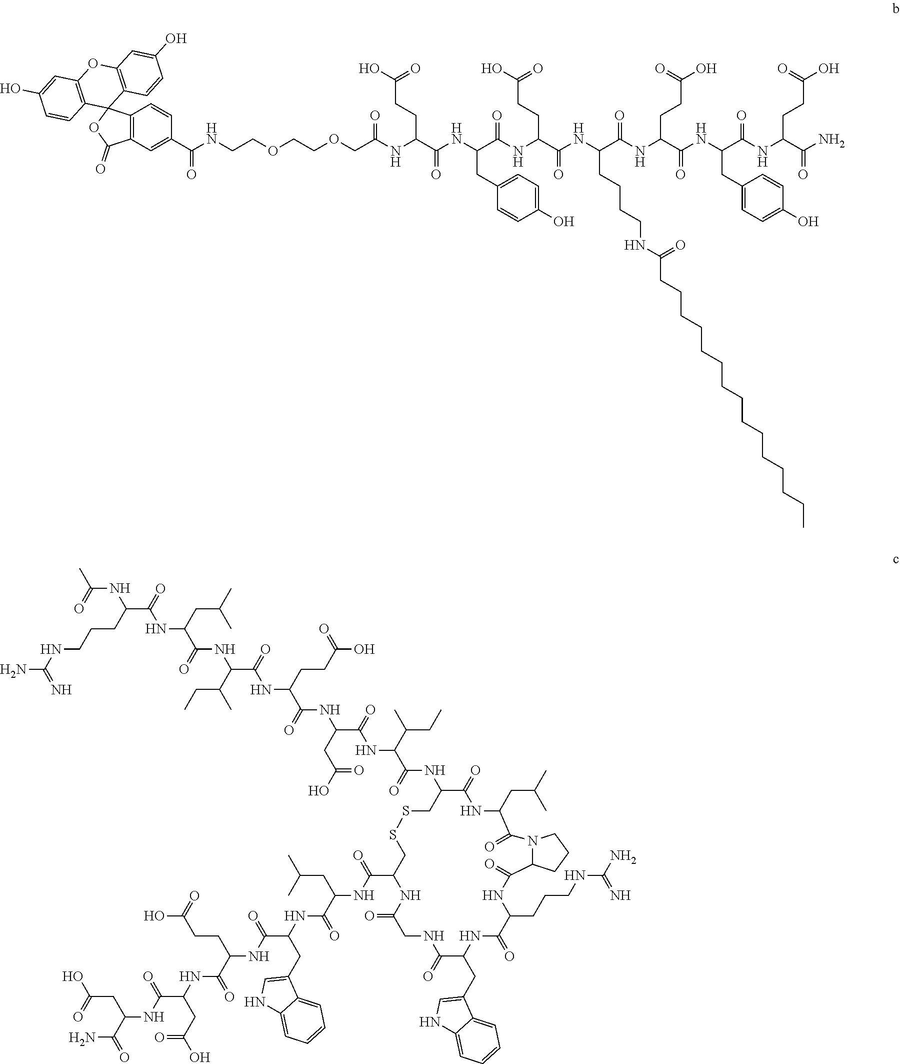

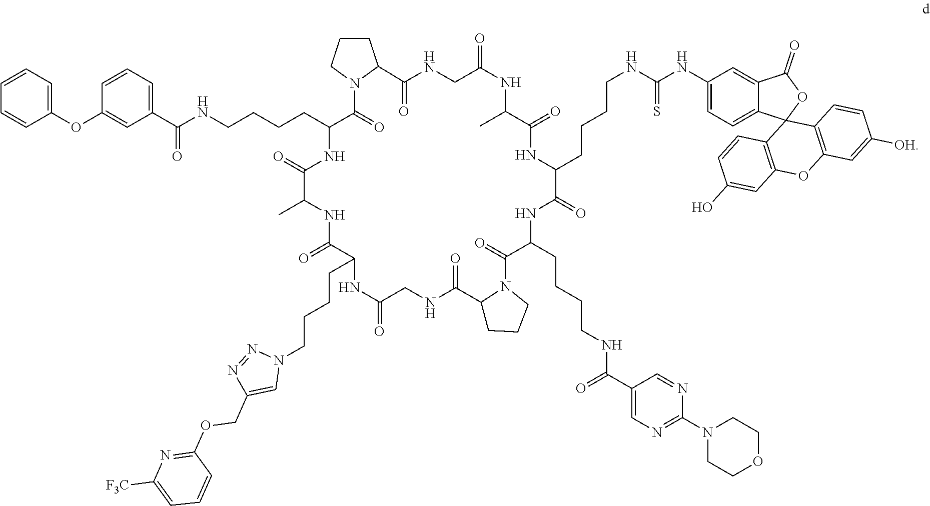 [SMILES: CC1NC(=O)CNC(=O)C2CCCN2C(=O)C(CCCCNC(=O)c2cccc(Oc3ccccc3)c2)NC(=O)C(C)NC(=O)C(CCCCn2cc(COc3cccc(C(F)(F)F)n3)nn2)NC(=O)CNC(=O)C2CCCN2C(=O)C(CCCCNC(=O)c2cnc(N3CCOCC3)nc2)NC(=O)C(CCCCNC(=S)Nc2ccc3c(c2)C(=O)OC32c3ccc(O)cc3Oc3cc(O)ccc32)NC1=O]

In certain embodiments, C′ is or comprises:

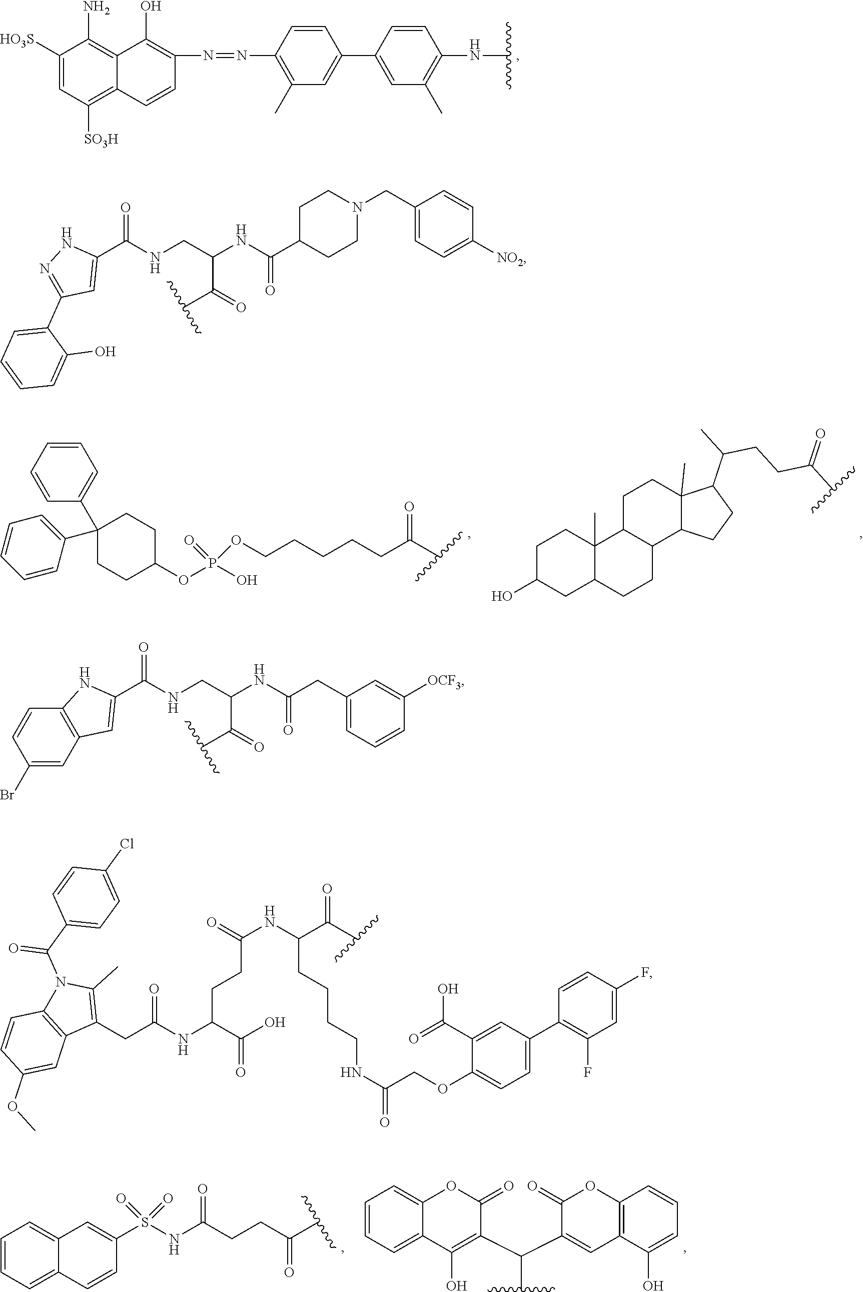

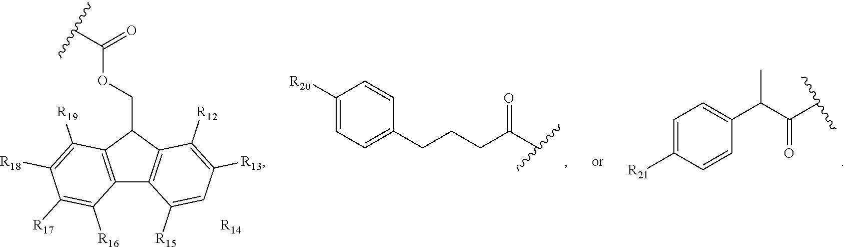

wherein, as applicable:
each of R₁₂₋₁₉ (where applicable) is independently —H, —C₁-C₆ alkyl, —F, —Cl, —Br, —I, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, or —SO₂F; and
each of R₂₀ and R₂₁ is independently —H, —C₁-C₆ alkyl, —F, —Cl, —Br, —I, —O—C₁₋₆ alkyl, —CN, —CHO, —B(OH)₂, —C═C—C(O)aryl, —C═C—S(O)₂aryl, —CO₂H, —SO₃H, —SO₂NH₂, —PO₃H₂, —SO₂F, or CF₃.

In some embodiments C′ is or comprises

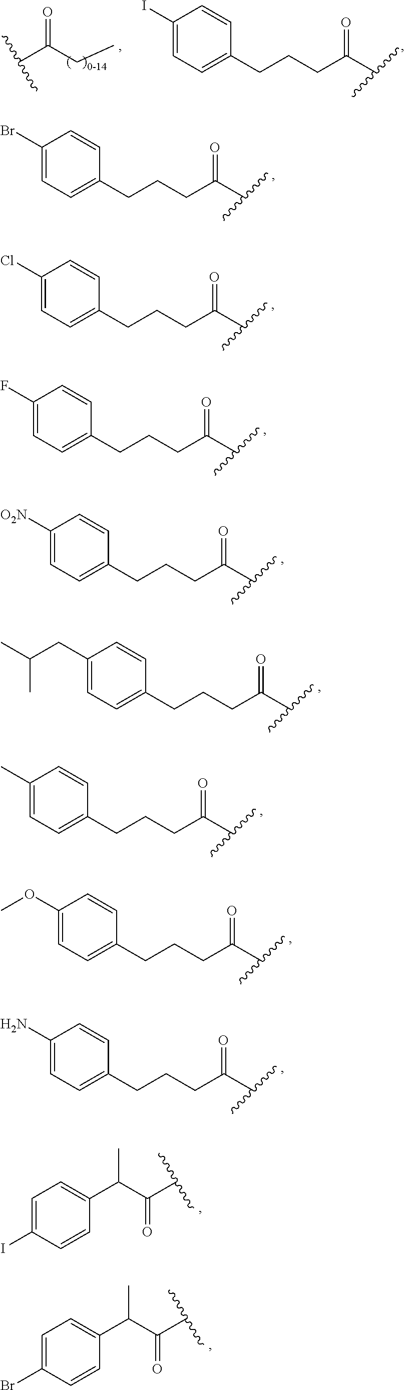

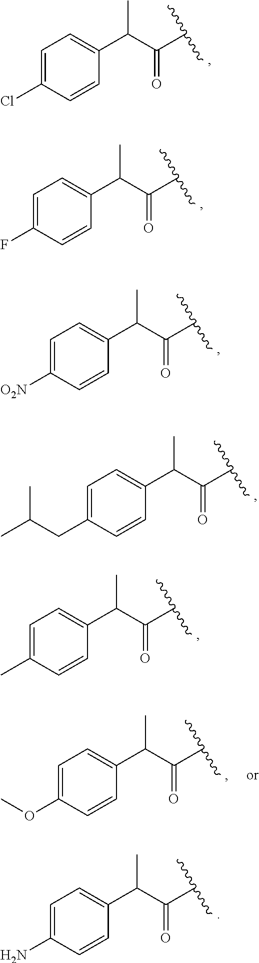

In some embodiments C′ is or comprises

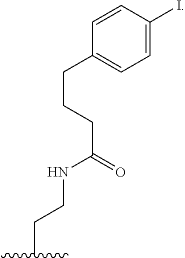 [SMILES: *CCNC(=O)CCCc1ccc(I)cc1]

In some embodiments C′ is or comprises

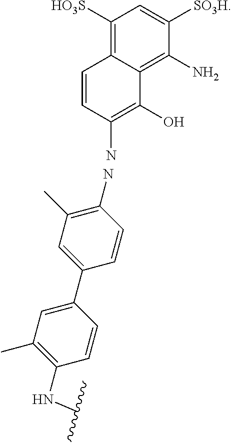

In certain embodiments, C′ of the compound comprises a (e.g., a radical of) (PEG)ₙ, wherein n is an integer 0 to 32, a peptide, a peptidoglycan or a saccharide.

C′ can be (PEG)ₙ, and n is an integer 0 to 32. C′ can be a peptide. C′ can be a peptidoglycan. C′ can be saccharide.

In some embodiments C′ is or comprises

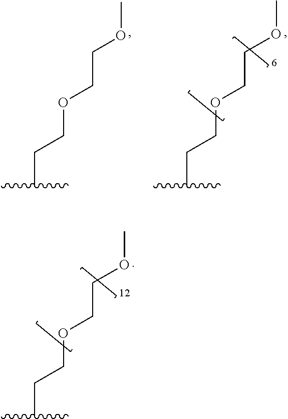

In some embodiments C′ is or comprises OH

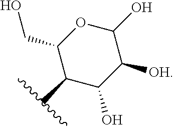 [SMILES: *[C@@H]1[C@@H](O)[C@H](O)C(O)O[C@H]1CO]

Also provided are compounds (e.g., conjugates) of formula (V):

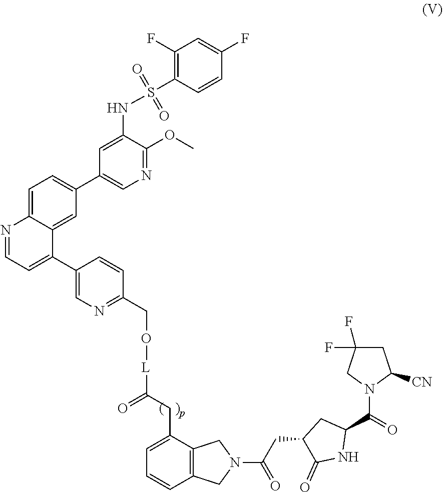

wherein
L is a linker comprising at least one carbon atom; and
p is 0, 1, 2, or 3.

For compounds of formula (V), L can be or comprise a group as shown in one of the embodiments shown previously for the linker L. In certain embodiments for compounds of formula (V), p can be 0. In certain embodiments for compounds of formula (V), p can be 1. In certain embodiments for compounds of formula (V), p can be 2. In certain embodiments for compounds of formula (V), p can be 3.

### Compounds/Conjugates

In some embodiments the compound is not

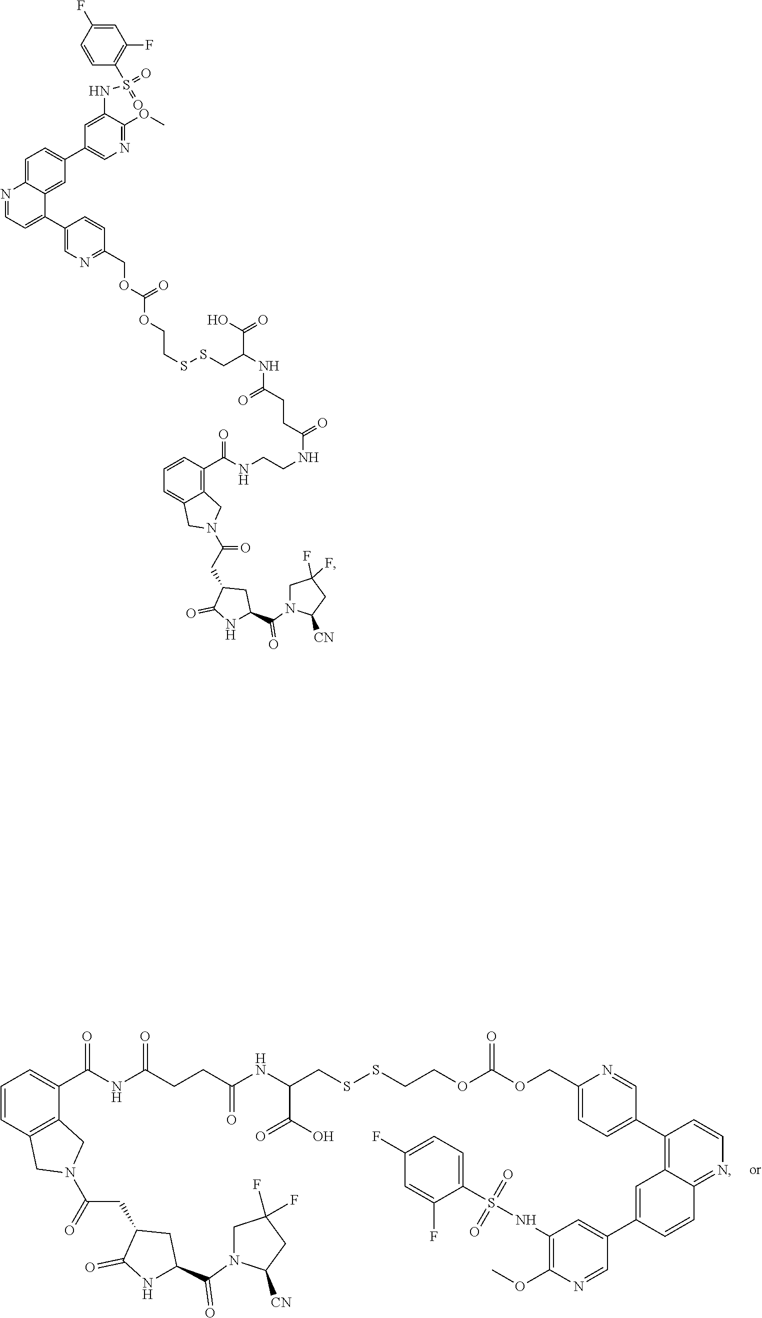 [SMILES: COc1ncc(-c2ccc3nccc(-c4ccc(COC(=O)OCCSSCC(NC(=O)CCC(=O)NCCNC(=O)c5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F ; COc1ncc(-c2ccc3nccc(-c4ccc(COC(=O)OCCSSCC(NC(=O)CCC(=O)NC(=O)c5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F]

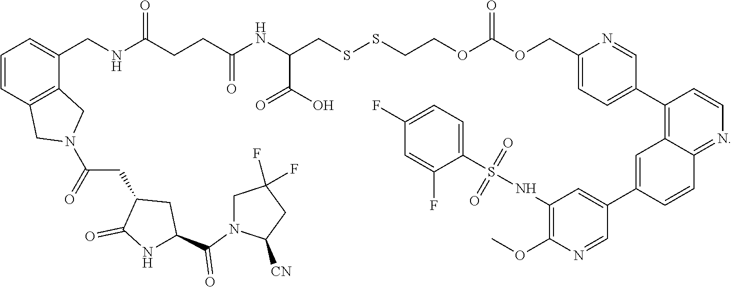 [SMILES: COc1ncc(-c2ccc3nccc(-c4ccc(COC(=O)OCCSSCC(NC(=O)CCC(=O)NCc5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F]

A-L-B′ can have the following structure:

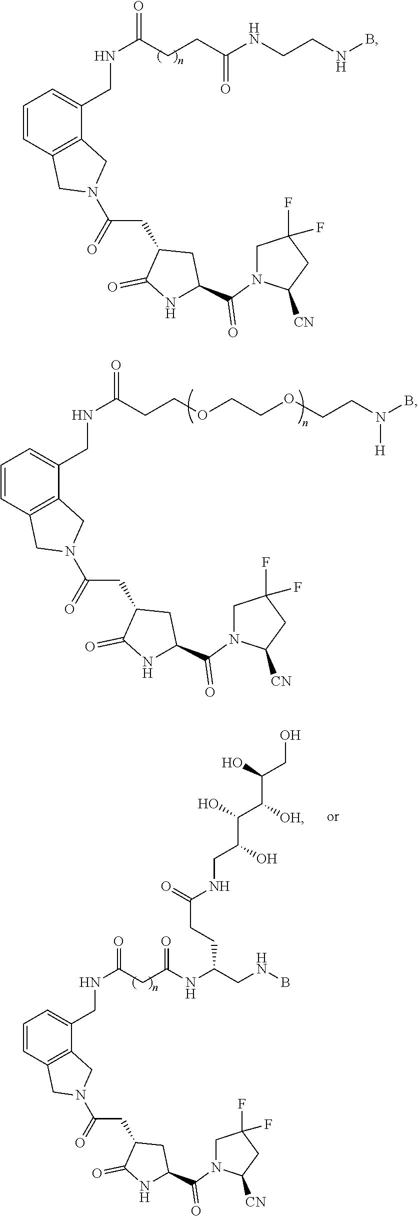

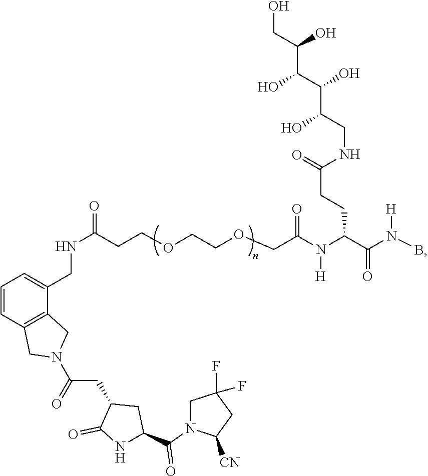

wherein:
n is an integer from 1-5.

A-L-B′ can have the following structure:

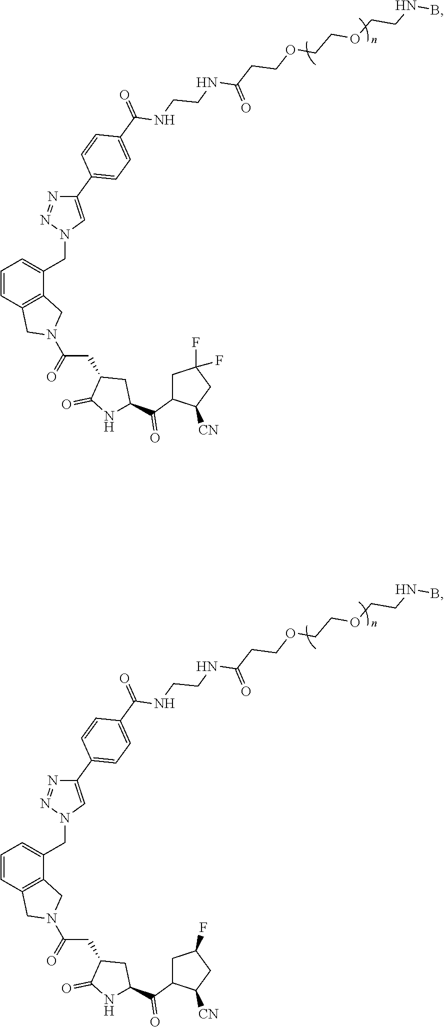

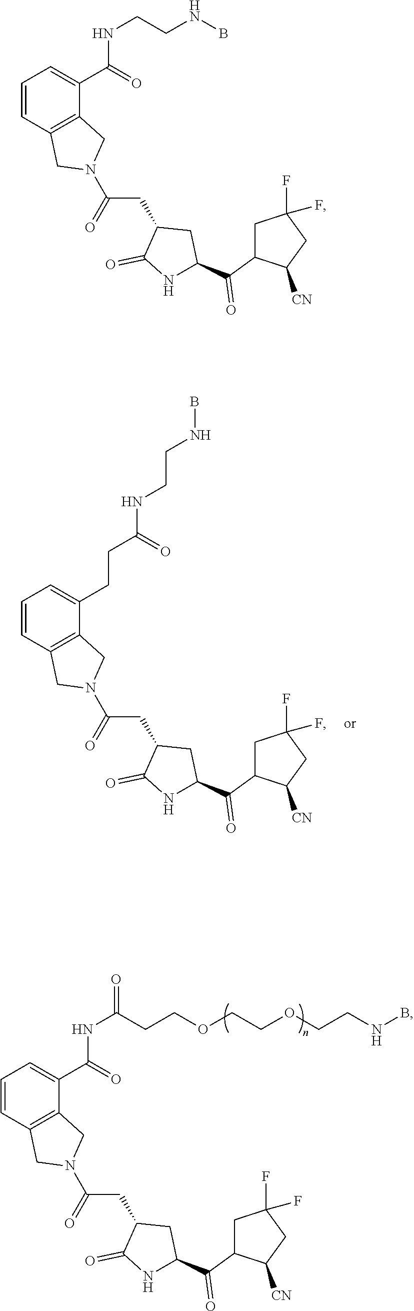

wherein:
n is an integer from 1-5.

The compound can have the formula:

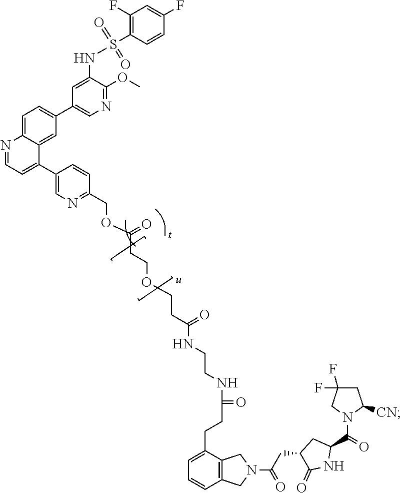

wherein t is 0 or 1 and u is an integer from 2-12.

The compound can have the formula

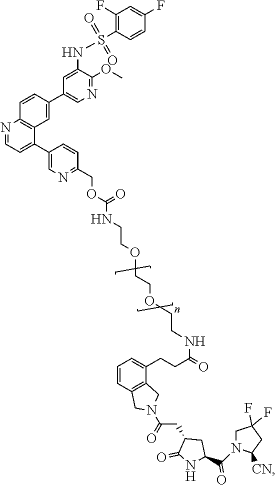

wherein n is an integer from 1 to 12.

The compound can have the formula

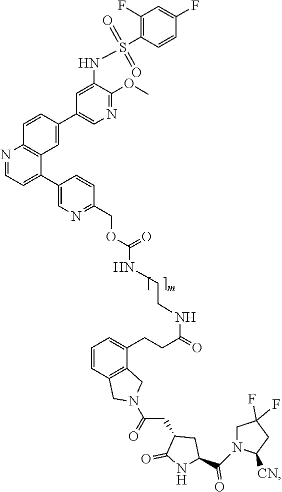

wherein m is an integer from 1 to 4.

The compound can have the formula

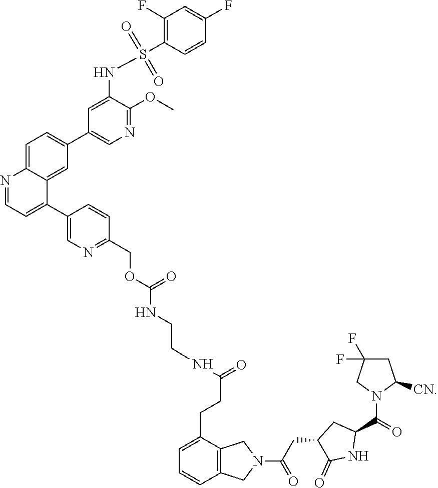 [SMILES: COc1ncc(-c2ccc3nccc(-c4ccc(COC(=O)NCCNC(=O)CCc5cccc6c5CN(C(=O)C[C@@H]5C[C@@H](C(=O)N7CC(F)(F)C[C@H]7C#N)NC5=O)C6)nc4)c3c2)cc1NS(=O)(=O)c1ccc(F)cc1F]

The compound can have the formula

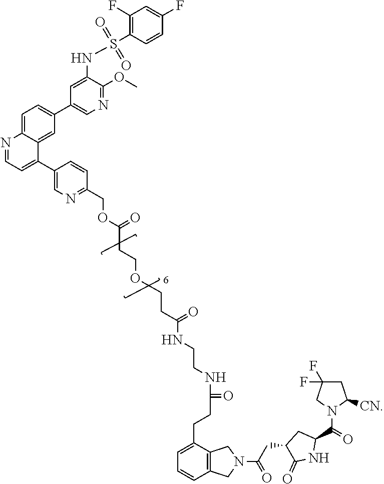

The compound can have the formula

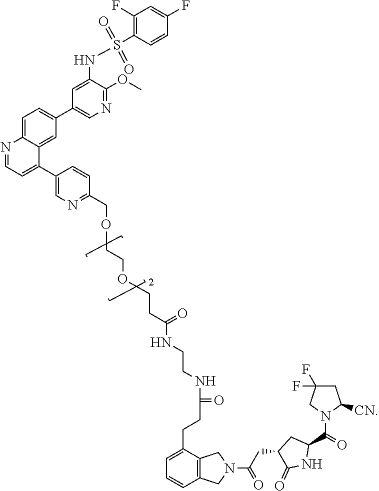

The compound can have the formula

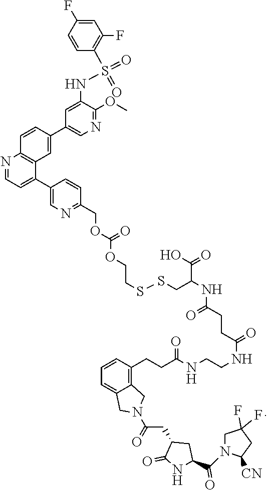 [SMILES: COc1ncc(-c2ccc3nccc(-c4ccc(COC(=O)OCCSSCC(NC(=O)CCC(=O)NCCNC(=O)CCc5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F]

The compound can have the formula

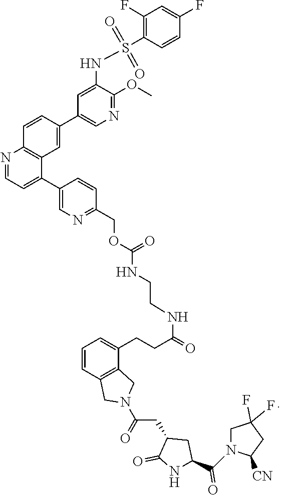 [SMILES: COc1ncc(-c2ccc3nccc(-c4ccc(COC(=O)NCCNC(=O)CCc5cccc6c5CN(C(=O)C[C@@H]5C[C@@H](C(=O)N7CC(F)(F)C[C@H]7C#N)NC5=O)C6)nc4)c3c2)cc1NS(=O)(=O)c1ccc(F)cc1F]

In certain embodiments, A-L-B′ of a compound hereof can have the following structure:

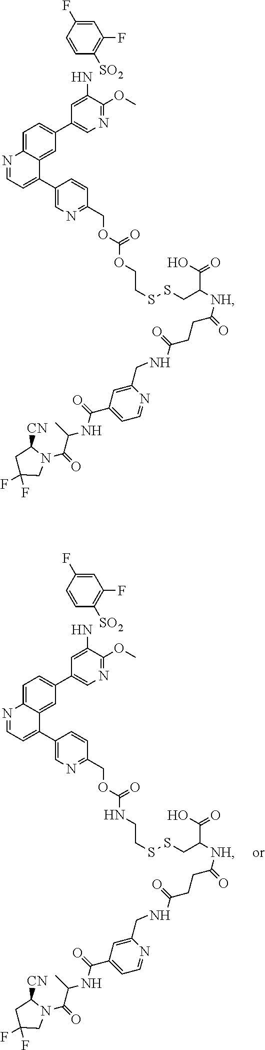

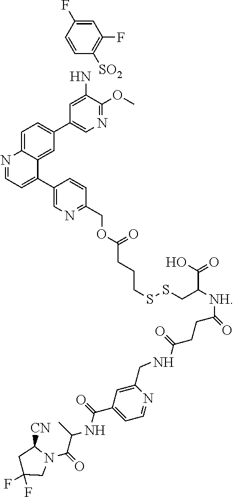

A-L-B′ can have the following structure:

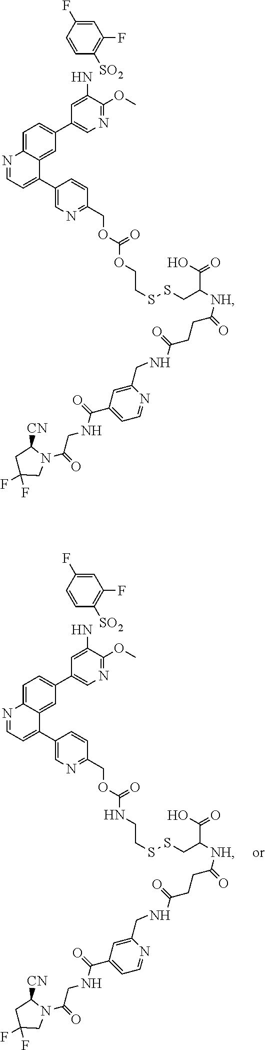

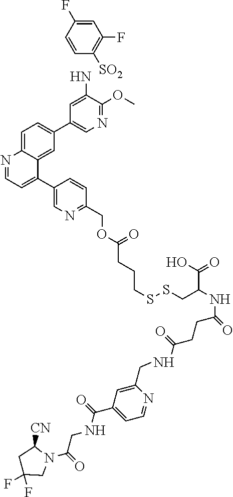

A-L-B′ can have the following structure

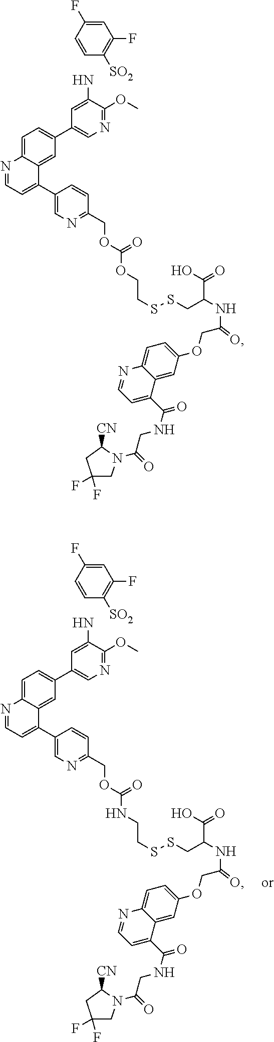

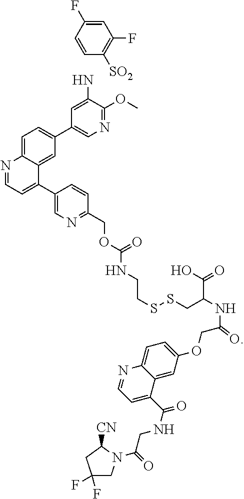

A-L-B′ can have the following structure:

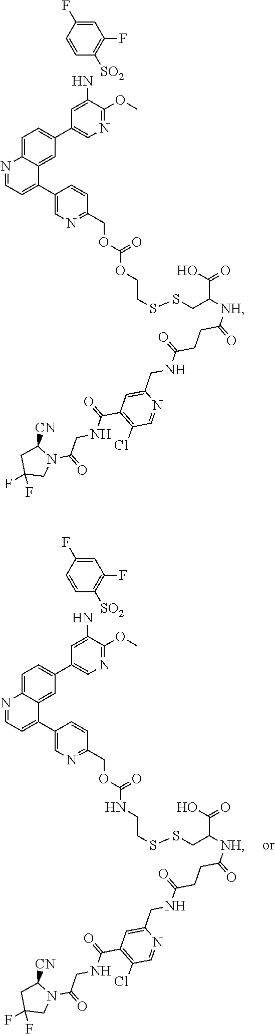

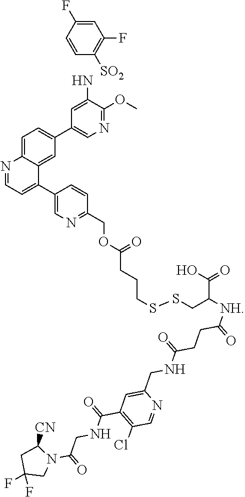

A-L-B′ can have the following structure:

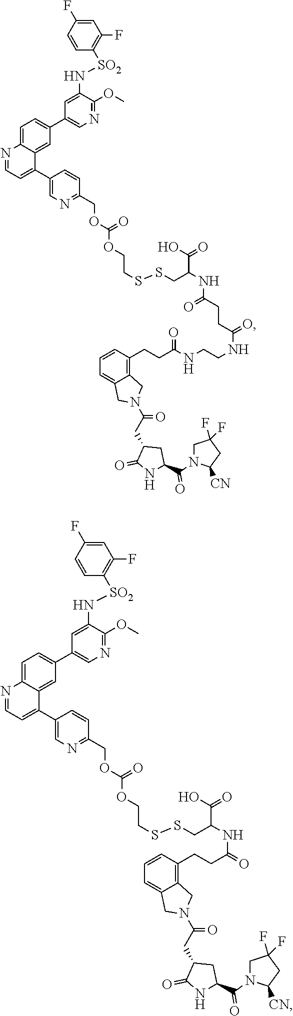

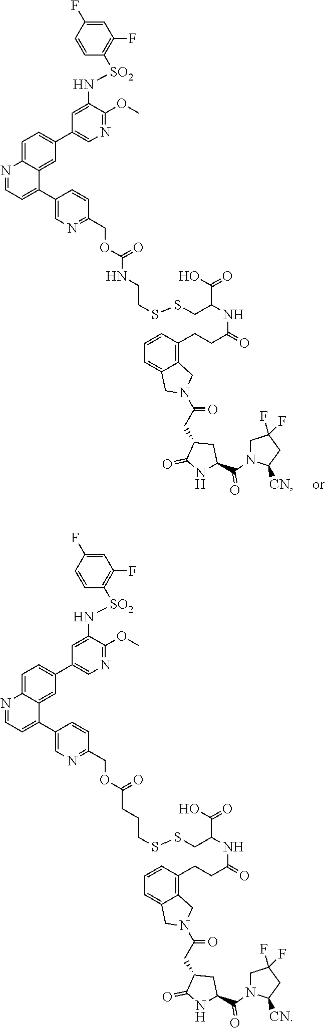

A-L-B′ can have the following structure:

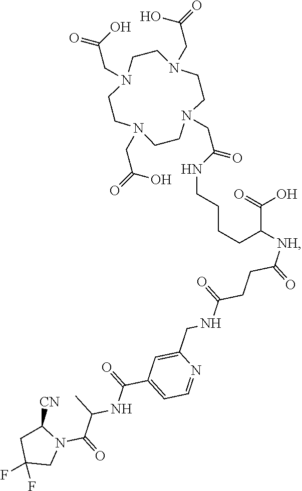 [SMILES: CC(NC(=O)c1ccnc(CNC(=O)CCC(=O)NC(CCCCNC(=O)CN2CCN(CC(=O)O)CCN(CC(=O)O)CCN(CC(=O)O)CC2)C(=O)O)c1)C(=O)N1CC(F)(F)C[C@H]1C#N]

optionally bound to an isotope (or metal) suitable for radio-imaging, radiotherapy or magnetic resonance imaging.

A-L-B′ can have the following structure:

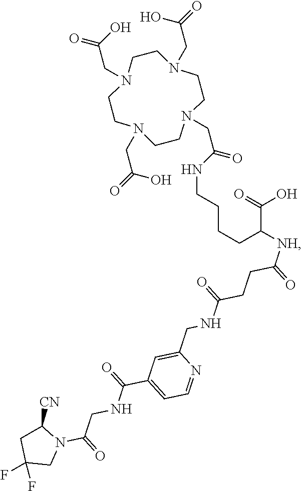 [SMILES: N#C[C@@H]1CC(F)(F)CN1C(=O)CNC(=O)c1ccnc(CNC(=O)CCC(=O)NC(CCCCNC(=O)CN2CCN(CC(=O)O)CCN(CC(=O)O)CCN(CC(=O)O)CC2)C(=O)O)c1]

optionally bound to an isotope (or metal) suitable for radio-imaging, radiotherapy or magnetic resonance imaging.

A-L-B′ can have the following structure:

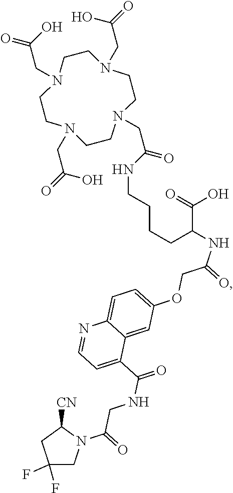

optionally bound to an isotope (or metal) suitable for radio-imaging, radiotherapy or magnetic resonance imaging.

A-L-B′ can have the following structure:

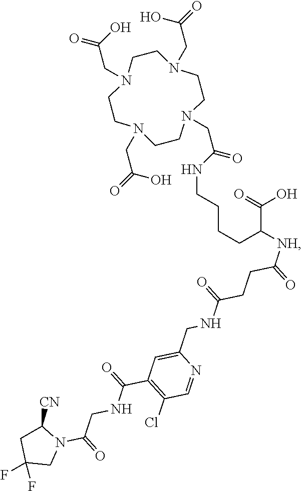 [SMILES: N#C[C@@H]1CC(F)(F)CN1C(=O)CNC(=O)c1cc(CNC(=O)CCC(=O)NC(CCCCNC(=O)CN2CCN(CC(=O)O)CCN(CC(=O)O)CCN(CC(=O)O)CC2)C(=O)O)ncc1Cl]

optionally bound to an isotope (or metal) suitable for radio-imaging, radiotherapy or magnetic resonance imaging.

A-L-B′ can have the following structure:

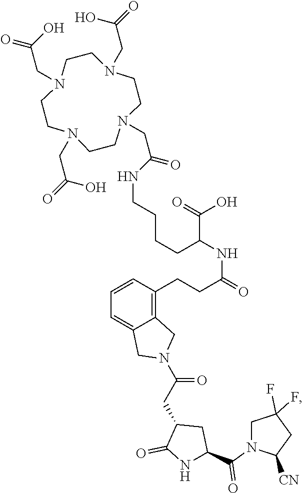 [SMILES: N#C[C@@H]1CC(F)(F)CN1C(=O)[C@@H]1C[C@@H](CC(=O)N2Cc3cccc(CCC(=O)NC(CCCCNC(=O)CN4CCN(CC(=O)O)CCN(CC(=O)O)CCN(CC(=O)O)CC4)C(=O)O)c3C2)C(=O)N1]

optionally bound to an isotope (or metal) suitable for radio-imaging, radiotherapy or magnetic resonance imaging.

A compound (e.g., conjugate) can have the following structure:

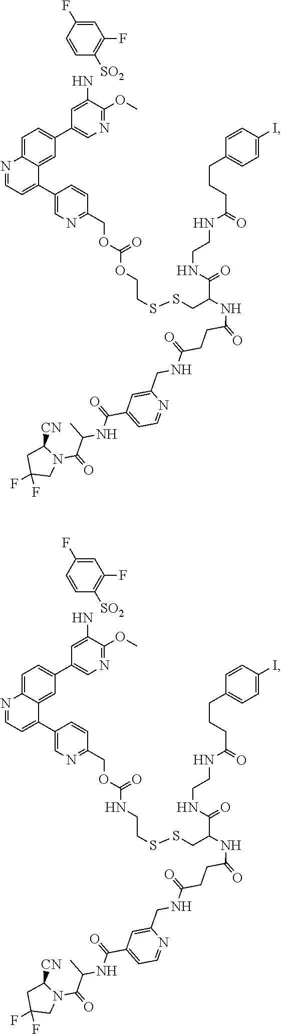

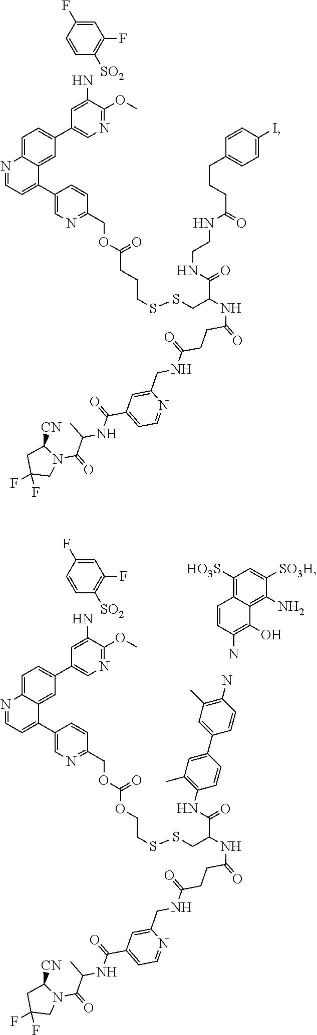

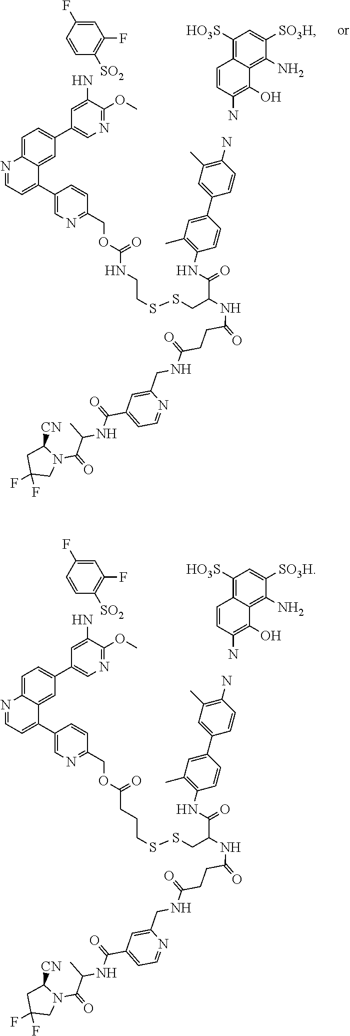

A compound (e.g., conjugate) can have the following structure:

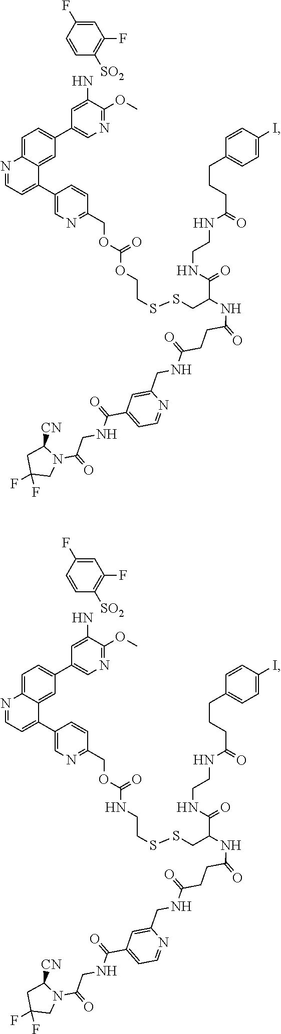

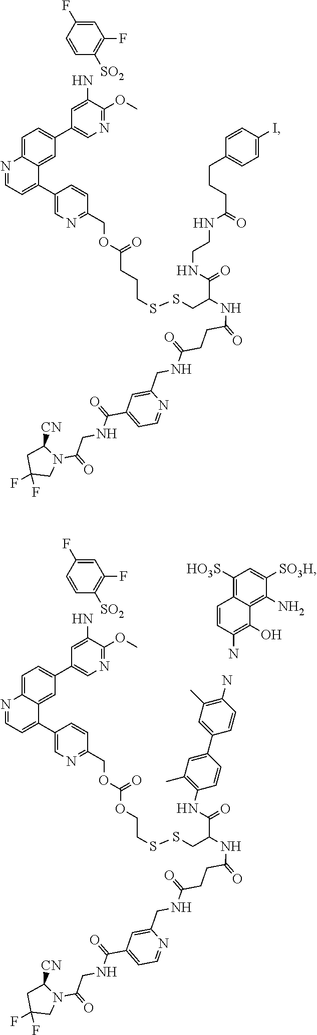

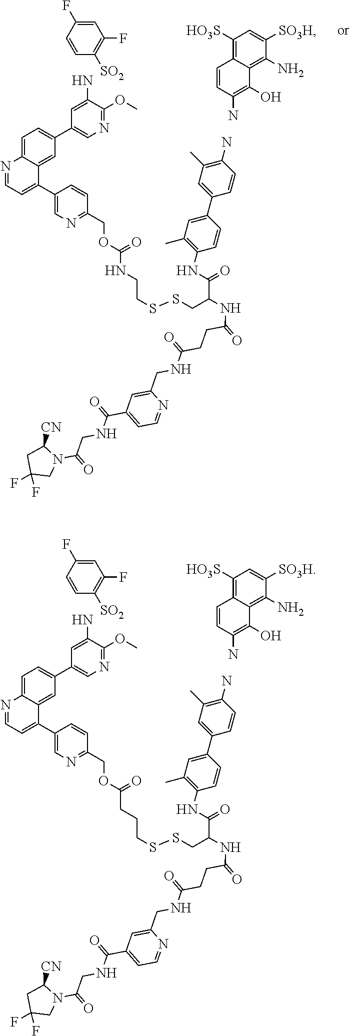

A compound (e.g., conjugate) can have the following structure:

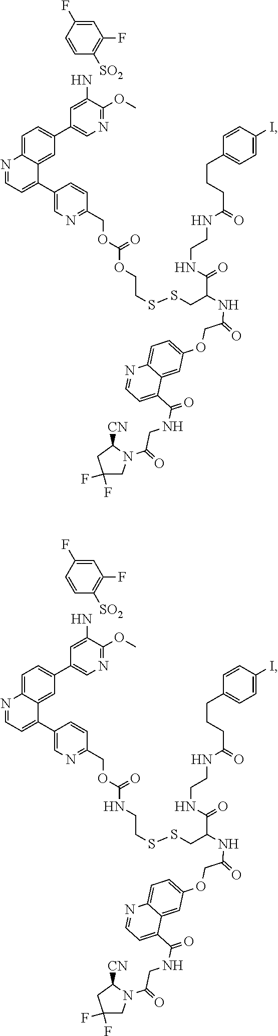

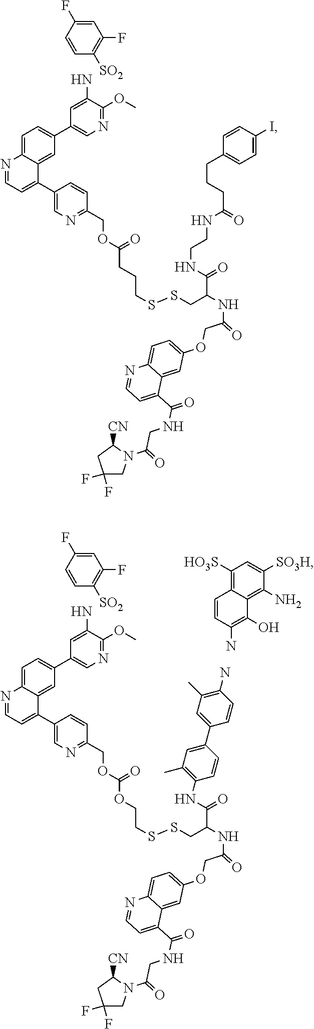

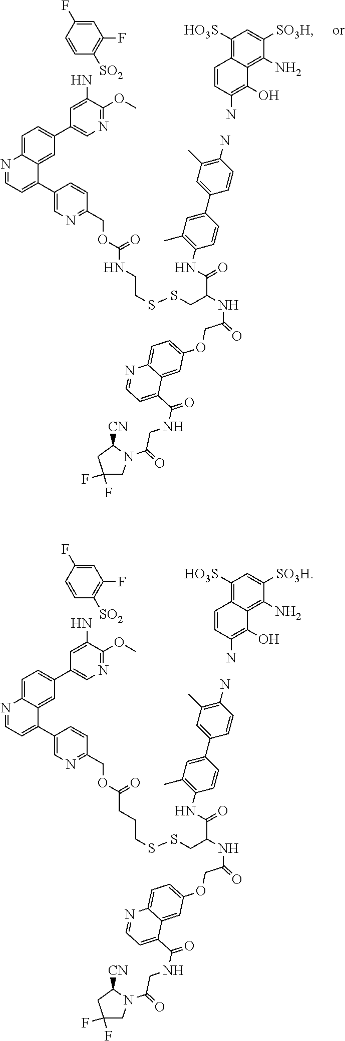

A compound (e.g., conjugate) can have the following structure:

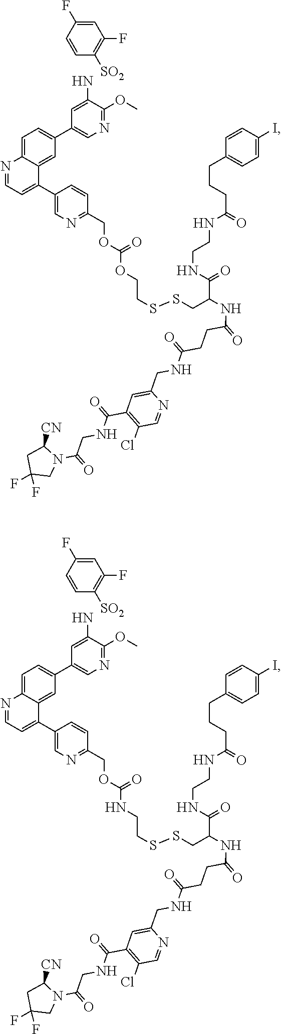

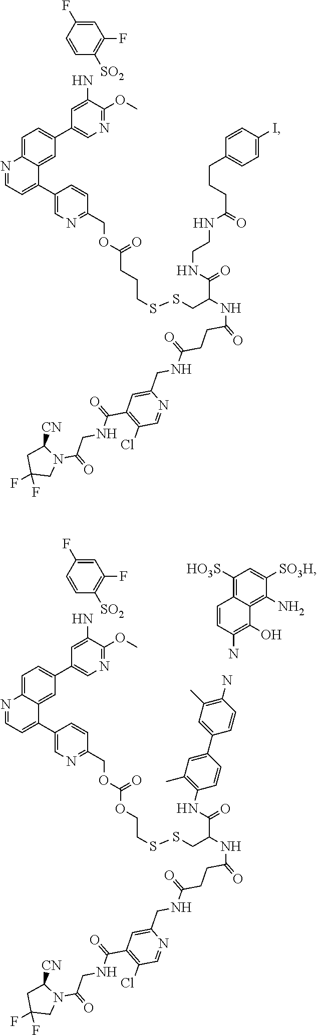

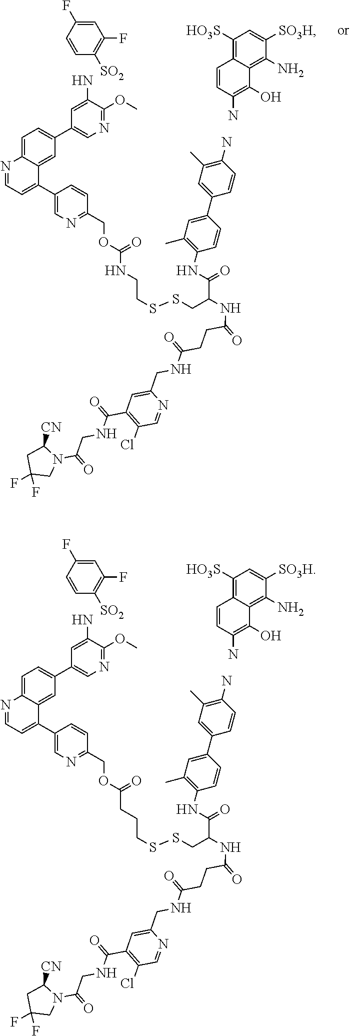

A compound (e.g., conjugate) can have the following structure:

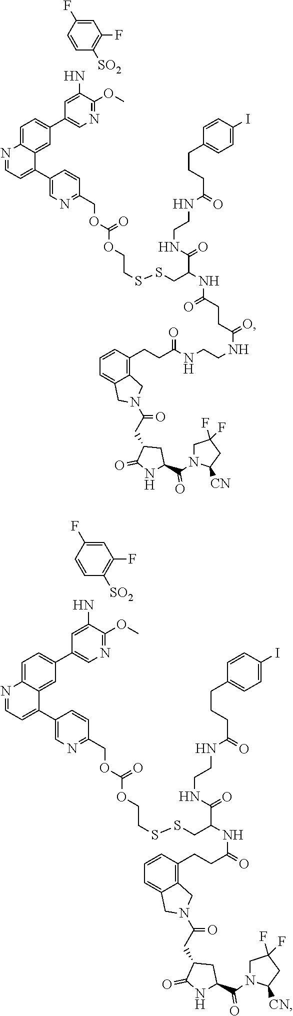

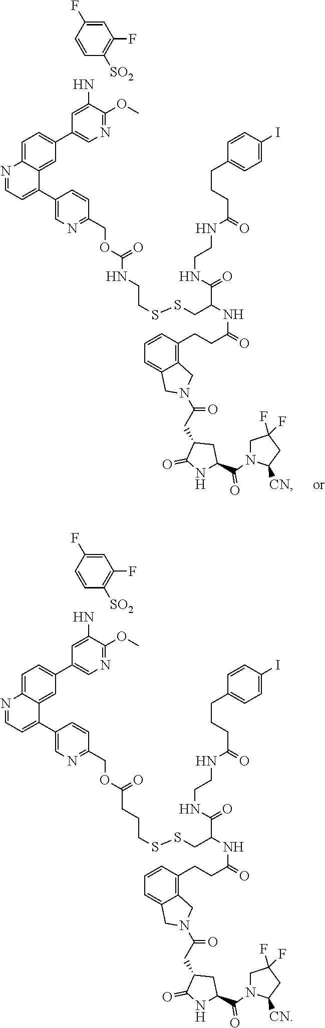

A compound (e.g., conjugate) can have the following structure:

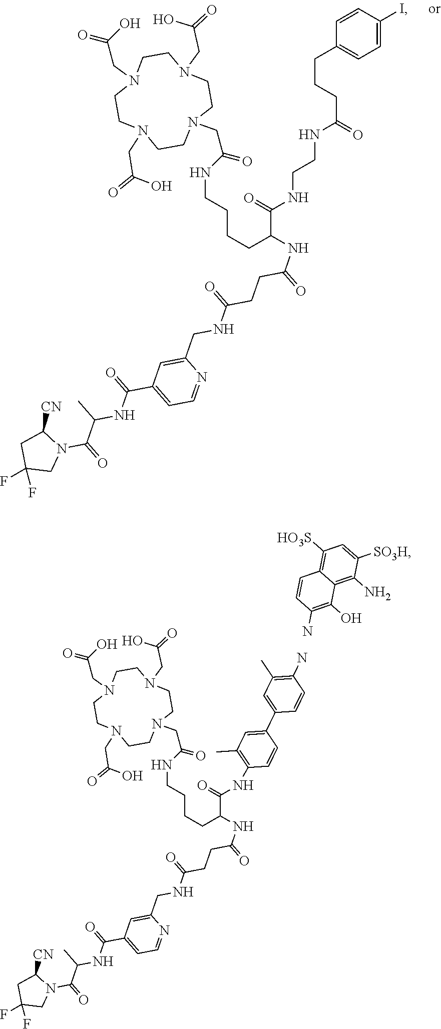

each optionally bound to an isotope (or metal) suitable for radio-imaging, radiotherapy or magnetic resonance imaging.

A compound (e.g., conjugate) can have the following structure:

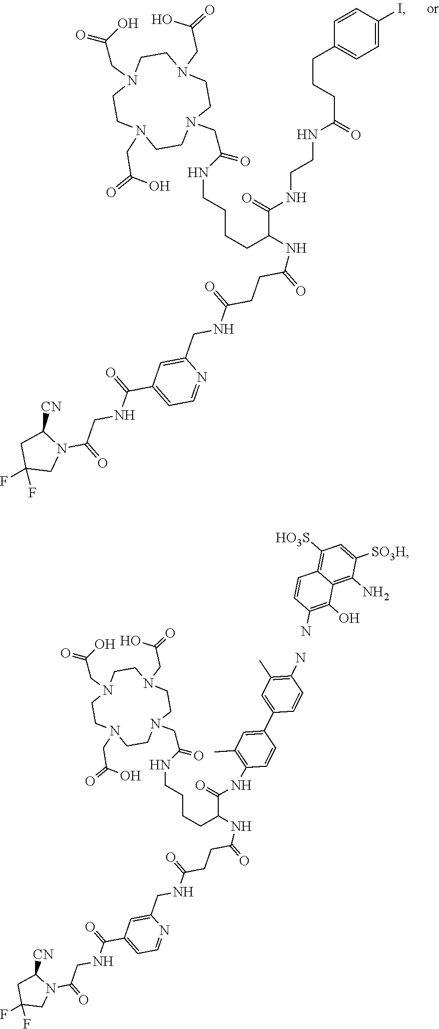

each optionally bound to an isotope (or metal) suitable for radio-imaging, radiotherapy or magnetic resonance imaging.

A compound (e.g., conjugate) can have the following structure:

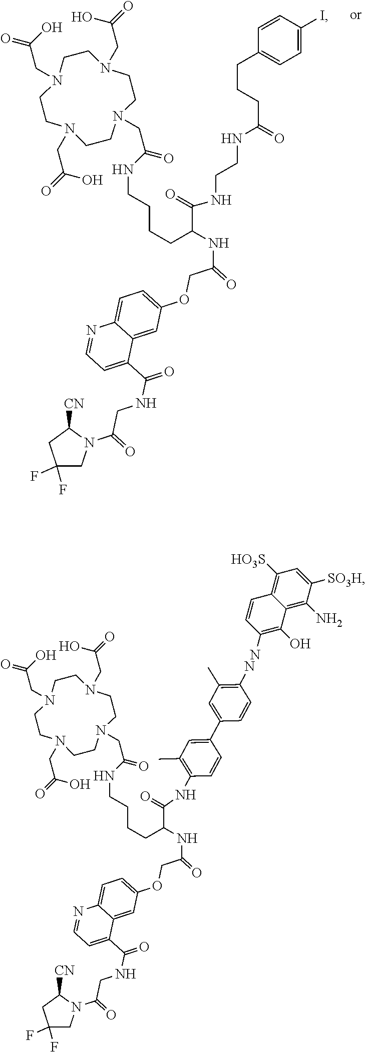

each optionally bound to an isotope (or metal) suitable for radio-imaging, radiotherapy or magnetic resonance imaging.

A compound (e.g., conjugate) can have the following structure:

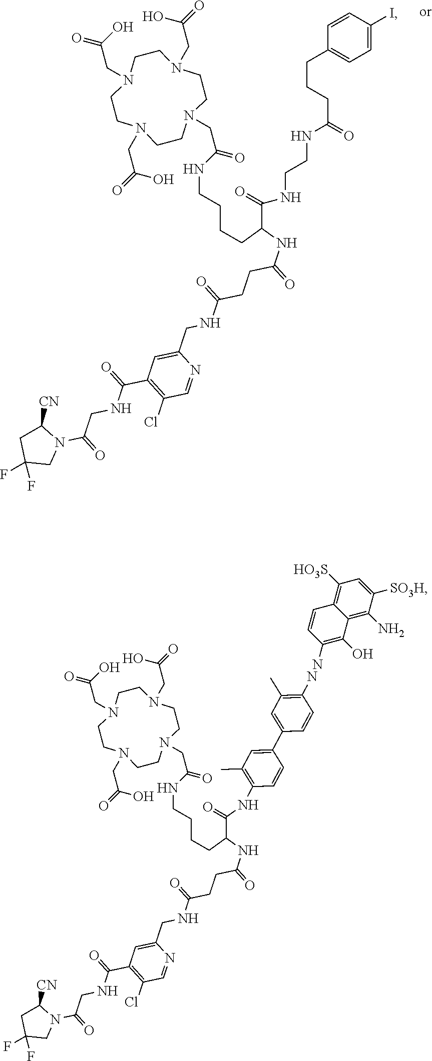

each optionally bound to an isotope (or metal) suitable for radio-imaging, radiotherapy or magnetic resonance imaging.

A compound (e.g., conjugate) can have the following structure:

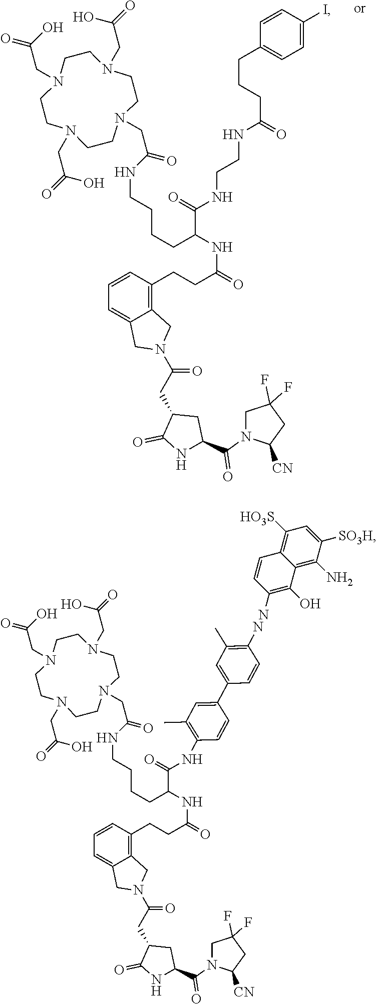

each optionally bound to an isotope (or metal) suitable for radio-imaging, radiotherapy or magnetic resonance imaging.

A compound (e.g., conjugate) can have the following structure:

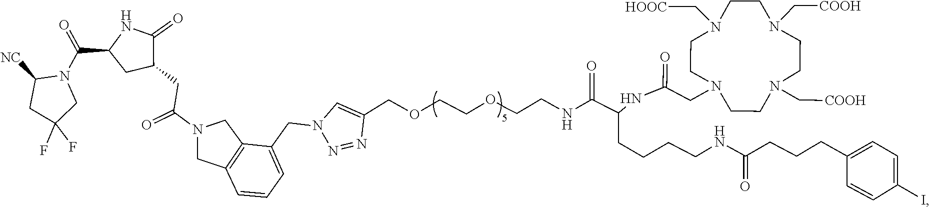

optionally bound to an isotope (or metal) suitable for radio-imaging, radiotherapy or magnetic resonance imaging.

A compound (e.g., conjugate) can have the following structure:

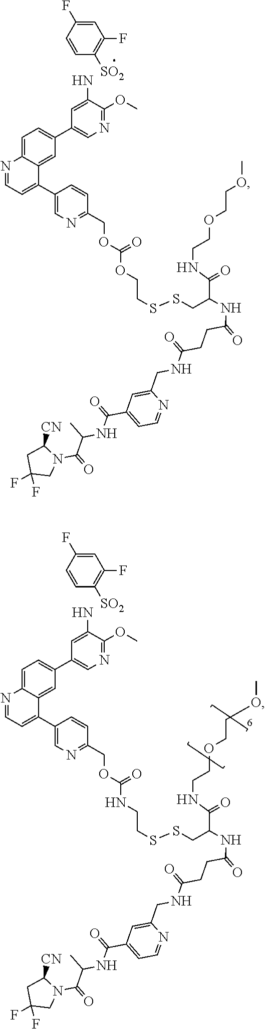

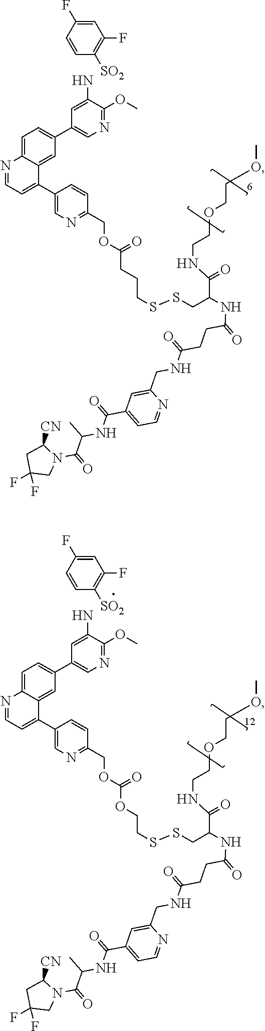

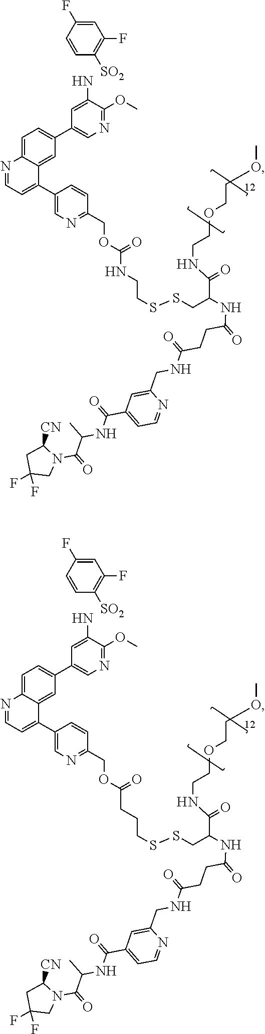

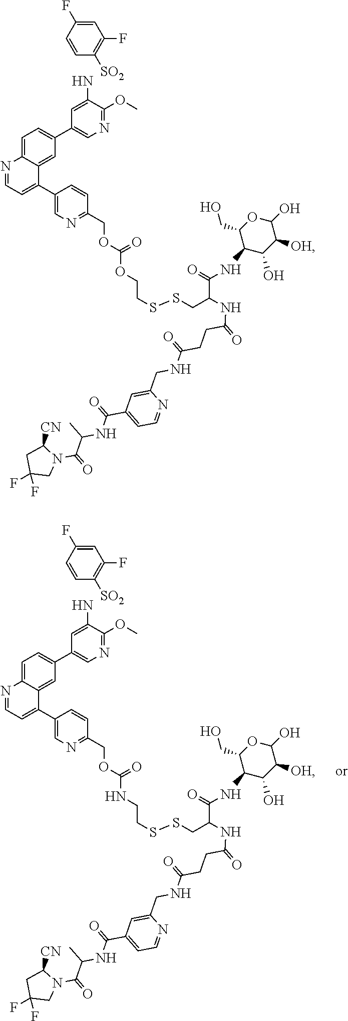

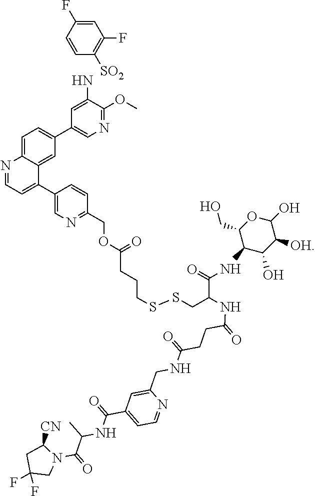

A compound (e.g., conjugate) can have the following structure:

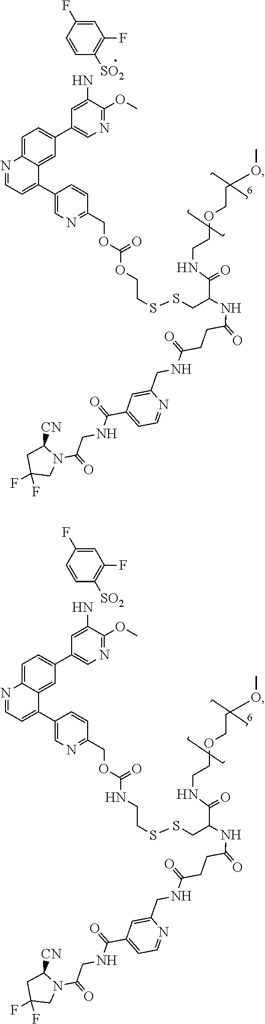

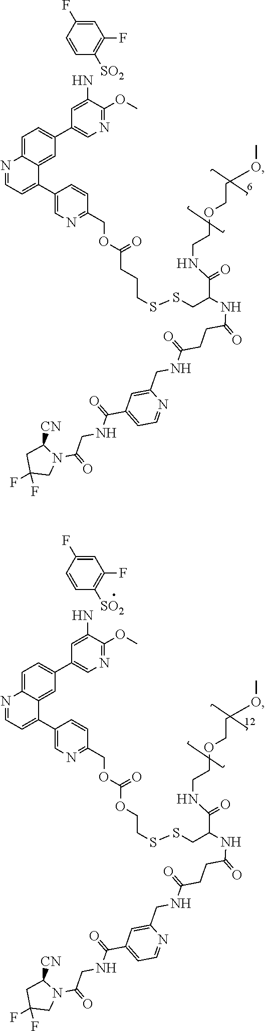

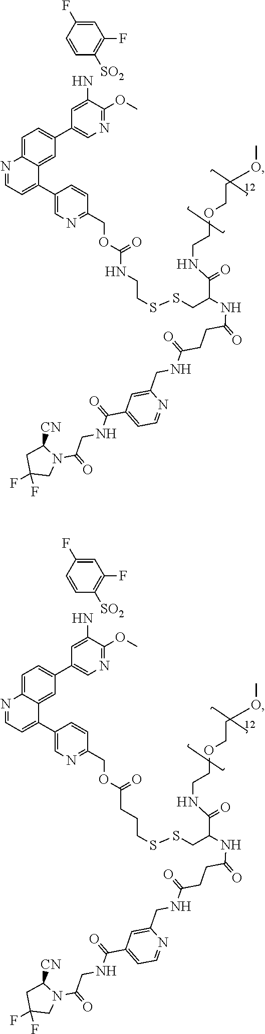

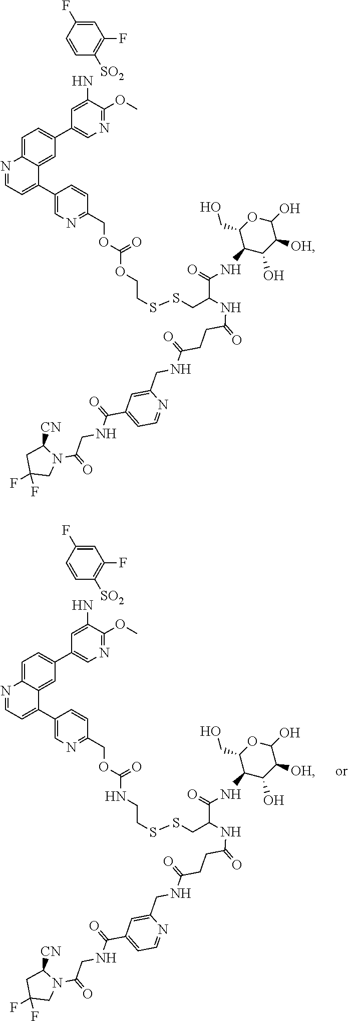

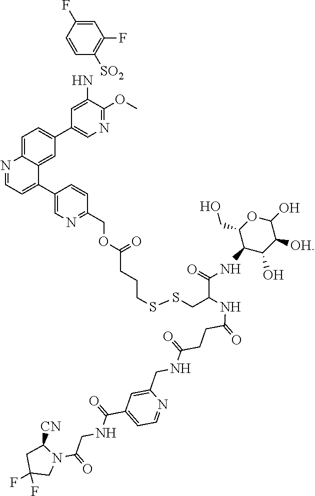

A compound (e.g., conjugate) can have the following structure:

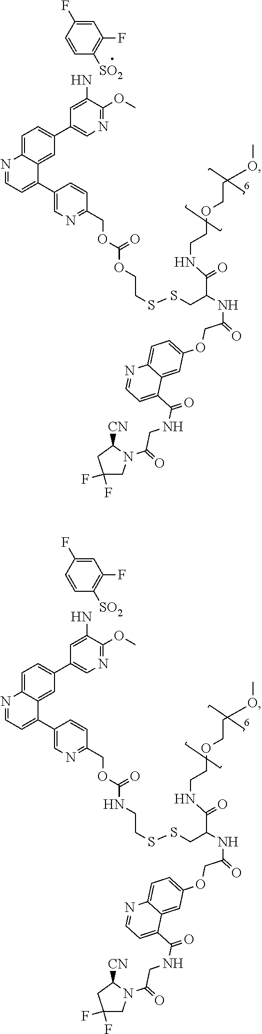

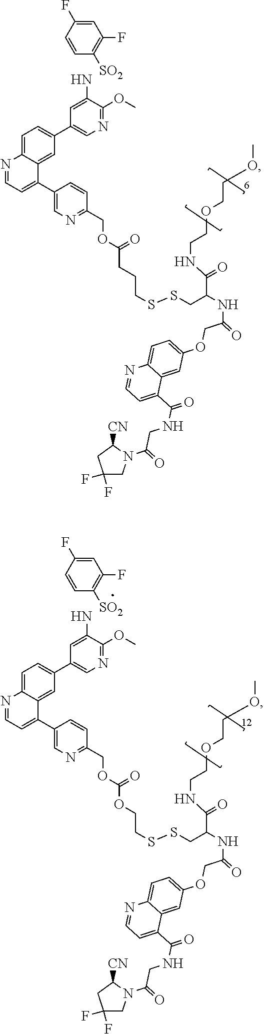

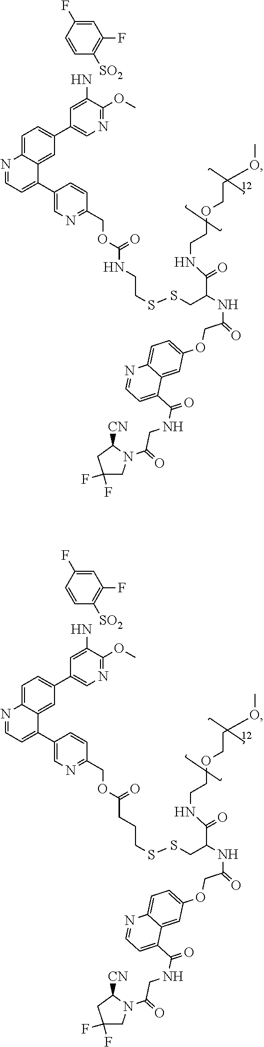

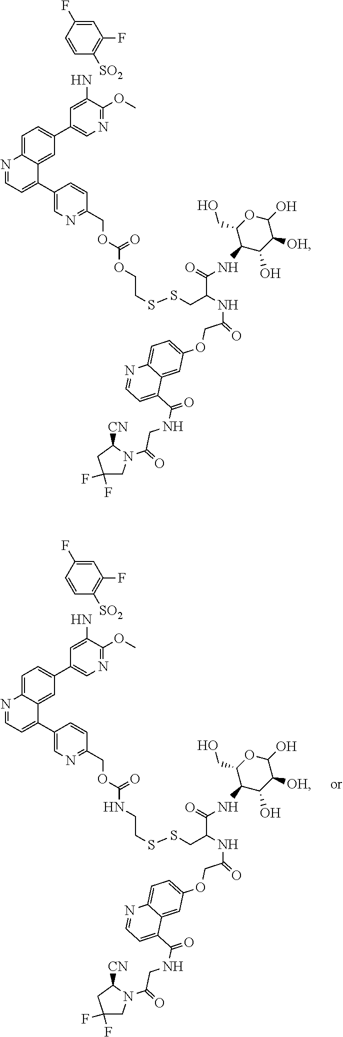

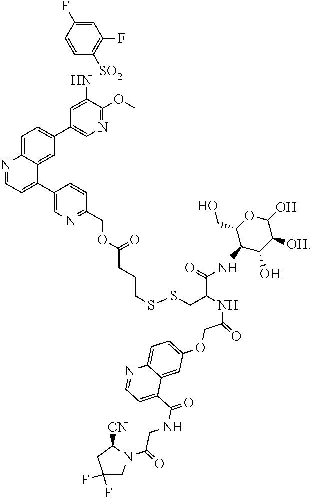

A compound (e.g., conjugate) can have the following structure:

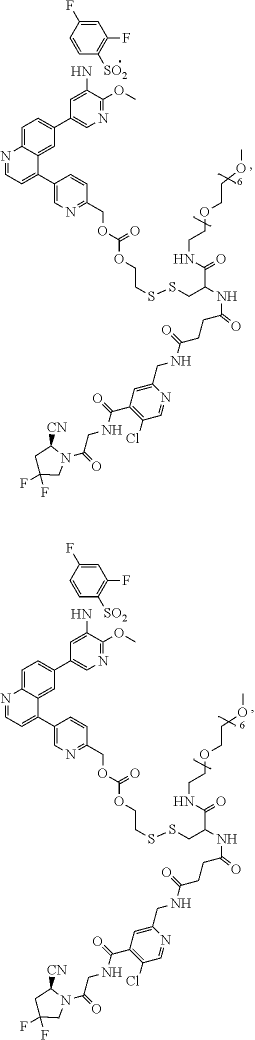

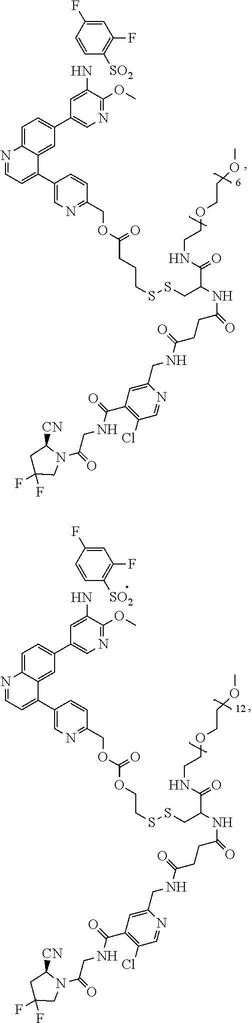

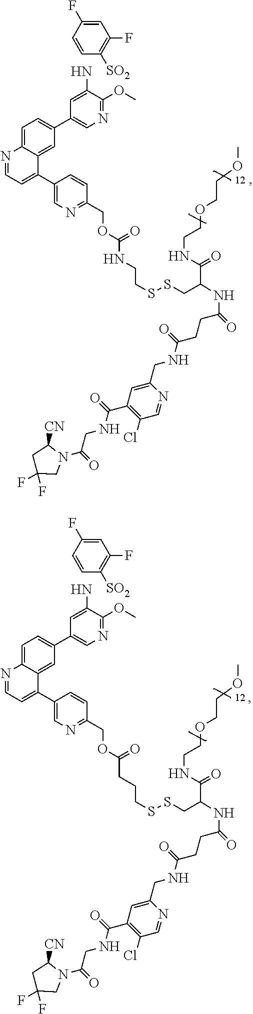

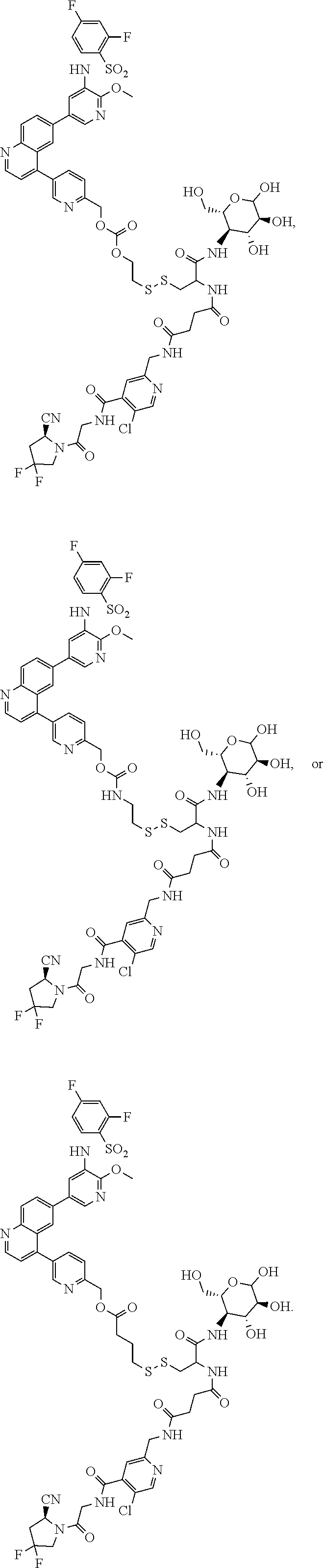

A compound (e.g., conjugate) can have the following structure:

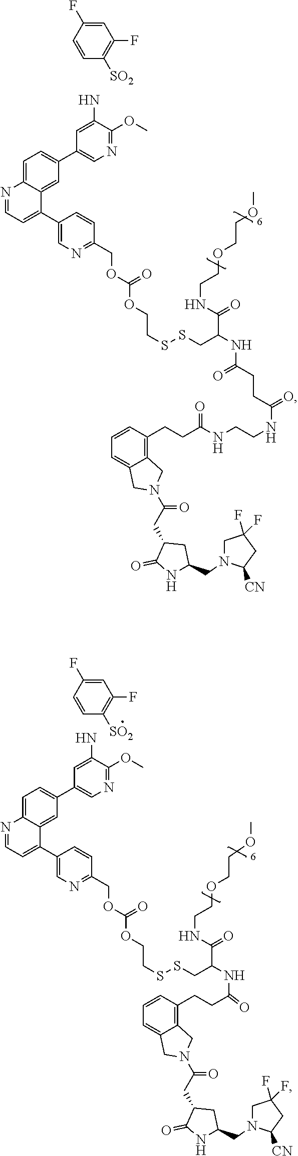

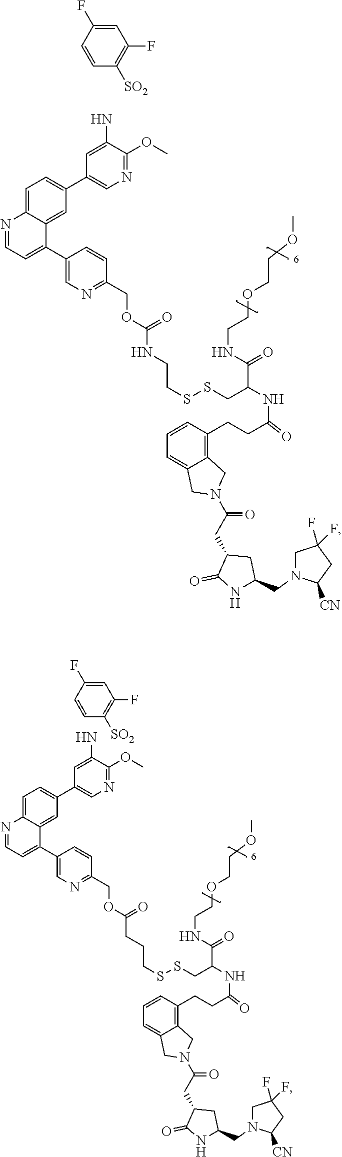

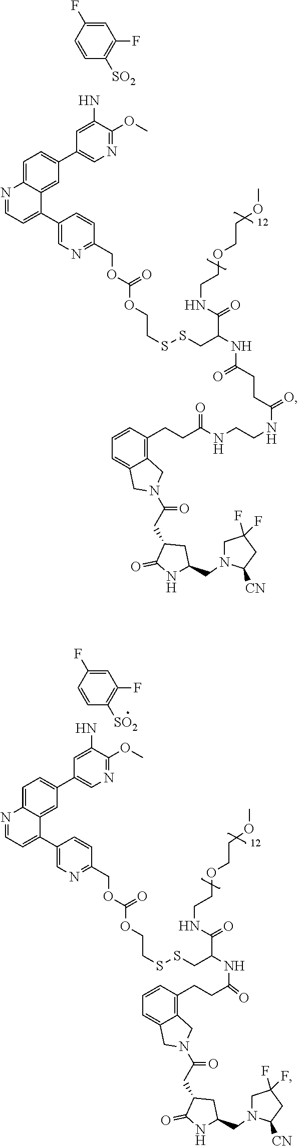

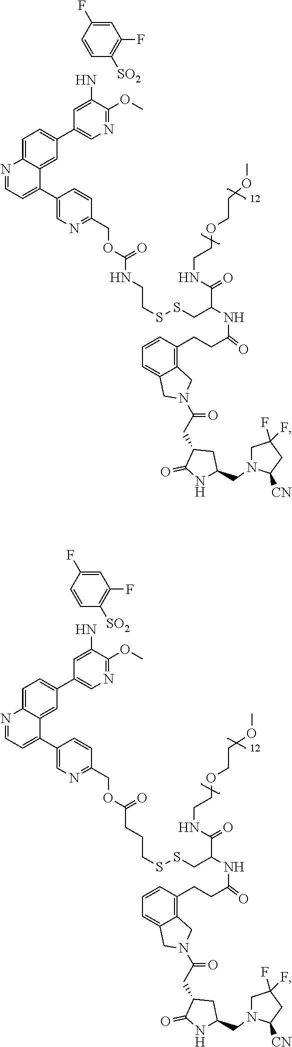

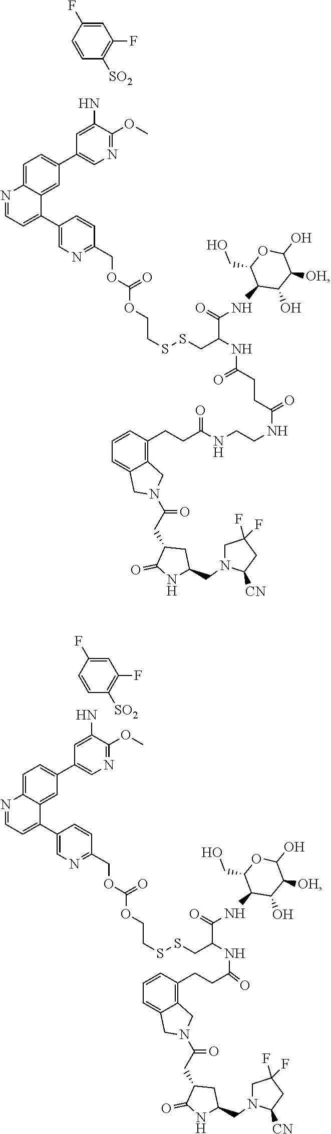

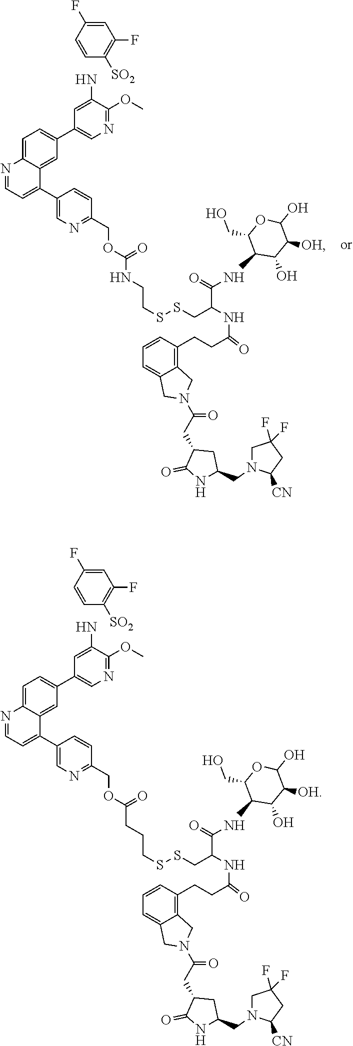

A compound (e.g., conjugate) can have the following structure:

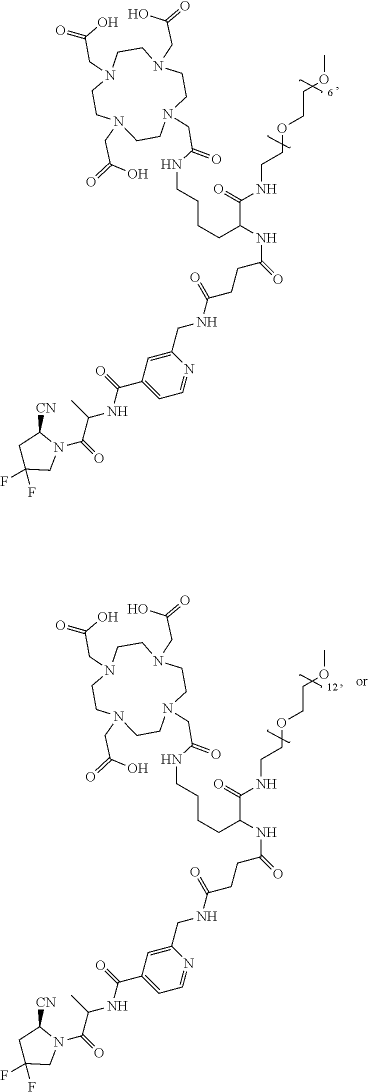

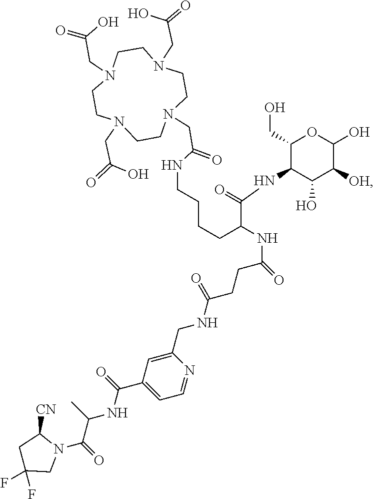 [SMILES: CC(NC(=O)c1ccnc(CNC(=O)CCC(=O)NC(CCCCNC(=O)CN2CCN(CC(=O)O)CCN(CC(=O)O)CCN(CC(=O)O)CC2)C(=O)N[C@@H]2[C@@H](O)[C@H](O)C(O)O[C@H]2CO)c1)C(=O)N1CC(F)(F)C[C@H]1C#N]

each optionally bound to an isotope (or metal) suitable for radio-imaging, radiotherapy or magnetic resonance imaging.

A compound (e.g., conjugate) can have the following structure:

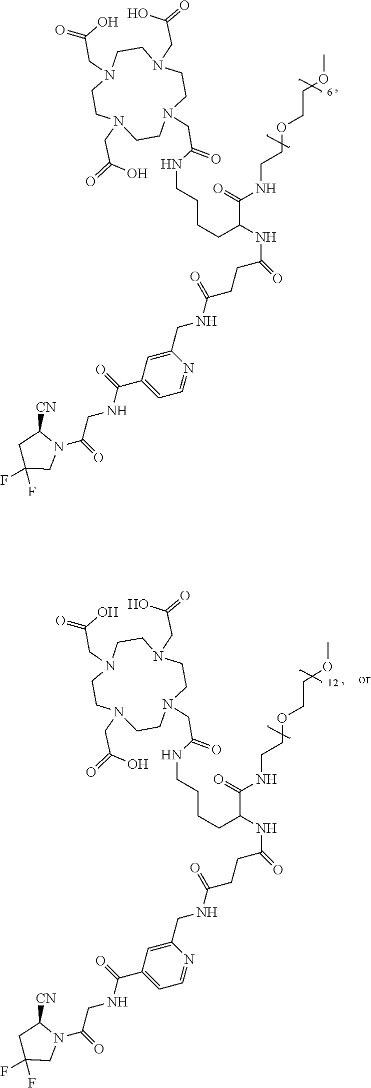

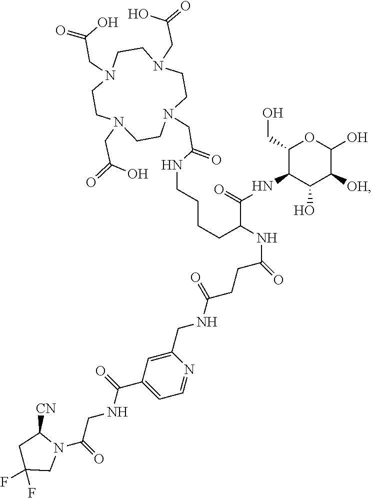 [SMILES: N#C[C@@H]1CC(F)(F)CN1C(=O)CNC(=O)c1ccnc(CNC(=O)CCC(=O)NC(CCCCNC(=O)CN2CCN(CC(=O)O)CCN(CC(=O)O)CCN(CC(=O)O)CC2)C(=O)N[C@@H]2[C@@H](O)[C@H](O)C(O)O[C@H]2CO)c1]

each optionally bound to an isotope (or metal) suitable for radio-imaging, radiotherapy or magnetic resonance imaging.

A compound (e.g., conjugate) can have the following structure:

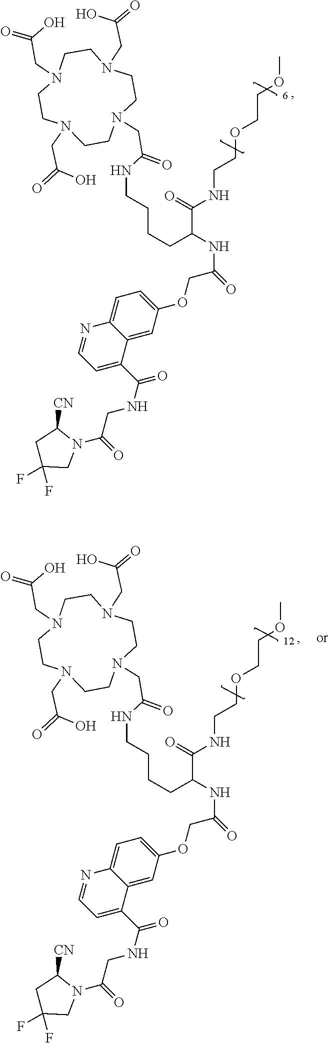

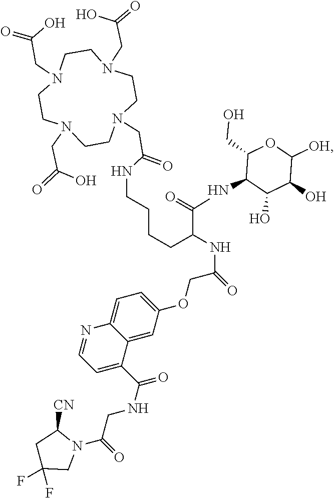 [SMILES: N#C[C@@H]1CC(F)(F)CN1C(=O)CNC(=O)c1ccnc2ccc(OCC(=O)NC(CCCCNC(=O)CN3CCN(CC(=O)O)CCN(CC(=O)O)CCN(CC(=O)O)CC3)C(=O)N[C@@H]3[C@@H](O)[C@H](O)C(O)O[C@H]3CO)cc12]

each optionally bound to an isotope (or metal) suitable for radio-imaging, radiotherapy or magnetic resonance imaging.

A compound (e.g., conjugate) can have the following structure:

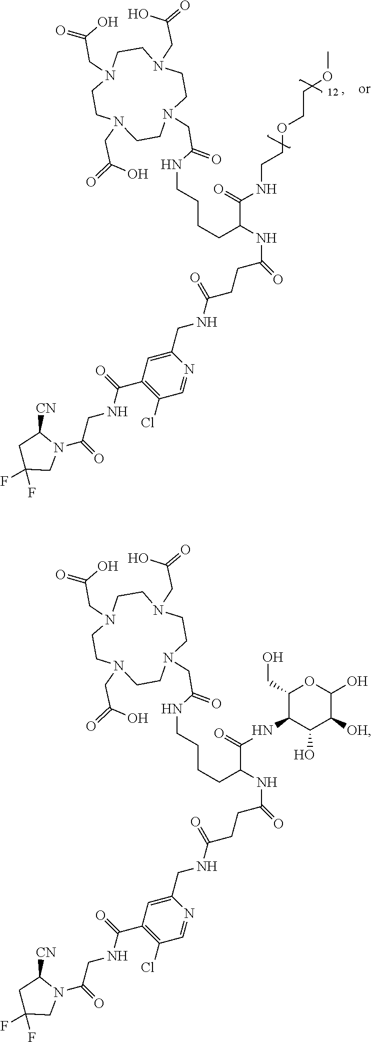

each optionally bound to an isotope (or metal) suitable for radio-imaging, radiotherapy or magnetic resonance imaging.

A compound (e.g., conjugate) can have the following structure:

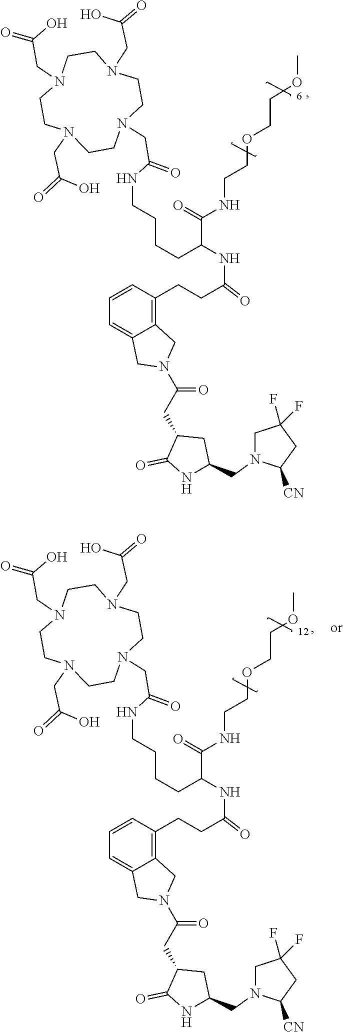

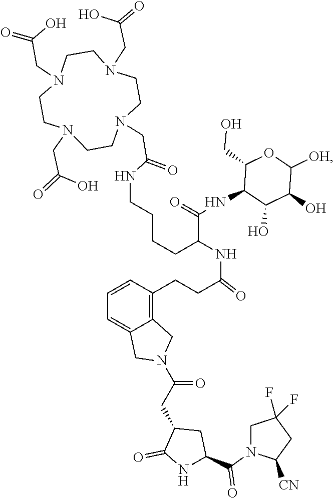

each optionally bound to an isotope (or metal) suitable for radio-imaging, radiotherapy or magnetic resonance imaging.

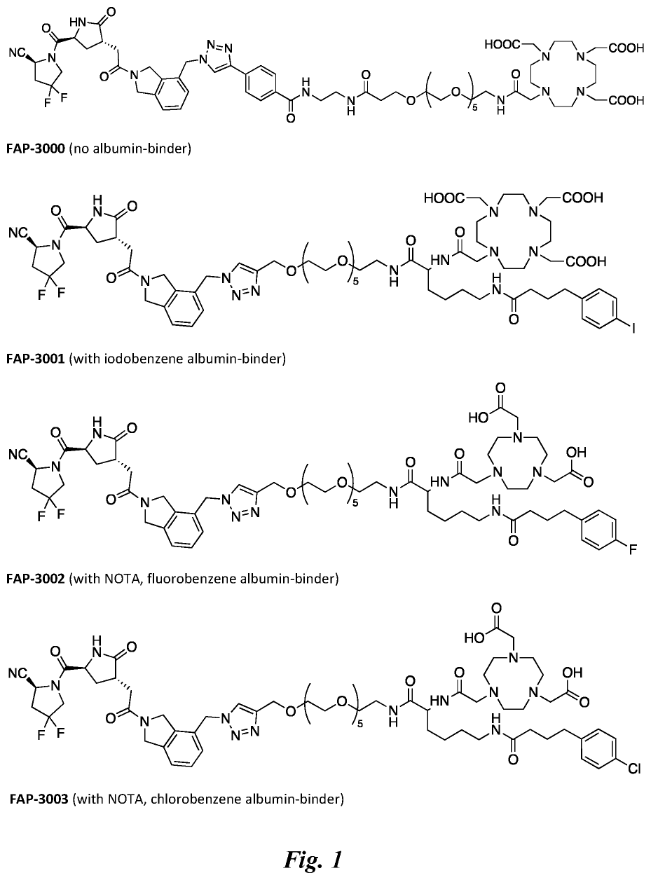

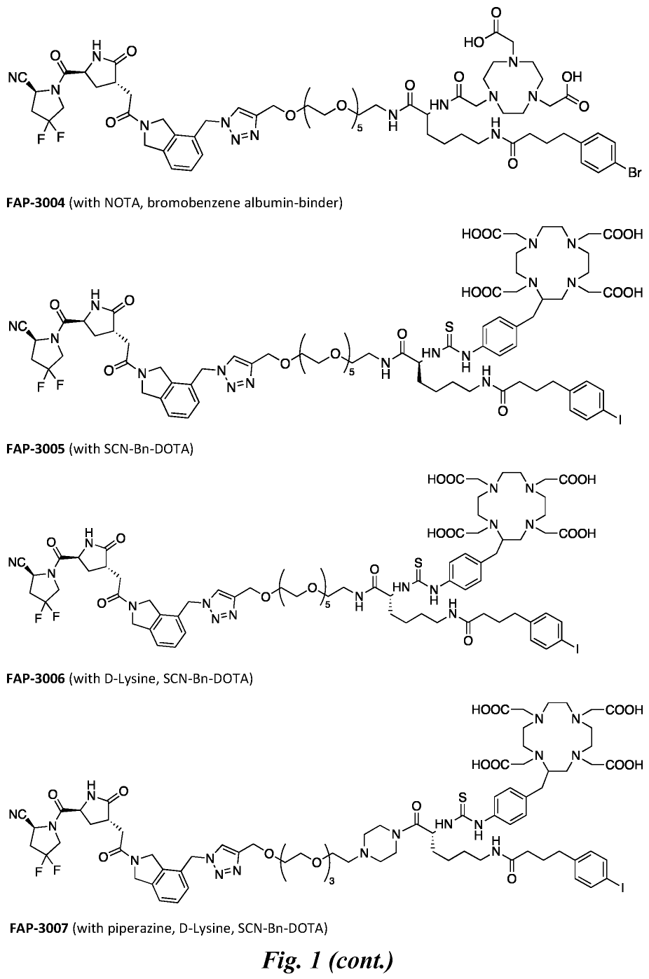

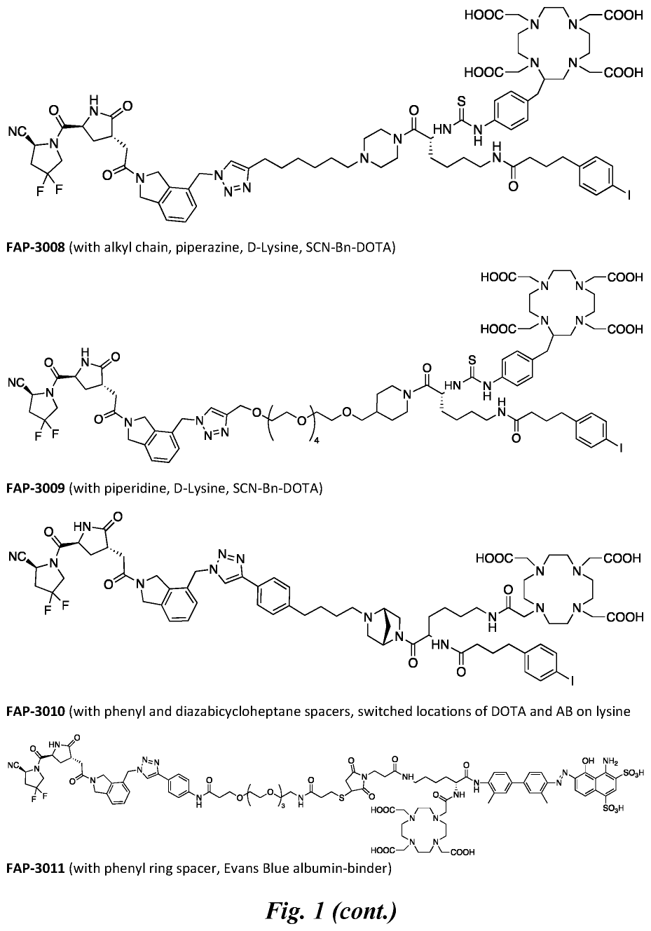

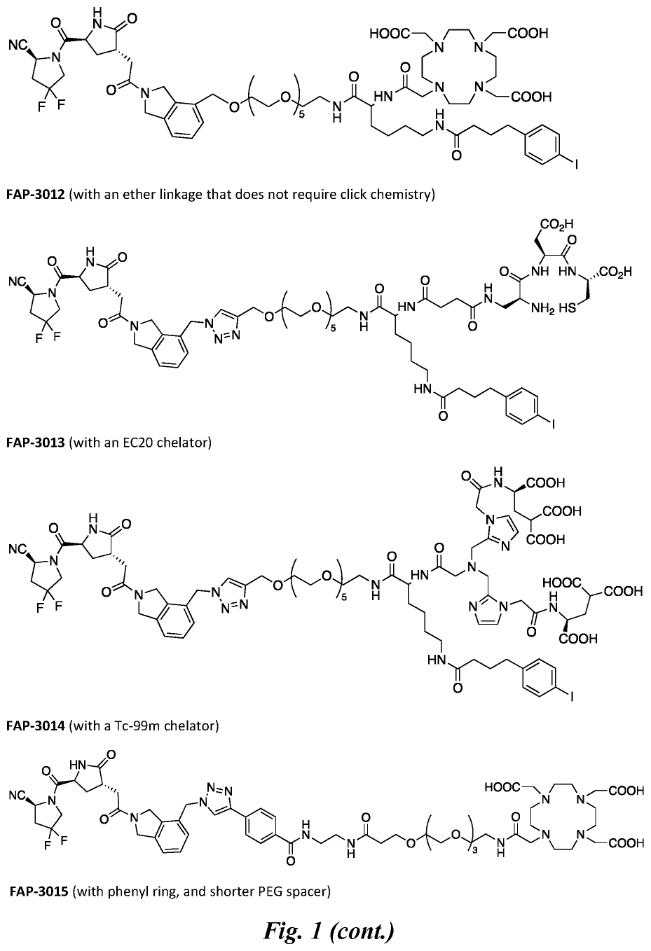

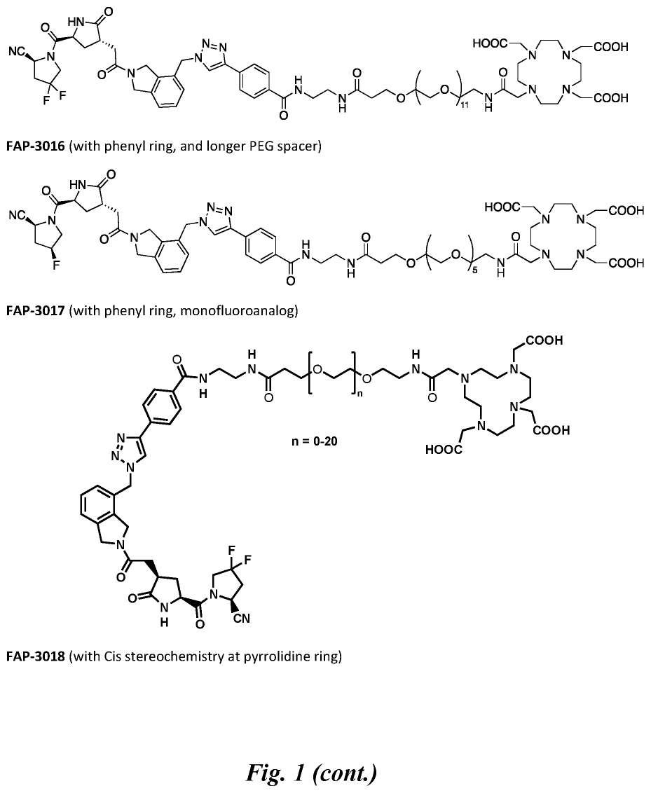

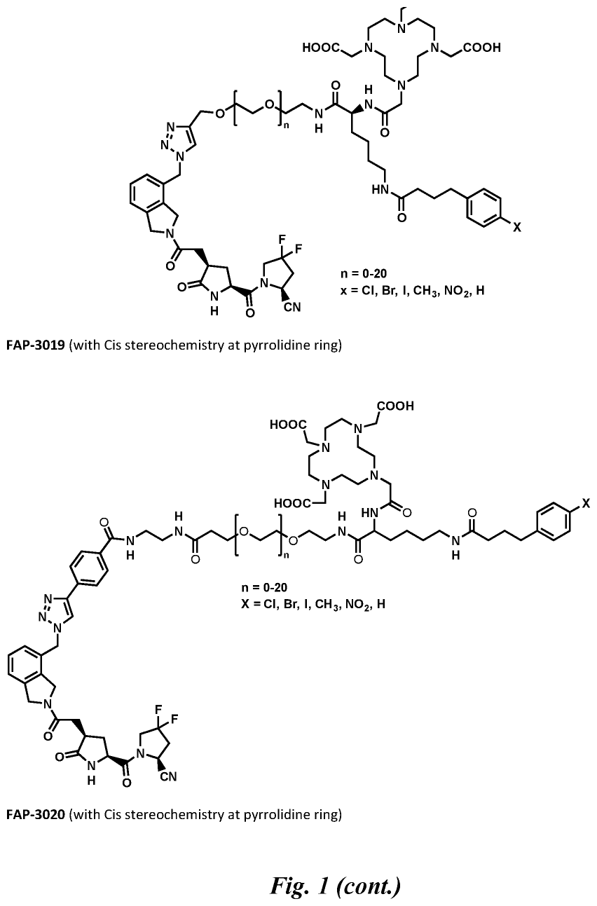

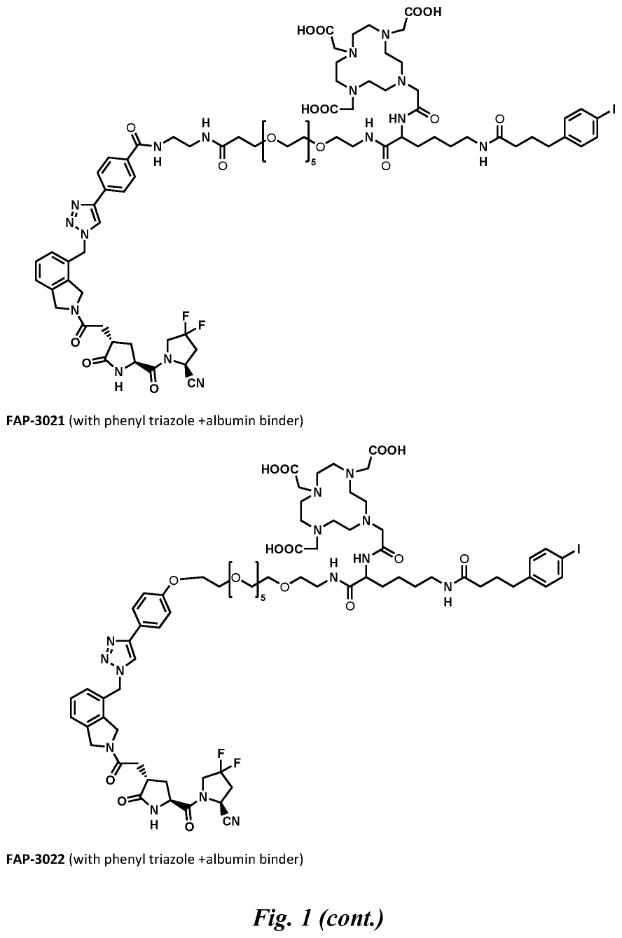

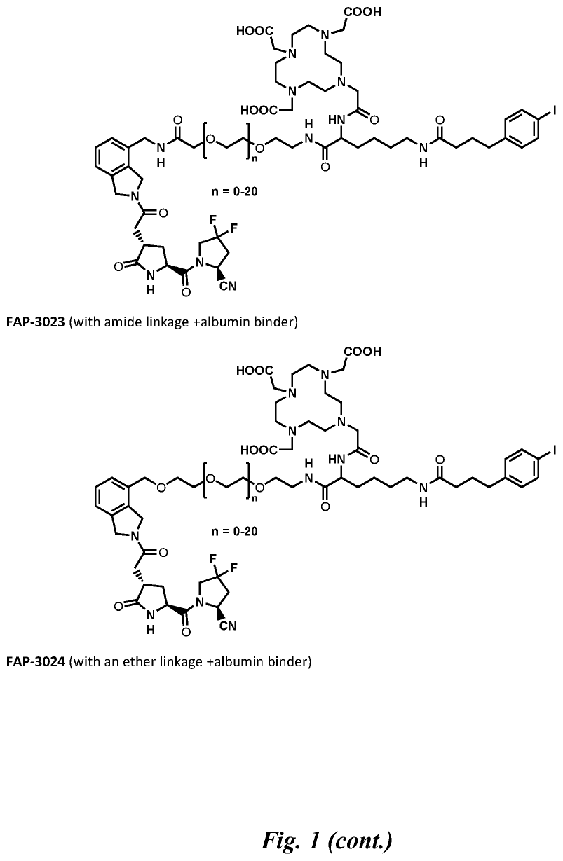

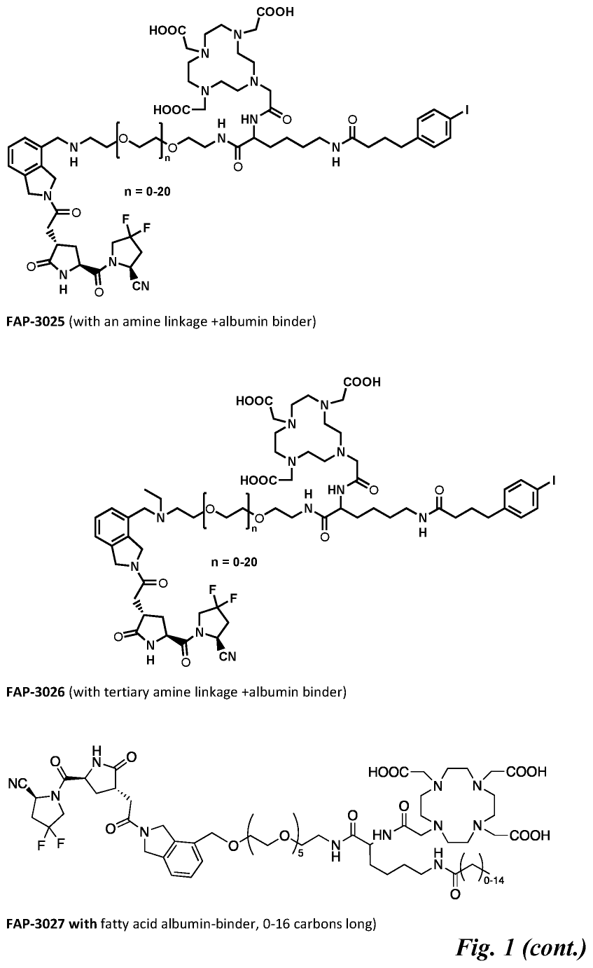

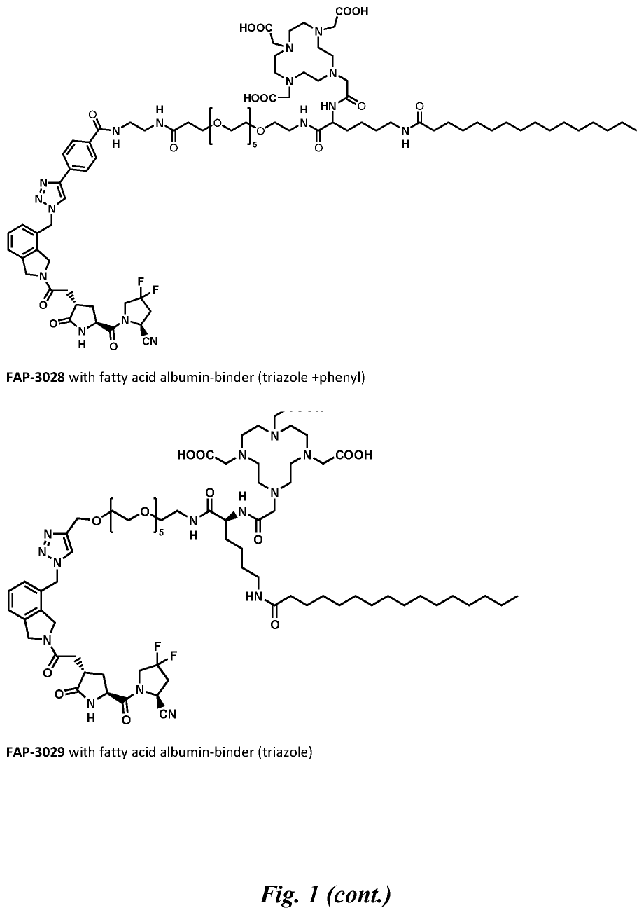

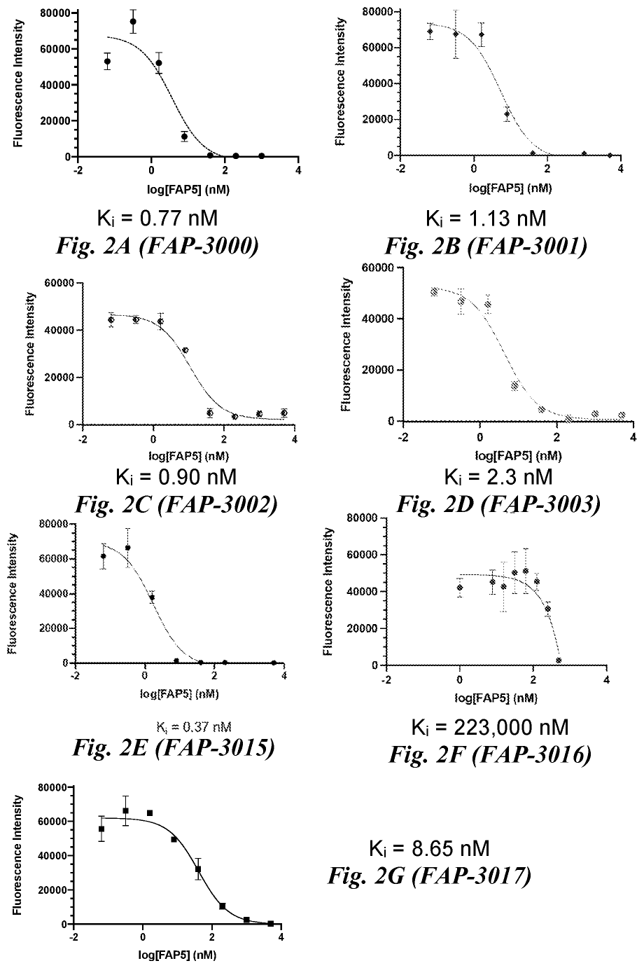

The compound can have the structure of any of compounds depicted in FIG. 1, each optionally bound to an isotope (or metal) suitable for radio-imaging, radiotherapy or magnetic resonance imaging.

The compound can have the formula:

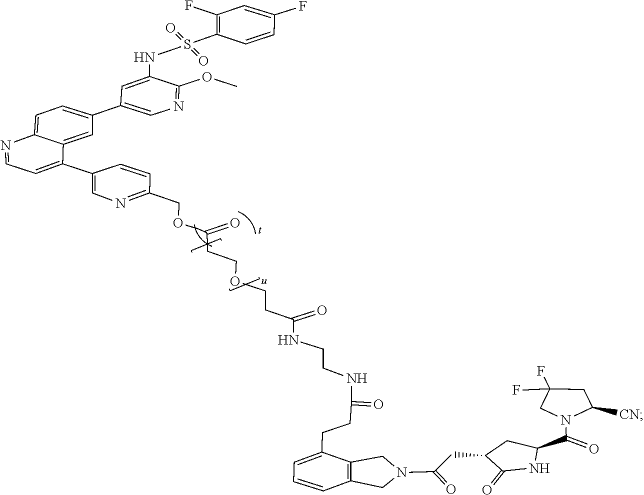

wherein t is 0 or 1 and u is an integer from 2-12.

The compound can have the formula

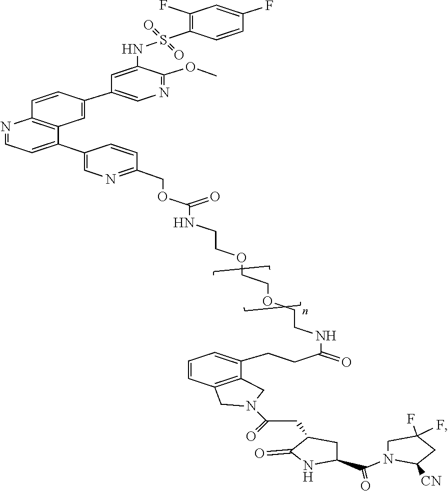

wherein n is an integer from 1 to 12.

The compound can have the formula

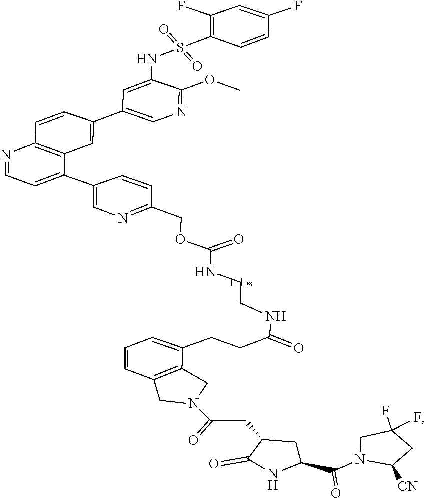

wherein m is an integer from 1 to 4.

The compound can have the formula

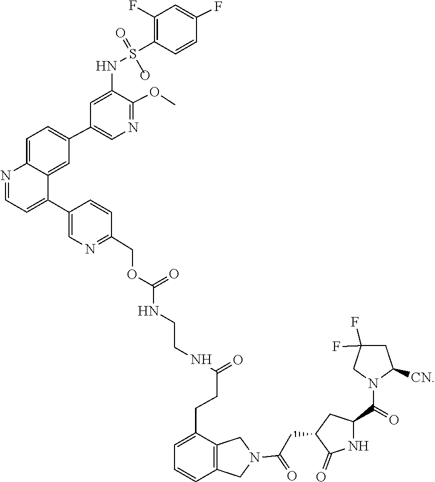

The compound can have the formula

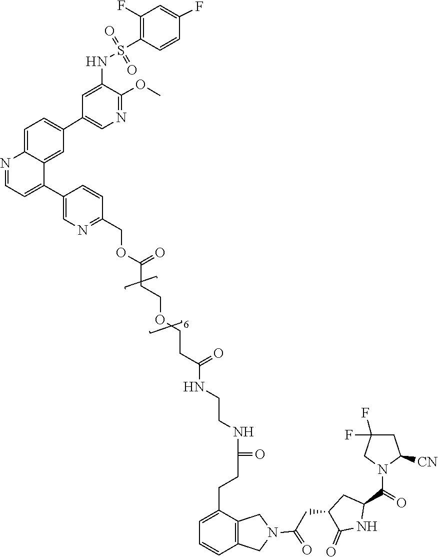

The compound can have the formula

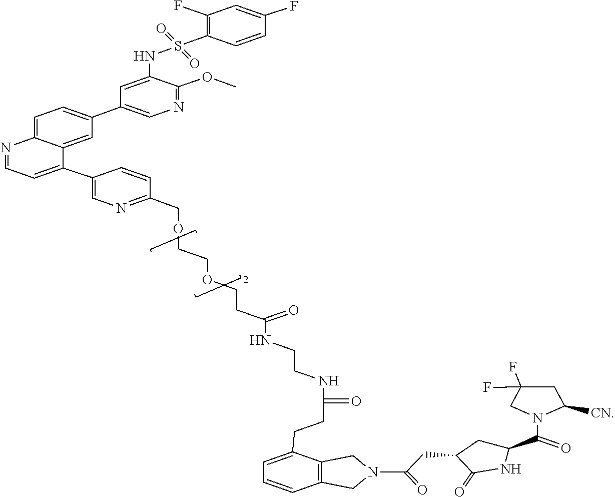

The compound can have the formula

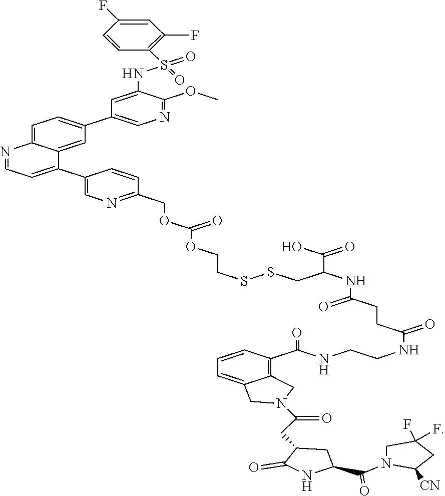 [SMILES: COc1ncc(-c2ccc3nccc(-c4ccc(COC(=O)OCCSSCC(NC(=O)CCC(=O)NCCNC(=O)c5cccc6c5CN(C(=O)C[C@@H]5C[C@@H](C(=O)N7CC(F)(F)C[C@H]7C#N)NC5=O)C6)C(=O)O)nc4)c3c2)cc1NS(=O)(=O)c1ccc(F)cc1F]

The compound can have the formula

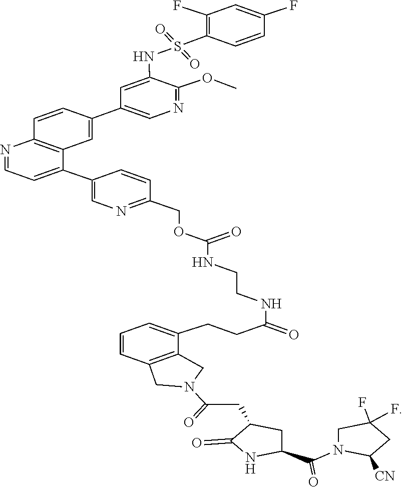 [SMILES: COc1ncc(-c2ccc3nccc(-c4ccc(COC(=O)NCCNC(=O)CCc5cccc6c5CN(C(=O)C[C@@H]5C[C@@H](C(=O)N7CC(F)(F)C[C@H]7C#N)NC5=O)C6)nc4)c3c2)cc1NS(=O)(=O)c1ccc(F)cc1F]

The compound can have any of the formula set forth in FIG. 20A.

In certain embodiments the compound (i.e., a conjugate) can have the following structure:

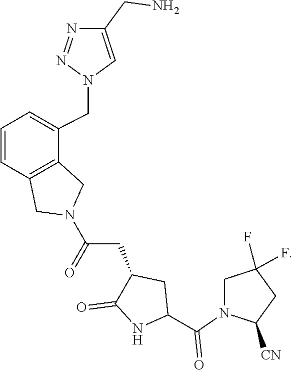 [SMILES: N#C[C@@H]1CC(F)(F)CN1C(=O)C1C[C@@H](CC(=O)N2Cc3cccc(Cn4cc(CN)nn4)c3C2)C(=O)N1]

In certain embodiments, the compound (i.e., conjugate) can have the following structure:

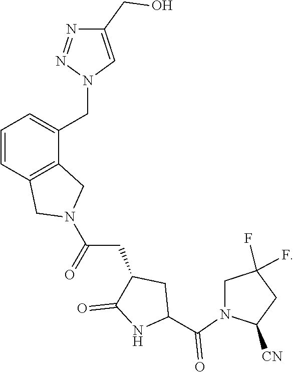 [SMILES: N#C[C@@H]1CC(F)(F)CN1C(=O)C1C[C@@H](CC(=O)N2Cc3cccc(Cn4cc(CO)nn4)c3C2)C(=O)N1]

In certain embodiments, the compound (i.e., conjugate) can have the following structure:

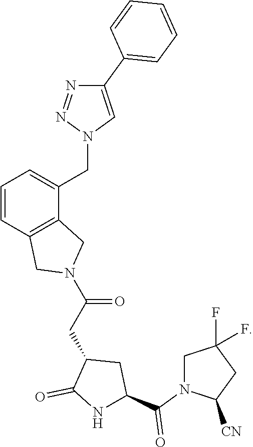 [SMILES: N#C[C@@H]1CC(F)(F)CN1C(=O)[C@@H]1C[C@@H](CC(=O)N2Cc3cccc(Cn4cc(-c5ccccc5)nn4)c3C2)C(=O)N1]

In certain embodiments, the compound (i.e., conjugate) can have the following structure:

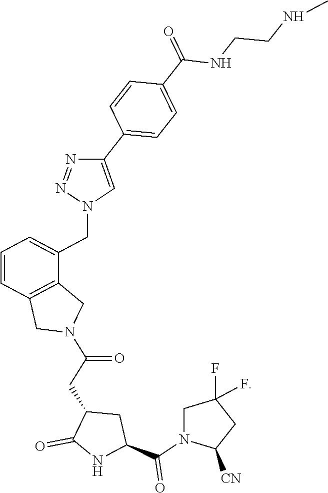 [SMILES: CNCCNC(=O)c1ccc(-c2cn(Cc3cccc4c3CN(C(=O)C[C@@H]3C[C@@H](C(=O)N5CC(F)(F)C[C@H]5C#N)NC3=O)C4)nn2)cc1]

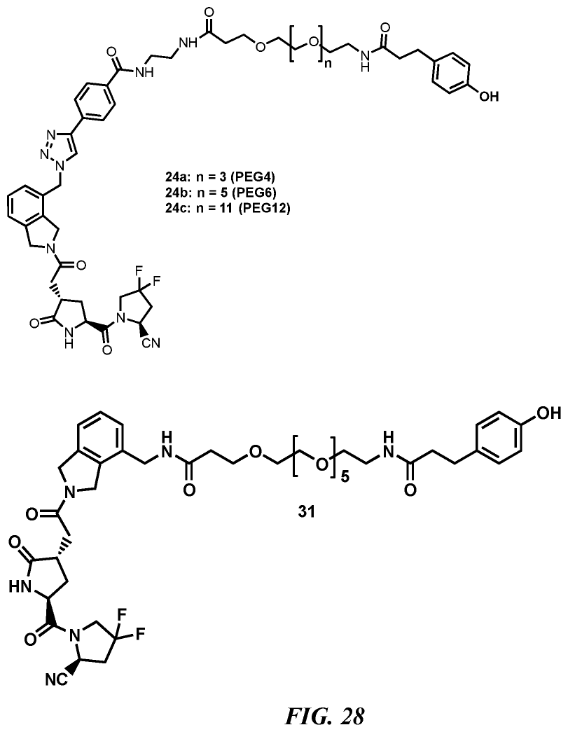

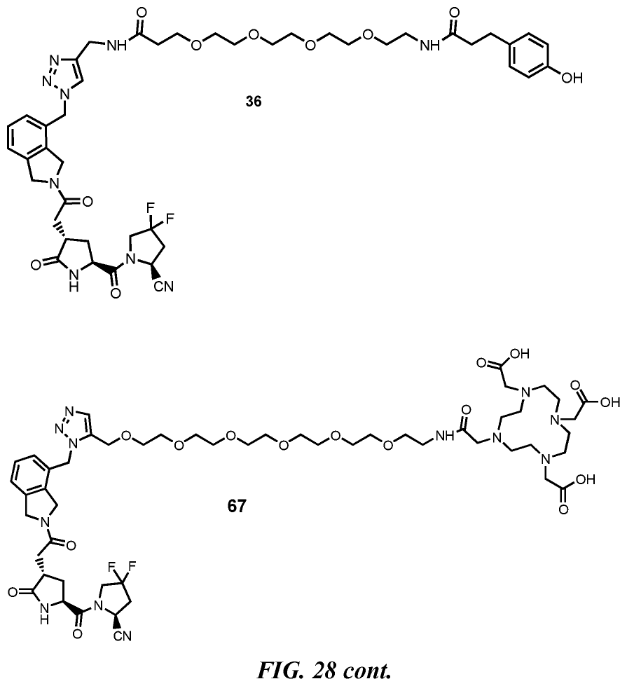

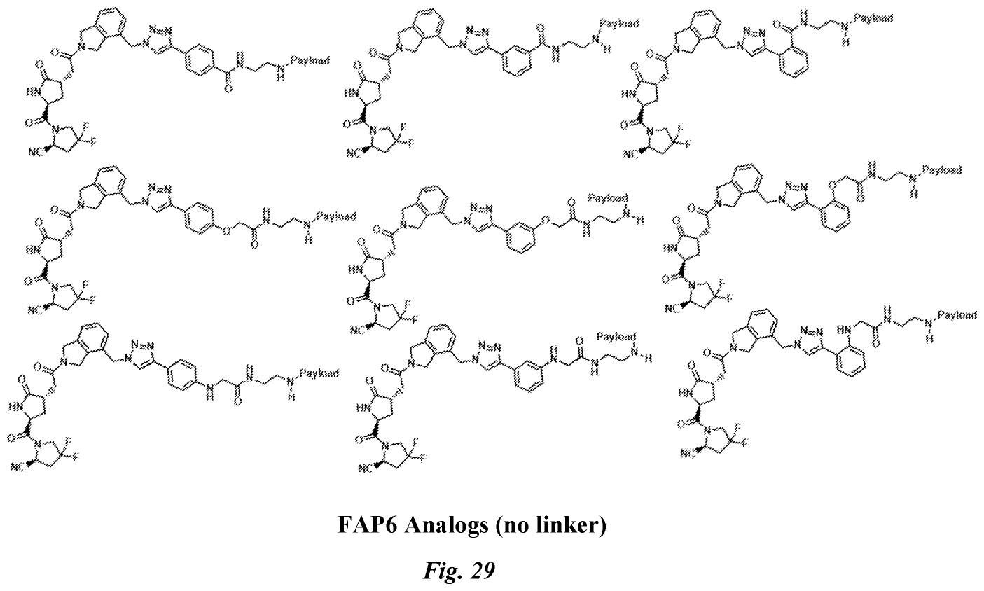

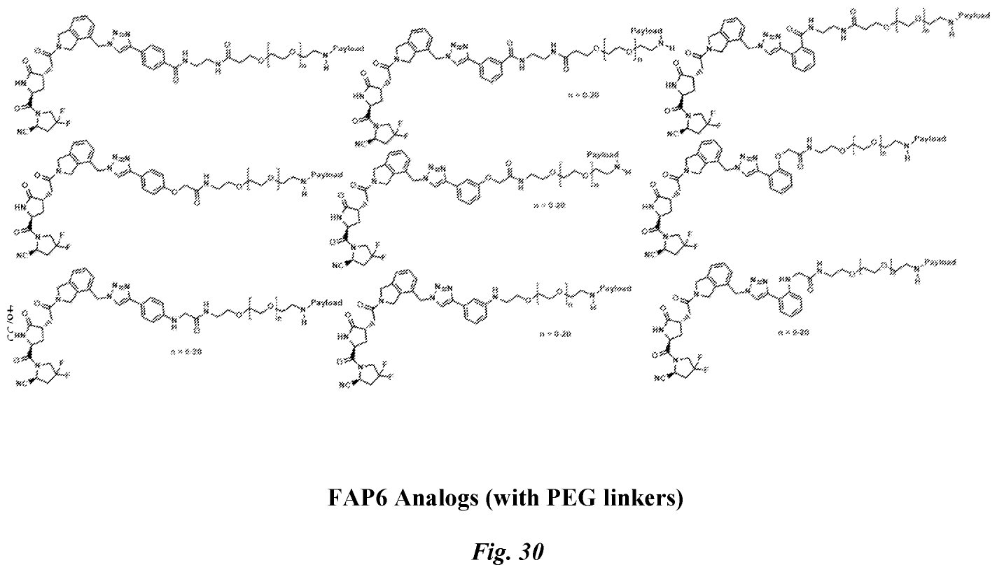

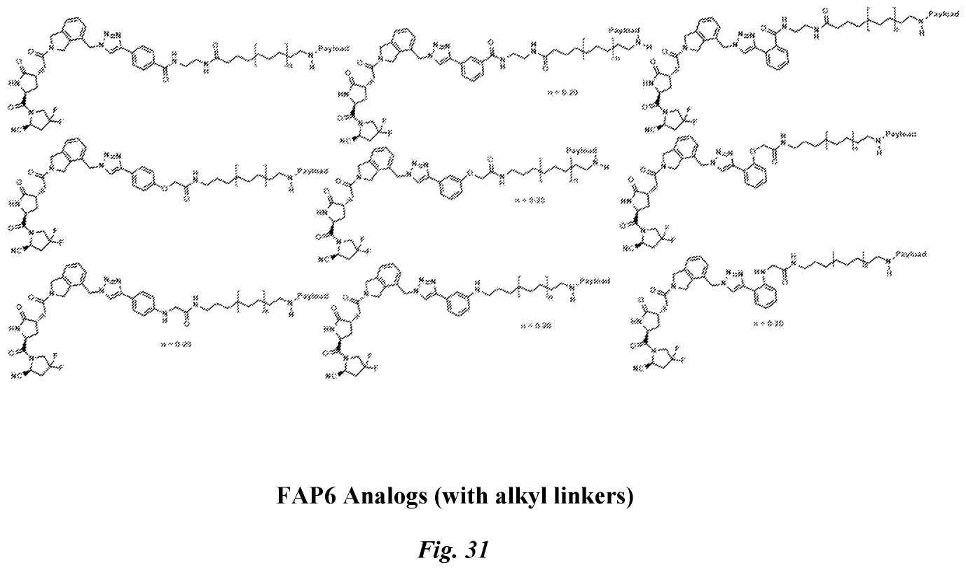

In certain embodiments, the compound (i.e., conjugate) can have any of the structures in FIGS. 28-31.

In certain embodiments, the compound can have any of the following structures:

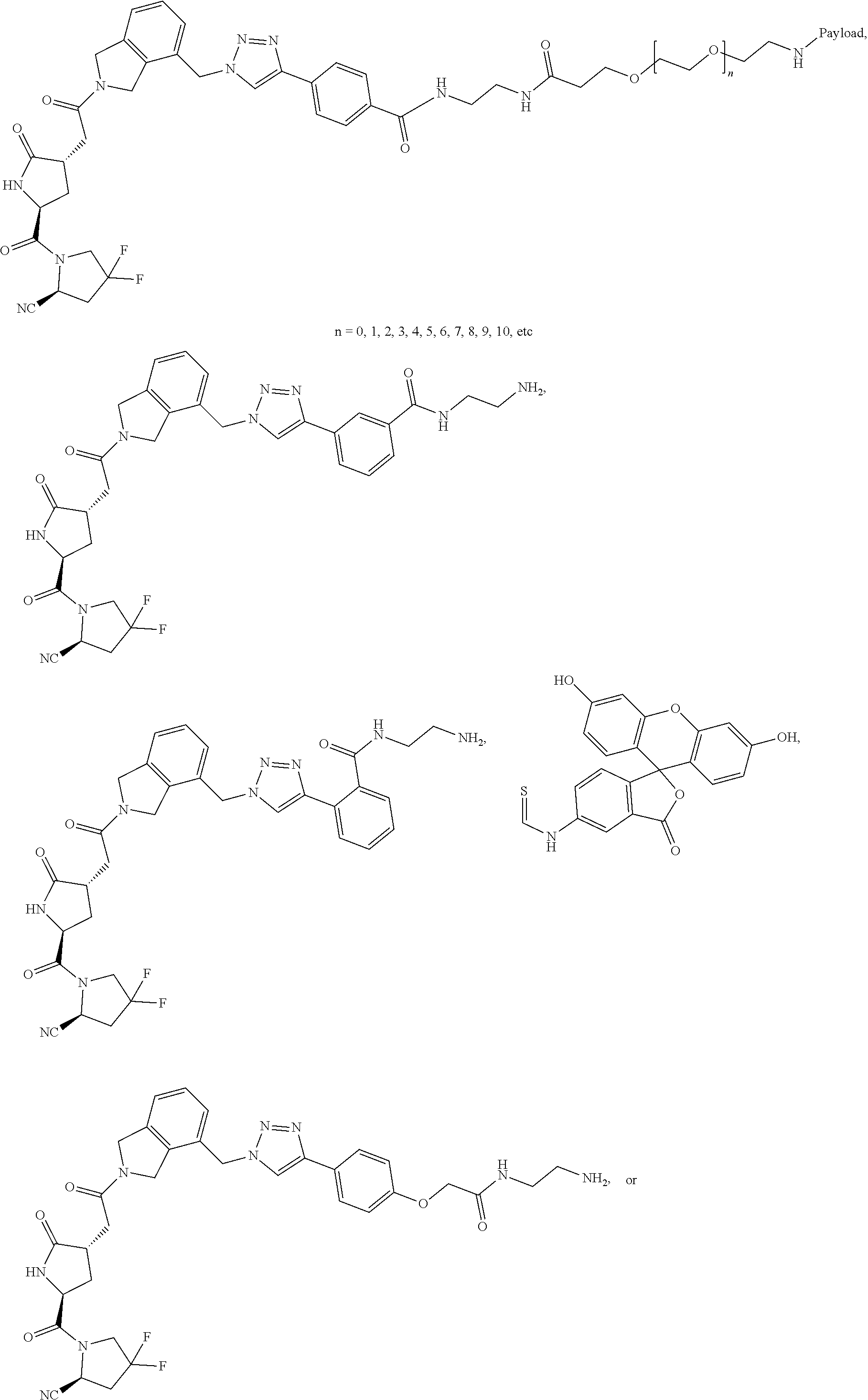

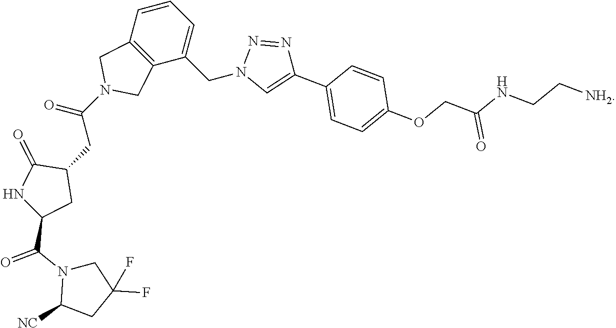 [SMILES: N#C[C@@H]1CC(F)(F)CN1C(=O)[C@@H]1C[C@@H](CC(=O)N2Cc3cccc(Cn4cc(-c5ccc(OCC(=O)NCCN)cc5)nn4)c3C2)C(=O)N1]

### Pharmaceutical Compositions, Routes of Administration, and Dosing

In certain embodiments, provided is a pharmaceutical composition comprising a compound and a pharmaceutically acceptable carrier. In certain embodiments, the pharmaceutical composition comprises a plurality of compounds and a pharmaceutically acceptable carrier.

In certain embodiments, a pharmaceutical composition further comprises at least one additional pharmaceutically active agent. The at least one additional pharmaceutically active agent can be an agent useful in the treatment of ischemia-reperfusion injury.

Pharmaceutical compositions can be prepared by combining one or more compounds with a pharmaceutically acceptable carrier and, optionally, one or more additional pharmaceutically active agents.

As used herein, an “effective amount” refers to any amount that is sufficient to achieve a desired biological effect. Combined with the teachings provided herein, by choosing among the various active compounds and weighing factors such as potency, relative bioavailability, patient body weight, severity of adverse side-effects and mode of administration, an effective prophylactic or therapeutic treatment regimen can be planned which does not cause substantial unwanted toxicity and yet is effective to treat the particular subject. The effective amount for any particular application can vary depending on such factors as the disease or condition being treated, the particular compound being administered, the size of the subject, or the severity of the disease or condition. One of ordinary skill in the art can empirically determine the effective amount of a particular compound and/or other therapeutic agent without necessitating undue experimentation. A maximum dose can be used, that is, the highest safe dose according to some medical judgment. Multiple doses per day can be used to achieve appropriate systemic levels of compounds. Appropriate systemic levels can be determined by, for example, measurement of the patient's peak or sustained plasma level of the drug. “Dose” and “dosage” are used interchangeably herein.

Generally, daily oral doses of a compound are, for human subjects, from about 0.01 milligrams/kg per day to 1,000 milligrams/kg per day. Oral doses in the range of 0.5 to 50 milligrams/kg, in one or more administrations per day, can yield therapeutic results. Dosage can be adjusted appropriately to achieve desired drug levels, local or systemic, depending upon the mode of administration. For example, intravenous administration can vary from one order to several orders of magnitude lower dose per day. If the response in a subject is insufficient at such doses, even higher doses (or effective higher doses by a different, more localized delivery route) can be employed to the extent that patient tolerance permits. Multiple doses per day are contemplated to achieve appropriate systemic levels of the compound.

A “therapeutically effective amount” (or “effective amount”) of a compound with respect to use in treatment, refers to an amount of the compound in a preparation which, when administered as part of a desired dosage regimen (to a mammal, such as a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

For any compound therapeutically effective amount can be initially determined from animal models. A therapeutically effective dose can also be determined from human data for compounds which have been tested in humans and for compounds which are known to exhibit similar pharmacological activities, such as other related active agents. Higher doses may be required for parenteral administration. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

For clinical use, any compound can be administered in an amount equal or equivalent to 0.2-2,000 milligram (mg) of compound per kilogram (kg) of body weight of the subject per day. The compounds can be administered in a dose equal or equivalent to 2-2,000 mg of compound per kg body weight of the subject per day. The compounds can be administered in a dose equal or equivalent to 20-2,000 mg of compound per kg body weight of the subject per day. The compounds can be administered in a dose equal or equivalent to 50-2,000 mg of compound per kg body weight of the subject per day. The compounds can be administered in a dose equal or equivalent to 100-2,000 mg of compound per kg body weight of the subject per day. The compounds can be administered in a dose equal or equivalent to 200-2,000 mg of compound per kg body weight of the subject per day. Where a precursor or prodrug of a compound is to be administered, it is administered in an amount that is equivalent to, i.e., sufficient to deliver, the above-stated amounts of the compound.

The formulations of the compounds can be administered to human subjects in therapeutically effective amounts. Typical dose ranges are from about 0.01 microgram/kg to about 2 mg/kg of body weight per day. The dosage of drug to be administered is likely to depend on such variables as the type and extent of the disorder, the overall health status of the particular subject, the specific compound being administered, the excipients used to formulate the compound, and its route of administration. Routine experiments can be used to optimize the dose and dosing frequency for any particular compound.

The compounds can be administered at a concentration in the range from about 0.001 microgram/kg to greater than about 500 mg/kg. For example, the concentration can be 0.001 microgram/kg, 0.01 microgram/kg, 0.05 microgram/kg, 0.1 microgram/kg, 0.5 microgram/kg, 1.0 microgram/kg, 10.0 microgram/kg, 50.0 microgram/kg, 100.0 microgram/kg, 500 microgram/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed.

The compounds can be administered at a dosage in the range from about 0.2 milligram/kg/day to greater than about 100 mg/kg/day. For example, the dosage can be 0.2 mg/kg/day to 100 mg/kg/day, 0.2 mg/kg/day to 50 mg/kg/day, 0.2 mg/kg/day to 25 mg/kg/day, 0.2 mg/kg/day to 10 mg/kg/day, 0.2 mg/kg/day to 7.5 mg/kg/day, 0.2 mg/kg/day to 5 mg/kg/day, 0.25 mg/kg/day to 100 mg/kg/day, 0.25 mg/kg/day to 50 mg/kg/day, 0.25 mg/kg/day to 25 mg/kg/day, 0.25 mg/kg/day to 10 mg/kg/day, 0.25 mg/kg/day to 7.5 mg/kg/day, 0.25 mg/kg/day to 5 mg/kg/day, 0.5 mg/kg/day to 50 mg/kg/day, 0.5 mg/kg/day to 25 mg/kg/day, 0.5 mg/kg/day to 20 mg/kg/day, 0.5 mg/kg/day to 15 mg/kg/day, 0.5 mg/kg/day to 10 mg/kg/day, 0.5 mg/kg/day to 7.5 mg/kg/day, 0.5 mg/kg/day to 5 mg/kg/day, 0.75 mg/kg/day to 50 mg/kg/day, 0.75 mg/kg/day to 25 mg/kg/day, 0.75 mg/kg/day to 20 mg/kg/day, 0.75 mg/kg/day to 15 mg/kg/day, 0.75 mg/kg/day to 10 mg/kg/day, 0.75 mg/kg/day to 7.5 mg/kg/day, 0.75 mg/kg/day to 5 mg/kg/day, 1.0 mg/kg/day to 50 mg/kg/day, 1.0 mg/kg/day to 25 mg/kg/day, 1.0 mg/kg/day to 20 mg/kg/day, 1.0 mg/kg/day to 15 mg/kg/day, 1.0 mg/kg/day to 10 mg/kg/day, 1.0 mg/kg/day to 7.5 mg/kg/day, 1.0 mg/kg/day to 5 mg/kg/day, 2 mg/kg/day to 50 mg/kg/day, 2 mg/kg/day to 25 mg/kg/day, 2 mg/kg/day to 20 mg/kg/day, 2 mg/kg/day to 15 mg/kg/day, 2 mg/kg/day to 10 mg/kg/day, 2 mg/kg/day to 7.5 mg/kg/day, or 2 mg/kg/day to 5 mg/kg/day.

The compounds can be administered at a dosage in the range from about 0.25 milligram/kg/day to about 25 mg/kg/day. For example, the dosage can be 0.25 mg/kg/day, 0.5 mg/kg/day, 0.75 mg/kg/day, 1.0 mg/kg/day, 1.25 mg/kg/day, 1.5 mg/kg/day, 1.75 mg/kg/day, 2.0 mg/kg/day, 2.25 mg/kg/day, 2.5 mg/kg/day, 2.75 mg/kg/day, 3.0 mg/kg/day, 3.25 mg/kg/day, 3.5 mg/kg/day, 3.75 mg/kg/day, 4.0 mg/kg/day, 4.25 mg/kg/day, 4.5 mg/kg/day, 4.75 mg/kg/day, 5 mg/kg/day, 5.5 mg/kg/day, 6.0 mg/kg/day, 6.5 mg/kg/day, 7.0 mg/kg/day, 7.5 mg/kg/day, 8.0 mg/kg/day, 8.5 mg/kg/day, 9.0 mg/kg/day, 9.5 mg/kg/day, 10 mg/kg/day, 11 mg/kg/day, 12 mg/kg/day, 13 mg/kg/day, 14 mg/kg/day, 15 mg/kg/day, 16 mg/kg/day, 17 mg/kg/day, 18 mg/kg/day, 19 mg/kg/day, 20 mg/kg/day, 21 mg/kg/day, 22 mg/kg/day, 23 mg/kg/day, 24 mg/kg/day, 25 mg/kg/day, 26 mg/kg/day, 27 mg/kg/day, 28 mg/kg/day, 29 mg/kg/day, 30 mg/kg/day, 31 mg/kg/day, 32 mg/kg/day, 33 mg/kg/day, 34 mg/kg/day, 35 mg/kg/day, 36 mg/kg/day, 37 mg/kg/day, 38 mg/kg/day, 39 mg/kg/day, 40 mg/kg/day, 41 mg/kg/day, 42 mg/kg/day, 43 mg/kg/day, 44 mg/kg/day, 45 mg/kg/day, 46 mg/kg/day, 47 mg/kg/day, 48 mg/kg/day, 49 mg/kg/day, or 50 mg/kg/day.

The compound or precursor thereof can be administered in concentrations that range from 0.01 micromolar to greater than or equal to 500 micromolar. For example, the dose can be 0.01 micromolar, 0.02 micromolar, 0.05 micromolar, 0.1 micromolar, 0.15 micromolar, 0.2 micromolar, 0.5 micromolar, 0.7 micromolar, 1.0 micromolar, 3.0 micromolar, 5.0 micromolar, 7.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 60.0 micromolar, 70.0 micromolar, 80.0 micromolar, 90.0 micromolar, 100.0 micromolar, 150.0 micromolar, 200.0 micromolar, 250.0 micromolar, 300.0 micromolar, 350.0 micromolar, 400.0 micromolar, 450.0 micromolar, to greater than about 500.0 micromolar or any incremental value thereof. It is to be understood that all values and ranges between these values and ranges are meant to be encompassed.

The compound or precursor thereof can be administered at concentrations that range from 0.10 microgram/mL to 500.0 microgram/mL. For example, the concentration can be 0.10 microgram/mL, 0.50 microgram/mL, 1 microgram/mL, 2.0 microgram/mL, 5.0 microgram/mL, 10.0 microgram/mL, 20 microgram/mL, 25 microgram/mL. 30 microgram/mL, 35 microgram/mL, 40 microgram/mL, 45 microgram/mL, 50 microgram/mL, 60.0 microgram/mL, 70.0 microgram/mL, 80.0 microgram/mL, 90.0 microgram/mL, 100.0 microgram/mL, 150.0 microgram/mL, 200.0 microgram/mL, 250.0 g/mL, 250.0 micro gram/mL, 300.0 microgram/mL, 350.0 microgram/mL, 400.0 microgram/mL, 450.0 microgram/mL, to greater than about 500.0 microgram/mL or any incremental value thereof. It is to be understood that all values and ranges between these values and ranges are meant to be encompassed.

The formulations can be administered in pharmaceutically acceptable solutions, which can routinely contain pharmaceutically acceptable concentrations of salt, buffering agents, preservatives, compatible carriers, adjuvants, and optionally other therapeutic ingredients. For use in therapy, an effective amount of the compound can be administered to a subject by any mode that delivers the compound to the desired surface. Administering a pharmaceutical composition can be accomplished by any means known to the skilled artisan. Routes of administration include, but are not limited to, intravenous, intramuscular, intraperitoneal, intravesical (urinary bladder), oral, subcutaneous, direct injection (for example, into a tumor or abscess), mucosal (e.g., topical to eye), inhalation, and topical.

For intravenous and other parenteral routes of administration, a compound can be formulated as a lyophilized preparation, as a lyophilized preparation of liposome-intercalated or -encapsulated active compound, as a lipid complex in aqueous suspension, or as a salt complex. Lyophilized formulations are generally reconstituted in suitable aqueous solution, e.g., in sterile water or saline, shortly prior to administration.

For oral administration, the compounds can be formulated readily by combining the active compound(s) with pharmaceutically acceptable carriers well-known in the art. Such carriers enable the compounds to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents can be added, such as the cross-linked PVP, agar, or alginic acid or a salt thereof such as sodium alginate. Optionally the oral formulations can also be formulated in saline or buffers, e.g., EDTA for neutralizing internal acid conditions, or can be administered without any carriers.

Also contemplated are oral dosage forms of the compounds. The compounds can be chemically modified so that oral delivery of the derivative is efficacious. Generally, the chemical modification contemplated is the attachment of at least one moiety to the compound itself, where said moiety permits (a) inhibition of acid hydrolysis; and (b) uptake into the blood stream from the stomach or intestine. Also desired is the increase in overall stability of the compounds and increase in circulation time in the body. Examples of such moieties include polyethylene glycol, copolymers of ethylene glycol and propylene glycol, carboxymethyl cellulose, dextran, polyvinyl alcohol, PVP and polyproline. Abuchowski and Davis, “Soluble Polymer-Enzyme Adducts,” In: Enzymes as Drugs, Hocenberg and Roberts, eds., Wiley-Interscience, New York, N.Y., pp. 367-383 (1981); Newmark et al., J Appl Biochem 4:185-189 (1982). Other polymers that could be used are poly-1,3-dioxolane and poly-1,3,6-tioxocane. For pharmaceutical usage, as indicated above, polyethylene glycol moieties are suitable.

The location of release of a compound hereof can be the stomach, the small intestine (e.g., the duodenum, the jejunum, or the ileum), or the large intestine. One skilled in the art has available formulations, which will not dissolve in the stomach, yet will release the material in the duodenum or elsewhere in the intestine. The release can avoid the deleterious effects of the stomach environment, either by protection of the compound or by release of the compound beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and shellac. These coatings can be used as mixed films.

A coating or mixture of coatings can also be used on tablets, which are not intended for protection against the stomach. This can include sugar coatings, or coatings which make the tablet easier to swallow. Capsules can consist of a hard shell (such as gelatin) for delivery of dry therapeutic (e.g., powder); for liquid forms, a soft gelatin shell can be used. The shell material of cachets could be thick starch or other edible paper. For pills, lozenges, molded tablets or tablet triturates, moist massing techniques can be used.

Therapeutic agent can be included in the formulation as fine multi-particulates in the form of granules or pellets of particle size about 1 mm. The formulation of the material for capsule administration could also be as a powder, lightly compressed plugs or even as tablets. Therapeutic agent could be prepared by compression.

Colorants and flavoring agents may all be included. For example, the compound can be formulated (such as by liposome or microsphere encapsulation) and then further contained within an edible product, such as a refrigerated beverage containing colorants and flavoring agents.

One may dilute or increase the volume of therapeutic agent with an inert material. These diluents can include carbohydrates, especially mannitol, a-lactose, anhydrous lactose, cellulose, sucrose, modified dextrans and starch. Certain inorganic salts also can be used as fillers, including calcium triphosphate, magnesium carbonate and sodium chloride. Some commercially available diluents are Fast-Flo, Emdex, STA-Rx 1500, Emcompress and Avicell.

Disintegrants can be included in the formulation of therapeutic agent into a solid dosage form. Materials used as disintegrates include, but are not limited to, starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrant is the insoluble cationic exchange resin. Powdered gums can be used as disintegrants and as binders and these can include powdered gums such as agar, Karaya or tragacanth. Alginic acid and its sodium salt are also useful as disintegrants.

Binders can be used to hold therapeutic agent together to form a hard tablet and include materials from natural products such as acacia, tragacanth, starch and gelatin. Others include methyl cellulose (MC), ethyl cellulose (EC) and carboxymethyl cellulose (CMC). PVP and hydroxypropylmethyl cellulose (HPMC) can both be used in alcoholic solutions to granulate therapeutic agent.

An anti-frictional agent can be included in the formulation of therapeutic to prevent sticking during the formulation process. Lubricants can be used as a layer between therapeutic agent and the die wall, and these can include, but are not limited to, stearic acid, including its magnesium and calcium salts, polytetrafluoroethylene (PTFE), liquid paraffin, vegetable oils and waxes. Soluble lubricants can also be used, such as sodium lauryl sulfate, magnesium lauryl sulfate, polyethylene glycol of various molecular weights, Carbowax 4000 and 6000.

Glidants, which can improve the flow properties of the drug during formulation and aid rearrangement during compression, can be added. The glidants can include starch, talc, pyrogenic silica and hydrated silicoaluminate.

To aid dissolution of therapeutic agent into the aqueous environment a surfactant can be added as a wetting agent. Surfactants can include anionic detergents, such as sodium lauryl sulfate, dioctyl sodium sulfosuccinate and dioctyl sodium sulfonate. Cationic detergents which can be used include benzalkonium chloride and benzethonium chloride. Potential non-ionic detergents that can be included in the formulation as surfactants include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the compound or derivative thereof either alone or as a mixture in different ratios.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. Microspheres formulated for oral administration can also be used. Such microspheres have been well defined in the art. All formulations for oral administration should be in dosages suitable for such administration.

For buccal administration, the compositions can take the form of tablets or lozenges formulated in conventional manner.

For topical administration, the compound can be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

For administration by inhalation, compounds can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

Also contemplated is pulmonary delivery of the compounds (or salts thereof). The compound is delivered to the lungs of a mammal while inhaling and traverses across the lung epithelial lining to the blood stream. Other reports of inhaled molecules include Adjei et al., Pharm Res 7:565-569 (1990); Adjei et al., Int J Pharmaceutics 63:135-144 (1990) (leuprolide acetate); Braquet et al., J Cardiovasc Pharmacol 13(suppl. 5):143-146 (1989) (endothelin-1); Hubbard et al., Annal Int Med 3:206-212 (1989) (al-antitrypsin); Smith et al., 1989, J Clin Invest 84:1145-1146 (a-1-proteinase); Oswein et al., 1990, “Aerosolization of Proteins,” Proceedings of Symposium on Respiratory Drug Delivery II, Keystone, Colorado, March, (recombinant human growth hormone); Debs et al., 1988, J Immunol 140:3482-3488 (interferon-gamma and tumor necrosis factor alpha) and Platz et al., U.S. Pat. No. 5,284,656 (granulocyte colony stimulating factor; incorporated herein by reference). A method and composition for pulmonary delivery of drugs for systemic effect is described in U.S. Pat. No. 5,451,569 (specifically incorporated herein by reference for its disclosure regarding same), issued Sep. 19, 1995, to Wong et al.

Contemplated for use are a wide range of mechanical devices designed for pulmonary delivery of therapeutic products, including but not limited to nebulizers, metered dose inhalers, and powder inhalers, all of which are familiar to those skilled in the art.

Nasal delivery of a pharmaceutical composition is also contemplated. Nasal delivery allows the passage of a pharmaceutical composition to the blood stream directly after administering therapeutic product to the nose, without the necessity for deposition of the product in the lung. Formulations for nasal delivery include those with dextran or cyclodextran.

The compounds, when it is desirable to deliver them systemically, can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethylcellulose, sorbitol, or dextran. Optionally, the suspension can also contain suitable stabilizers or agents which increase the solubility of the compounds to allow for the preparation of highly concentrated solutions.

Alternatively, the active compounds can be in powder form for constitution with a suitable vehicle, e.g., sterile pyrogen-free water, before use.

The compounds can also be formulated in rectal or vaginal compositions such as suppositories or retention enemas, e.g., containing conventional suppository bases such as cocoa butter or other glycerides.

In addition to the formulations described above, a compound can also be formulated as a depot preparation. Such long-acting formulations can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R, Science 249:1527-1533 (1990).

The compound and optionally one or more other therapeutic agents can be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

Pharmaceutical compositions contain an effective amount of a compound as described herein and optionally one or more other therapeutic agents included in a pharmaceutically acceptable carrier. The term “pharmaceutically acceptable carrier” means one or more compatible solid or liquid fillers, diluents or encapsulating substances which are suitable for administration to a human or other vertebrate animal. The term “carrier” denotes an organic or inorganic ingredient, natural or synthetic, with which the active ingredient is combined to facilitate the application. The components of the pharmaceutical compositions also can be commingled with the compounds, and with each other, in a manner such that there is no interaction which would substantially impair the desired pharmaceutical efficiency.

Therapeutic agent(s), including specifically, but not limited to, a compound, can be provided in particles. “Particles” as used herein means nanoparticles or microparticles (or in some instances larger particles) that can consist in whole or in part of the compound or the other therapeutic agent(s) as described herein. The particles can contain therapeutic agent(s) in a core surrounded by a coating, including, but not limited to, an enteric coating. Therapeutic agent(s) also can be dispersed throughout the particles. Therapeutic agent(s) also can be adsorbed into the particles. The particles can be of any order release kinetics, including zero-order release, first-order release, second-order release, delayed release, sustained release, immediate release, and any combination thereof, etc. The particle can include, in addition to therapeutic agent(s), any of those materials routinely used in the art of pharmacy and medicine, including, but not limited to, erodible, nonerodible, biodegradable, or nonbiodegradable material or combinations thereof. The particles can be microcapsules which contain the compound in a solution or in a semi-solid state. The particles can be of virtually any shape.

Both non-biodegradable and biodegradable polymeric materials can be used in the manufacture of particles for delivering therapeutic agent(s). Such polymers can be natural or synthetic polymers. The polymer is selected based on the period of time over which release is desired. Bioadhesive polymers of particular interest include bioerodible hydrogels described in Sawhney et al., Macromolecules 26:581-587 (1993), the teachings of which are specifically incorporated by reference herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Therapeutic agent(s) can be contained in controlled-release systems. The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) is used in its conventional sense to refer to a drug formulation that provides for gradual release of a drug over an extended period of time, and that can result in substantially constant blood levels of a drug over an extended time period. The term “delayed release” is used in its conventional sense to refer to a drug formulation in which there is a time delay between administration of the formulation and the release of the drug therefrom. “Delayed release” may or may not involve gradual release of drug over an extended period of time, and thus may or may not be “sustained release.”

Use of a long-term sustained release implant can be particularly suitable for treatment of chronic conditions. “Long-term” release, as used herein, means that the implant is constructed and arranged to deliver therapeutic levels of the active ingredient for at least 7 days, and up to 30-60 days. Long-term sustained release implants are well-known to those of ordinary skill in the art and include some of the release systems described above.

### Methods of Treatment

A method for treating an inflammatory disease or disorder is also provided. The method for treating an inflammatory disease or disorder can be by modulating the activity of activated fibroblasts. As used herein, the term “modulate” and its variants means to change or induce an alteration in a particular biological activity. Modulation includes, but is not limited to, stimulating or inhibiting an activity (e.g., by activating a receptor so as to initiate a signal transduction cascade, to inhibit a receptor from propagating a signaling pathway, by activating an endogenous inhibitor that attenuates a biological activity, or by inhibiting the activity of a protein that inhibits a particular biological function.

The method can comprise administering a compound (e.g., a conjugate) of any formula provided herein. The method can comprise administering of any of the pharmaceutical compositions described herein. The method can comprise administering a therapeutically effective amount of a compound (e.g., a conjugate) of any formula provided herein (whether as part of a pharmaceutical composition or otherwise).

In some embodiments the inflammatory disease or disorder is selected from the group consisting of Crohn's disease, lupus, inflammatory bowel disease (IBS), Addison's disease, Grave's disease, Sjogren's syndrome, celiac disease, Hashimoto's thyroiditis, myasthenia gravis, autoimmune vasculitis, reactive arthritis, psoriatic arthritis, pernicious anemia, ulcerative colitis, rheumatoid arthritis, type 1 diabetes, multiple sclerosis, or fibrotic disease, graft vs. host disease (GVHD), transplant rejection, fatty liver disease, asthma, osteoporosis, sarcoidosis, ischemia-reperfusion injury, prosthesis osteolysis, glomerulonephritis, scleroderma, psoriasis, with autoimmune myocarditis, spinal cord injury, central nervous system, viral infection, influenza, coronavirus infection, cytokine storm syndrome, bone damage, inflammatory brain disease, and atherosclerosis.

A method for treating cancer is provided. The method of treating cancer can be by modulating the activity of activated fibroblasts. The method can comprise administering a compound (e.g., a conjugate) of any formula provided herein and/or any of the pharmaceutical compositions provided herein. The method can comprise contacting a CAF (e.g., a CAF of a cancer patient) with a compound (e.g., a conjugate) of any formula provided herein. In some embodiments the cancer is selected from the group consisting of lung cancer, bone cancer, pancreatic cancer, skin cancer, cancer of the head, cancer of the neck, cutaneous melanoma, intraocular melanoma, uterine cancer, ovarian cancer, endometrial cancer, leiomyosarcoma, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland cancer of the parathyroid gland, non-small cell lung cancer, small cell lung cancer, cancer of the adrenal gland, sarcoma of soft tissue, cancer of the urethra, cancer of the penis, prostate cancer, chronic leukemia, acute leukemia, lymphocytic lymphomas, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, cholangiocarcinoma, Hurthle cell thyroid cancer, and adenocarcinoma of the gastroesophageal junction. In some embodiments the cancer is selected form the group consisting of lung cancer, breast cancer, colorectal cancer, cervical cancer, and brain cancer (e.g., glioblastoma).

A method for treating fibrosis is also provided. The method of treating fibrosis can be by modulating the activity of activated fibroblasts. The method can comprise administering (e.g., a therapeutically effective amount of) a compound (e.g., a conjugate) of any formula provided herein (e.g., as part of a pharmaceutical composition provided herein or otherwise). In some embodiments, the fibrosis is selected from the group consisting of pulmonary fibrosis, renal fibrosis, and hepatic fibrosis. In some embodiments the fibrosis is idiopathic pulmonary fibrosis.

The method for treating fibrosis or cancer can further comprise administering chemotherapy or radiotherapy to the subject. In certain embodiments, the method for treating fibrosis or cancer comprises administering (e.g., a therapeutically effective amount of) a compound (e.g., a conjugate) of any formula provided herein (e.g., as part of a pharmaceutical composition provided herein or otherwise) alone. In certain embodiments, the method for treating fibrosis or cancer comprises administering (e.g., a therapeutically effective amount of) a compound (e.g., a conjugate) of any formula provided herein (e.g., as part of a pharmaceutical composition provided herein or otherwise) to a subject in combination with one or more additional therapies. Such additional therapies can include, without limitation, an immunotherapy, a DNA damage response pathway inhibitor, chemotherapy, and/or a surgery.

A method for imaging cancer or fibrosis in a subject with the cancer or the fibrosis is provided. In certain embodiments, the method comprises administering, to the subject, an effective amount (e.g., a therapeutically effective amount) of a compound (e.g., a conjugate) of any compound provided herein (e.g., as part of a pharmaceutical composition provided herein or otherwise). In certain embodiments, the method further comprises imaging the subject. In certain embodiments, the method further comprises generating an image of the cancer or fibrosis in the subject (e.g., after or concurrently with administration of the compound).

Also provided are compositions and methods for optical imaging. The compositions and methods can be for fluorescence-guided surgery. The compositions and methods can be for radio-imaging.

The disclosure also relates to compositions and methods for magnetic resonance imaging (MRI).

The methods above comprise the steps of: providing to the patient in need thereof a effective amount of conjugate A-L-B, wherein A comprises a FAPα targeting moiety, such as a moiety with a molecular weight below 10,000; L comprises one or more linkers, which can form chemical bonds with at least A and B′; and B′ comprises an optical dye (e.g., a fluorescent dye), a photodynamic therapeutic agent, a radio-imaging agent, a radiotherapeutic agent, a chemotherapeutic agent, an antifibrotic agent, or an anticancer agent that is effective against cancer cells, cancer-associated fibroblasts, myofibroblasts, and/or other tumor microenvironment factors.

The radio-imaging agent can be a magnetic resonance (MR) contrast agent. The MR contrast agent can comprise iron oxide particles. The iron oxide particles can be nanoparticles. The iron oxide particles can be paramagnetic particles, superparamagnetic particles (SPIO), or ultrasmall superparamagnetic particles (USPIO).

### Methods for Improving the Affinity of a Ligand for FAP

Methods for improving the affinity of a ligand for FAP are also provided. In certain embodiments, a method for improving the affinity of a ligand for FAP, such ligand comprising an isoindoline scaffold, comprises introducing a triazole moiety into the isoindoline or another scaffold of the ligand by molecular modeling to achieve a higher Schrodinger molecular docking score, whereupon the affinity of the ligand for FAP is improved. In certain embodiments, the method can comprise incorporating an ethyldiamino aryl triazole moiety into a FAP ligand. In certain embodiments, the method can comprise introducing a triazole moiety and a phenyl ring into a FAP ligand by molecular modeling to achieve a higher Schrodinger molecular docking score.

### Definitions

Unless defined otherwise, all technical and scientific terms used herein have the same meaning as commonly understood by one of skill in the chemical and biological arts. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl.

The term “prophylactic or therapeutic” treatment is art-recognized and includes administration to the patient of one or more compound of the disclosure. If it is administered prior to clinical manifestation of the unwanted condition (e.g., disease or other unwanted state of the host animal) then the treatment is prophylactic, (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “patient”, “individual” or “subject” refers to a mammal in need of a particular treatment. A patient or subject can be a primate, canine, feline, or equine. A patient or subject can be a bird. The bird can be a domesticated bird, such as chicken. The bird can be a fowl. A patient or subject can be a human.

“Oxo” refers to the ═O radical.

“Alkyl” generally refers to a straight or branched hydrocarbon chain radical consisting solely of carbon and hydrogen atoms, such as having from one to fifteen carbon atoms (e.g., C₁-Cis alkyl). Disclosures provided herein of an “alkyl” are intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₁-C₅ alkyl). An alkyl can comprise one to four carbon atoms (e.g., C₁-C₄ alkyl). An alkyl can comprise one to three carbon atoms (e.g., C₁-C₃ alkyl). An alkyl can comprise one to two carbon atoms (e.g., C₁-C₂ alkyl). An alkyl can comprise one carbon atom (e.g., C₁ alkyl). An alkyl can comprise five to fifteen carbon atoms (e.g., C₅-C₁₅ alkyl). An alkyl can comprise five to eight carbon atoms (e.g., C₅-C₈ alkyl). An alkyl can comprise two to five carbon atoms (e.g., C₂-C₅ alkyl). An alkyl can comprise three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Alkoxy” refers to a radical bonded through an oxygen atom of the formula —O-alkyl, where alkyl is an alkyl chain as defined above.

“Alkylene” or “alkylene chain” generally refers to a straight or branched divalent alkyl group linking the rest of the molecule to a radical group, such as having from one to twelve carbon atoms, for example, methylene, ethylene, propylene, i-propylene, n-butylene, and the like.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

“Aralkyl” or “aryl-alkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. A carbocyclyl can comprise three to ten carbon atoms. A carbocyclyl can comprise five to seven carbon atoms. The carbocyclyl is attached to the rest of the molecule by a single bond. Carbocyclyl or cycloalkyl is saturated (i.e., containing single C—C bonds only) or unsaturated (i.e., containing one or more double bonds or triple bonds). Examples of saturated cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbomenyl, decalinyl, 7,7-dimethyl-bicyclo[2.2.1]heptanyl, and the like.

“Carbocyclylalkyl” refers to a radical of the formula —R^{c}-carbocyclyl where R^{c} is an alkylene chain as defined above.

“Chelating group” or “chelating group” as used herein refers to a polydentate chemical group which can bind to a central metal atom with multiple binding interactions by using two or more binding sites on the chelating group. The combination of chelating group and metal atom is a chelate. The binding of the chelating group to the metal atom can be by non-covalent interactions or bonding; in some embodiments the binding of a chelating group to a metal atom is by multiple coordinate bonds. Chelating groups include but are not limited to DOTA, NOTA, and EDTA.

“Metal suitable for radio-imaging, radiotherapy or magnetic resonance imaging” or “isotope suitable for radio-imaging, radiotherapy or magnetic resonance imaging” includes but is not limited to ¹⁸F, ³²P, ⁴⁴Sc, ⁴⁷Sc, ⁵²Mn, ⁵⁵Co, ⁶⁴Cu, ⁶⁷Cu, ⁶⁷Ga, ⁶⁸Ga, ⁸⁶Y, ⁸⁹Sr, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁶⁹Er, ¹⁷⁷Lu, ¹⁸⁶Re, ¹⁸⁸Re, ²¹¹At, ²¹²Pb, ²¹²Bi, ²¹³Bi, ²²³Ra, ²²⁴Ra, ²²⁵Ab, ²²⁵Ac, and ²²⁷Th. In some embodiments, the metal (or isotope) suitable for radio-imaging, radiotherapy or magnetic resonance imaging is ¹⁷⁷Lu. In some embodiments, the metal (or isotope) suitable for radio-imaging, radiotherapy or magnetic resonance imaging is ¹¹¹In.

“Halo” or “halogen” refers to bromo, chloro, fluoro or iodo substituents.

“Haloalkyl” refers to an alkyl radical, as defined above, that is substituted by one or more halogen radicals, as defined above, for example, trifluoromethyl, difluoromethyl, fluoromethyl, 2,2,2-trifluoroethyl, 1-fluoromethyl-2-fluoroethyl, and the like.

The term “heteroalkyl” refers to an alkyl group as defined above in which one or more skeletal carbon atoms of the alkyl are substituted with a heteroatom (with the appropriate number of substituents or valences—for example, —CH₂— can be replaced with —NH— or —O—). For example, each substituted carbon atom is independently substituted with a heteroatom, such as wherein the carbon is substituted with a nitrogen, oxygen, selenium, or other suitable heteroatom. In some instances, each substituted carbon atom is independently substituted for an oxygen, nitrogen (e.g. —NH—, —N(alkyl)-, or —N(aryl)- or having another substituent contemplated herein), or sulfur (e.g. —S—, —S(═O)—, or —S(═O)₂—). A heteroalkyl is attached to the rest of the molecule at a carbon atom of the heteroalkyl. A heteroalkyl is attached to the rest of the molecule at a heteroatom of the heteroalkyl. A heteroalkyl is a C₁-C₁₈ heteroalkyl. A heteroalkyl is a C₁-C₁₂ heteroalkyl. A heteroalkyl is a C₁-C₆ heteroalkyl. A heteroalkyl is a C₁-C₄ heteroalkyl. Heteroalkyl can include alkoxy, alkoxyalkyl, alkylamino, alkylaminoalkyl, aminoalkyl, heterocycloalkyl, heterocycloalkyl, and heterocycloalkylalkyl, as defined herein.

“Heteroalkylene” refers to a divalent heteroalkyl group defined above which links one part of the molecule to another part of the molecule.

“Heterocyclyl” refers to a stable 3- to 18-membered non-aromatic ring radical that can comprise two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes aromatic, fused, and/or bridged ring systems. The heteroatoms in the heterocyclyl radical are optionally oxidized. The heterocyclyl radical is partially or fully saturated. Disclosures provided herein of an “heterocyclyl” are intended to include independent recitations of heterocyclyl comprising aromatic and non-aromatic ring structures, unless otherwise stated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, 1,3-benzodioxolyl, 1,4-benzodioxanyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, indolinyl, isoindolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“Heteroaryl” refers to a radical derived from a 3- to 18-membered aromatic ring radical that can comprise two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, benzofuranyl, benzooxazolyl, benzo[d]thiazolyl, benzothiadiazolyl, benzo[b][1,4]dioxepinyl, benzo[b][1,4]oxazinyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-TH-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e., thienyl).

The term “radical” as used herein refers to a fragment of a molecule, wherein that fragment has an open valence for bond formation. A monovalent radical has one open valence such that it can form one bond with another chemical group. Unless otherwise specified, a radical of a molecule (e.g., a radical of a FAP ligand) is created by removal of one hydrogen atom from that molecule to create a monovalent radical with one open valence at the location where the hydrogen atom was removed. Where appropriate, a radical can be divalent, trivalent, etc., wherein two, three or more hydrogen atoms or other groups have been removed to create a radical which can bond to two, three, or more chemical groups. Where appropriate, a radical open valence may be created by removal of other than a hydrogen atom (e.g., a halogen), or by removal of two or more atoms (e.g., a hydroxyl group), as long as the atoms removed are a small fraction (20% or less of the atom count) of the total atoms in the molecule forming the radical. In some embodiments, a radical is formed from a folate, antifolate, or folate analog by removal of a hydroxyl group.

The term “releasable linker” as used herein refers to a linker that includes at least one cleavable bond that can be cleaved under extracellular physiological conditions (e.g., a pH-labile, acid-labile, oxidatively-labile, or enzyme-labile bond). Releasable groups also include photochemically-cleavable groups. Examples of photochmically-cleavable groups include 2-(2-nitrophenyl)-ethan-2-ol groups and linkers containing o-nitrobenzyl, desyl, trans-o-cinnamoyl, m-nitrophenyl or benzylsulfonyl groups (see, for example, Dorman and Prestwich, Trends Biotech. 18:64-77 (2000); and Greene and Wuts, Protective Groups in Organic Synthesis, 2nd ed., John Wiley & Sons, New York (1991)). The cleavable bond or bonds may be present in the interior of a cleavable linker and/or at one or both ends of a cleavable linker. It should be appreciated that such physiological conditions resulting in bond cleavage include standard chemical hydrolysis reactions that occur, for example, at physiological pH, or as a result of compartmentalization into a cellular organelle such as an endosome having a lower pH than cytosolic pH. Illustratively, the bivalent linkers described herein can undergo cleavage under other physiological or metabolic conditions, such as by the action of a glutathione mediated mechanism. It is appreciated that the lability of the cleavable bond may be adjusted by including functional groups or fragments within the bivalent linker L that are able to assist or facilitate such bond cleavage, also termed anchimeric assistance. The lability of the cleavable bond can also be adjusted by, for example, substitutional changes at or near the cleavable bond, such as including alpha branching adjacent to a cleavable disulfide bond, increasing the hydrophobicity of substituents on silicon in a moiety having a silicon-oxygen bond that may be hydrolyzed, homologating alkoxy groups that form part of a ketal or acetal that may be hydrolyzed, and the like. In addition, it is appreciated that additional functional groups or fragments may be included within the bivalent linker L that are able to assist or facilitate additional fragmentation of the compounds after bond breaking of the releasable linker, when present.

It will be understood by one of ordinary skill in the relevant arts that other suitable modifications and adaptations to the compositions and methods described herein are readily apparent from the description contained herein in view of information known to the ordinarily skilled artisan and can be made without departing from the scope of the disclosure or any embodiment thereof.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In the above description, numerous specific details are set forth to provide a thorough understanding of the present disclosure. Particular examples can be implemented without some or all of these specific details and it is to be understood that this disclosure is not limited to particular biological systems, particular cancers, or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein can be applied to compounds and/or composition components that have configurations other than as specifically described herein. Indeed, it is expressly contemplated that the components of the composition and compounds of the present disclosure can be tailored in furtherance of the desired application thereof.

When ranges are used herein for physical properties, such as molecular weight, or chemical properties, such as chemical formulae, all combinations and sub-combinations of ranges and specific embodiments therein are intended to be included.

Additionally, the term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “comprising” (and related terms such as “comprise” or “comprises” or “having” or “including”) is not intended to exclude that in other certain embodiments, for example, an embodiment of any compound, composition of matter, composition, method, or process, or the like, described herein, may “consist of” or “consist essentially of” the described features. The term “substantially” can allow for a degree of variability in a value or range, for example, within 90%, within 95%, or within 99% of a stated value or of a stated limit of a range.

Where a method of therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

### EXAMPLES

The following examples illustrate certain specific embodiments of the present disclosure and are not meant to limit the scope of the claimed invention in any way.

### Example 1A

### Synthesis of FAP5-Carbamate-PI3K Inhibitor (PI3Ki)

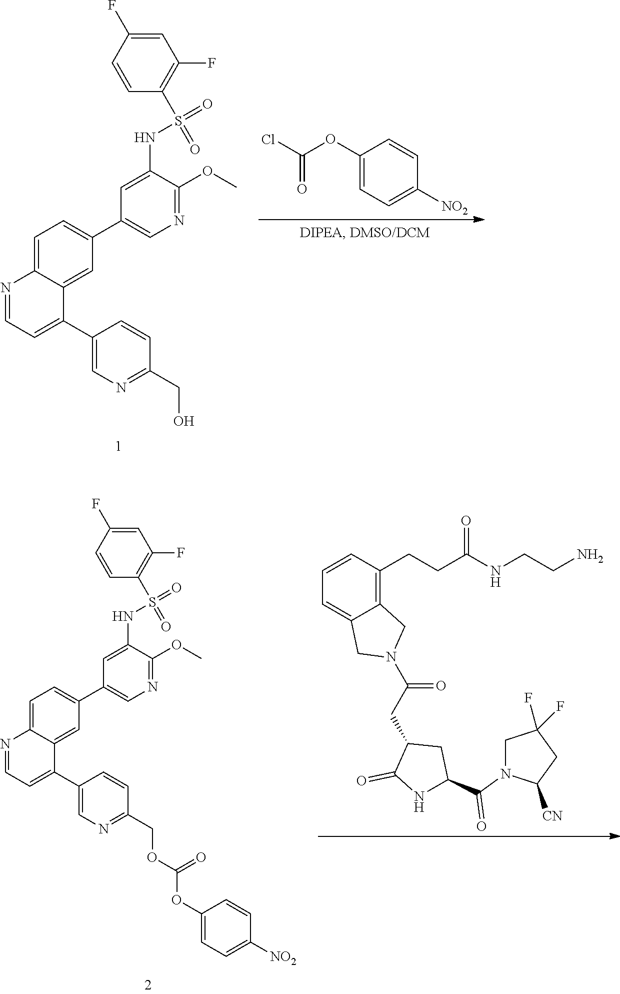 [SMILES: COc1ncc(-c2ccc3nccc(-c4ccc(CO)nc4)c3c2)cc1NS(=O)(=O)c1ccc(F)cc1F ; O=C(Cl)Oc1ccc([N+](=O)[O-])cc1 ; COc1ncc(-c2ccc3nccc(-c4ccc(COC(=O)Oc5ccc([N+](=O)[O-])cc5)nc4)c3c2)cc1NS(=O)(=O)c1ccc(F)cc1F ; N#C[C@@H]1CC(F)(F)CN1C(=O)[C@@H]1C[C@@H](CC(=O)N2Cc3cccc(CCC(=O)NCCN)c3C2)C(=O)N1]

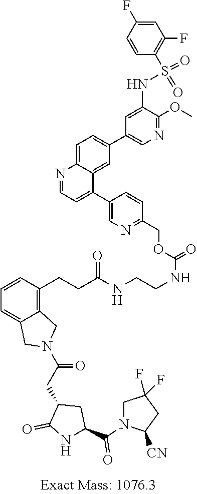 [SMILES: COc1ncc(-c2ccc3nccc(-c4ccc(COC(=O)NCCNC(=O)CCc5cccc6c5CN(C(=O)C[C@@H]5C[C@@H](C(=O)N7CC(F)(F)C[C@H]7C#N)NC5=O)C6)nc4)c3c2)cc1NS(=O)(=O)c1ccc(F)cc1F]

Under N₂ atmosphere, 4-nitrophenyl carbonochloridate (20 mg, 0.1 mmol, 1.0 eq) was dissolved in dimethylsulfoxide (DMSO) (1 mL). Then diispropylethylamine (DIPEA) (15.48 mg, 0.12 mmol, 1.2 eq) was added followed by Compound 1 (58.85 mg, 0.11 mmol, 1.1 eq). The mixture was kept for 30 minutes. The reaction was monitored with liquid chromatography-mass spectrometry (LC-MS) until the starting material was completely consumed, and then with ethyl acetate (EA) (2 mL) and washed three times with water (3×2 mL). The product was dried with sodium sulfate, concentrated under reduced pressure, and purified through combi with Hex/EA as eluent. Compound 2 was obtained in 42 mg as yellow oil.

Under N₂ atmosphere, Compound 2 (6.99 mg, 0.01 mmol, 1.0 eq) was dissolved in DMSO (1 mL), then FAP5 free amine (5.16 mg, 0.01 mmol, 1.0 eq) was added followed by DIPEA (1.93 mg, 0.015 mmol, 1.5 eq). The mixture was kept for 2 hours. After purification, the final compound was provided in 1.4 mg as white powder. Purification condition: reverse phase C-18 column, ACN/NH4HCO3, pH=7, flow rate 8 mL/minute.

### Example 1B

### Comparison of Different FAP5-PI3K Inhibitors

Primary HLF with passage number #˜8 were seeded in Dulbecco's Modified Eagle Medium (DMEM) with 10% fetal bovine serum (FBS) (˜100,000 cells/well) in 12 well plates to achieve ˜80% confluency. The cells were then starved in DMEM with 0.4% FBS for 24 hours, followed by stimulation by 10 ng/mL transforming growth factor-beta 1 (TGF-β1) for 24 hours.

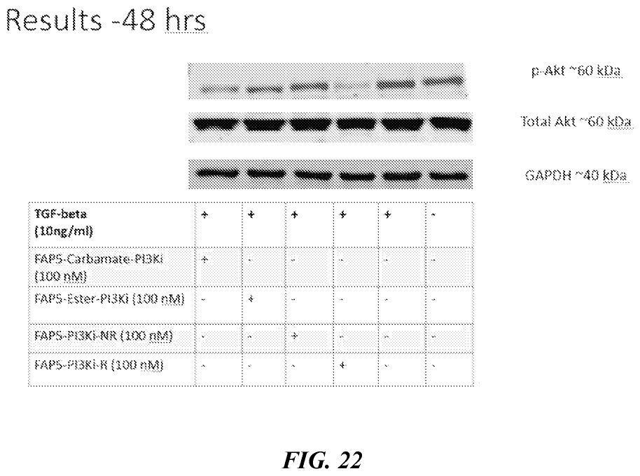

After stimulation, cells were incubated with the FAP5-PI3K inhibitors for 2 hours, followed by replacement of culture media containing 2 ng/mL TGF-β1 and incubated for 24 hours (see FIG. 21) and 48 hours (see FIG. 22). All wells are added with 0.1% of DMSO as vehicle.

Cells were detached using trypsin and lysed with lysis buffer, proteins from cells were extracted for western blot analysis.

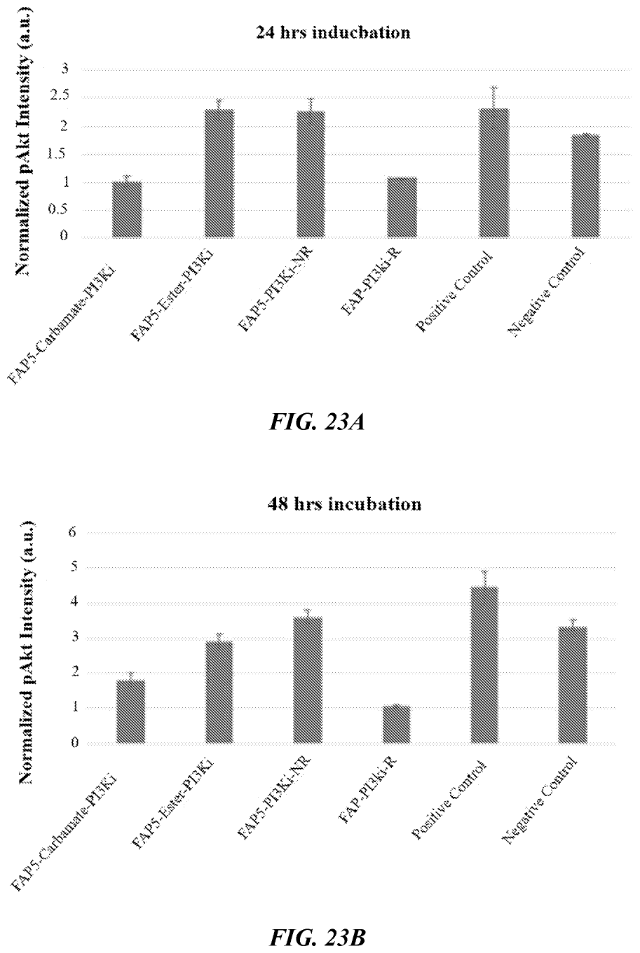

FIGS. 23A and 23B shows a comparison of the inhibition of phosphorylation of Akt by the FAP5-PI3K inhibitors at 24 hours of incubation (FIG. 23A) and at 48 hours of incubation (FIG. 23B). pAkt was normalized to tAkt, positive controls were treated with only TGF-β1 or without treatment, and the negative control was treated without TGF-β1 and without treatment. Of the compounds with non-releasable linkers, FAP5-Carbamate-PI3ki consistently performed better than FAP5-Ester-PI3Ki and FAP5-PI3Ki-NR.

### Example 1C

### Synthesis of FAP-3000

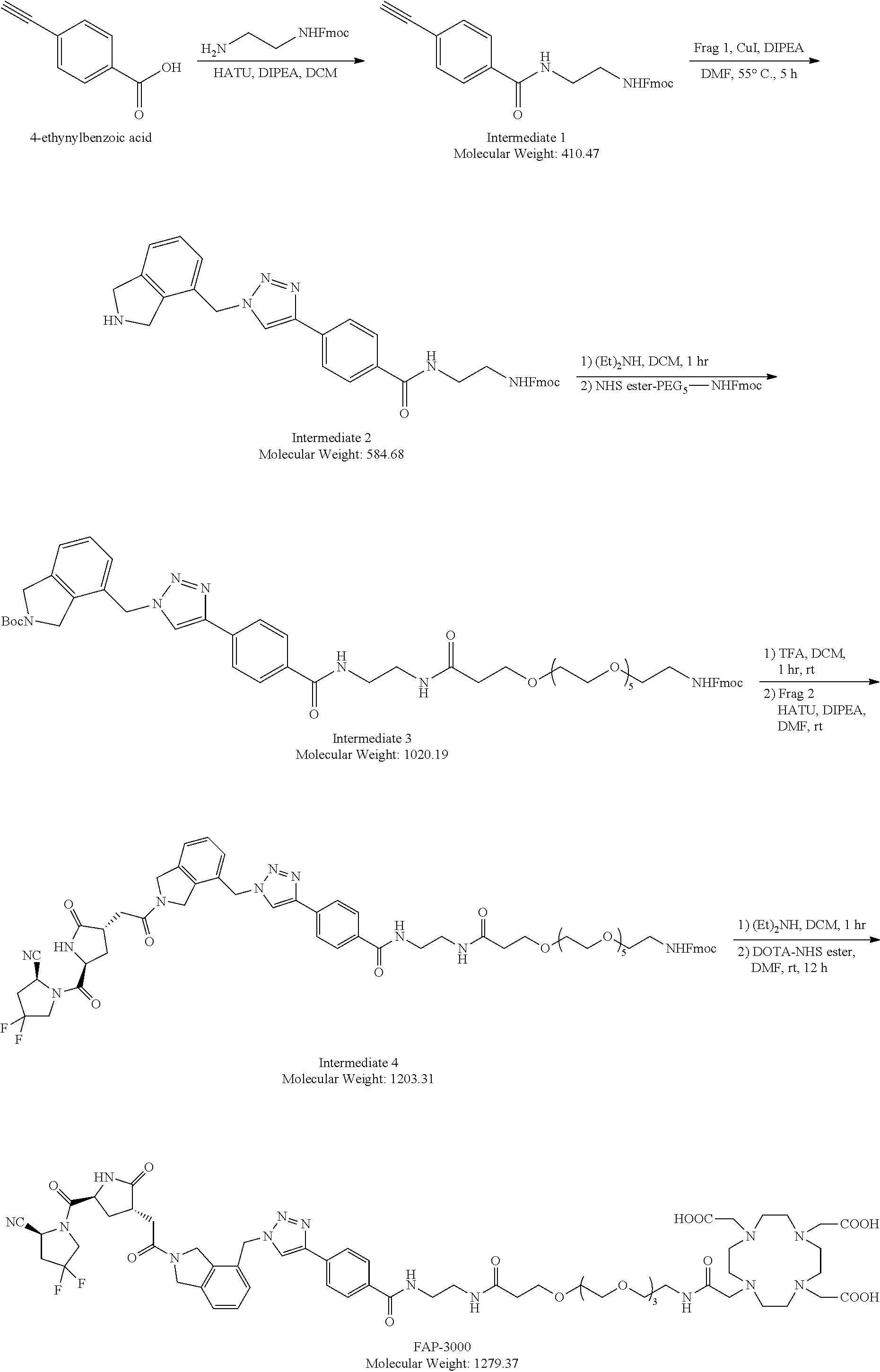

4-methyl isoindoline-4-carboxylate hydrochloride was purchased from PharmaBlock (Hatfield, PA). Boc-L-pyroglutamic acid benzyl ester was purchased from Accela ChemBio (San Diego, CA). 4-(p-iodophenyl)butyric acid was purchased from AstaTech, Inc (Bristol, PA). NHS-ester-PEG₆-NHFmoc and Propargyl-PEG₆-amine were purchased from BroadPharm. DOTA-NHS ester was purchased from Macrocyclics. Fmoc-Lys-OH was purchased from AAPPTec (Louisville, KY). 4,4-Difluoro-L-prolinamide hydrochloride and HATU were purchased from Chem-Impex International (Chicago, IL). 4-Ethynylbenzoic acid and mono-Fmoc ethylene diamine hydrochloride were purchased from AA Blocks LLC (San Diego, CA). Di-tert-butyl dicarbonate was purchased from Oakwood Chemical (Estill, SC). Palladium, 10% on carbon, was purchased from Alfa Aesar (Haverhill, MA). Sodium borohydride, N-bromosuccinimide, triphenylphosphine, sodium azide, lithium bis(trimethylsilyl)amide, tert-butyl-bromoacetate, 1,8-diazabicyclo[5.4.0]undec7-ene, pyridine, imidazole, phosphoryl chloride, diethyl either, DIPEA, trifluoroacetic acid (TFA), tetrahydrofuran (THF), n, n-dimethylformamide (DMF), dichloromethane (DCM), methanol (MeOH), DMSO, and all other reagents were purchased from Sigma-Aldrich (St. Louis, MO). All synthesized molecules were purified using either flash chromatography (CombiFlash RF, Teledyne) or RP-HPLC (Agilent 1200 Instrument) with an XBridge OBD preparative column (19×150 mm, 5 μm) purchased from Waters (Milford, MA). Low-resolution mass spectrometry-liquid chromatography/mass spectrometry (LRMS-LC/MS) was performed with an Agilent 1220 Infinity LC with a reverse-phase XBridge Shield RP18 column (3.0×50 mm, 3.5 μm).

The synthesis of key intermediates Fragment 1 and Fragment 2 are reported elsewhere in the literature.

Briefly, 4-Ethynylbenzoic acid was dissolved in anhydrous DMF containing HATU (1 eq) and anhydrous DIPEA (3 eq) for 10 minutes. Mono-Fmoc ethylene diamine was dissolved in anhydrous DMF and DIPEA (1.5 eq), then added to the reaction mixture. The resulting solution was stirred under inert atmosphere for 2 hours. The desired Intermediate 1 was precipitated with ice cold water, and then filtered and dried under vacuum. The Intermediate 1 was dissolved in anhydrous DMF, then CuI (0.5 eq) and DIPEA (2.0 eq) were added. The reaction mixture was heated to 55° C. and stirred for 5 hours. The crude product was extracted with ethyl acetate (EtOAc), washed with ice cold brine, then purified by flash chromatography [A=Hex, B=EtOAc, solvent gradient 0% B to 100% B in 20 minutes] to yield the product Intermediate 2. LC/MS (m/z): [M+H]+ calculated for C₄₀H₄₀N₆O₅, 684.8; observed mass 685.

The Intermediate 2 was dissolved in DCM and diethylamine (20 eq) then stirred for 1 hour. LC/MS (m/z): [M+H]+ calculated for C₂₅H₃₀N₆O₃, 462.6; observed mass 463. Upon completion of the deprotection, the product was isolated via rotary evaporation, washed several times with ice cold diethyl ether, then put on high vacuum for several hours, after which it was used without further purification. NHS ester-PEG6-NHFmoc (1.1 eq) and DIPEA (3 eq) were added and stirred for several hours. The crude product was washed, then purified by flash chromatography [A=DCM, B=MeOH, solvent gradient 0% B to 30% B in 25 minutes] to yield Intermediate 3. LC/MS (m/z): [M+H]+ calculated for C₅₅H₆₉N₆O₂₁, 1020.2; observed mass 1021.

Intermediate 3 was dissolved in ACN and cooled to 0° C. An equal volume of TFA was added and the reaction mixture was stirred at room temperature for 1 hour. Progress of the reaction was monitored via LC/MS. LC/MS (m/z): [M+H]⁺ calculated for C₅₀H₆₁N₇O₁₀, 920.1; observed mass 921. Upon completion of the deprotection, TFA was removed by rotary evaporation and the deprotected product was used without further purification. Fragment 2 was dissolved in anhydrous DMF and DIPEA (3 eq) for 10 minutes. The previously deprotected product was dissolved in anhydrous DMF containing excess DIPEA, then added to the reaction mixture and stirred under inert atmosphere for 2 hours. The crude product was extracted with EtOAc, washed with ice cold brine, then purified by flash chromatography [A=DCM, B=MeOH, solvent gradient 0% B to 30% B in 25 minutes] to yield the Intermediate 4. [M+H]⁺ calculated for C₆₂H₇₂F₂N₁₀O₁₃, 1203.3; observed mass 1204.

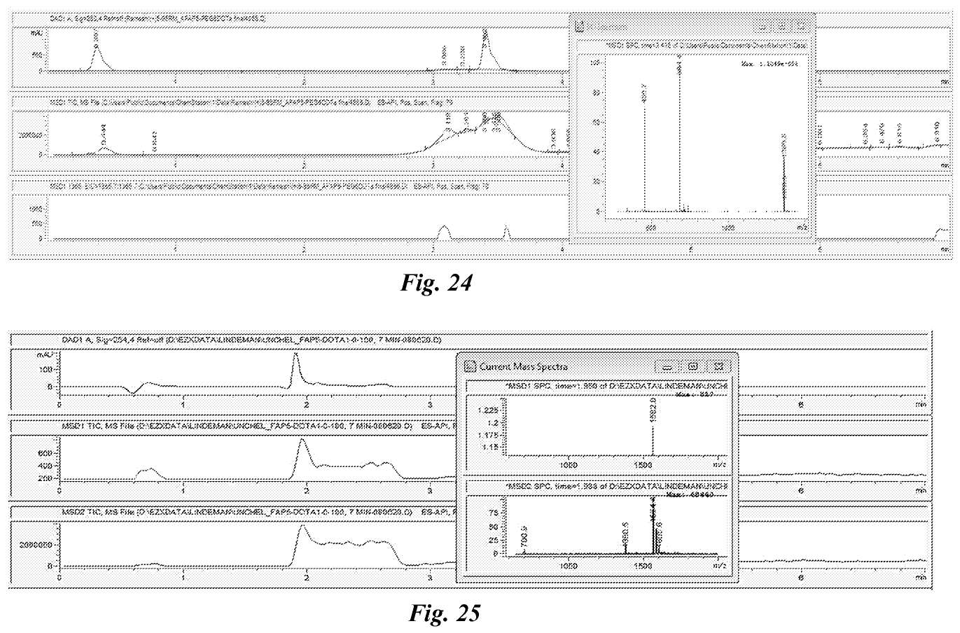

Intermediate 4 was dissolved in DCM and diethylamine (20 eq) then stirred for 1 hour. LC/MS (m/z): [M+H]+ calculated for C47H62F2N10O11, 981.1; observed mass 982. Upon completion of the deprotection, the product was isolated via rotary evaporation, washed several times with ice cold diethyl ether, then put on a high vacuum for several hours, after which it was used without further purification. The deprotected product was dissolved in anhydrous DMF and DIPEA (3 eq) with DOTA-NHS ester (1.2 eq) and stirred under inert atmosphere for 12 hours. The crude product was purified via RP-HPLC [A=20 mM ammonium acetate buffer (pH 5.0) and B=CH3CN, solvent gradient 5% B to 55% B in 45 minutes] to yield the final desired product FAP-3000 (see FIG. 24). [M+H]+ calculated for C₆₃H₈₈F₂N₁₄O₁₄O₁₈, 1367.47; observed mass 1368.

### Example 1D

### Synthesis of FAP-3001

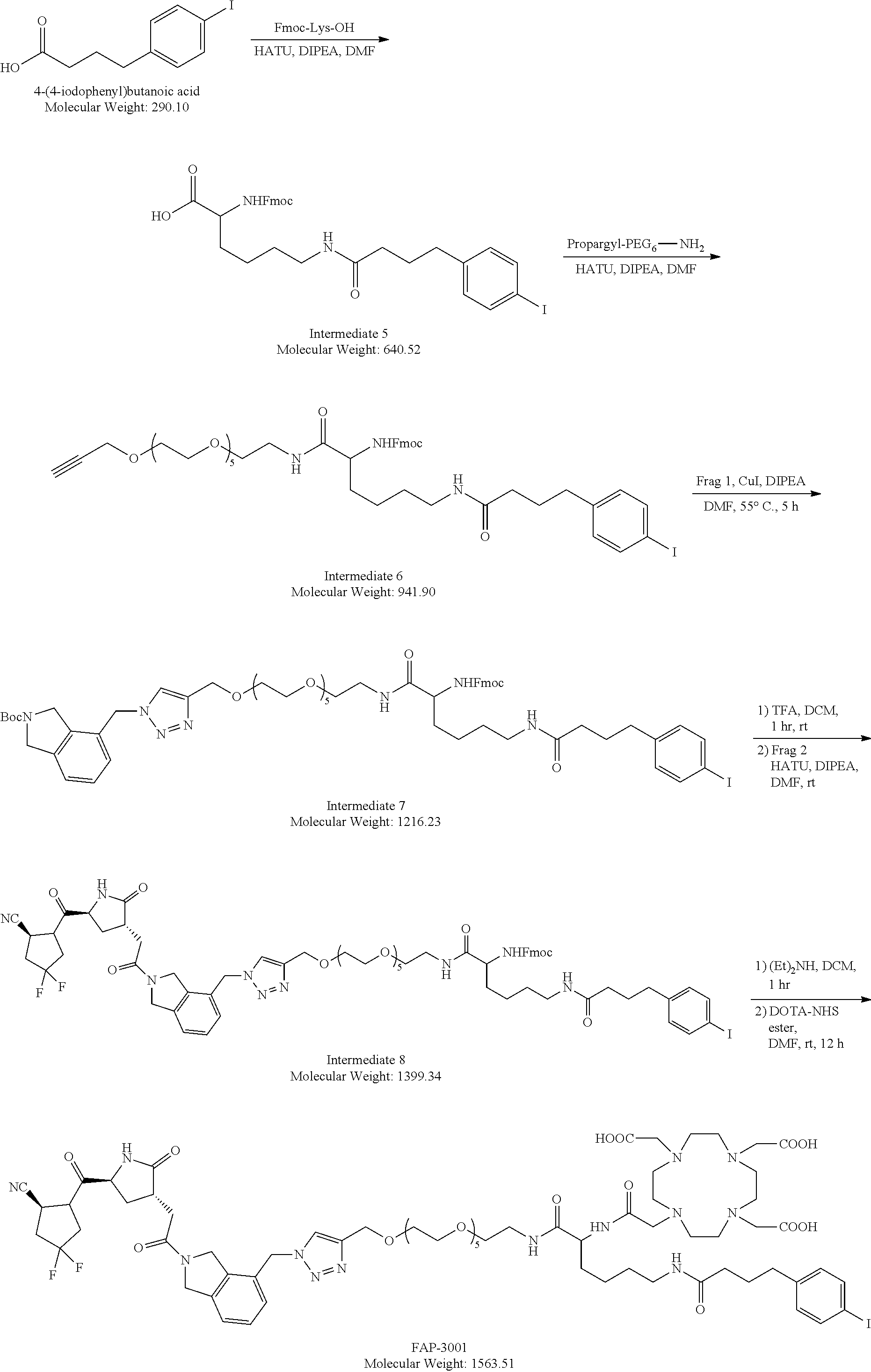

4-(p-iodophenyl)butyric acid was dissolved in anhydrous DMF with HATU (1 eq) and anhydrous DIPEA (3 eq) for 1 hour. Fmoc-Lys-OtBu HCl (1 eq) was dissolved in anhydrous DMF and DIPEA (1.5 eq), then added to the reaction mixture. The resulting solution was stirred under inert atmosphere for 2 hours. The product was extracted with EtOAc, washed with ice cold brine, then purified by flash chromatography [A=DCM, B=MeOH, solvent gradient 0% B to 20% B in 25 minutes] to yield Intermediate 5. LC/MS (m/z): [M+H]⁺ calculated for C₃₁H₃₃IN₂O₅, 696.6; observed mass 697.

Intermediate 5 was dissolved in anhydrous DMF with HATU (1 eq) and anhydrous DIPEA (3 eq) for 10 minutes. Propargyl-PEG₆-amine was dissolved in anhydrous DMF and DIEPA (1.5 eq), then added to the reaction mixture. The resulting solution was stirred under inert atmosphere for 3 hours. The product was extracted with EtOAc, washed with ice cold brine, then purified by flash chromatography [A=DCM, B=MeOH, solvent gradient 0% B to 20% B in 25 minutes] to yield Intermediate 6. LC/MS (m/z): [M+H]⁺ calculated for C₄₆H₆₀IN₃O₁₀, 941.9; observed mass 943. Progress of the reaction was monitored by LC/MS.

Fragment 1 (1.2 eq) was dissolved in anhydrous DMF with Intermediate 6, then CuI (0.5 eq) and DIPEA (2.0 eq) were added. The reaction mixture was heated to 55° C. and stirred for 5 hours. The product was extracted with EtOAc, washed with ice cold brine, then purified by flash chromatography [A=DCM, B=MeOH, solvent gradient 0% B to 20% B in 25 minutes] to yield Intermediate 7. LC/MS (m/z): [M+H]⁺ calculated for C₆₀H₇₈IN₇O₁₂, 1216.2; observed mass 1217.

Intermediate 7 was dissolved in ACN and cooled to 0° C. An equal volume of TFA was added and the reaction mixture was stirred at room temperature for 1 h. Progress of the reaction was monitored via LC/MS. LC/MS (m/z): [M+H]⁺ calculated for C₅₅H₇₀IN₇O₁₀, 1116.11; observed mass 1117. Upon completion of the deprotection, TFA was removed by rotary evaporation and the deprotected amine-bearing product was used without further purification. Fragment 2 was dissolved in anhydrous DMF and DIPEA (3 eq) for 10 minutes. The previously deprotected product was dissolved in anhydrous DMF and excess DIPEA, then added to the reaction mixture and stirred under inert atmosphere for 2 hours. The crude product was extracted with EtOAc, washed with ice cold brine, then purified by flash chromatography [A=DCM, B=MeOH, solvent gradient 0% B to 30% B in 25 minutes] to yield Intermediate 8. LC/MS (m/z): [M+H]⁺ calculated for C₆₇H₈₁IN₁₀O₁₃, 1399.3; observed mass 1400.

Intermediate 8 was dissolved in DCM and diethylamine (20 eq) then stirred for overnight. Progress of the reaction was monitored via LC/MS. LC/MS (m/z): [M+H]⁺ calculated for C₅₂H₇₁F₂IN₁₀O₁₀, 1177.11; observed mass 1178. Upon completion of the deprotection, the amine-bearing product was isolated via rotary evaporation, washed several times with ice cold ethyl acetate, washed once with ice cold ether, then placed under high vacuum for several hours, after which it was used without further purification. The deprotected product was dissolved in anhydrous DMF and DIPEA (3 eq) with DOTA-NHS ester (1.2 eq) and stirred under inert atmosphere for 12 h. The final desired product FAP-3001 was purified via RP-HPLC [A=20 mM ammonium acetate buffer (pH 5.0) and B=CH₃CN, solvent gradient 5% B to 55% B in 45 minutes] to yield FAP-3001 (see FIG. 25). LC/MS (m/z): [M+H]⁺ calculated for C₆₈H₉₇F₂IN₁₄O₁₈, 1563.6; observed mass 1564.

Other structures presented herein were synthesized in a similar fashion to the two syntheses provided.

### Example 2

### FAP Conjugates Binding Affinities

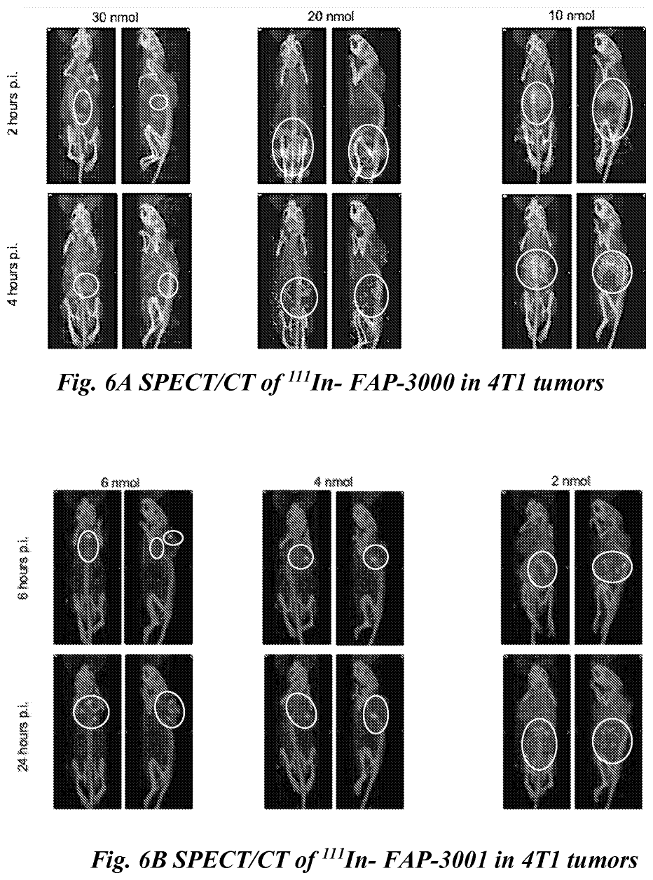

250,000 HEK-FAP cells were seeded into amine-coated 24 well plates and allowed to reach confluence. 10 nM of FAP-Rhodamine was displaced by increasing concentrations of FAP-targeted conjugates at 4° C. for 1 hour using HEK-FAP cells. Cells were then washed 3 times with phosphate buffered saline (PBS), dissolved in 1% sodium lauryl sulfate (SDS) and transferred to a 96 well clear bottom, black wall plate before analysis via a Synergy Neo2 microplate reader. All samples were performed in triplicate, with standard error of the mean (SEM) bars shown (see FIGS. 6A and 6B). A K_{d} of 2.67 nM was used to calculate the inhibition constants.

### Example 3

### Radiolabeled FAP Conjugates Binding Curves

Radiolabeling of DOTA-bearing conjugates was performed as followed. Conjugates were diluted in ammonium acetate (0.5 M, pH 8.0) to reach a final DOTA concentration of 0.5 mM. To generate an ¹¹¹In radioimaging agent, ¹¹¹In (¹¹¹InCl₃) was added to obtain a specific activity of up to 4.0 MBq/nmol, as indicated, while for production of the corresponding radiotherapeutic agent, ¹⁷⁷Lu (¹⁷⁷LuCl₃) was added to obtain a specific activity of up to 11.0 MBq/nmol. The resulting solutions were then heated to 90° C. for 10-20 minutes and the radiochemical purities of labeled products were analyzed by radio-HPLC (20 mM ammonium acetate buffer (pH 7) (A) and acetonitrile (B) in a linear gradient from 5% B to 95% B over 15 minutes). Radiochemical purities were found to exceed 95% in all studies. After confirmation of successful radiolabeling, sodium-diethylenetriamine pentaacetate solution (5 mM, pH 7.0) was added at a final concentration of ˜0.2 mM to complex any unreacted traces of radioactive isotope.

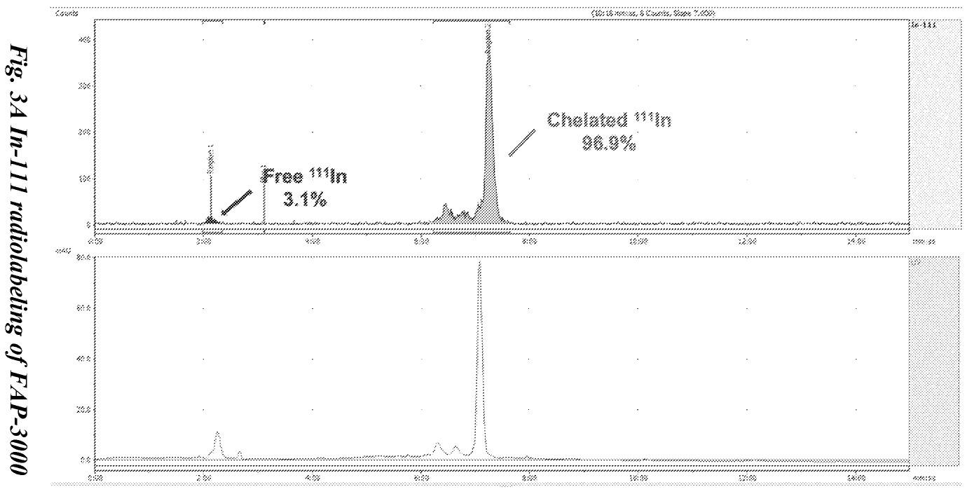

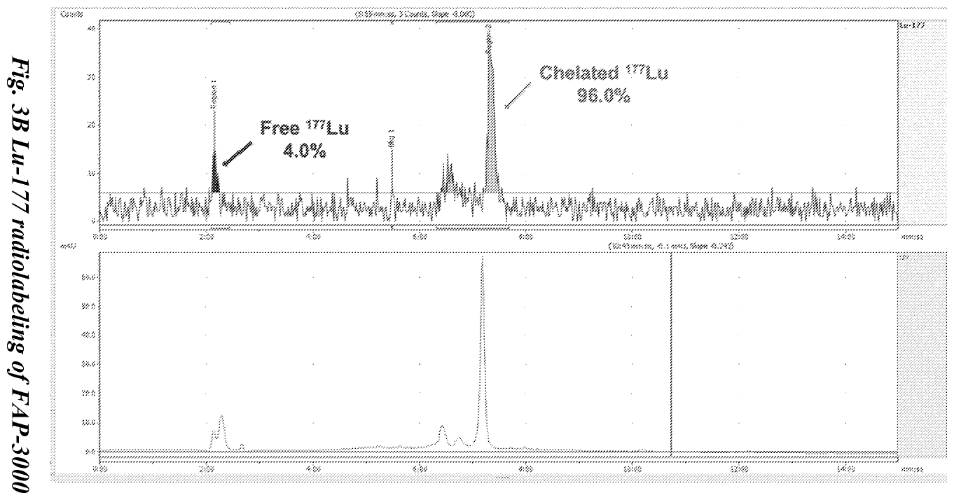

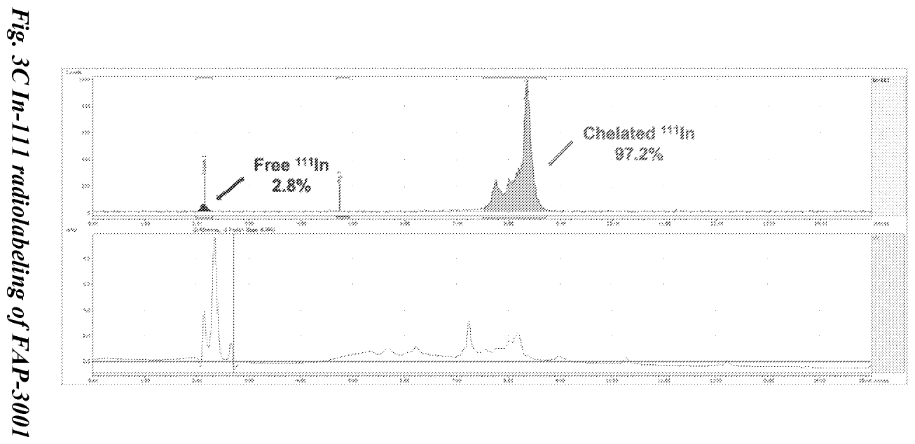

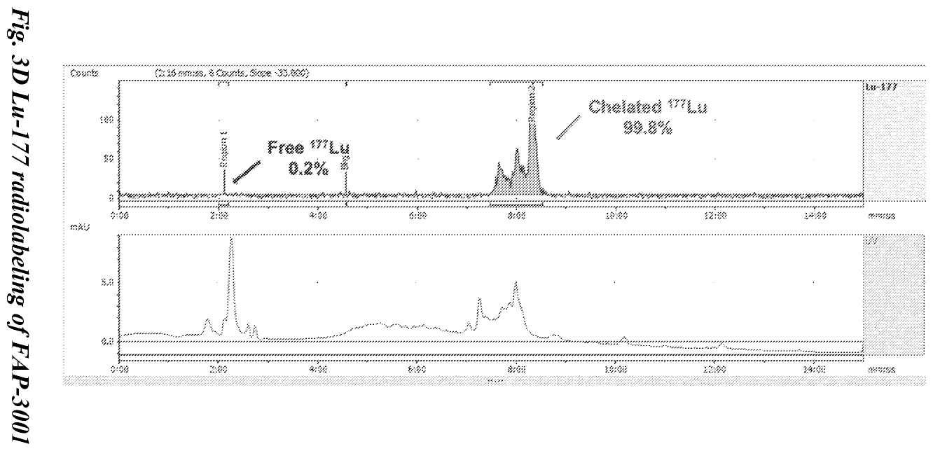

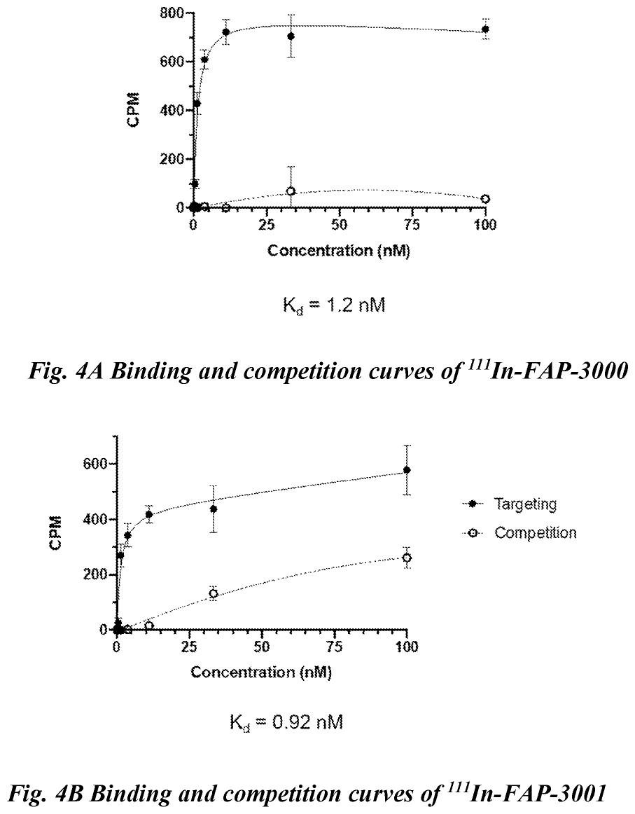

Cancer-associated fibroblasts (Hs894 cell line) were incubated for 1 hour at room temperature with increasing concentrations of ¹¹¹In-FAP-3000 or ¹¹¹In-FAP-3001 in the absence or presence of 100× excess of FAP competition ligand, washed 3 times with PBS, then dissolved in 1.0 M NaOH and analyzed via gamma counter (see FIGS. 4A and 4B). All samples were performed in triplicate with SEM bars.

### Example 4

### Dosing Studies in HT29 Tumors

Nu/nu mice were inoculated on their shoulder with 5×10⁶ cells of HT29 human colorectal cancer. Dosing studies were performed using increasing amounts of either FAP-3000 or FAP-3001 radiolabeled with In-111 in HT29 tumor-bearing nude mice and imaged with an MILabs VECTor⁴⁺ instrument. Animals were anesthetized with isoflurane and scanned at various time points post injection. The emission scan was conducted for 20-60 minutes using the MILabs VECTor/CT system. The CT scans were acquired with an X-ray source set at 60 kV and 615 μA. The SPECT images were reconstructed with U-SPECT II software and ¹¹¹In γ-energy windows of 171 and 241 keV. A POS-EM algorithm was used with 16 subsets and 4 iterations on a 0.8 mm voxel grid. The CT images were reconstructed using NRecon software. The datasets were fused and filtered using PMOD software (version 3.2).

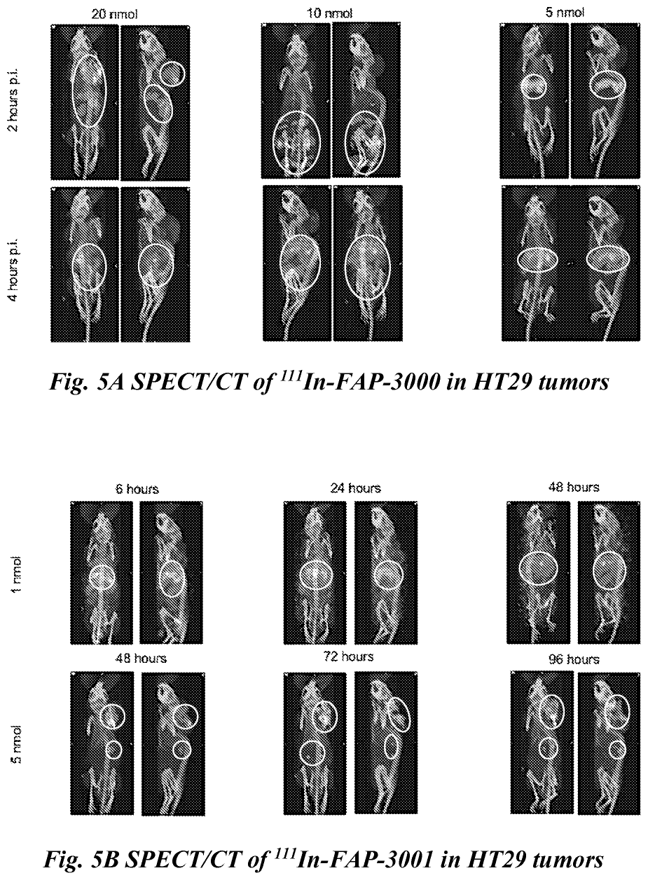

HT29 tumor-bearing nude mice were dosed with 0.3 mCi of In-111 chelated by either 20 nmol, 10 nmol, or 5 nmol of FAP-3000 via intravenous tail vein injection. FIG. 5A depicts mice that received FAP-3000 using single-photon emission computed tomography/computed tomography (SPECT/CT), with images taken at 2 hours post injection and 4 hours post injection.

HT29 tumor-bearing nude mice were dosed with 0.3 mCi of In-111 chelated by either 5 nmol or 1 nmol of FAP-3001 via intravenous tail vein injection. FIG. 5B depicts images of such mice using SPECT/CT, taken at various times post injection (6 hours, 24 hours, 48 hours, 72 hours, and 96 hours).

### Example 5

### Dosing Studies in 4T1 Tumors

Balb/cJ mice were inoculated on their shoulder with 1×10⁵ cells of 4T1 cells in sterile PBS. Dosing studies were performed by dosing 4T1 tumor-bearing Balb/c mice with 0.3 mCi of In-111 chelated by either 30 nmol, 20 nmol, and 10 nmol of FAP-3000 or 6 nmol, 4 nmol, and 2 nmol of FAP-3001 via intravenous tail vein injection and SPECT/CT images were taken at various intervals post injection. FIG. 6A shows images from the mice that received ¹¹In-FAP-3000. FIG. 6B shows images from the mice that received ¹¹¹In-FAP-3001.

### Example 6

### Retention Studies

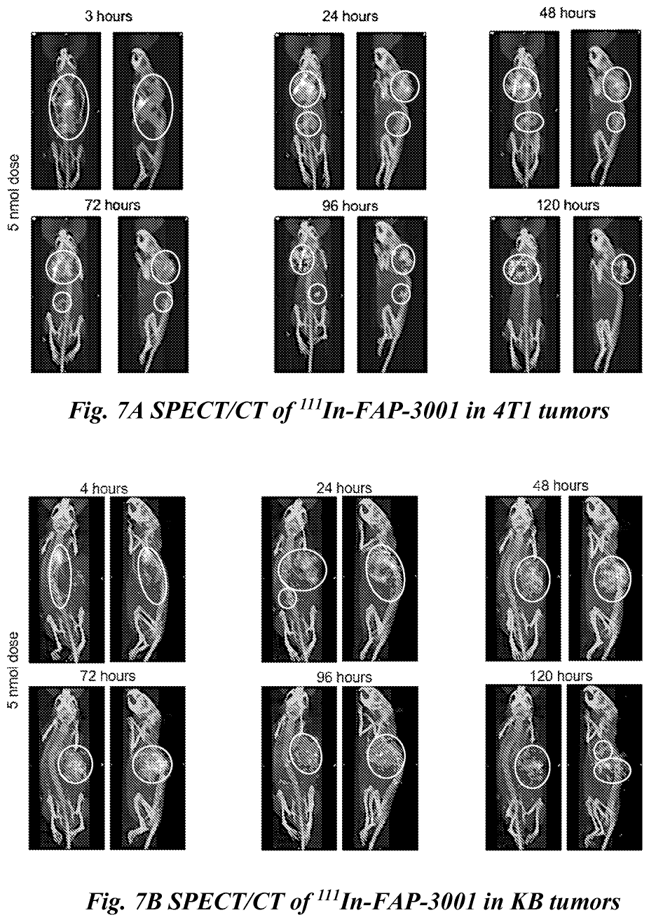

Retention studies were performed using FAP-3001 in both 4T1 tumor-bearing Balb/c mice and KB tumor-bearing mice. Both sets of mice were injected with 5 nmol of FAP-3001 radiolabeled with 0.3 mCi of indium-111 and injected by intravenous tail vein injection and SPECT/CT images were taken at various intervals post injection. FIG. 7A shows images from the 4T1 tumor-bearing Balb/c mice and FIG. 7B shows images from the KB tumor-bearing mice.

### Example 7

### Effect of Albumin-Binder on FAP Biodistribution

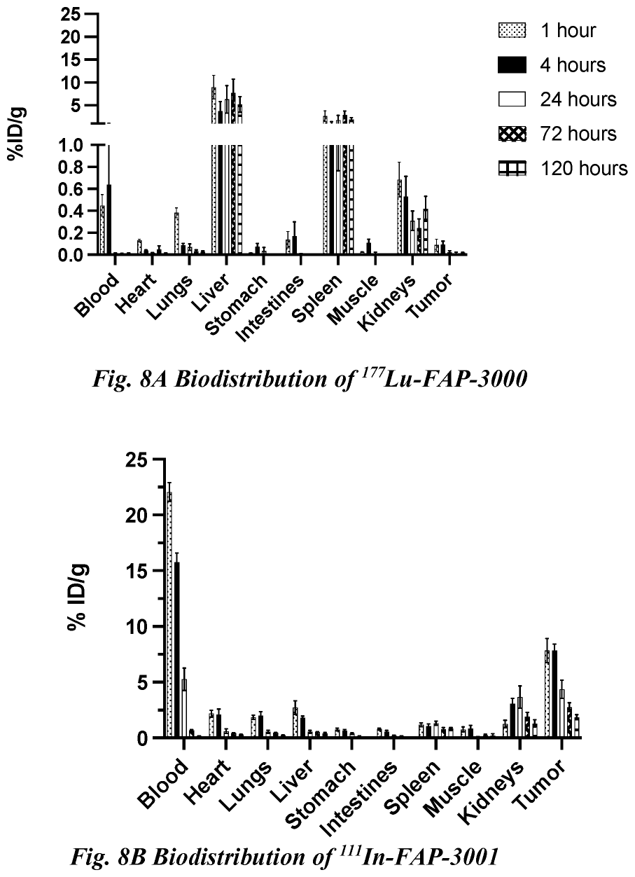

A biodistribution study was initiated when the 4T1 tumor volume reached about 200 mm³. Mice were injected with 1 dose of 5 nmol of FAP-3000 radiolabeled with 100 μCi of lutetium-177 (¹⁷⁷Lu-FAP-3000) or FAP-3001 radiolabeled with 10 μCi of indium-111 (¹¹¹In-FAP-3001) via intravenous tail vein injection (see FIGS. 8A and 8B, respectively) and measured with a Packard Cobra Gamma Counter. Each desired time point comprised 4-5 mice per conjugate that were asphyxiated by CO₂. Organs of interest were immediately harvested and washed in cold PBS before gamma counter measurements. Results were normalized as percentages of the injected dose and per gram of tissue.

### Example 8

### ¹⁷⁷Lu-FAP-3001 Radiotherapy Study in 4T1 Tumors

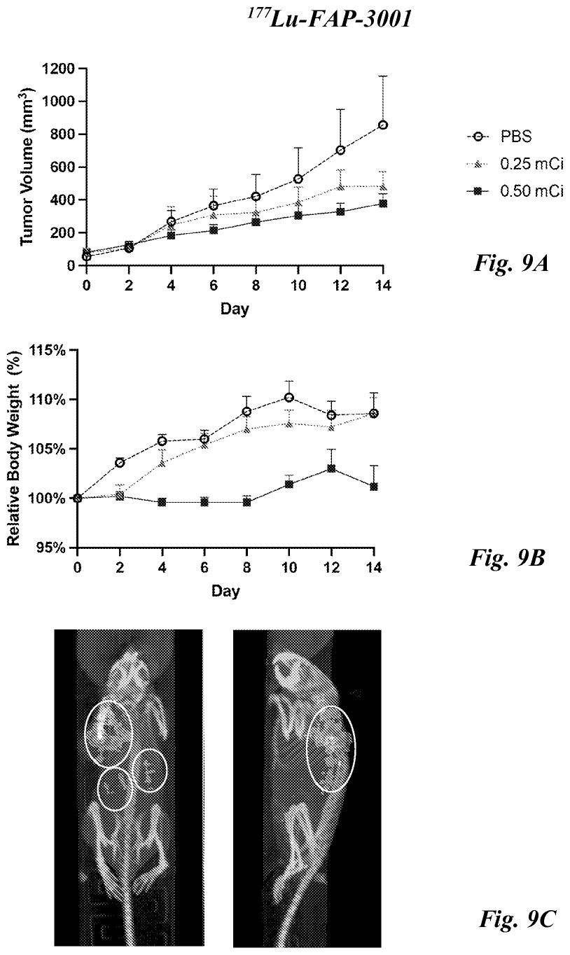

Therapy was initiated when the 4T1 tumor volume reached about 50 mm³. Control mice were injected with 5% ethanol (EtOH) in sterile PBS, whereas the treated mice were injected with 1 dose of 5 nmol of FAP-3001 radiolabeled with either 0.25 mCi or 0.50 mCi of lutetium-177 (¹⁷⁷Lu-FAP-3001) via intravenous tail vein injection. Body weight of the mice was monitored as a measure of gross toxicity. FIG. 9A shows tumor growth data from the study, FIG. 9B shows relative body weight data from the study, and FIG. 9C shows a SPECT/CT scan of one of the treated mice 24 hours post injection.

### Example 9

### ¹⁷⁷Lu-FAP-3000 Radiotherapy Study in KB Tumors

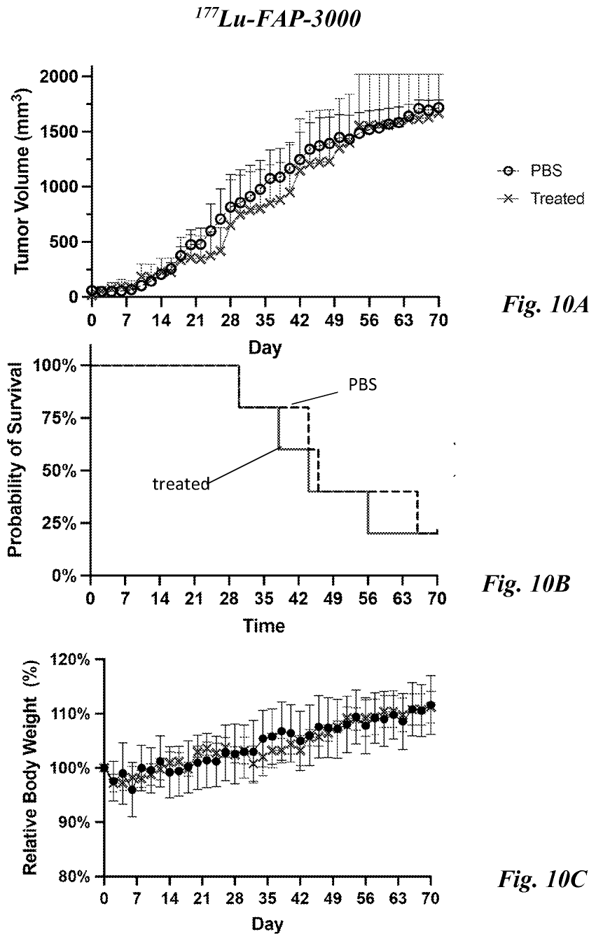

Therapy was initiated when the KB tumor volume reached about 50 mm³. Control mice were injected with 5% EtOH in sterile PBS, whereas the treated mice were injected with 1 dose of 5 nmol of FAP-3000 radiolabeled with 0.50 mCi of lutetium-177 (¹⁷⁷Lu-FAP-3000) via intravenous tail vein injection. Body weight of the mice were monitored as a measure of gross toxicity. FIG. 10A shows tumor growth data from the study (using KB tumor growth chart in nude mice for comparison), FIG. 10B depicts a survival curve of the study, and FIG. 10C shows relative body weight data of the mice from the study.

### Example 10

### ¹⁷⁷Lu-FAP-3001 Radiotherapy Study in KB Tumors

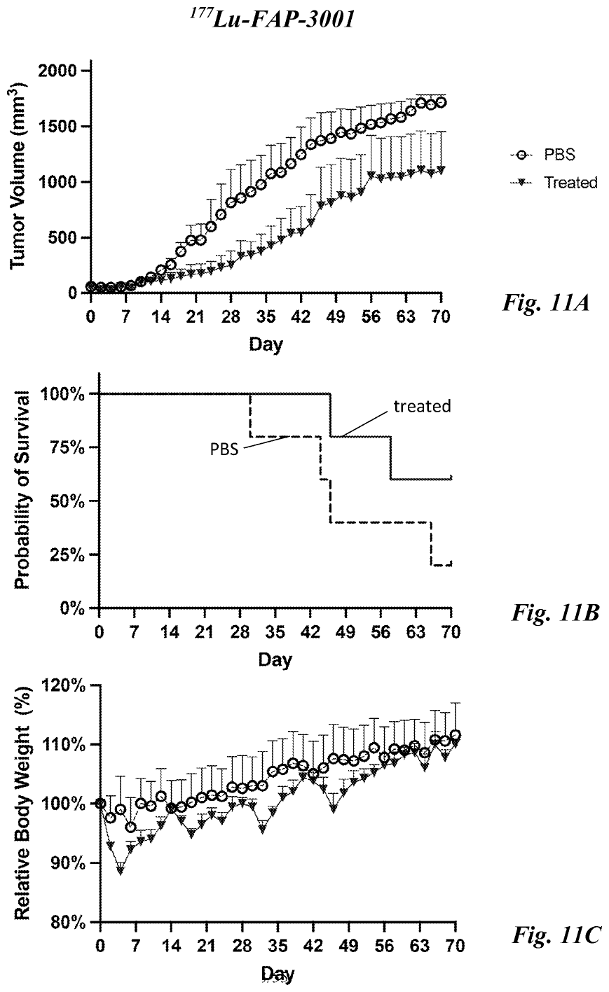

Therapy was initiated when the KB tumor volume reached about 50 mm³. Control mice were injected with 5% EtOH in sterile PBS, whereas the treated mice were injected with one dose of 5 nmol of FAP-3001 radiolabeled with 0.50 mCi of lutetium-177 (¹⁷⁷Lu-FAP-3001) via intravenous tail vein injection. Body weight of the mice were monitored as a measure of gross toxicity. FIG. 11A shows tumor growth data from the study (using KB tumor growth chart in nude mice for comparison), FIG. 11B depicts a survival curve of the study, and FIG. 11C shows relative body weight data of the mice from the study.

### Example 11

### ¹⁷⁷Lu-FAP-3001 Radiotherapy Study in HT29 Tumors

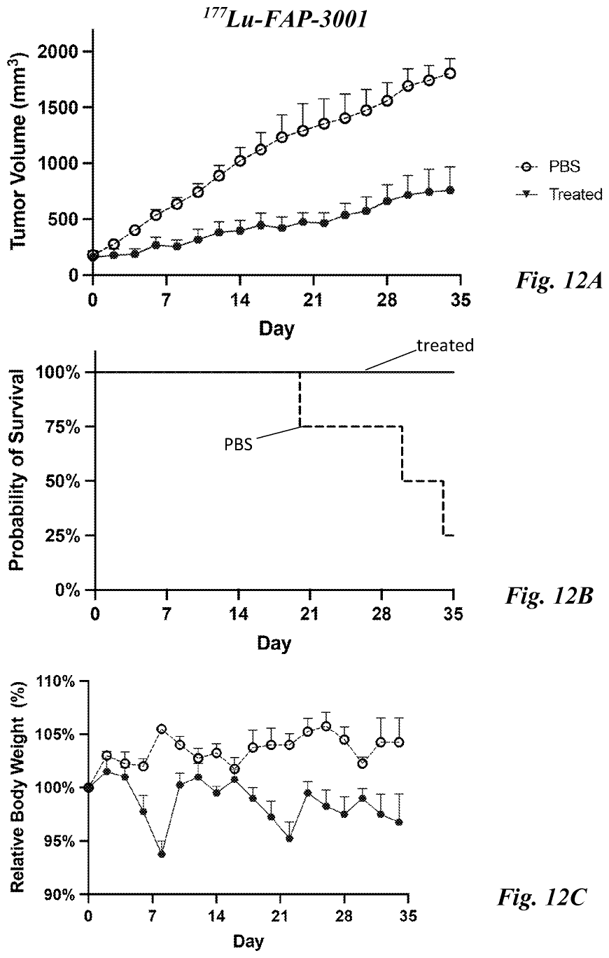

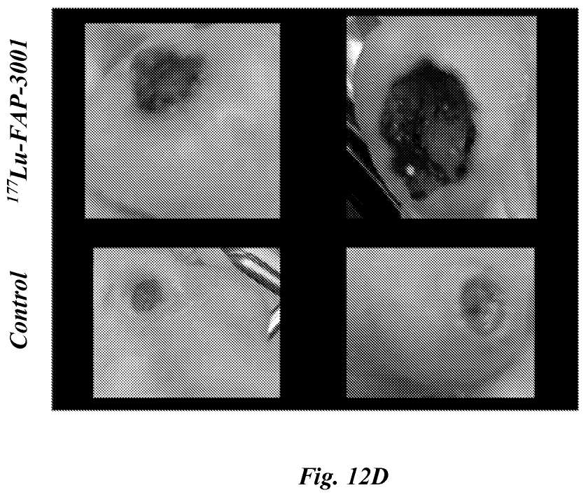

Therapy was initiated when the HT29 tumor volume reached about 150 mm³. Control mice were injected with 5% EtOH in sterile PBS, whereas the treated mice were injected with 1 dose of 5 nmol of FAP-3001 radiolabeled with 0.25 mCi of lutetium-177 (¹⁷⁷Lu-FAP-3001) via intravenous tail vein injection. Body weight of the mice were monitored as a measure of gross toxicity. FIG. 12A shows tumor growth data from the study (using KB tumor growth chart in nude mice for comparison), FIG. 12B depicts a survival curve of the study, and FIG. 12C shows relative body weight data of the mice from the study. FIG. 12D shows photograph images of the collapsed tumors post euthanasia.

### Example 12

### ¹⁷⁷Lu-FAP-3001 Radiotherapy Study in U87MG Tumors

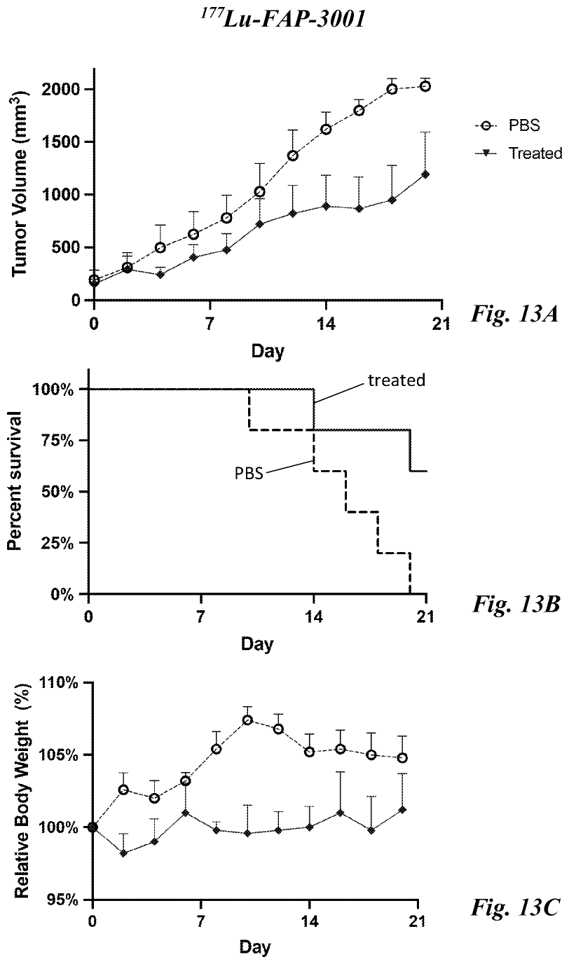

Therapy was initiated when the U87MG tumor volume reached about 190 mm³. Control mice were injected with 5% EtOH in sterile PBS, whereas the treated mice were injected with 1 dose of 5 nmol of FAP-3001 radiolabeled with 0.50 mCi of lutetium-177 (¹⁷⁷Lu-FAP-3001) via intravenous tail vein injection. Body weight of the mice were monitored as a measure of gross toxicity. FIG. 13A shows tumor growth data from the study (using KB tumor growth chart in nude mice for comparison), FIG. 13B depicts a survival curve of the study, and FIG. 13C shows relative body weight data of the mice from the study.

### Example 13

### ¹⁷⁷Lu-FAP-3001 Radiotherapy Toxicology Stains

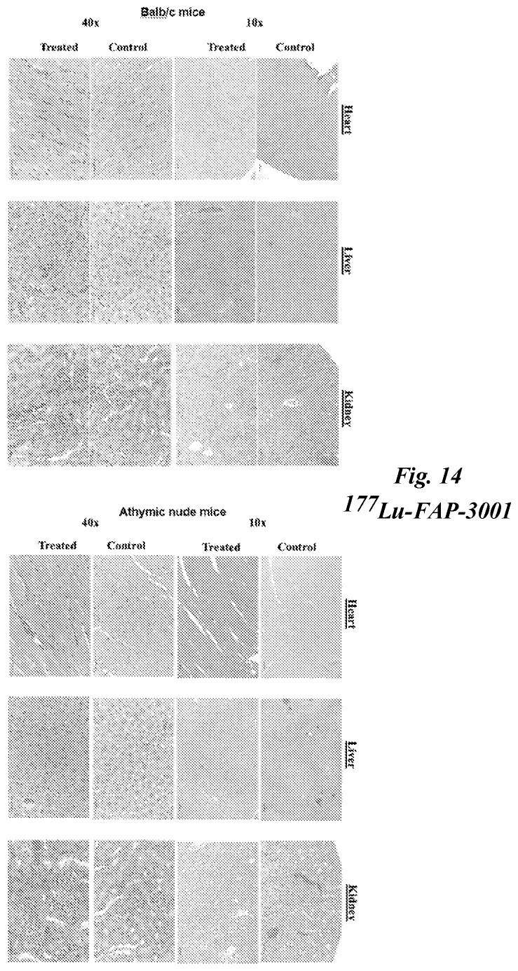

Mice were randomly selected from control and treated groups (0.5 mCi doses) for further toxicology evaluation. Organs of interest were harvested immediately post euthanasia, washed, and fixed in a 10% formalin buffered solution for 48-72 hours. Organs were then maintained in a 70% ethanol solution until radioactivity had fully decayed, after which they were submitted to the Histology Laboratory at Purdue University to be embedded in paraffin, sectioned, and stained with hematoxylin and eosin (H&E) (see FIG. 14). Tissue sections (n=4-8 per organ per mouse) were examined in a blinded manner for lesions by a licensed pathologist at the Purdue University Department of Comparative Pathobiology.

Table 1 is a summary of the data observed from such study (see also FIG. 14).

[symbol]

TABLE 1
Toxicology Study Summary

| Species | Group | Organ | # Examined | DL |
|---|---|---|---|---|
| Balb/c | Control | Liver | 22 | 0 |
| | (n = 4) | R. Kidney | 16 | 0 |
| | | L. Kidney | 16 | 0 |
| | | Myocardium | 16 | 0 |
| | Treated | Liver | 24 | 0 |
| | (n = 4) | R. Kidney | 16 | 0 |
| | | L. Kidney | 16 | 0 |
| | | Myocardium | 16 | 0 |
| Athymic [symbol] | Control | Liver | 8 | 0 |
| | (n = 2) | R. Kidney | 8 | 0 |
| | | L. Kidney | 8 | 0 |
| | | Myocardium | 8 | 0 |
| | Treated | Liver | 38 | 0 |
| | (n = 7) | R. Kidney | 28 | 0 |
| | | L. Kidney | 28 | 0 |
| | | Myocardium | 28 | 0 |

[symbol]

indicates data missing or illegible when filed

### Example 14

### ¹⁷⁷Lu-FAP-3001 Radiotherapy Study in 4T1—High Doses

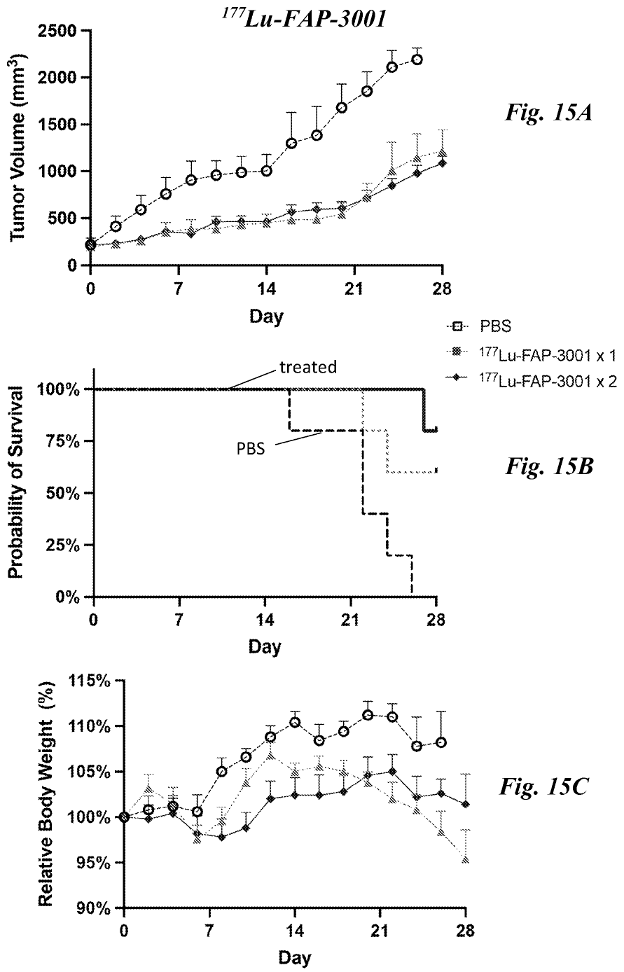

Therapy was initiated when the 4T1 tumor volume reached about 200 mm³. Control mice were injected with 5% EtOH in sterile PBS, whereas the treated mice were injected with 1 dose of 5 nmol of FAP-3001 radiolabeled with 1.50 mCi of lutetium-177 (¹⁷⁷Lu-FAP-3001) on day 0 or 2 doses of 5 nmol of FAP-3001 radiolabeled with 1.50+0.6 mCi of lutetium-177 (¹⁷⁷Lu-FAP-3001 on days 0 and 3, respectively, via intravenous tail vein injection. Body weight of the mice were monitored as a measure of gross toxicity. FIG. 15A shows tumor growth data from the study, FIG. 15B depicts a survival curve of the study, FIG. 15C shows relative body weight data of the mice from the study, FIG. 15D shows SPECT/CT scan of one of the mice that received a single dose of the ¹⁷⁷Lu-FAP-3001.

### Example 15

### ¹⁷⁷Lu-FAP-3001 Radiotherapy Study in 4T1-Blind Measurements

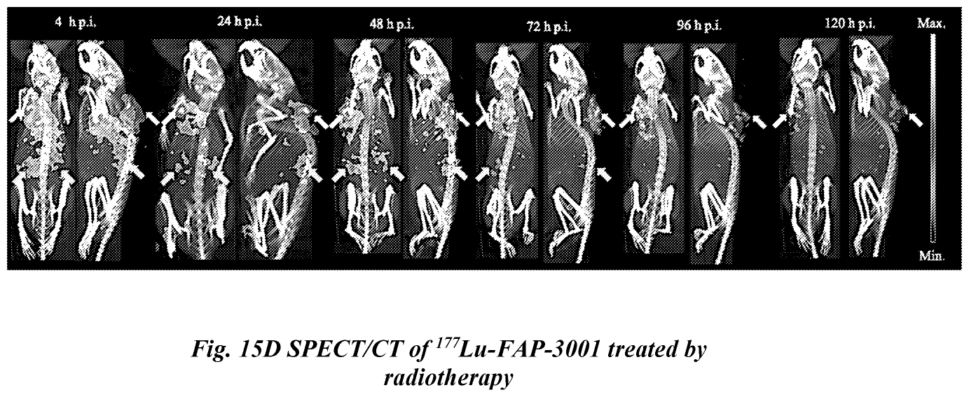

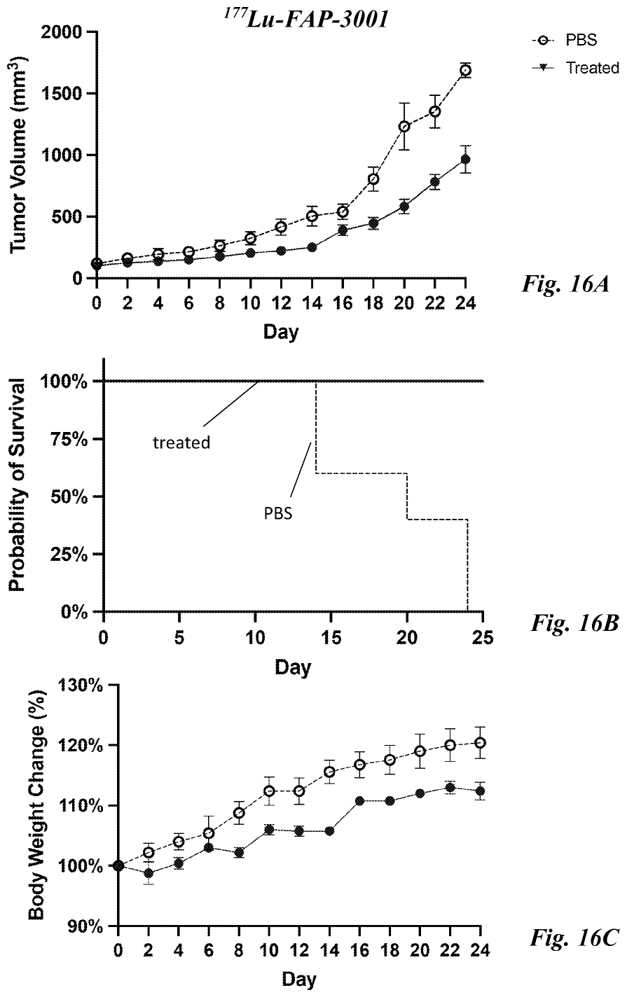

Therapy was initiated when the 4T1 tumor volume reached about 100 mm³. Control mice were injected with 5% EtOH in sterile PBS, whereas the treated mice were injected with 1 dose of 5 nmol of FAP-3001 radiolabeled with 1.50 mCi of lutetium-177 (¹⁷⁷Lu-FAP-3001) on day 0 via intravenous tail vein injection. Two assistants measured the tumors with a caliper every other day in blind fashion (i.e., did not know which mice were treated and which were control). Body weight of the mice were monitored as a measure of gross toxicity. FIG. 16A shows tumor growth data from the study 7, FIG. 16B depicts a survival curve of the study, and FIG. 16C shows relative body weight data of the mice from the study.

### Example 16

### ¹⁷⁷Lu-FAP-3001 vs. ¹⁷⁷Lu-FAP-3005 Radiotherapy Study in 4T1—Blind Measurements

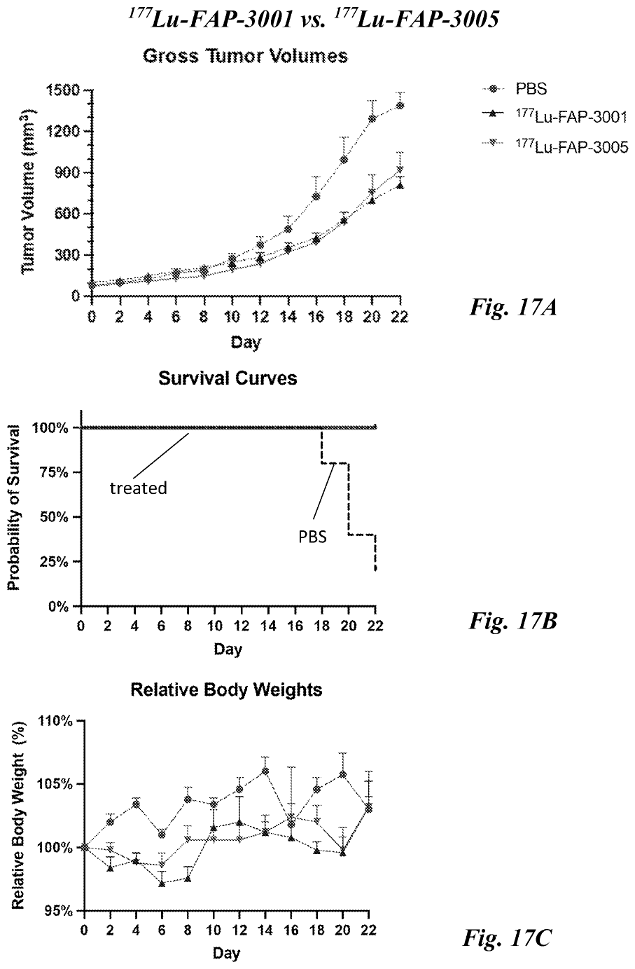

Therapy was initiated when the 4T1 tumor volume reached about 100 mm³. Control mice were injected with 5% EtOH in sterile PBS, whereas the treated mice were injected with 1 dose of 5 nmol of FAP-3001 or FAP-3005 radiolabeled with 1.50 mCi of lutetium-177 (¹⁷⁷Lu-FAP-3001 or ¹⁷⁷Lu-FAP-3005, respectively) on day 0 via intravenous tail vein injection. Two assistants measured the tumors with a caliper every other day in blind fashion (i.e., did not know which mice were treated and which were control). Body weight of the mice were monitored as a measure of gross toxicity. FIG. 17A shows tumor growth data from the study 7, FIG. 17B depicts a survival curve of the study, and FIG. 17C shows relative body weight data of the mice from the study.

### Example 17

### ¹⁷⁷Lu-FAP-3001 High Dose Radiotherapy Toxicology Stains

Mice were randomly selected from control and treated mice at various time points post-injection (1.5 mCi doses) for further toxicology evaluation. Organs of interest were harvested immediately post euthanasia, washed, and fixed in a 10% formalin buffered solution for 48-72 hours. Organs were then maintained in a 70% ethanol solution until radioactivity had fully decayed, after which they were submitted to the Histology Laboratory at Purdue University to be embedded in paraffin, sectioned, and stained with H&E (see FIG. 14). Tissue sections (n=1-4 per organ per mouse) were examined in a blinded manner for lesions by a licensed pathologist at the Purdue University Department of Comparative Pathobiology. A table summarizing the results and representative tissue slices are provided in FIG. 18A and FIG. 18B, respectively.

### Example 18

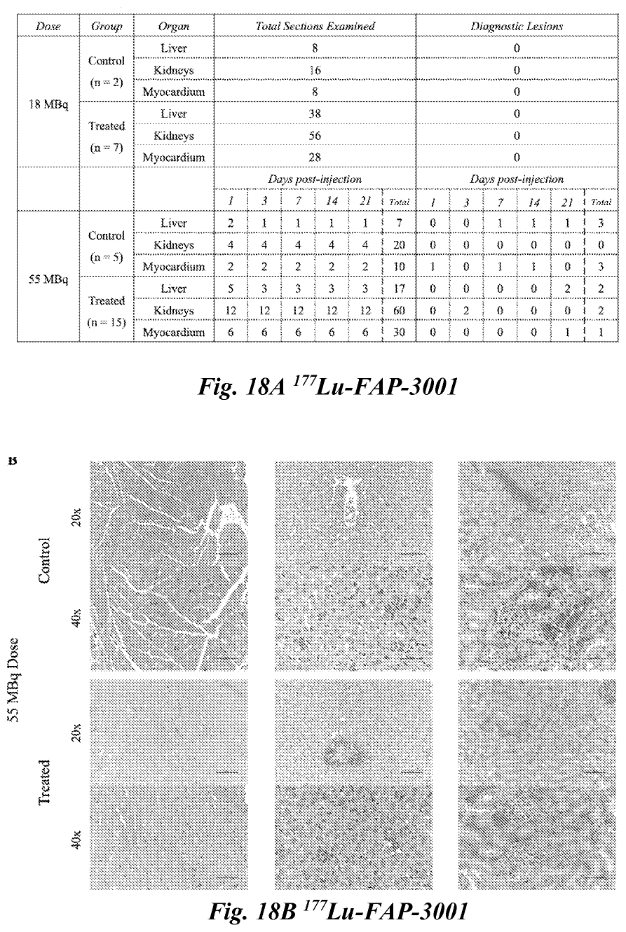

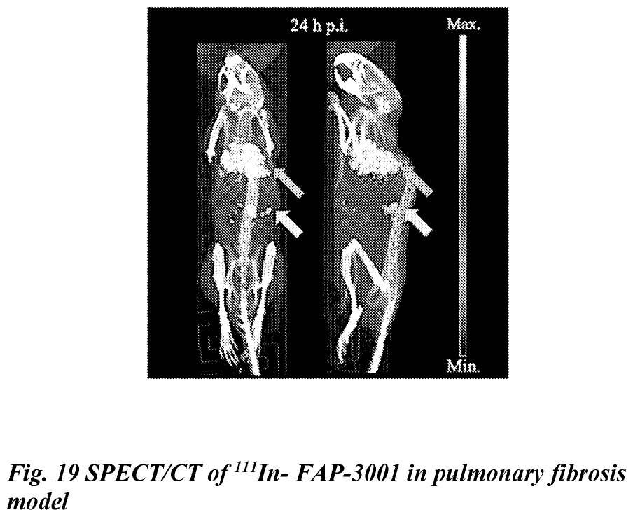

SPECT/CT scan was taken 24 hours post intravenous injection of ¹¹¹In-FAP-3001 (5 nmol radiolabeled with 0.3 mCi) in a murine pulmonary fibrosis model at day 14 post induction of the disease by intratracheal injection of bleomycin into a C57/BL6 mouse. See FIG. 19.

### Example 19

### High Affinity FAP Ligand and Molecular Docking

Designing a drug conjugate requires particular choice of the linker attachment point so that the binding affinity of the modified drug remains like that of the parent drug. Molecular docking experiments were utilized to identify a suitable linker attachment point in the known FAP inhibitor 1:

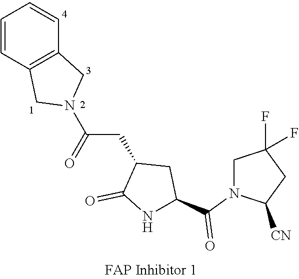 [SMILES: N#C[C@@H]1CC(F)(F)CN1C(=O)[C@@H]1C[C@@H](CC(=O)N2Cc3ccccc3C2)C(=O)N1]

The crystal structure of human FAP [PDB ID: 1Z68] was retrieved from Protein Data Bank (a global, open access digital data resource) and further prepared for docking using the protein preparation toolbox included in Schrodinger software package (Schrodinger, LLC, New York, New York). The protein structure was preprocessed using the default protocol in Schrodinger and a pH value of 7.4+/−0.5 was used to generate heteroatomic states using the Epik module. The protein structure was further refined to optimize the intramolecular hydrogen bonds and a restrained energy minimization was performed using the OPLS4 force field.

Structures of three FAP ligands (FAP inhibitor 1, intermediate 2′, and FAP-4000 (see structures of intermediate 2′ and FAP-4000 below)) were uploaded in Maestro (Schrodinger, LLC, New York, NY) and prepared for Glide docking using the LigPrep program (Schrodinger, LLC, New York, NY). Three-dimensional geometry of the ligand was optimized using the OPLS4 force field and used for docking.

The standard induced fit docking (IFD) protocol in the Schrodinger software package was used to dock the ligands of interest into the binding pocket of FAP. First, a receptor grid box was generated by specifying the amino acid residues in FAP reported to be involved in binding interactions. The IFD protocol utilizes the Glide docking protocol to generate up to 20 poses for each ligand which are further refined using the Prime Refinement module. The residues within 5 Å of ligand poses were refined and the side chains of the residues were optimized. Upon refinement of the binding site after initial docking, the ligands were redocked into structures that are within 30.0 kcal/mol of the best structure and within the top 20 structures overall. The standard precision model was used in the Glide redocking step.

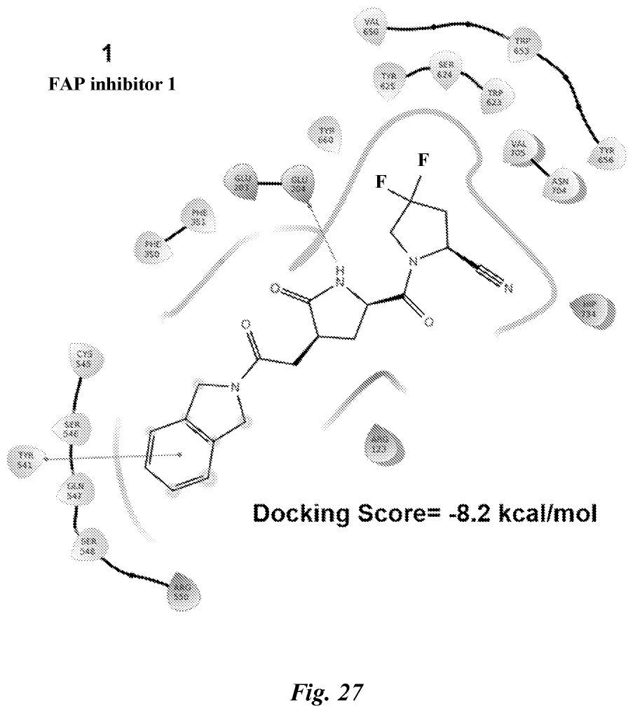

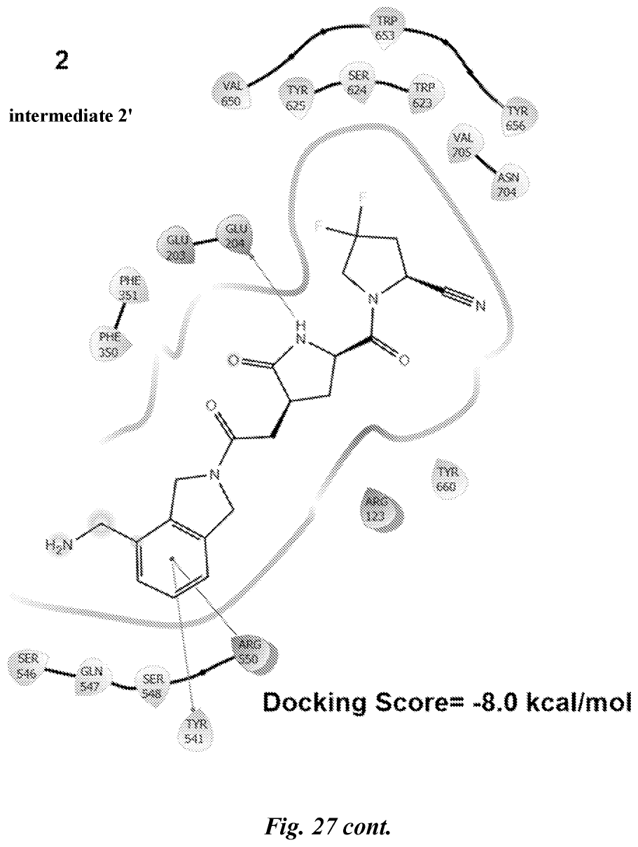

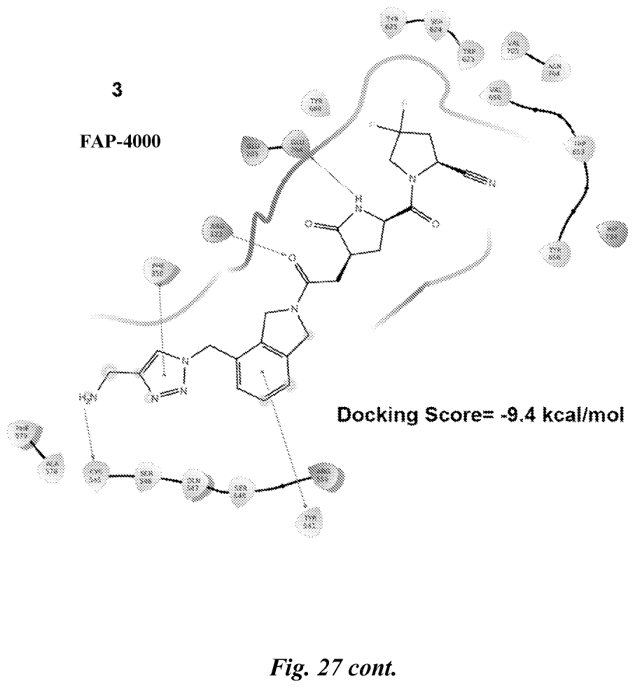

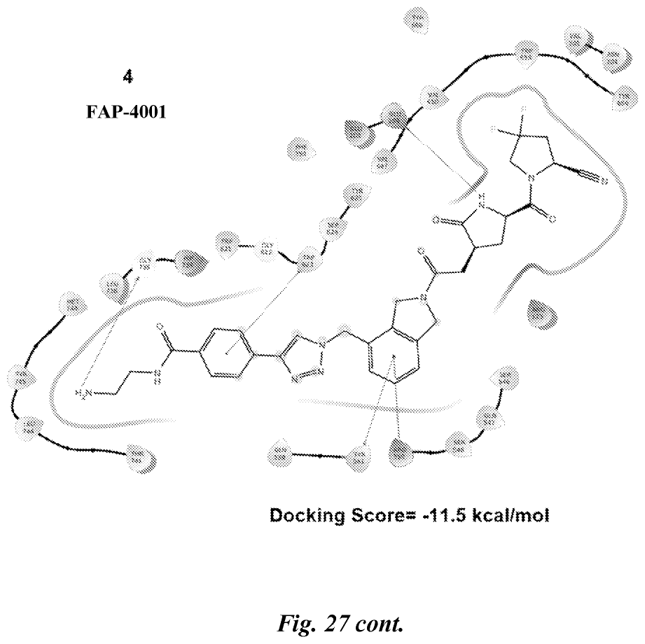

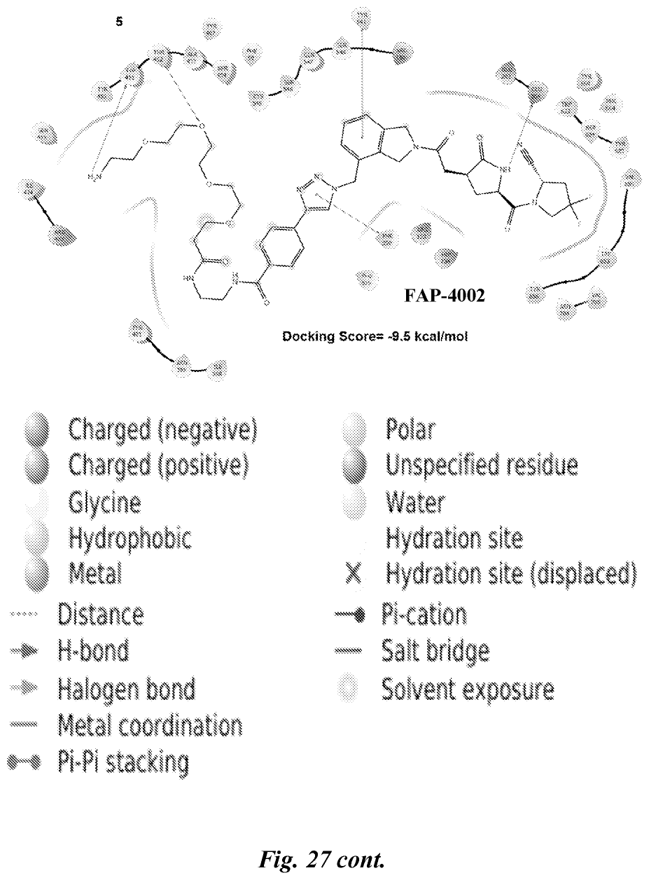

Referring to FIG. 27, in the docked pose of the FAP inhibitor 1 with FAP, Glu204 and Tyr541 residues were found to be involved in non-covalent interactions, where Glu204 forms a hydrogen bond with the N—H group of the pyrrolidine ring and Tyr541 forms a pi-pi interaction with the 2,3-dihydro isoindole ring of inhibitor 1. The shaded regions in the 2,3-dihydro isoindole ring marks the atoms that are solvent exposed (FIG. 27).

FAP inhibitor 1 was then further functionalized at the C-4 position of the 2,3-dihydro isoindole ring. A methylene amine (—CH₂NH₂) was attached at the C-4 position of the 2,3-dihydro isoindole ring of FAP inhibitor 1, resulting in intermediate 2′. When intermediate 2′ was docked against a FAP protein, the interactions thereof with the FAP protein were similar to that of the parent FAP inhibitor 1 with an additional cation-pi interaction of the 2,3-dihydro isoindole ring with Arg550. The methylene amine (—CH₂NH₂) in intermediate 2′ remained solvent exposed.

Because the presence of nearby aromatic amino acid residues was observed in the deep binding pocket of FAP, a hydrophobic spacer was explored to facilitate the formation of new stabilizing interactions with these aromatic amino acid residues. As such, the methylene amine group in intermediate 2′ was replaced with a triazole ring containing a solvent exposed methylene amine (—CH₂NH₂) group (FAP-4000) to result in a pi-pi interaction of the triazole ring with Phe350. In addition, the N—H of methyl amine formed a hydrogen bond with Cys545 and the Arg123 formed a hydrogen bond with the amide oxygen adjacent to the 2,3-dihydro isoindole ring (see FIG. 27). The increased number of intermolecular interactions resulted in an increased docking score from −8.0 kcal/mol (FAP inhibitor 1) to −9.4 kcal/mol (FAP-4000).

Because the C-1 position (—CH₂NH₂ unit) of the triazole ring of FAP-4000 was found to be solvent exposed in the docking pose, the methylene amine in FAP-4000 was then replaced with aminoethylbenzamide, to result in FAP-4001. When FAP-4001 was docked against the FAP protein, the docking score further increased to −11.5 kcal/mol. Specifically, the phenyl ring in FAP 4001 formed a pi-pi interaction with Trp623 and the 2,3-dihydro isoindole ring formed an additional cation-pi interaction with Arg550.

The —NH₂ moiety in FAP-4001 was then utilized for attaching a PEG₄-amine linker and the modified structure was again docked in the same binding pocket by setting up a larger grid box to accommodate the ligand-PEG₄-amine drug conjugate (FAP 4002). The interaction of the ligand with Glu204 and Tyr541 residues was conserved in the docked pose of FAP 4002 with FAP, and the docking score was −9.5 kcal/mol. The triazole ring was involved in a pi-pi interaction with Phe350 as shown in FIG. 27.

Accordingly, the data support the addition of a spacer and a linker to the parent (free) drug without interfering with critical non-covalent interactions of the parent drug, which helps the drug maintain its binding affinity even after conjugation.

### Example 20

### Synthesis of FAP-targeted Dye Compounds

The free amine functionality in FAP-4002 was utilized for synthesis of a FAP-targeted dye conjugate (FAP-4003) pursuant to Scheme 1 below:

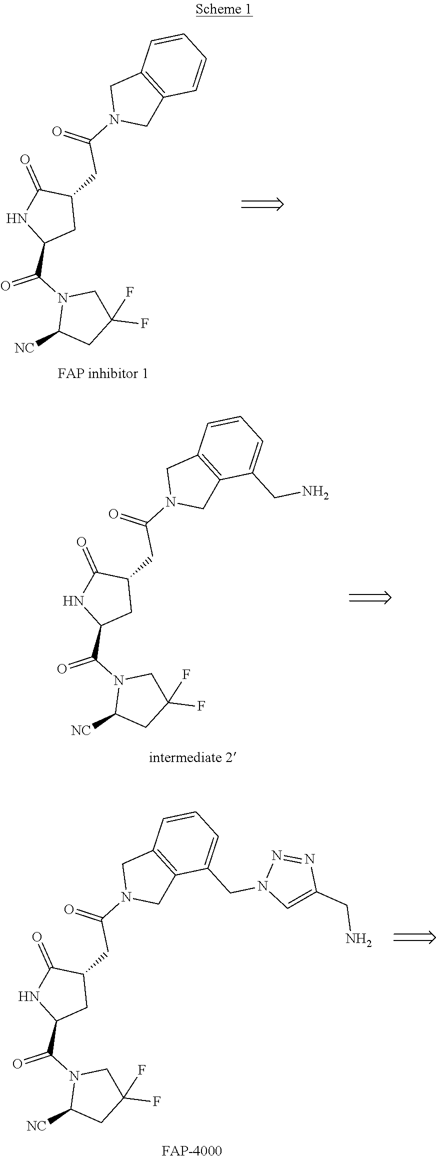 [SMILES: N#C[C@@H]1CC(F)(F)CN1C(=O)[C@@H]1C[C@@H](CC(=O)N2Cc3ccccc3C2)C(=O)N1 ; N#C[C@@H]1CC(F)(F)CN1C(=O)[C@@H]1C[C@@H](CC(=O)N2Cc3cccc(CN)c3C2)C(=O)N1 ; N#C[C@@H]1CC(F)(F)CN1C(=O)[C@@H]1C[C@@H](CC(=O)N2Cc3cccc(Cn4cc(CN)nn4)c3C2)C(=O)N1]

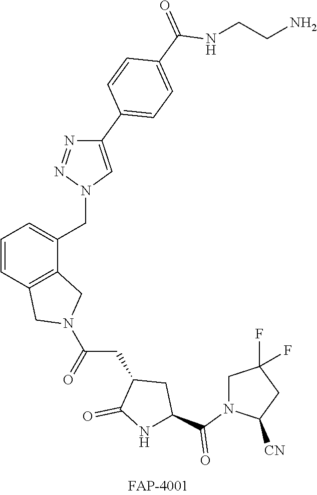 [SMILES: N#C[C@@H]1CC(F)(F)CN1C(=O)[C@@H]1C[C@@H](CC(=O)N2Cc3cccc(Cn4cc(-c5ccc(C(=O)NCCN)cc5)nn4)c3C2)C(=O)N1]

Synthesis of FAP-targeted fluorescent compounds was then initiated from intermediates 5′, 6′ and 7′ as shown in Scheme 2.

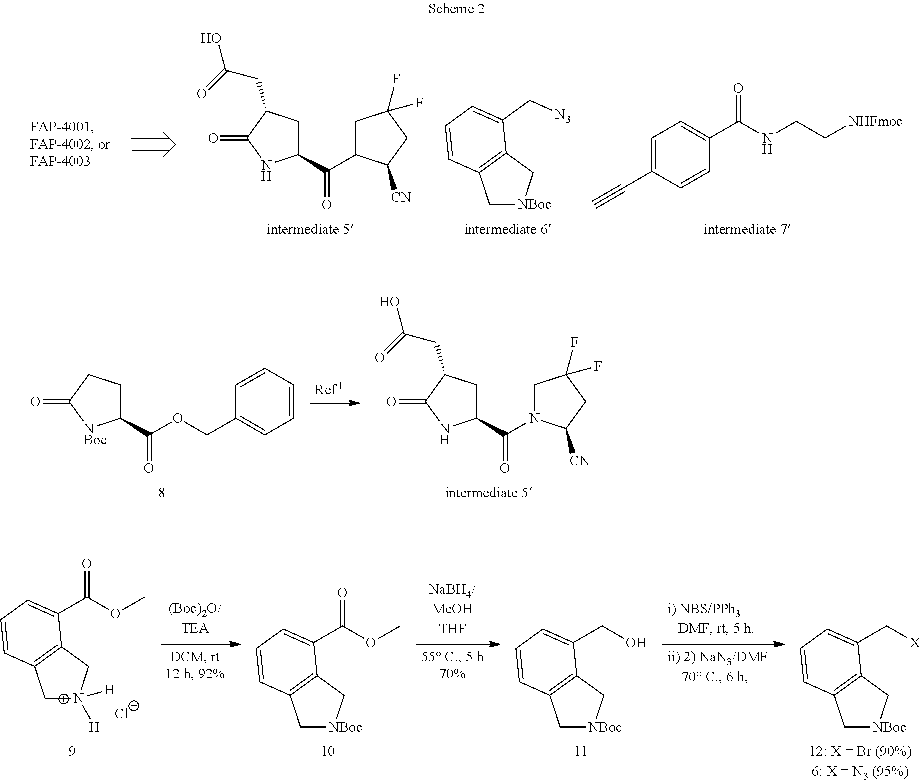

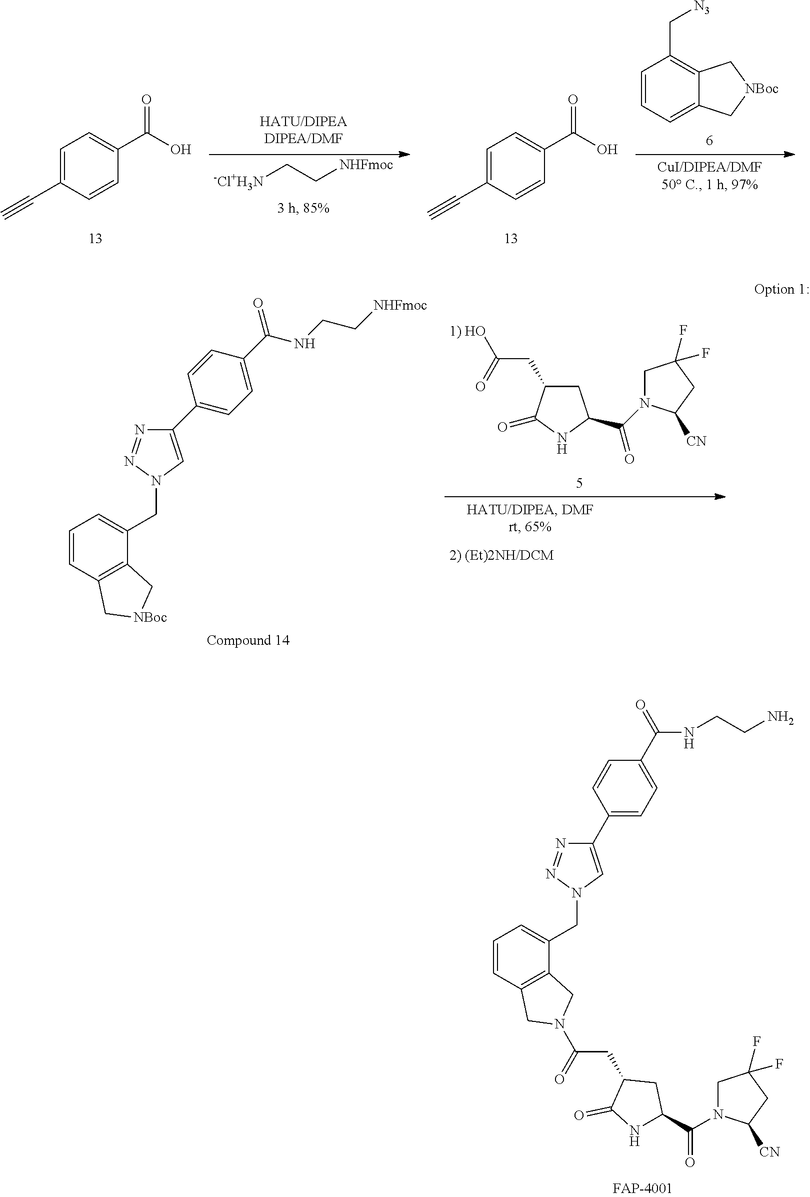

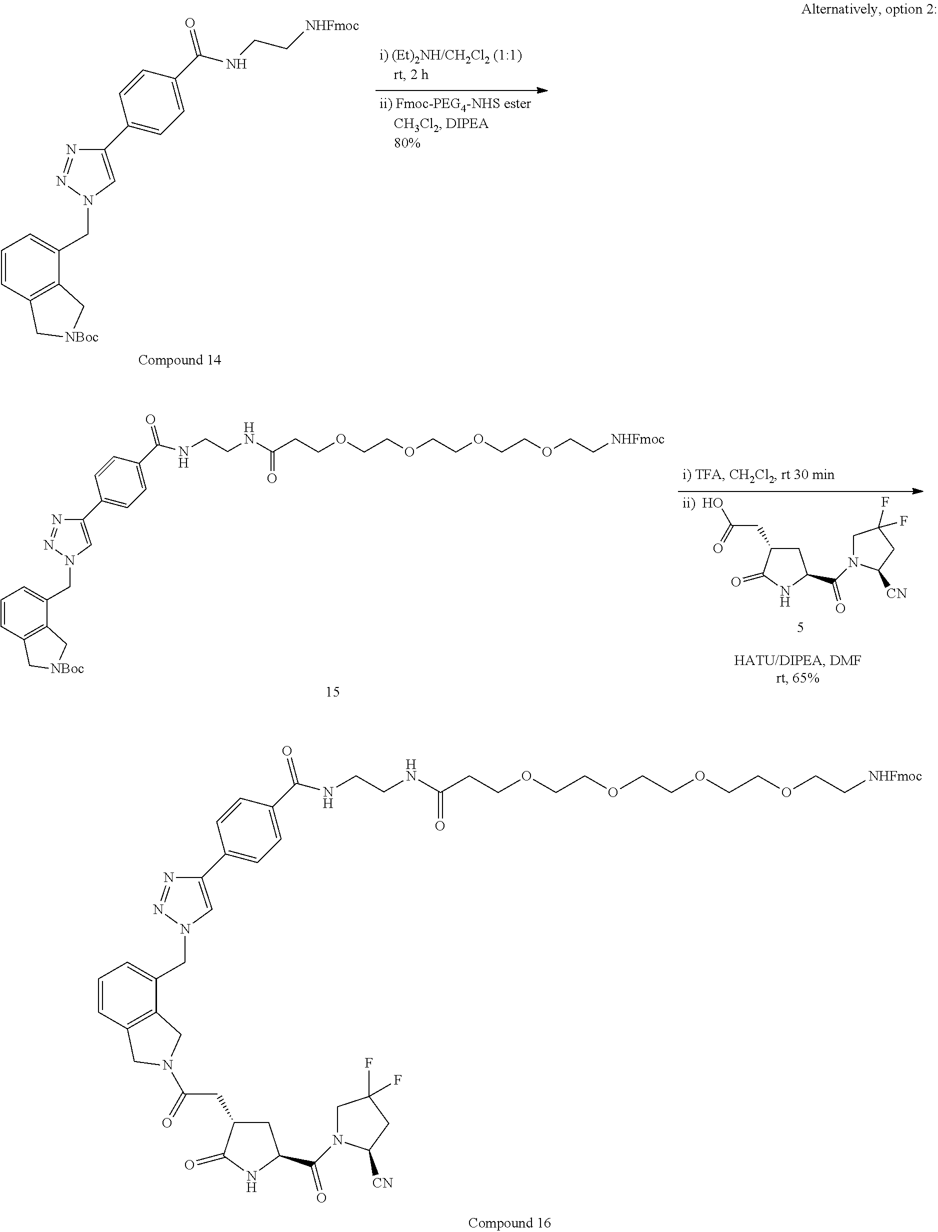

Briefly, intermediate 5′ was synthesized from commercially available Boc-L-pyroglutamic acid benzyl ester 8. Intermediate 6′ was prepared from methyl 2,3-dihydro-1H-isoindole-4-carboxylate hydrochloride 9 by tert-butyloxycarbonyl (Boc) protection in dichloromethane to yield compound 10 in quantitative yield. The methyl ester in compound 10 was then reduced with NaBH₄ by heating in methanol and tetrahydrofuran to provide the corresponding alcohol 11 in 90% yield. Bromination of 11 with NBS/PPh₃ in DMF at room temperature followed by nucleophilic substitution of the corresponding bromide 12 with NaN₃ in DMF at 70° C. for 12 hours afforded the key azide intermediate 6′ in 95% yield. Coupling of mono-Fmoc ethylenediamine hydrochloride with 4-ethynylbenzoic acid 13 in the presence of HATU/DIPEA in dry DMF yielded the alkyne intermediate 7′. The classical Cu(I)-catalyzed azide-alkyne cycloaddition (CuAAC) reaction of intermediate 6′ with intermediate 7′ in DMF at 55° C. for 5 hours then provided the click product 14 with the phenyl triazole appendage, and treatment of compound 14 with trifluoroacetic acid (TFA) in DCM, followed by the addition of activated acid 5 and subsequent Fmoc deprotection using diethylamine in DCM provided FAP-4001 (see Scheme 2, option 1).

Treatment of the same compound 14 with diethylamine in dichloromethane for 30 minutes, followed by coupling of the resultant amine with Fmoc-PEG₄-NHS ester in CH₂Cl₂ in the presence of DIPEA generated intermediate 15 in 90% yield (see Scheme 2, option 2). Treatment of intermediate 15′ with TFA in CH₂Cl₂ followed by coupling of the resultant amine with key acid intermediate 5′ in the presence of HATU/DIPEA in DMF yielded the FAP-targeted ligand (compound 16) in 65% yield.

Now referring to Scheme 3, to generate the final FAP-targeted fluorescent dye used below, the Fmoc protecting group in compound 16 was deprotected with diethylamine in dichloromethane to generate the free amine 17 (compound 17). The coupling of free amine 17 (compound 17) with 3-(4-hydroxyphenyl) propionic acid provided FAP-4002. Following this with displacement of the chloride from the near infrared dye (ClS0456) under basic conditions provided a FAP-targeted near infrared dye FAP-4003 in 65% yield.

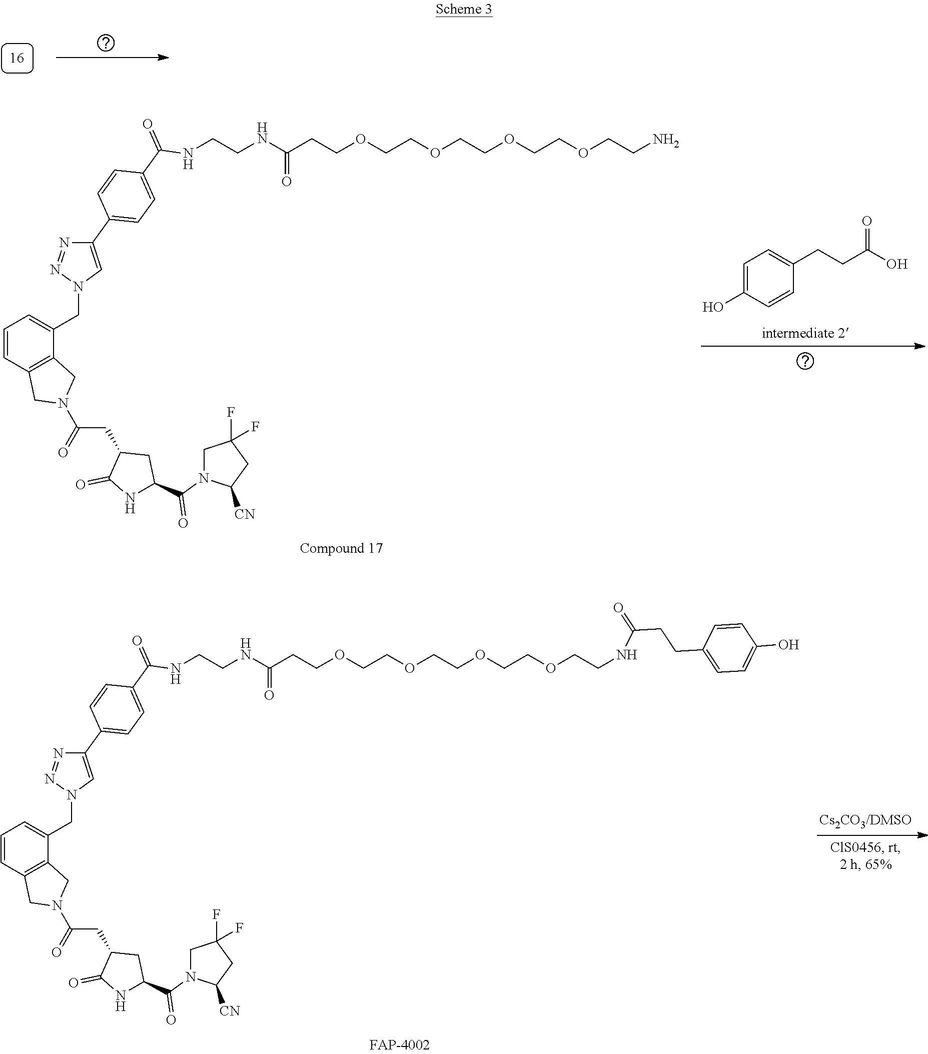 [SMILES: N#C[C@@H]1CC(F)(F)CN1C(=O)[C@@H]1C[C@@H](CC(=O)N2Cc3cccc(Cn4cc(-c5ccc(C(=O)NCCNC(=O)CCOCCOCCOCCOCCN)cc5)nn4)c3C2)C(=O)N1 ; O=C(O)CCc1ccc(O)cc1 ; N#C[C@@H]1CC(F)(F)CN1C(=O)[C@@H]1C[C@@H](CC(=O)N2Cc3cccc(Cn4cc(-c5ccc(C(=O)NCCNC(=O)CCOCCOCCOCCOCCNC(=O)CCc6ccc(O)cc6)cc5)nn4)c3C2)C(=O)N1]

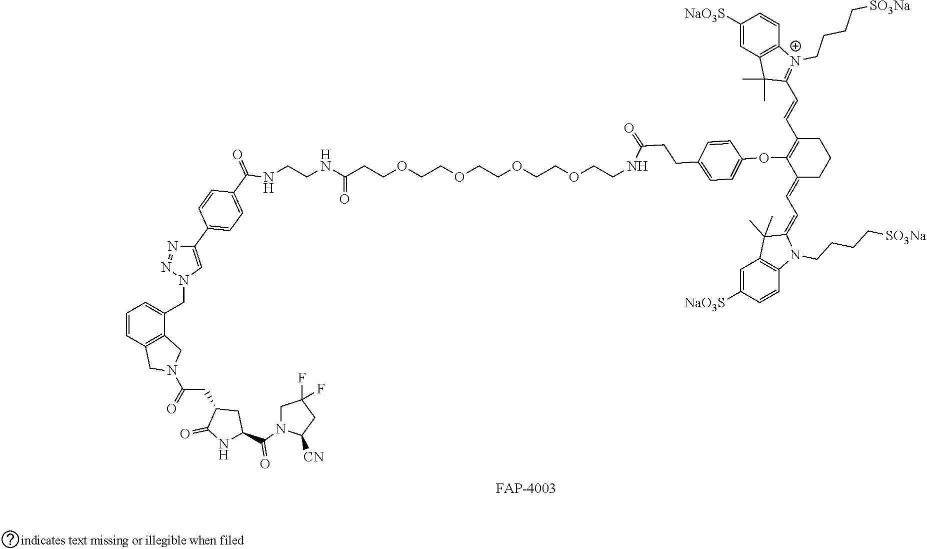

### Example 21

### Binding Internalization of Compounds

The following compounds were analyzed in terms of affinity for binding FAP: FAP inhibitor 1; FAP targeting ligand with linker (FTL-PEG₄-NH₂) (compound 17); a conjugate of compound 17 with 4-hydroxyphenyl propionic acid (FAP-4002); a conjugate of compound 17 with tetramethyl-rhodamine (FAP-4004); and a conjugate of FAP-4002 with ClS0456 (FAP-4003).

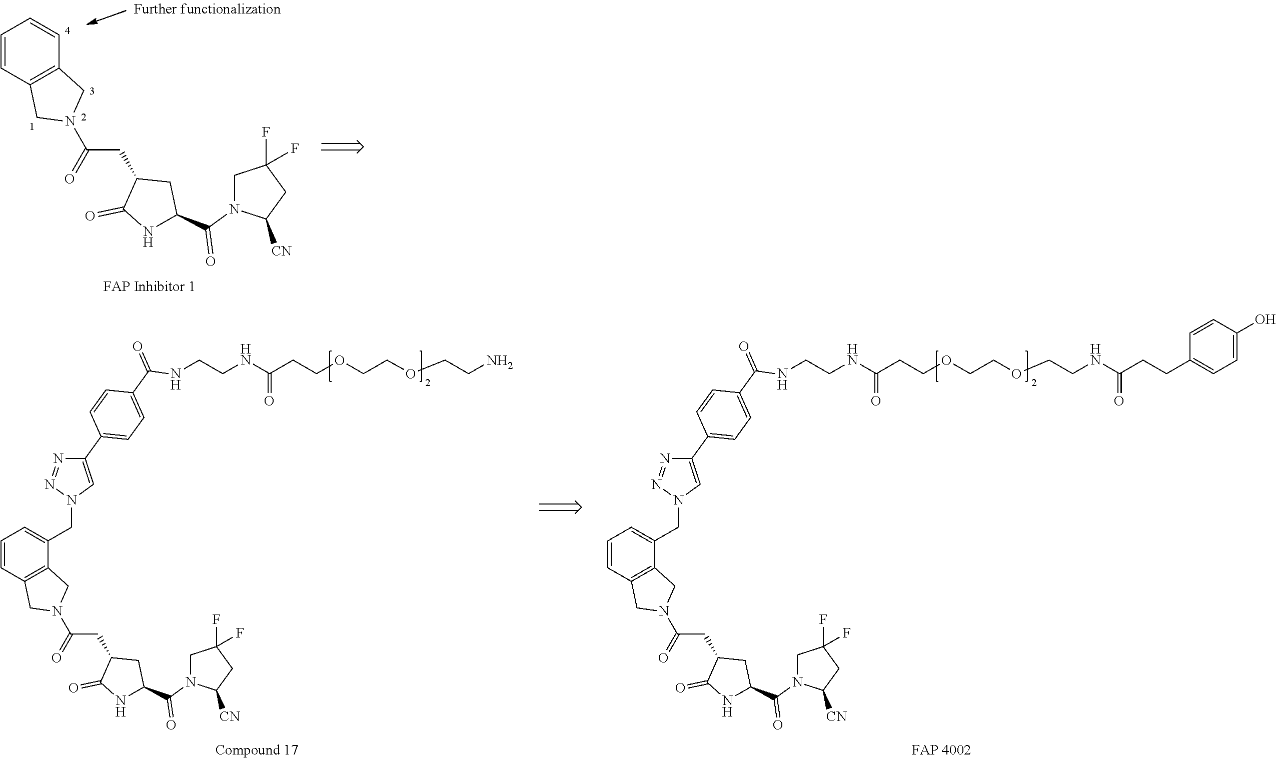

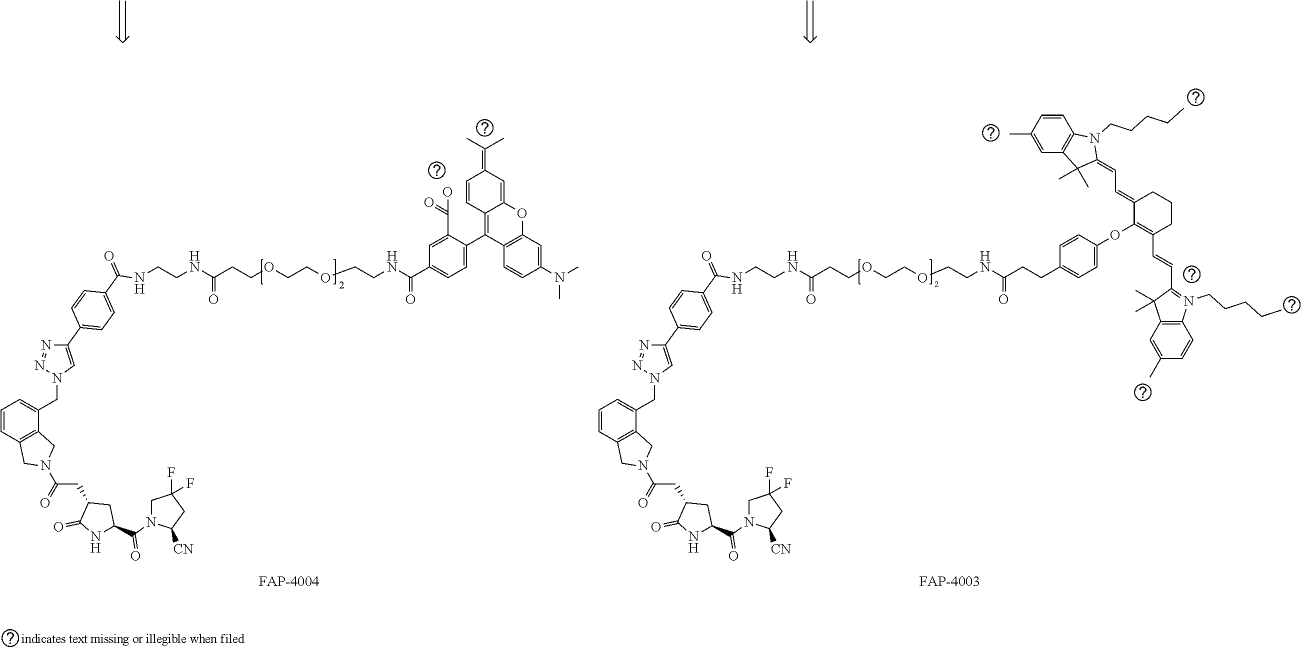

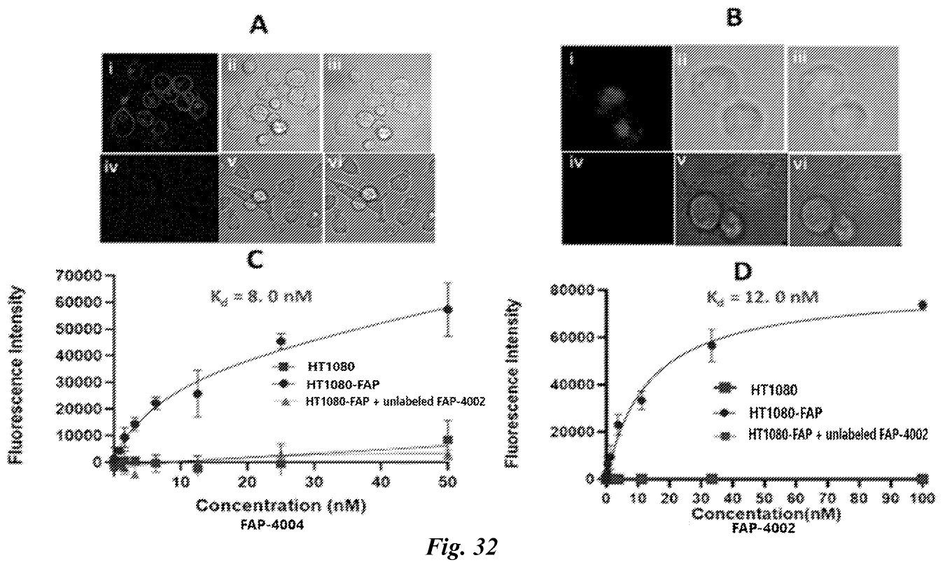

HT1080-FAP cells were incubated for 1 hour at 37° C. with 25 nm of FAP-4004 (see FIG. 32A) or FAP-4003 (see FIG. 32B) and examined by confocal miscopy and Wide-field Nikon microscopy, respectively. The binding affinities and specificities of each compound were also quantitated by measuring the fluorescence following incubation of either HT1080-FAP cells or HT1080 cells for 1 hour at 4° C. in the presence of increasing concentrations of FAP-4004 (see FIG. 32C) or FAP-4003 (see FIG. 32D) in the presence or absence of 100-fold excess of unlabeled FAP-4002.

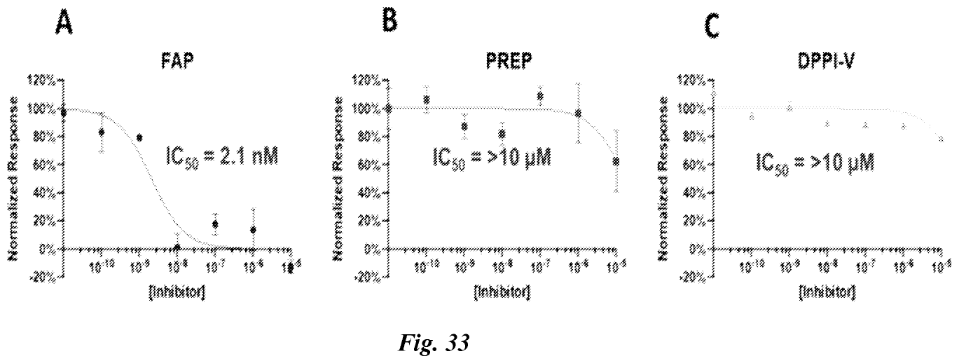

The ability of FAP-targeted FAP-4002 to inhibit the closely related dipeptidyl peptidases FAP, PREP, and DPP-IV were also assessed. FAP, PREP, and DPP-IV were incubated with FAP-targeted FAP-4002 for 10 minutes at room temperature prior to adding fluorogenic substrate. After allowing the reaction to proceed for 30 minutes, the reaction was terminated and the change in fluorescence was quantitated as a measure of catalytic activity. All assays were performed in triplicate, with SEM bars shown (see FIGS. 33A-33C).

### SYNTHESIS EXAMPLES

The following examples describe the materials and methods used to synthesize the intermediates and compounds described in Examples 21-23 and Schemes 1-3 in further detail. Following synthesis, all intermediates and compounds/products were characterized by LC-MS and nuclear magnetic resonance spectroscopy (NMR).

### Example 22

### Synthesis of 2-(tert-butyl) 4-methyl isoindoline-2,4-dicarboxylate (Compound 10)

At room temperature, 20 mL of DCM was added to a stirred solution of methyl 2,3-dihydro-1H-isoindole-4-carboxylate hydrochloride 9 (1.00 g, 5.64 mmol). (Boc)₂O (4.9 mL, 22.59 mmol) was then added in one portion to the mixture followed by triethylamine (2.9 mL, 22.59 mmol) dropwise. Stirring was continued there for 12 hours, then the reaction mixture was diluted with water (30 mL) and extracted into DCM (2×25 mL).

The organic layer was dried over anhydrous MgSO4, filtered, and the filtrate was evaporated under reduced pressure. The obtained crude residue was purified by CombiFlash using Hexanes+ethyl acetate, and the mobile phase provided compound 10 (1.4 g, 92%) as white solid. ¹H-NMR (CDCl₃, 500 MHZ) δ=7.92 (dd, J₁=15.0 Hz, J₂=10.0 Hz, 1H); 7.44-7.32 (m, 1H); 7.33 (m, 1H); 4.94 (d, J=12.6 Hz, 2H); 4.67 (d, J=17.0 Hz, 2H); 3.90 (s, 3H); 1.51 (s, 9H) ppm. ¹³C-NMR (CDCl₃, 125 MHZ) δ=166.41, 154.50, 154.33, 139.93, 138.96, 138.83, 138.56, 129.37, 128.96, 127.61, 127.17, 126.89, 125.55, 125.14, 79.80, 79.73, 53.80, 53.46, 52.05, 51.79, 51.49 28.55 ppm. LCMS for 4: LC/MS (m/z): calculated for C₁₅H₂₁NO₄ [M+H]: 278.14, found 278.13 g/mol.

### Example 23

### Synthesis of tert-butyl 4-(hydroxymethyl)isoindoline-2-carboxylate (Alcohol 11)

Under N₂ atmosphere, sodium bromide (1.37 g, 36.101 mmol) was added to a stirred solution of compound 10 (1.0 g, 3.61 mmol) in THF (10.0 mL) at room temperature. Thereafter, methanol (10 mL) was added to the reaction mixture slowly for 5 minutes. The reaction was warmed to 55° C. and continuously stirred for 5 hours.

The reaction mixture was then cooled to 0° C., slowly quenched with saturated aqueous ammonium chloride, and extracted into EtOAc (60 mL). The organic phase was collected, dried over sodium sulfate, and the solvent distilled to provide crude residue, which was purified by CombiFlash to yield alcohol 11 (700 mg, 70%) as white gummy solid.

¹H-NMR (CDCl₃, 500 MHZ) δ=7.28-7.25 (m, 2H); 7.19-7.13 (m, 1H); 4.68-4.62 (m, 6H); 1.51 (s, 9H) ppm. ¹³C-NMR (CDCl₃, 125 MHZ) δ=154.64, 137.60, 137.39, 135.62, 135.32, 143.92, 127.81, 125.90, 125.67, 122.05, 121.60, 79.87, 63.18, 62.80, 52.25, 51.95, 51.05, 50.81, 28.58, 28.54 ppm. LC-MS for 11. LC-MS (m/z): calculated for C₁₄H₂₀NO₃ [M+H]: 250.14, found 250.14 g/mol.

### Example 24

### Synthesis of tert-butyl 4-(bromomethyl)isoindoline-2-carboxylate (Bromide 12)

PPh₃ (790 mg, 3.01 mmol) was added to a stirred solution of compound 11 (500 mg, 2.00 mmol) in DMF (10 mL), followed by freshly recrystallized N-bromosuccinimide (NBS) (532 mg, 3.01 mmol). The reaction mixture was stirred at room temperature under N₂ atmosphere for 4-5 hours, then diluted with water (40 mL) and extracted into ethyl acetate (2×25 mL). The organic layer was washed with water and brine, then dried over anhydrous sodium sulphate and filtered. The filtrate was evaporated under reduced pressure to obtain crude residue, which was purified by CombiFlash to provide the bromide 12 (450 mg, 90%) as a white solid.

¹H-NMR (CDCl₃, 500 MHZ) δ=7.28-7.17 (m, 3H); 4.71 (m, 4H); 4.42 (d, J=8.5 Hz, 2H); 1.52 (s, 9H) ppm. ¹³C-NMR (CDCl₃, 125 MHZ) δ=154.46, 138.38, 138.03, 136.78, 136.48, 132.44, 132.17, 128.20, 128.13, 127.8, 123.18, 122.90, 79.98, 79.88, 52.4, 52.18, 50.74, 50.63, 28.58, 28.54 ppm. LC-MS for 12: LC-MS: (m/z): calculated for C₁₄H₁₉BrNO₂ [M+H]: 312.05, found 312.05 g/mol.

### Example 25

### Synthesis of Tert-butyl 4-(azidomethyl)isoindoline-2-carboxylate (Intermediate 6′)

NaN₃ (420 mg, 6.430 mmol) was added to a stirred solution of bromide compound 12 (400 mg, 1.286 mmol) in DMF, and the mixture was continuously stirred at 75° C. for 6 hours. The reaction mixture was then diluted with water and extracted into ethyl acetate, and the organic layer was washed with water, brined and dried over anhydrous sodium sulphate, and filtered. The filtrate was evaporated under reduced pressure and purified by CombiFlash to result in azide intermediate 6′(300 mg, 95%) as white solid.

¹H-NMR (CDCl₃, 500 MHZ) δ=7.32-7.25 (m, 2H); 7.22-7.20 (m, 1H); 4.72-4.66 (m, 4H); 4.31 (s, 2H); 1.53 (s, 9H) ppm. ¹³C-NMR (CDCl₃, 125 MHZ) δ=154.42, 138.28, 137.95, 136.34, 135.95, 130.18, 129.92, 128.08, 127.40, 127.30, 122.96, 122.65, 79.96, 52.59, 52.48, 52.39, 52.15, 51.05, 50.89, 28.56 ppm. LC-MS for 7: LC-MS (m/z): calculated for C₁₄H₁₉N₄O₂ [M+H]: 275.14, found 275.14 g/mol.

### Example 26

### Synthesis of (9H-fluoren-9-yl)methyl (2-(4-ethynylbenzamido)ethyl)carbamate (Intermediate 7′)

Under N₂ atmosphere, HATU (1.4 gm, 3.76 mmol) was added to a stirred solution of 4-Ethynylbenzoic acid compound 13 (500 mg, 3.424 mmol) in dry DMF (10 mL) at room temperature, followed by DIPEA (1.7 mL, 10.27 mmol). The mixture was continuously stirred for 10 minutes to activate the acid.

N-Fmoc-ethylenediamine (1.0 g, 3.76 mmol) was then added to the reaction mixture, and the mixture was continuously stirred for an additional 3 hours. Thereafter, the reaction mixture was diluted with water (50 mL) and the obtained precipitate filtered through a Buckner funnel to result in a white solid. The white solid was washed with water (2×50 mL) again and dried under vacuum for 1 hour to provide intermediate 7′ (1.2 gm, 85%).

¹H-NMR (CD₃OD+CDCl₃, 500 MHZ) δ=7.76-7.73 (m, 4H); 7.59 (d, J=7.5 Hz, 2H); 7.50 (d, J=8.3 Hz, 1H); 7.35 (t, J=7.4 Hz, 2H); 7.25 (m, 2H); 4.44 (bs, 2H); 4.33 (d, J=7.0 Hz, 1H); 4.16 (t, J=7.0 Hz, 1H); 3.50 (s, 1H); 3.48 (t, J=6.1 Hz, 2H); 3.34 (t, J=6.0 Hz, 2H) ppm. ¹³C-NMR (CD₃OD+CDCl₃, 125 MHZ) δ=168.27, 158.16, 143.81, 141.20, 134.02, 131.83, 127.49, 127.06, 126.85, 125.62, 124.83, 119.65, 83.34, 79.69, 77.92, 66.61, 47.06, 40.04 ppm. and LC-MS for 7: LC-MS (m/z): calculated for C₂₆H₂₃N₂O₃ [M+H]: 411.16, found 411.16 g/mol. HRMS-ESI: calcd for C₂₆H₂₃N₂O₃ [M+H]+: 411.1708, found 411.1710.

### Example 27

### Synthesis of tert-butyl4-((4-(4-((2-((((9H-fluoren-9-yl)methoxy)carbonyl)amino)ethyl) carbamoyl)phenyl)-1H-1,2,3-triazol-1-yl)methyl)isoindoline-2-carboxylate (Compound 14)

CuI (0.5 eq) was added to a mixture of azide intermediate 6 (1.0 eq) and alkyne intermediate 7 (1.2 eq) in dry DMF (5.0 mL), followed by the addition of DIPEA (2.0 eq). The reaction mixture was stirred at 55° C. under nitrogen atmosphere for 1 hour, transferred to room temperature, diluted with saturated aqueous ammonium chloride (20 mL), and vigorously stirred for 15 minutes. Solid residue formed in the reaction mixture was filtered and washed with water (2×20 mL) and dried under vacuum for 1 hour to provide compound 14 as brown solids.

Compound 14 was utilized in studies without purification in some instances. An amount of compound 14 was also purified using a EtOAC/Hexanes mixture as mobile phase to provide compound 14 as white solid (97%).

¹H-NMR (CDCl₃, 500 MHZ) δ=7.79 (bs, 3H); 7.78-7.70 (m, 2H); 7.68 (d, J=7.9 Hz, 2H); 7.52 (d, J=7.3 Hz, 2H); 7.73-7.720 (m, 7H); 7.15-7.13 (m, 1H); 5.50 (s, 2H); 4.67-4.57 (m, 4H); 4.34 (d, J=6.7 Hz, 2H); 4.12 (t, J=6.7 Hz, 1H); 3.48 (t, J=5.7 Hz, 1H); 3.35-3.33 (m, 2H); 2.77 (s, 2H); 2.71 (bs, 1H); 1.46 (s, 9H) ppm. ¹³C-NMR (CDCl₃, 125 MHZ) δ=173.47, 167.26, 156.65, 154.33, 144.98, 141.27, 138.65, 138.46, 136.59, 135.92, 129.26, 129.03, 128.58, 127.82, 127.67, 127.44, 127.06, 125.61, 125.12, 123.58, 123.26, 119.95, 80.18, 80.08, 70.49, 70.13, 67.14, 66.58, 50.98, 50.80, 47.25, 40.91, 39.06, 36.85, 28.52 ppm. LC-MS for 14: LC-MS (m/z): calcd for C₄₀H₄₁N₆O₅ [M+H]: 685.31, found 685.31 g/mol. HRMS-ESI: calcd for C₄₀H₄₁N₆O₅ [M+H]+: 685.3138, found 685.3139.

### Example 28

### Synthesis of Synthesis of tert-butyl 4-((4-(4-((1-(9H-fluoren-9-yl)-3,19-dioxo-2,7,10,13,16-pentaoxa-4,20-diazadocosan-22-yl)carbamoyl)phenyl)-1H-1,2,3-triazol-1-yl)methyl)isoindoline-2-carboxylate (Intermediate 15′)

(Et)₂NH (1.0 mL) was added to a stirred solution of compound 14 (400 mg, 0.584 mmol) in a DCM+MeOH mixture (1+0.5 mL) and continuously stirred for 2 hours. The reaction mixture was evaporated under reduced pressure and crude residue obtained and purified via CombiFlash using MeOH+CH₂Cl₂ as mobile to provide the free amine of compound 14 (which was retained and used in the studies described herein).

A portion of the amine compound was dissolved in DCM (1 mL for 1 mmol), and Fmoc-NH(PEG)₄ NHS ester (1.2 eq) and DIPEA (2.0 eq) were added thereto with stirring at room temperature under nitrogen atmosphere for 1 hour. The reaction mixture evaporated under reduced pressure and the obtained crude residue was purified by CombiFlash using DCM+MeOH as mobile phase to provide intermediate 15′ in 80% as white solid.

¹H-NMR (CDCl₃, 500 MHZ) δ=7.86 (m, 4H); 7.74 (d, J=7.5 Hz, 2H); 7.68 (d, J=3.7 Hz, 1H); 7.57 (m, 3H); 7.37 (t, J=7.5 Hz, 2H); 7.28 (m, 4H); 7.16 (m, 2H); 5.67 (bs, 1H); 5.51 (d, J=6.7 Hz, 2H); 4.71 (s, 2H); 4.65 (m, 2H); 4.37 (d, J=7.2 Hz, 1H); 4.19 (t, J=7.0 Hz, 1H); 3.72-3.34 (m, 22H); 2.44 (s, 2H); 1.51 (s, 9H) ppm. ¹³C-NMR (CDCl₃, 125 MHZ) δ=173.47, 167.26, 156.65, 154.33, 143.98, 141.27, 138.65, 138.46, 136.59, 135.92, 133.77, 133.19, 129.26, 129.03, 128.58, 127.82, 127.67, 127.44, 127.06, 125.61, 125.12, 123.58, 123.26, 119.95, 80.18, 80.08, 70.49, 70.42, 70.13, 70.06, 67.14, 66.58, 52.11, 51.92, 51.73, 50.90, 50.81, 47.25, 41.59, 40.90, 39.06, 36.85, 28.52 ppm. LC-MS for 15: LC-MS (m/z): calcd for C₅₁H₆₂N₇O₁₀ [M+H]: 932.45, found 932.45 g/mol. HRMS-ESI: calcd for C₅₁H₆₁N₇O₁₀Na [M+Na]+: 954.4377, found 954.4433.

### Example 29

### Synthesis of (9H-fluoren-9-yl)methyl (1-(4-(1-((2-(2-((3S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6-dioxo-9,12,15,18-tetraoxa-2,5-diazaicosan-20-yl)carbamate (Compound 16)

TFA (0.5 ml) was added to a stirred solution of intermediate 15 (250 mg, 0.268 mmol) in DCM (1.0 mL) at room temperature. The mixture was continuously stirred for 30 minutes, and thereafter evaporated and dried under vacuum. In a separate round bottom flask, acid intermediate 5 (100 mg, 0.333 mmol) was dissolved in DMF (0.5 mL) followed by HATU (151 mg, 0.399 mmol) and DIPEA (0.170 mL, 0.999 mmol). The reaction mixture was stirred under nitrogen atmosphere at room temperature for 10 minutes for activation of the acid functionality.

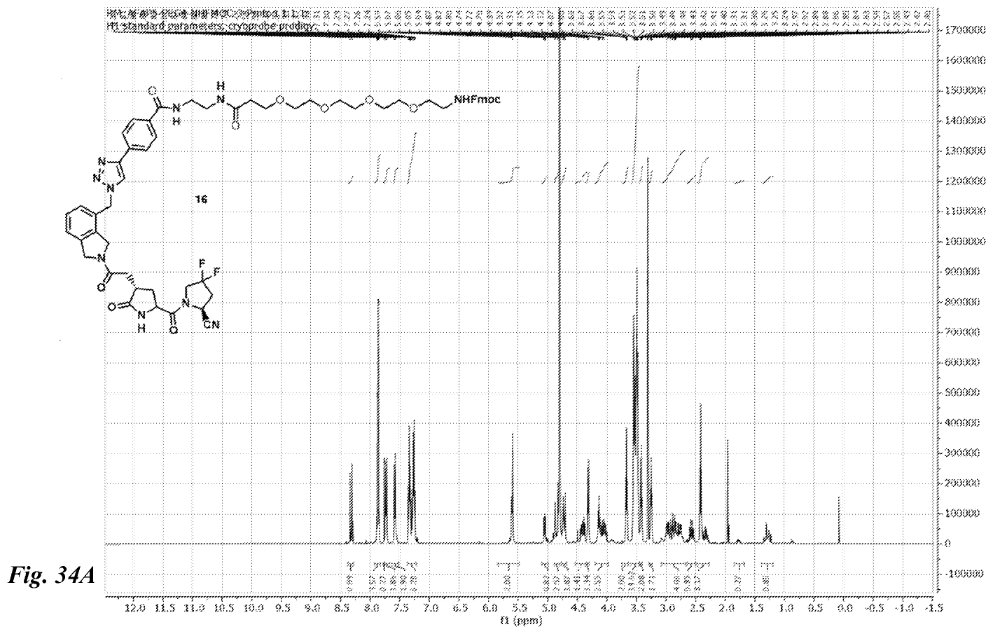

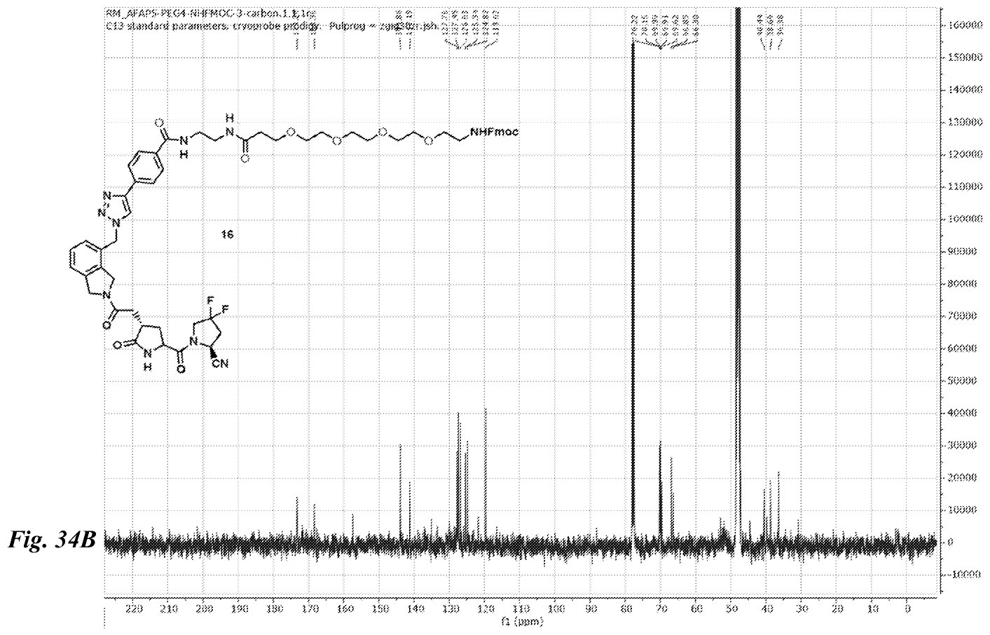

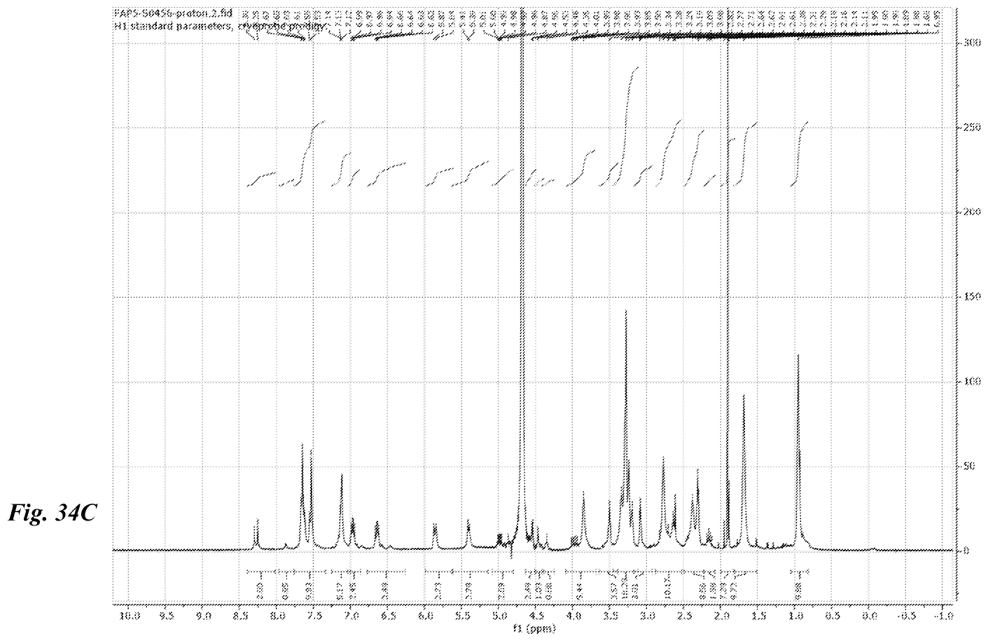

The amine in situ generated from intermediate 15 (220 mg, 0.268 mmol) was dissolved in DMF (1 mL), added to above reaction mixture and stirred continuously for 2 hours. The reaction mixture was then diluted with water (15 mL) and stirred at room temperature for 15 minutes. The black turbidity formed in the reaction mixture was filtered and redissolved in a mixture of methanol and dichloromethane. The organic layer was evaporated and the obtained crude residue was purified via reverse-phase preparative high performance liquid chromatography (HPLC) (A=20 mm ammonium acetate buffer (pH=7), B=acetonitrile, solvent gradients 5% B to 95%) in 60 minutes to compound 16 as white solid (175 mg, 65%). FIGS. 34A-34B show data confirming the production of compound 16 and FIG. 34C shows H¹ NMR (D₂O) data confirming the production of FAP-4003.

¹H-NMR (CD₃OD+CDCl₃, 500 MHZ) δ=8.32 (d, J=15.0 Hz, 1H), 7.89-7.85 (m, 4H), 7.73 (d, J=10.0 Hz, 2H); 7.58 (d, J=5.0 Hz, 2H); 7.35-7.24 (m, 8H); 5.61-5.59 (m, 2H); 5.05 (dd, J1=9.1 Hz, J2=3.9 Hz, 1H); 4.85 (m, 2H); 4.72 (d, J=17.2 Hz, 2H); 4.45-4.37 (m, 1H); 4.31 (d, J=6.8 Hz, 2H); 4.15-4.02 (m, 3H); 3.67 (t, J=6.0 Hz, 2H); 3.55-3.48 (m, 20H); 3.31-3.25 (m, 6H); 3.0-2.74 (m, 5H); 2.60-2.54 (m, 1H); 2.43-2.30 (m, 4H) ppm. ¹³C-NMR (CD₃OD+CDCl₃, 125 MHZ) δ=173.28, 168.36, 156.66, 143.88, 141.19, 127.76, 127.45, 126.83, 125.34, 124.82, 121.72, 119.62, 70.72, 70.19, 69.99, 69.91, 69.62, 66.85, 66.30, 52.80, 52.06, 44.58, 40.44, 39.86, 38.69, 37.60 36.38 ppm. LC-MS for 16: LC-MS (m/z): calculated C₅₈H₆₅F₂N₁₀O₁₁ [M+H]: 1115.47, found 1115.47 g/mol. HRMS-ESI: calculated for C₅₈H₆₅F₂N₁₀O₁₁ [M+H]: 1115.4802, found 1115.4817.

### Example 30

### Synthesis of sodium 2-((E)-2-((E)-2-(4-(1-(4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)phenyl)-1,6,22-trioxo-9,12,15,18-tetraoxa-2,5,21-triazatetracosan-24-yl)phenoxy)-3-(2-((E)-3,3-dimethyl-5-sulfonato-1-(4-sulfonatobutyl)indolin-2-ylidene)ethylidene)cyclohex-1-en-1-yl)vinyl)-3,3-dimethyl-1-(4-sulfonatobutyl)-3H-indol-1-ium-5-sulfonate (FAP-4003)

Diethyl amine (Et)₂NH (2.0 mL) was added to a stirred solution of compound 16 (250.0 mg, 0.2244 mmol) in DCM+MeOH (1+1 mL) and continuously stirred at room temperature for 2 hours. The reaction mixture was evaporated under reduced pressure, and the obtained crude residue was redissolved in EtOAc (10 mL). The resulting precipitate was filtered through a Buckner funnel to provided free anime (intermediate 2) as a brown solid, LC-MS for 2: LC-MS (m/z): calcd for C₄₃H₅₅F₂N₁₀O₉ [M+H]: 893.40, found 893.40 g/mol. HRMS-ESI: calculated for C₄₃H₅₅F₂N₁₀O₉ [M+H]+: 893.4121, found 893.4160. This material was used for the synthesis of FAP-targeted dye compound 4 as described below.

4-(1-((2-(2-((3S,5S)-5-((S)-2-cyano-4,4-difluoropyrrolidine-1-carbonyl)-2-oxopyrrolidin-3-yl)acetyl)isoindolin-4-yl)methyl)-1H-1,2,3-triazol-4-yl)-N-(22-(4-hydroxyphenyl)-4,20-dioxo-7,10,13,16-tetraoxa-3,19-diazadocosyl)benzamide (intermediate 3) was then prepared by adding 3-(4-Hydroxyphenyl) propionic acid (14.0 mg, 0.084 mmol), HATU (38.0 mg, 0.101 mmol) and DIPEA (42 μl, 0.252 mmol) to a stirred solution of amine (intermediate 2) (50 mg, 0.056 mmol) in DMF (1.0 mL) and subjecting the mixture to continuous stirring at room temperature for 30 minutes. The reaction mixture was then quenched by water (5 mL) and the obtained crude residue was purified by using UHPLC (A=20 Mm ammonium acetate buffer (pH=7), B=acetonitrile, solvent gradients 5% B to 95% in 60 min) to provide intermediate 3 as white solid (34 mg, 60%). LC/MS for 3: LC-MS (m/z): calculated for C₅₂H₆₃F₂N₁₀O₁₁ [M+H]+: 1041.46, found: 1041.46. HRMS-ESI: calculated for C₅₂H₆₃F₂N₁₀O₁₁ [M+H]+: 1041.4645, found 1041.4650.

To a stirred solution of intermediate 3 (5.0 mg, 0.00480 mmol) in anhydrous DMSO (500 μl), ClS0456 Dye (4.6 mg, 0.0048 mmol) was added at room temperature, under argon atmosphere, followed by Cs₂CO₃ (15.0 mg, 0.0480 mmol). The mixture was continuously stirred at room temperature for an additional 3-4 hours and progress of the reaction was monitored by LCMS.

The reaction mixture was then diluted with water and purified by using UHPLC (A=20 Mm ammonium acetate buffer (pH=7), B=0 acetonitrile, solvent gradients 5% B to 35% in 60 minutes) to provide intermediate 5 as fluffy green solid (6.0 mg, 66%). LC-MS for 4: LC/MS (m/z): [M+H] calcd for C₉₀H₁₁₀F₂N₁₂O₂₃S₄[M+H]: 1892.66, found: 1892.60, [M+H]/2: 946.0, and [M+H]/3: 630.0 g/mol. ¹H-NMR (D₂O, 500 MHZ) δ=8.27 (m, 1H); 7.62 (m, 10H); 7.12 (d, J=6.2 Hz, 5H); 6.97 (dd, J=14.7 Hz, 7.9 Hz, 2H); 6.64 (dd, J=14.1 Hz, 8.1 Hz, 2H); 5.86 (dd, J=14.3 Hz, 3H); 5.40 (d, J=9.8 Hz, 3H); 4.97 (m, 3H); 4.54 (d, J=7.5 Hz, 3H); 4.46 (s, 1H); 4.35 (s, 1H); 3.97 (m, 1H); 3.85 (s, 6H); 3.50 (s, 6H); 3.26 (m, 20H); 3.09 (m, 3H); 2.77 (s, 10H); 2.62 (m, 4H); 2.33 (m, 9H); 2.15 (m, 2H); 1.90 (d, J=0.9 Hz, 7H); 1.68 (s, 10H); 0.95 (s, 10H) ppm.

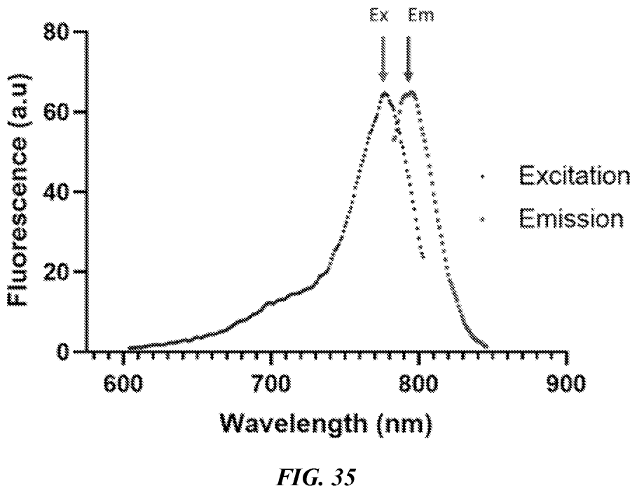

FIG. 35 shows the excitation and emission spectra of a 1 μM solution of FAP-4003 solution in PBS, pH 7.4, following synthesis.

1. A compound represented by the structure of formula (X):
Aₘ-L-B′ (X)
wherein:
A is a radical of a fibroblast activation protein alpha (FAPα) ligand of the formula X-B: 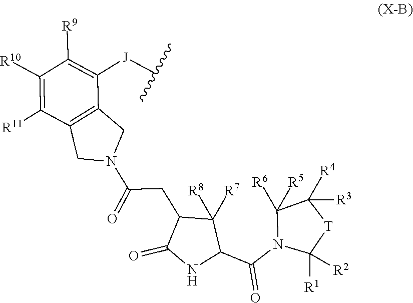
wherein:
T is substituted or unsubstituted methylene (—CH₂—), substituted or unsubstituted amino (—NH—), —O—, or —S— (e.g., wherein the substitution of T is C₁-C₃ alkyl, haloalkyl, or halo);
J is C(R^{J})₀₋₃, wherein each R^{J} is independently H or alkyl, or two or more R^{J} are taken together to form oxo;
R¹ and R² are independently selected from the group consisting of —H, —CN, —CHO, —B(OH)₂, —C(O)alkyl, —C(O)aryl-, —C═C—C(O)aryl, —C═C—S(O)₂aryl, —CO₂H, —SO₃H, —SO₂NH₂, —PO₃H₂, —SO₂F, —CONH₂, and 5-tetrazolyl;
R³ and R⁴ are independently selected from the group consisting of —H, —OH, F, Cl, Br, I, —C₁₋₆alkyl, —O—C₁₋₆alkyl, and —S—C₁₋₆alkyl;
R⁵, R⁶, R⁷, and R⁸ are independently selected from group consisting of H, alkyl, and halo; and
R⁹, R¹⁰, and R¹¹ are independently selected from group consisting of H, —C₁₋₆alkyl, —C₁₋₆haloalkyl, —O—C₁₋₆alkyl, —S—C₁₋₆ alkyl, F, Cl, Br and I;
L is a linker connecting A to B′;
B′ is a radical of a therapeutic agent, a radio-imaging agent, a radiotherapeutic agent, a magnetic resonance imaging agent, a chemotherapeutic agent, an antifibrotic agent, or an anticancer agent; and
m=1-6.

2. The compound of claim 1, further comprising C′, wherein:
L connects C′ to the one or more A groups and B′; and
C′ is a radical of an albumin binding ligand, a polyethylene glycolₙ (PEG)ₙ wherein n is an integer from 0 to 32, a peptide, a peptidoglycan or a saccharide.

3-6. (canceled)

7. The compound of claim 1, wherein A is represented by the structure of formula X-Y: 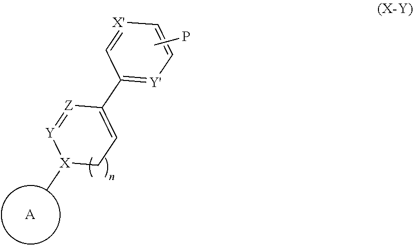
in which 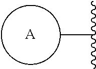
has the formula X-B,
n=0-4,
X, Y and Z are independently selected from O, N and S, with the proviso that at least one of X and Y is N or Z is N,
X′ and Y′ are independently selected from O, N, and S, with the proviso that at least one of X′ and Y′ is N or Z is N, and
P is a point of attachment to L or B′ of formula (X) and is selected from the group consisting of —H, —OH, —NH₂, —COOH, —CONH₂, —CHO, —N₃, —CN, —B(OH)₂, —C(O)alkyl, —C(O)aryl, —C═C—C(O)aryl, and —C═C—S(O)₂aryl.

8-9. (canceled)

10. The compound of claim 1, wherein B′ is a radical of:
a phosphoinositide 3-kinase (PI3K) inhibitor,
a chelating group optionally bound to an isotope (or metal), or a group covalently bound to an isotope (or metal), said isotope or metal being suitable for radio-imaging, radiotherapy or magnetic resonance imaging,
an anti-cancer agent,
an anti-fibrotic agent, or
a dye (e.g., a fluorescent dye).

11. (canceled)

12. The compound of claim 1, wherein A is

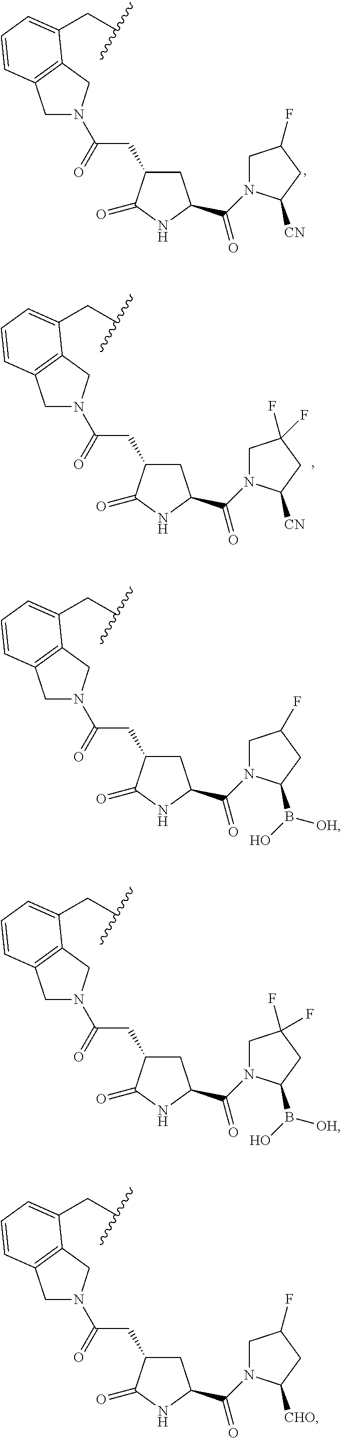 [SMILES: *Cc1cccc2c1CN(C(=O)C[C@@H]1C[C@@H](C(=O)N3CC(F)C[C@H]3C#N)NC1=O)C2 ; *Cc1cccc2c1CN(C(=O)C[C@@H]1C[C@@H](C(=O)N3CC(F)(F)C[C@H]3C#N)NC1=O)C2 ; *Cc1cccc2c1CN(C(=O)C[C@@H]1C[C@@H](C(=O)N3CC(F)C[C@H]3B(O)O)NC1=O)C2 ; *Cc1cccc2c1CN(C(=O)C[C@@H]1C[C@@H](C(=O)N3CC(F)(F)C[C@H]3B(O)O)NC1=O)C2 ; *Cc1cccc2c1CN(C(=O)C[C@@H]1C[C@@H](C(=O)N3CC(F)C[C@H]3C=O)NC1=O)C2]

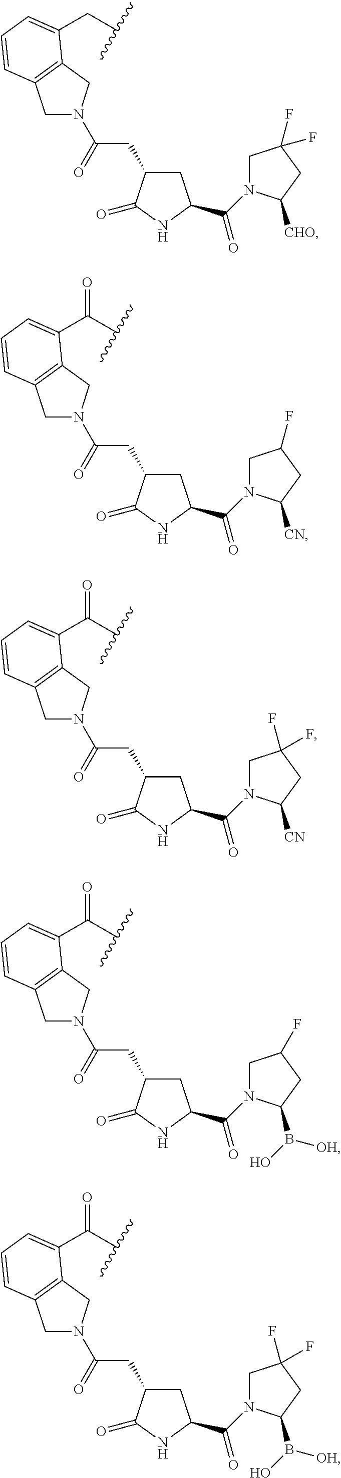 [SMILES: *Cc1cccc2c1CN(C(=O)C[C@@H]1C[C@@H](C(=O)N3CC(F)(F)C[C@H]3C=O)NC1=O)C2 ; *C(=O)c1cccc2c1CN(C(=O)C[C@@H]1C[C@@H](C(=O)N3CC(F)C[C@H]3C#N)NC1=O)C2 ; *C(=O)c1cccc2c1CN(C(=O)C[C@@H]1C[C@@H](C(=O)N3CC(F)(F)C[C@H]3C#N)NC1=O)C2 ; *C(=O)c1cccc2c1CN(C(=O)C[C@@H]1C[C@@H](C(=O)N3CC(F)C[C@H]3B(O)O)NC1=O)C2 ; *C(=O)c1cccc2c1CN(C(=O)C[C@@H]1C[C@@H](C(=O)N3CC(F)(F)C[C@H]3B(O)O)NC1=O)C2]

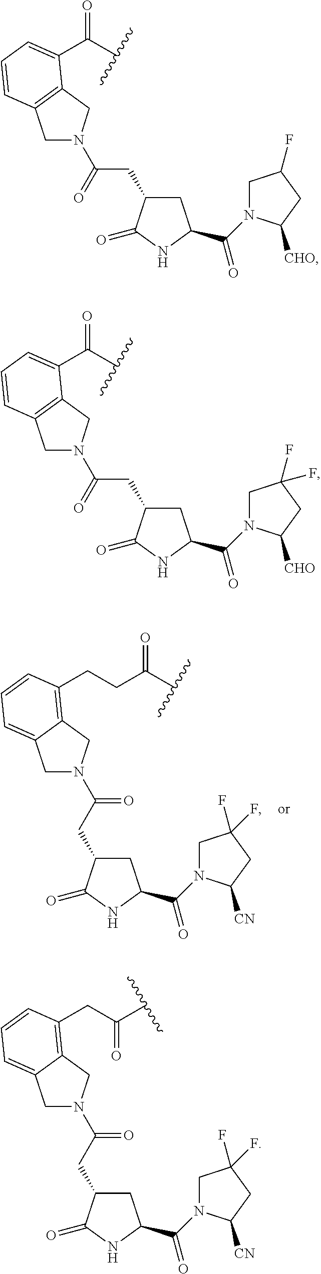 [SMILES: *C(=O)c1cccc2c1CN(C(=O)C[C@@H]1C[C@@H](C(=O)N3CC(F)C[C@H]3C=O)NC1=O)C2 ; *C(=O)c1cccc2c1CN(C(=O)C[C@@H]1C[C@@H](C(=O)N3CC(F)(F)C[C@H]3C=O)NC1=O)C2 ; *C(=O)CCc1cccc2c1CN(C(=O)C[C@@H]1C[C@@H](C(=O)N3CC(F)(F)C[C@H]3C#N)NC1=O)C2 ; *C(=O)Cc1cccc2c1CN(C(=O)C[C@@H]1C[C@@H](C(=O)N3CC(F)(F)C[C@H]3C#N)NC1=O)C2]

13. The compound of claim 1, wherein A is

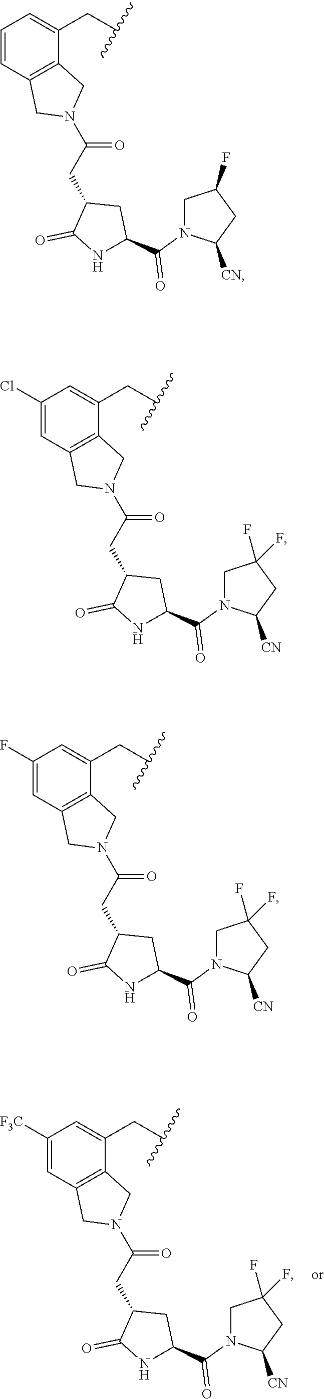 [SMILES: *Cc1cccc2c1CN(C(=O)C[C@@H]1C[C@@H](C(=O)N3C[C@@H](F)C[C@H]3C#N)NC1=O)C2 ; *Cc1cc(Cl)cc2c1CN(C(=O)C[C@@H]1C[C@@H](C(=O)N3CC(F)(F)C[C@H]3C#N)NC1=O)C2 ; *Cc1cc(F)cc2c1CN(C(=O)C[C@@H]1C[C@@H](C(=O)N3CC(F)(F)C[C@H]3C#N)NC1=O)C2 ; *Cc1cc(C(F)(F)F)cc2c1CN(C(=O)C[C@@H]1C[C@@H](C(=O)N3CC(F)(F)C[C@H]3C#N)NC1=O)C2]

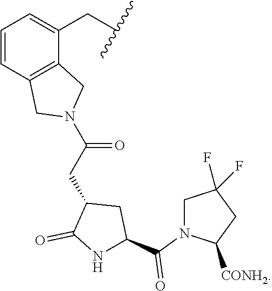 [SMILES: *Cc1cccc2c1CN(C(=O)C[C@@H]1C[C@@H](C(=O)N3CC(F)(F)C[C@H]3C(N)=O)NC1=O)C2]

14. The compound of claim 1, wherein A comprises

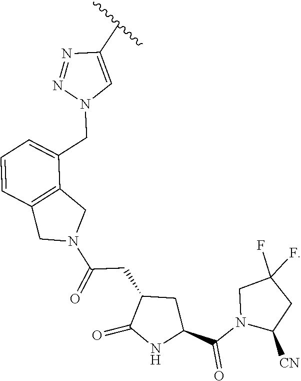 [SMILES: *c1cn(Cc2cccc3c2CN(C(=O)C[C@@H]2C[C@@H](C(=O)N4CC(F)(F)C[C@H]4C#N)NC2=O)C3)nn1]

15. The compound of claim 1, wherein A comprises 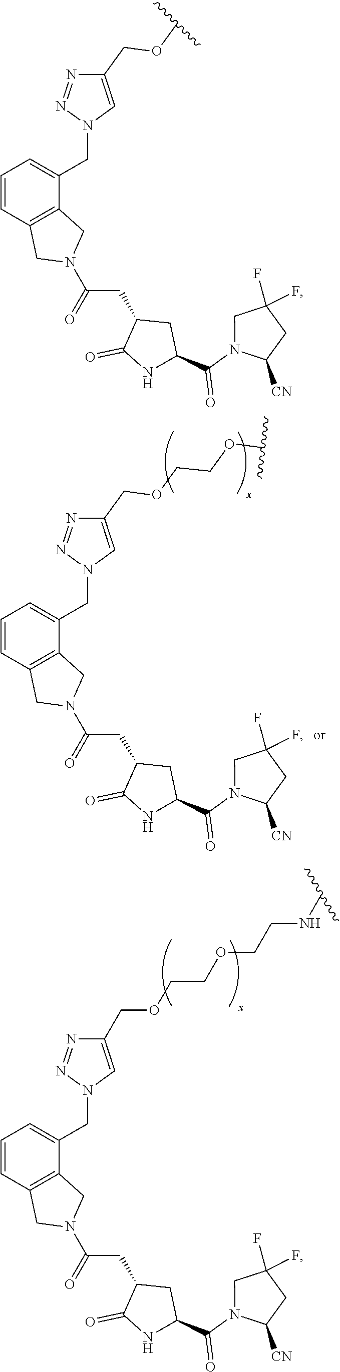
wherein x is 1-20.

16. The compound of claim 1, wherein A comprises

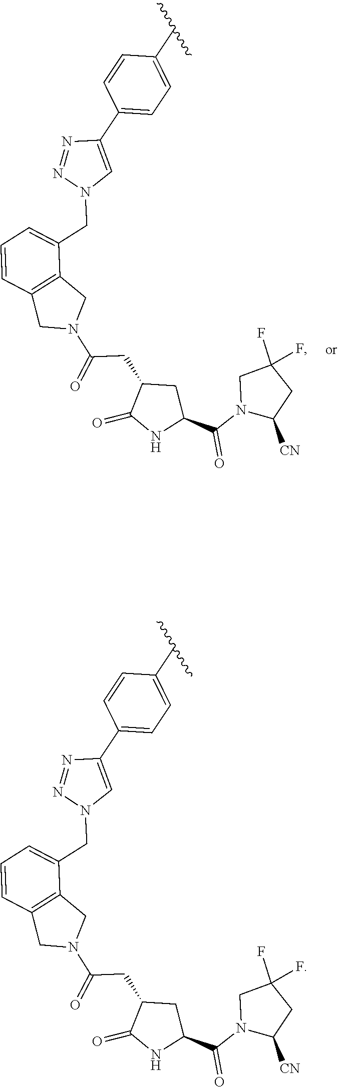 [SMILES: *c1ccc(-c2cn(Cc3cccc4c3CN(C(=O)C[C@@H]3C[C@@H](C(=O)N5CC(F)(F)C[C@H]5C#N)NC3=O)C4)nn2)cc1 ; *c1ccc(-c2cn(Cc3cccc4c3CN(C(=O)C[C@@H]3C[C@@H](C(=O)N5CC(F)(F)C[C@H]5C#N)NC3=O)C4)nn2)cc1]

17. The compound of claim 1, wherein B′ is a radical of a chelating group optionally bound to an isotope (or metal) suitable for radio-imaging, radiotherapy or magnetic resonance imaging.

18. The compound of claim 1, wherein B′ is a radical of, wherein B′ is selected from 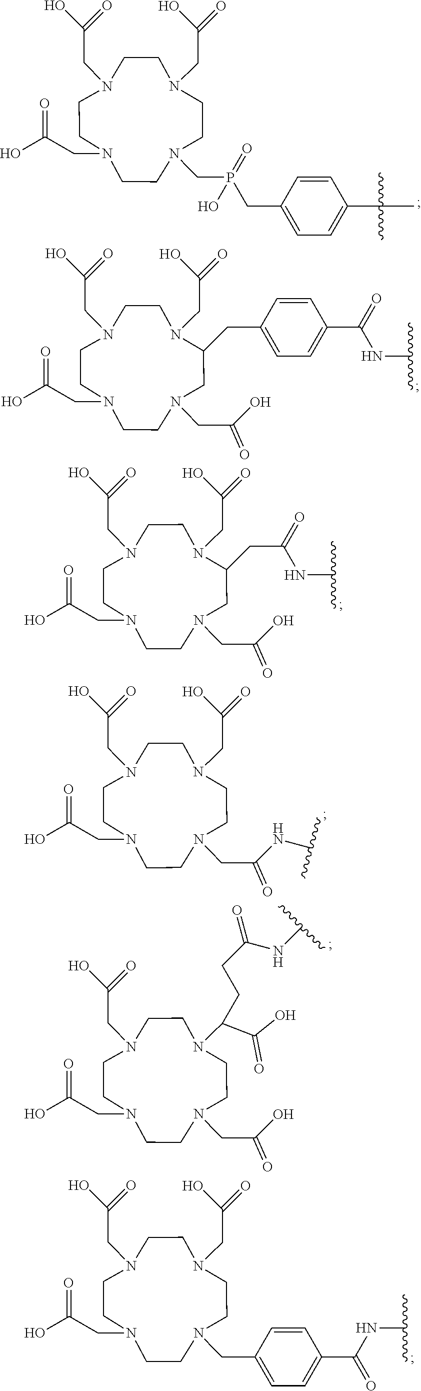 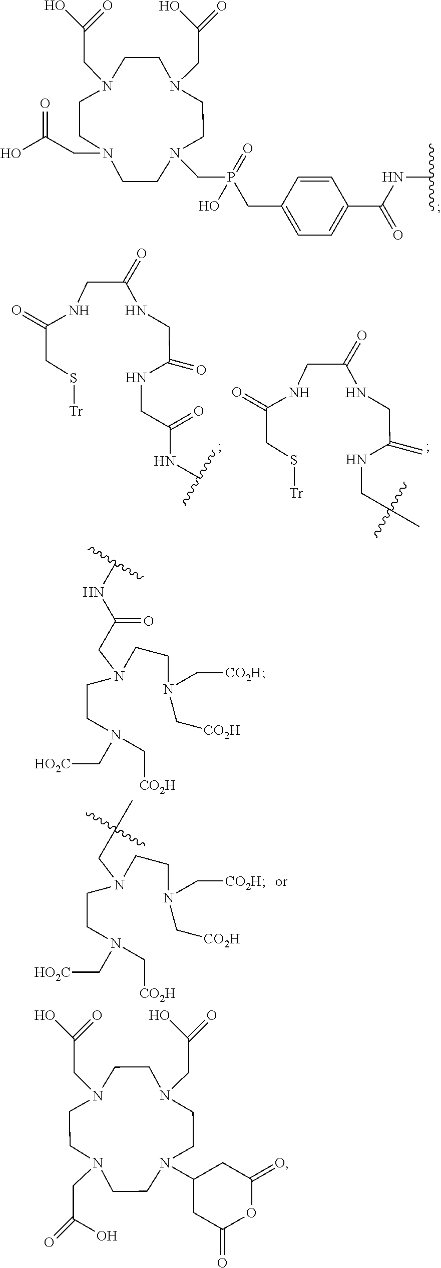
each optionally bound to an isotope (or metal) suitable for radio-imaging, radiotherapy or magnetic resonance imaging.

19. The compound of claim 1, wherein B′ is a radical of, wherein B′ is selected from 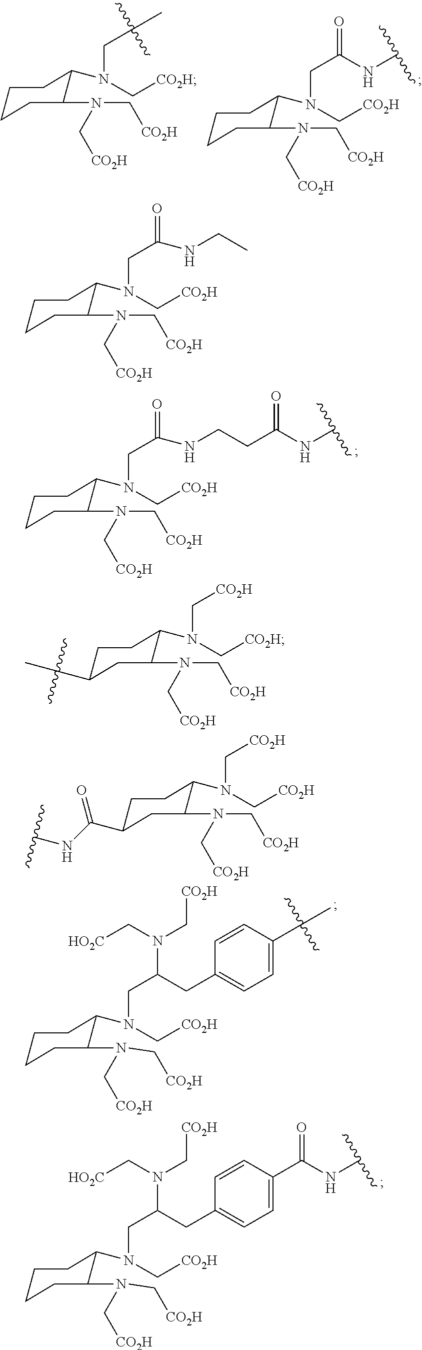 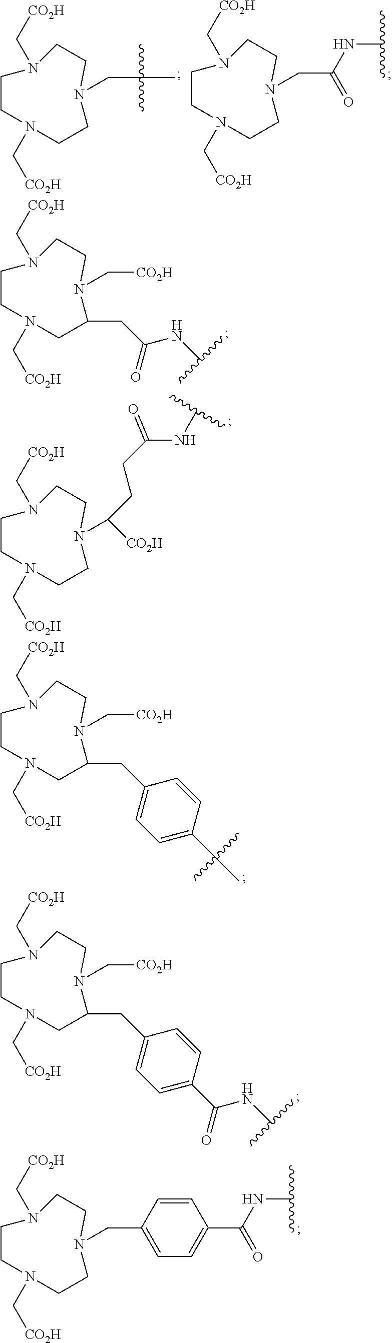 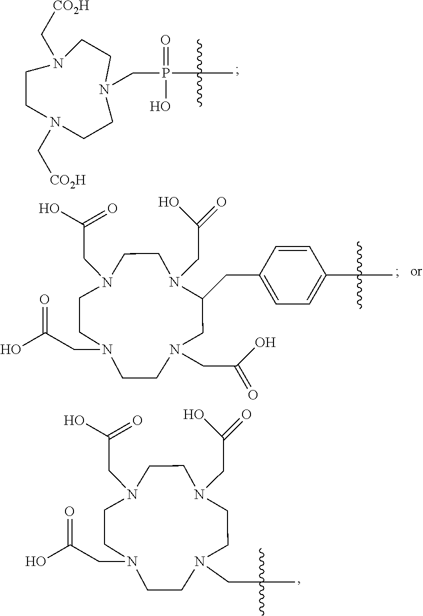
each optionally bound to an isotope (or metal) suitable for radio-imaging, radiotherapy or magnetic resonance imaging.

20. (canceled)

21. The compound of claim 1, wherein B′ comprises a radical of DOTA, a radical of an isotope- (or metal-) chelated DOTA, a radical of 1,4,7,10-tetraazacyclododecane-1,4,7,10-tetraacetic acid, or

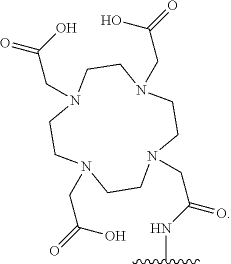

22. The compound of claim 1, wherein B′ is a radical of a group covalently bound to an isotope suitable for radio-imaging, radiotherapy or magnetic resonance imaging, the group selected from

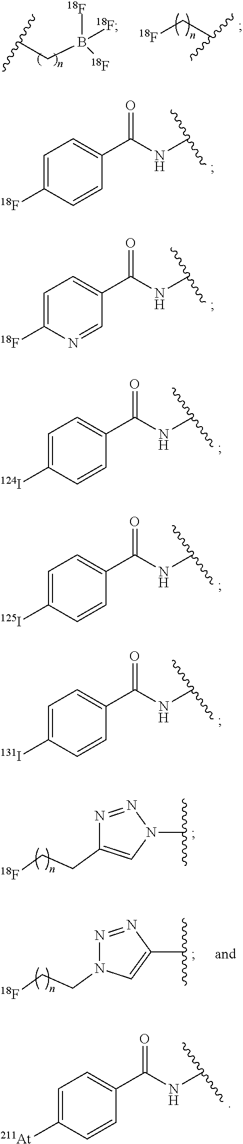

23-27. (canceled)

28. The compound of claim 1, wherein A has a binding affinity to FAPα from about 1 nM to about 25 nM.

29. The compound of claim 1, wherein L comprises a non-releasable linker.

30. The compound of claim 1, wherein L comprises a releasable linker.

31. The compound of claim 1, wherein L comprises a PEGₙ and n=0-36, a peptide, or a peptidoglycan.

32. The compound of claim 1, wherein L comprises

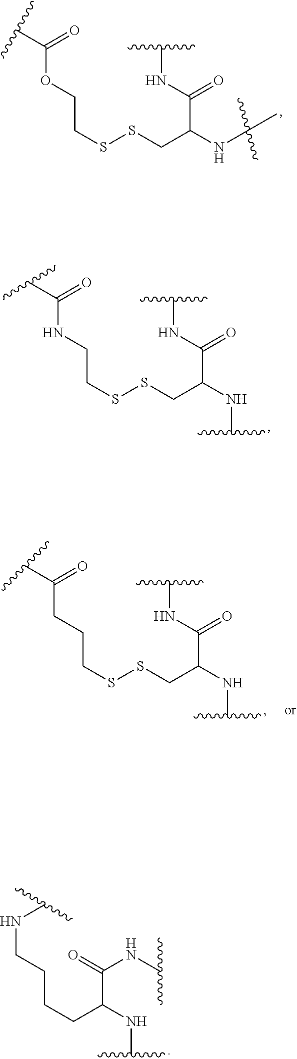

33-42. (canceled)

43. The compound of claim 2, wherein C′ is a radical of an albumin binding ligand and has the following structure:

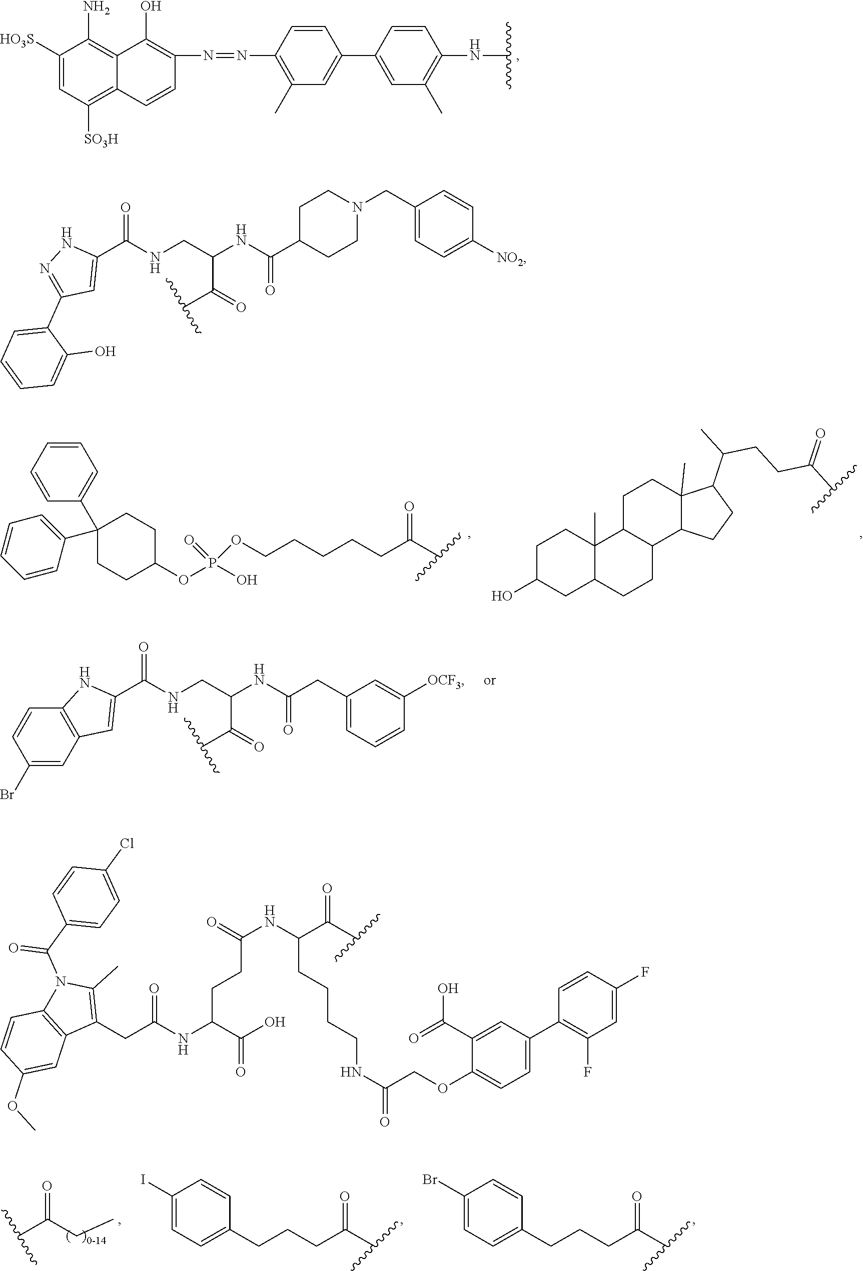

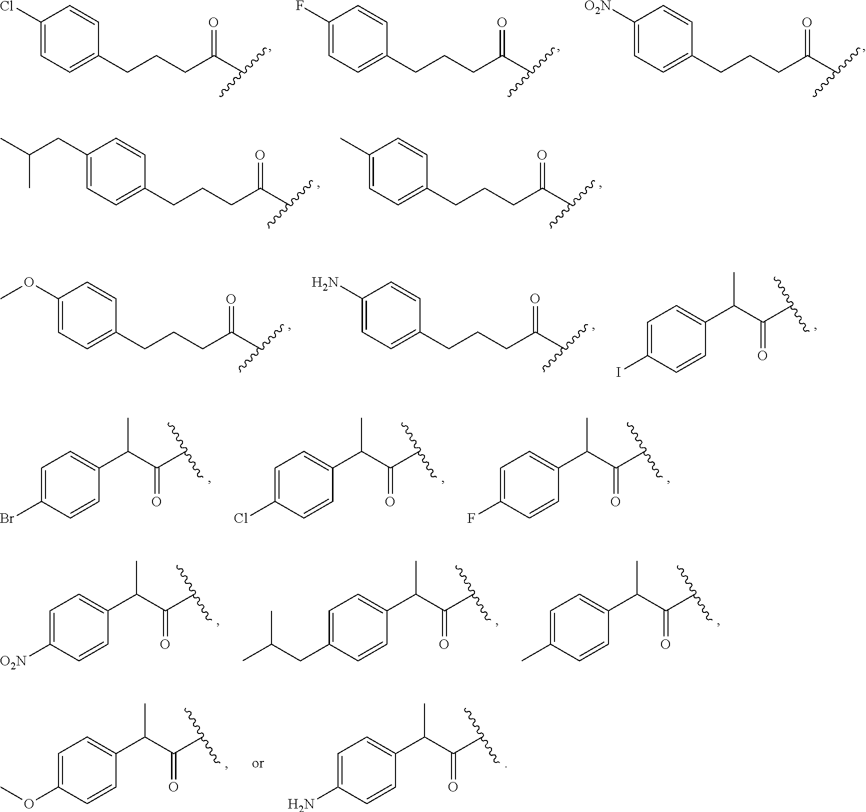

44. (canceled)

45. The compound of claim 2, wherein C′ is a radical of

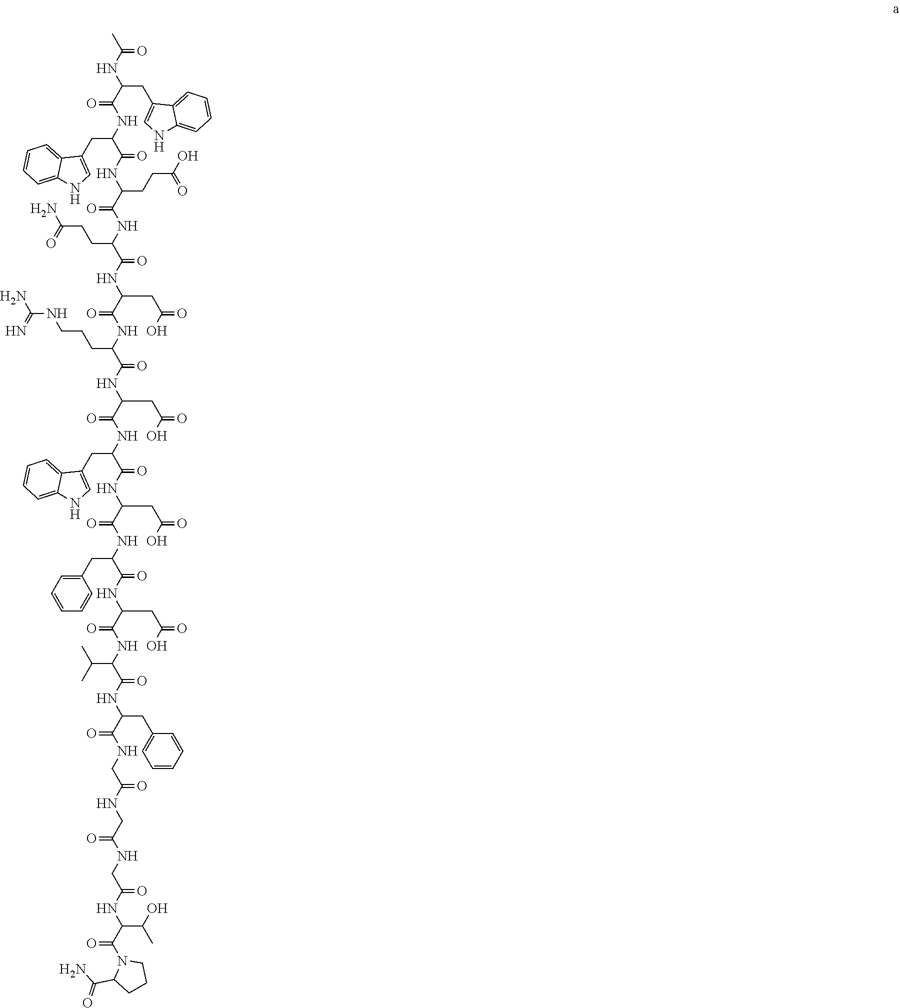

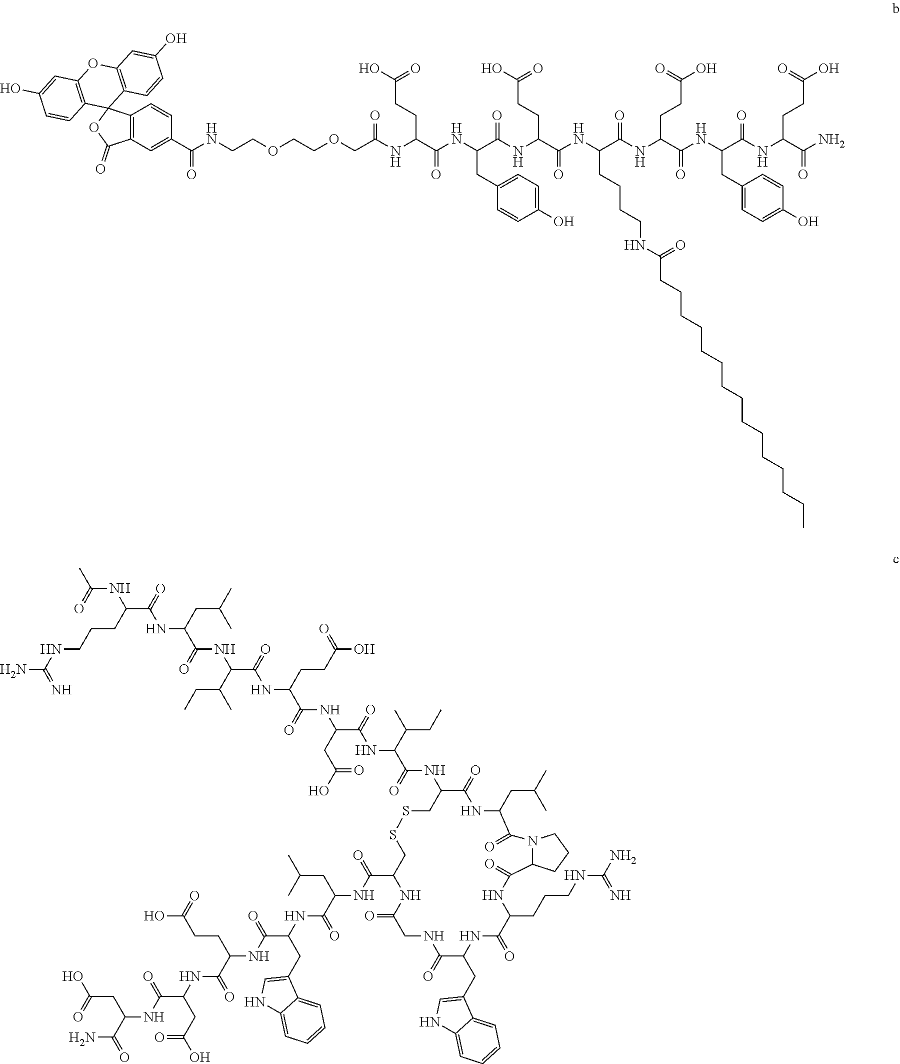

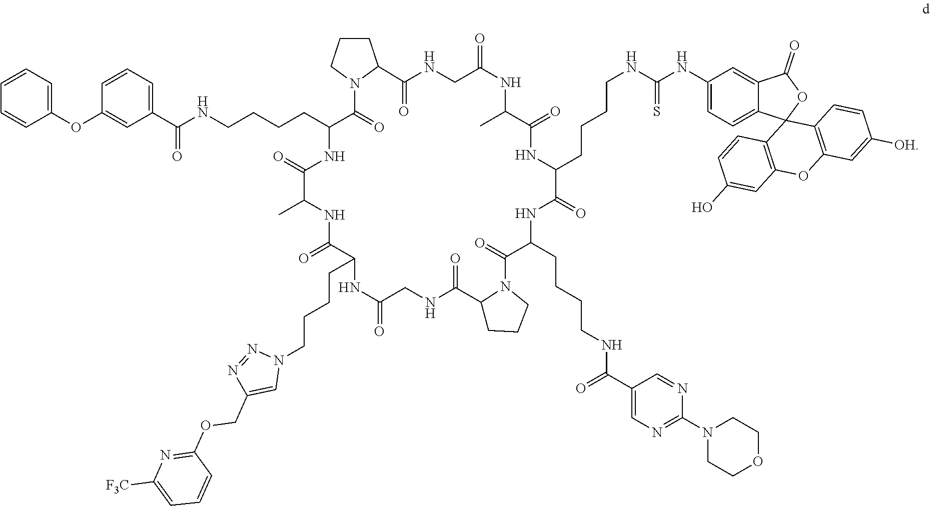

46. The compound of claim 2, wherein C′ is a radical of an albumin-binding small protein scaffold comprising ABD035, ABDCon, DARPins, dsFv CA645, nanobody, and VNAR (E06).

47. The compound of claim 2, wherein C′ is PEGₙ and n is an integer from 0 to 32, a peptide, a peptidoglycan, or a saccharide.

48. The compound of claim 2, wherein C′ is

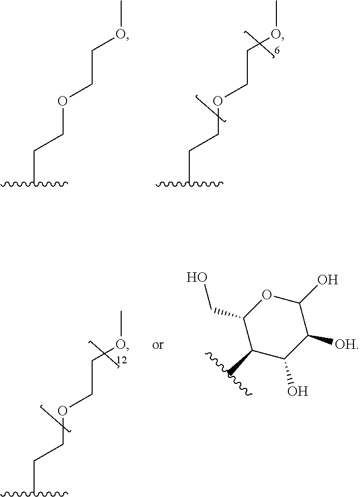

49. (canceled)

50. The compound of claim 1, represented by the structure of formula (V): 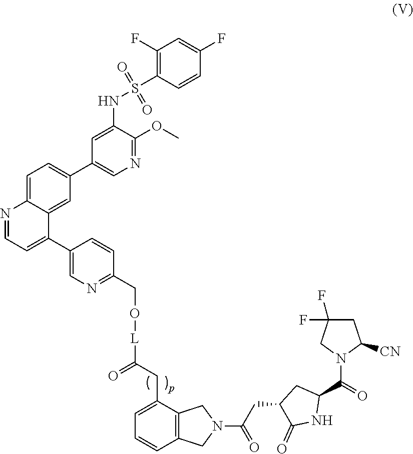
wherein:
L is a linker comprising at least one carbon atom; and
p is 0, 1, 2, or 3.

51. (canceled)

52. The compound of claim 50, wherein L comprises: 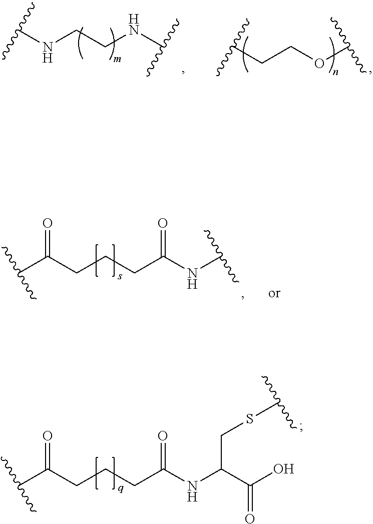
wherein
m is an integer from 1 to 9;
n is an integer from 1 to 32;
q is an integer from 0 to 4; and
s is an integer from 0 to 4.

53. The compound of claim 50, wherein the compound is: 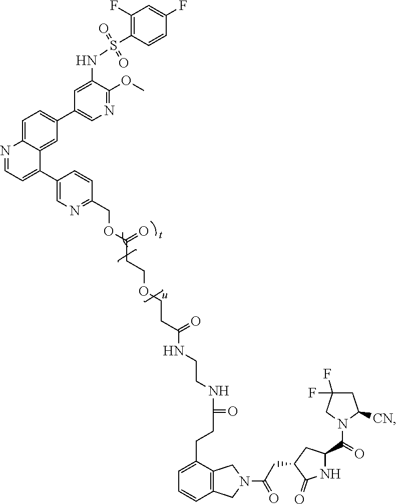
wherein t is 0 or 1 and u is an integer from 2-12.

54. The compound of claim 50, wherein the compound is 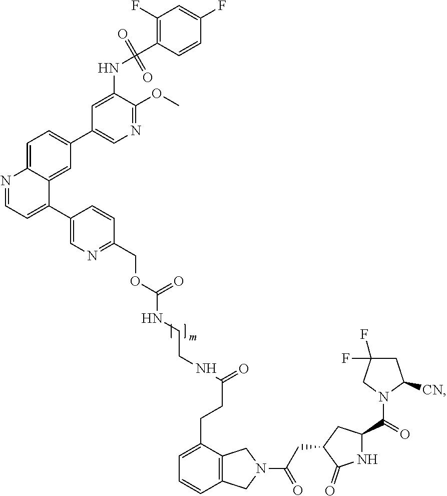
wherein m is an integer from 1 to 4.

55. (canceled)

56. The compound of claim 50, wherein the compound is

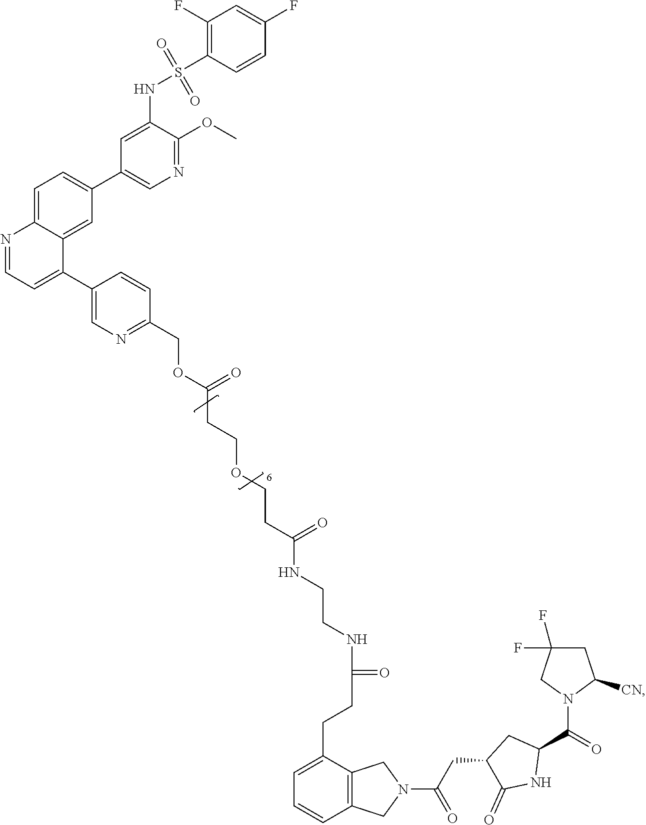

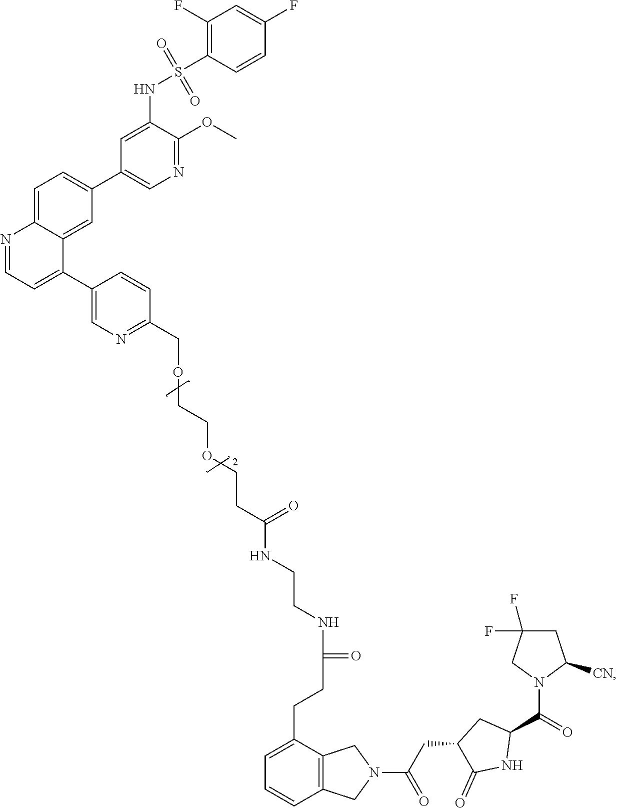

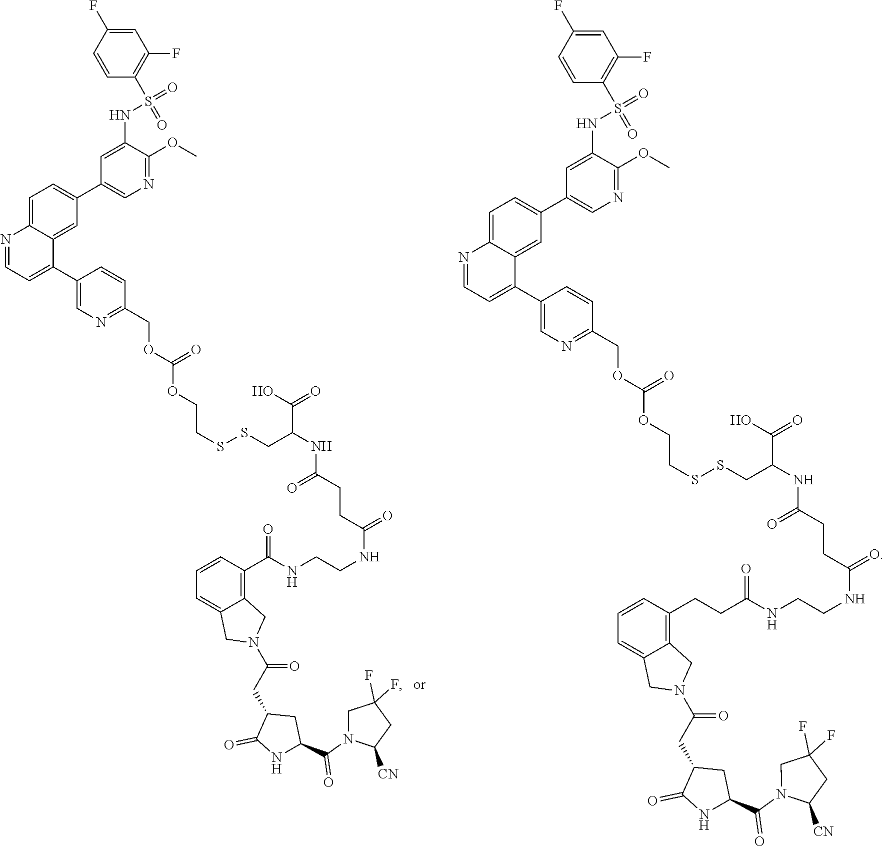

57-63. (canceled)

64. The compound of claim 1 having the following structure: 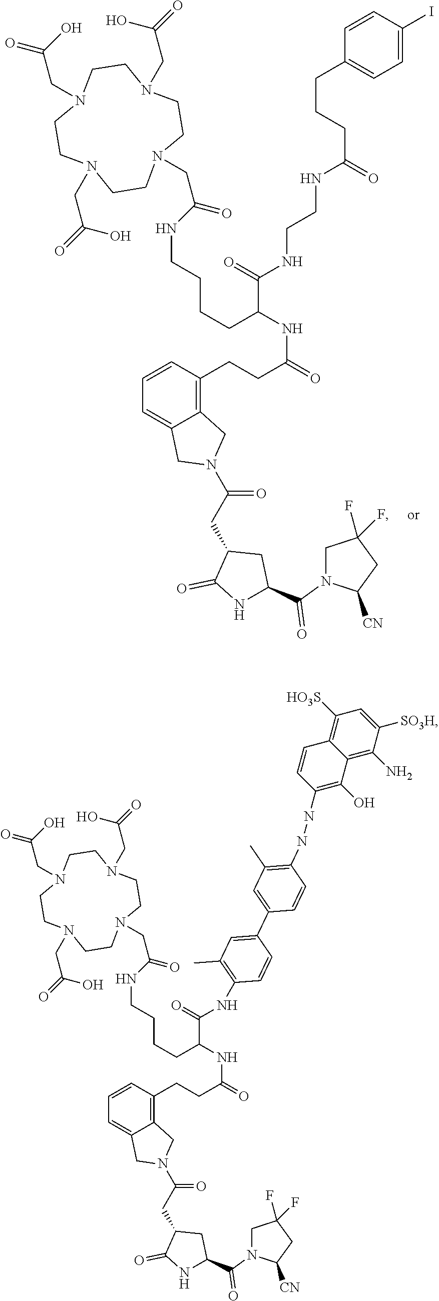 [SMILES: N#C[C@@H]1CC(F)(F)CN1C(=O)[C@@H]1C[C@@H](CC(=O)N2Cc3cccc(CCC(=O)NC(CCCCNC(=O)CN4CCN(CC(=O)O)CCN(CC(=O)O)CCN(CC(=O)O)CC4)C(=O)NCCNC(=O)CCCc4ccc(I)cc4)c3C2)C(=O)N1 ; Cc1cc(-c2ccc(NC(=O)C(CCCCNC(=O)CN3CCN(CC(=O)O)CCN(CC(=O)O)CCN(CC(=O)O)CC3)NC(=O)CCc3cccc4c3CN(C(=O)C[C@@H]3C[C@@H](C(=O)N5CC(F)(F)C[C@H]5C#N)NC3=O)C4)c(C)c2)ccc1NNc1ccc2c(S(=O)(=O)O)cc(S(=O)(=O)O)c(N)c2c1O]
each optionally bound to an isotope (or metal) suitable for radio-imaging, radiotherapy or magnetic resonance imaging.

65. The compound of claim 1 of the following structure: 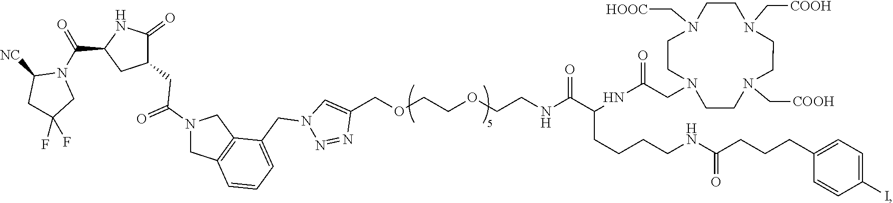
optionally bound to an isotope (or metal) suitable for radio-imaging, radiotherapy or magnetic resonance imaging.

66. The compound of claim 1 of the following structure: 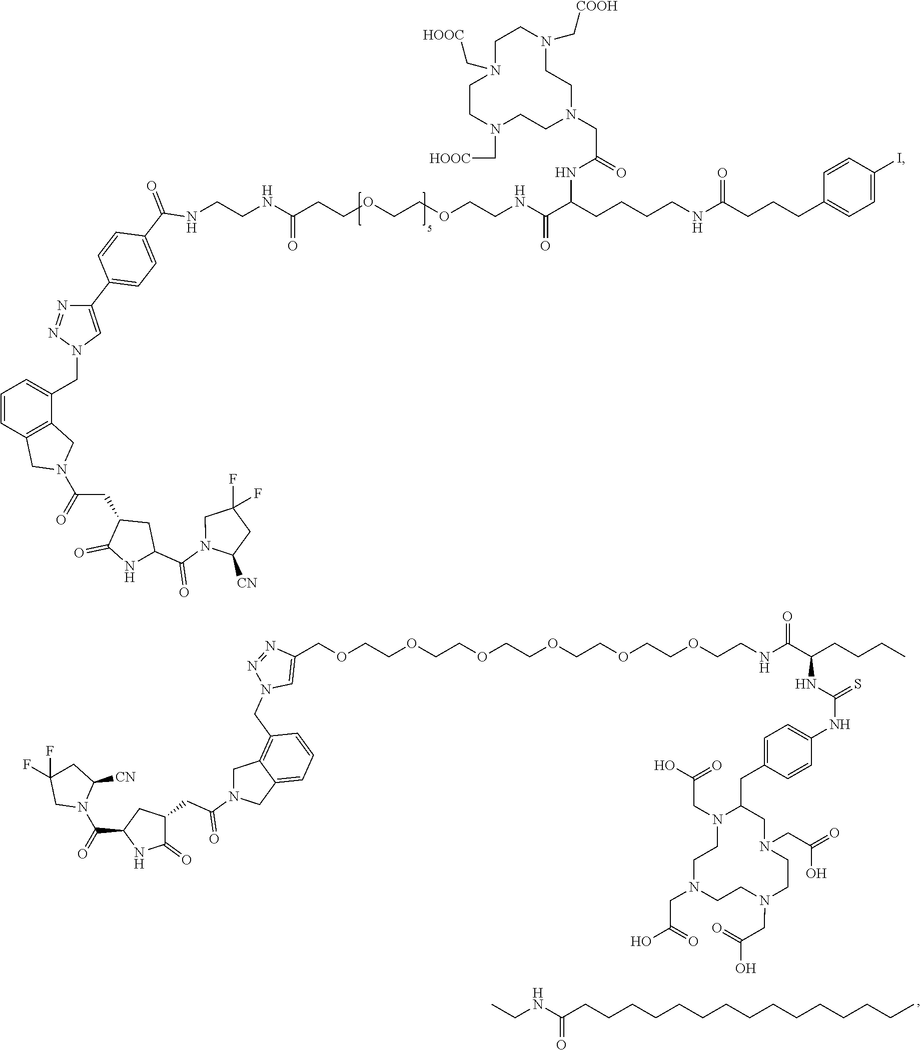 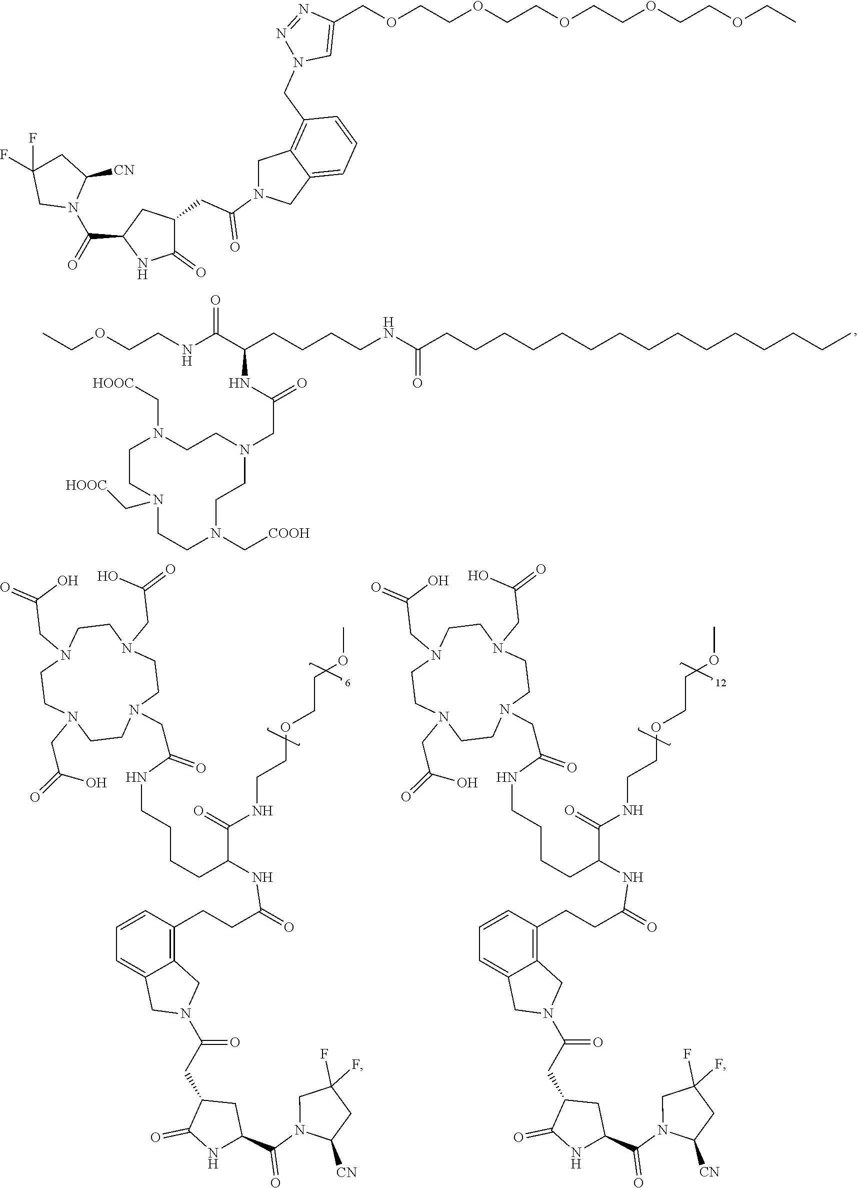 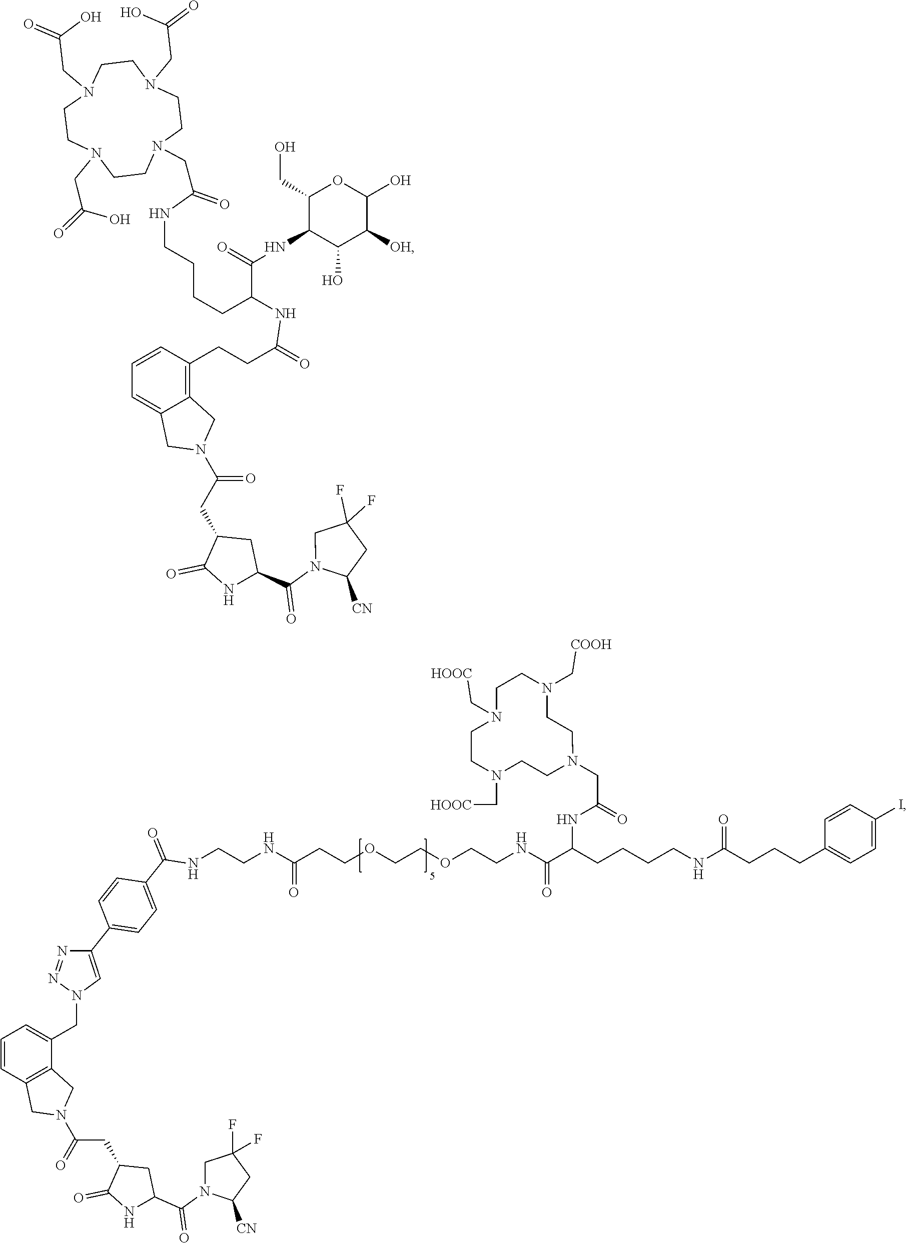 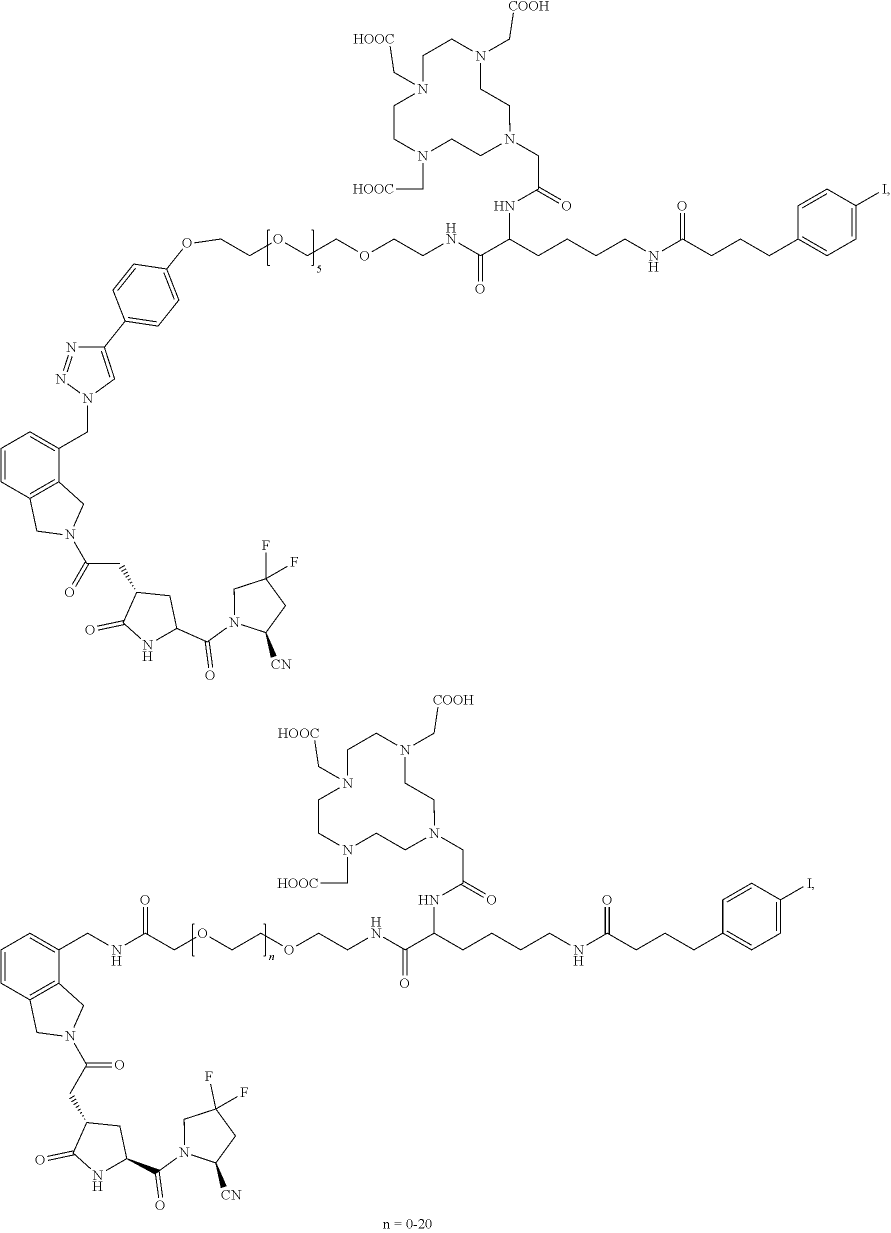 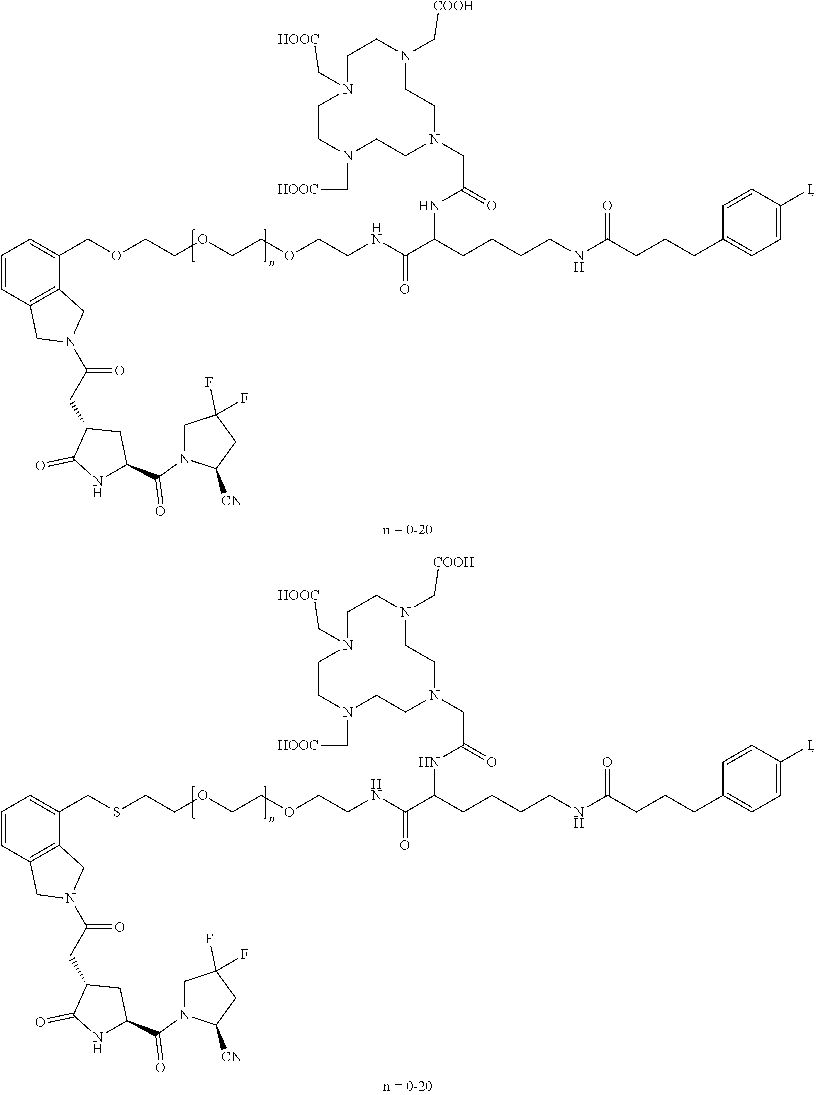 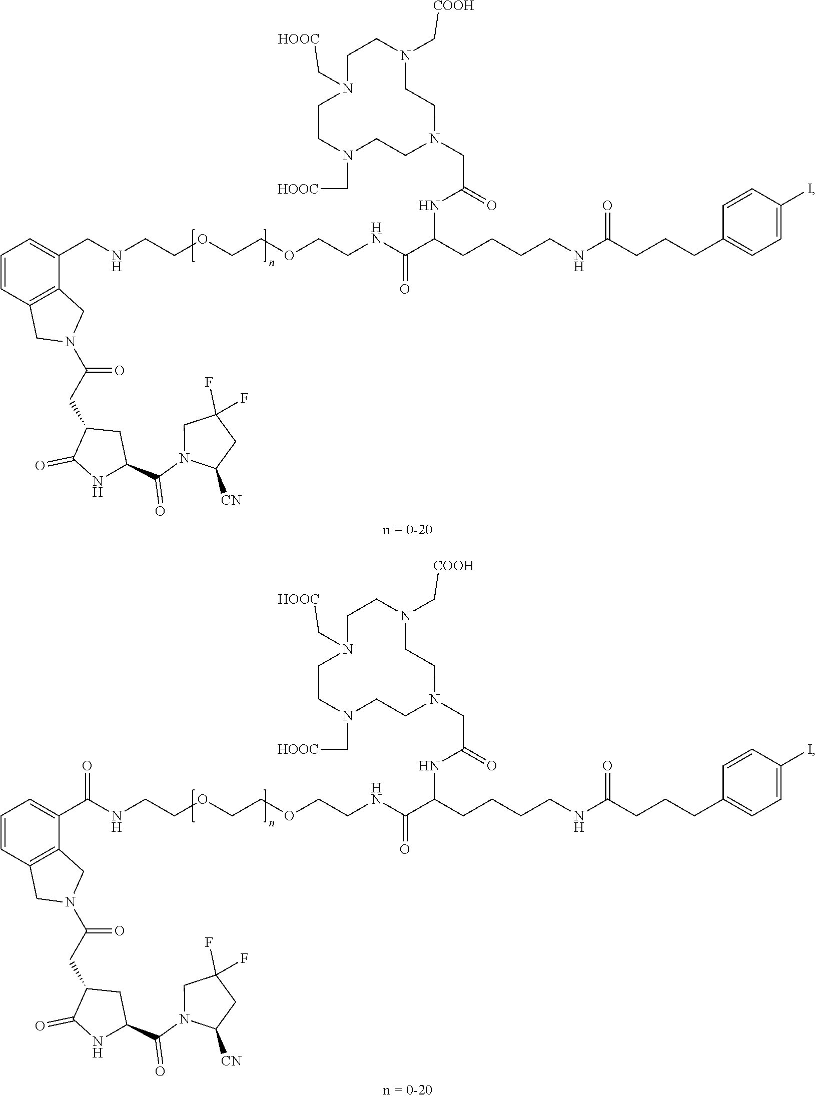 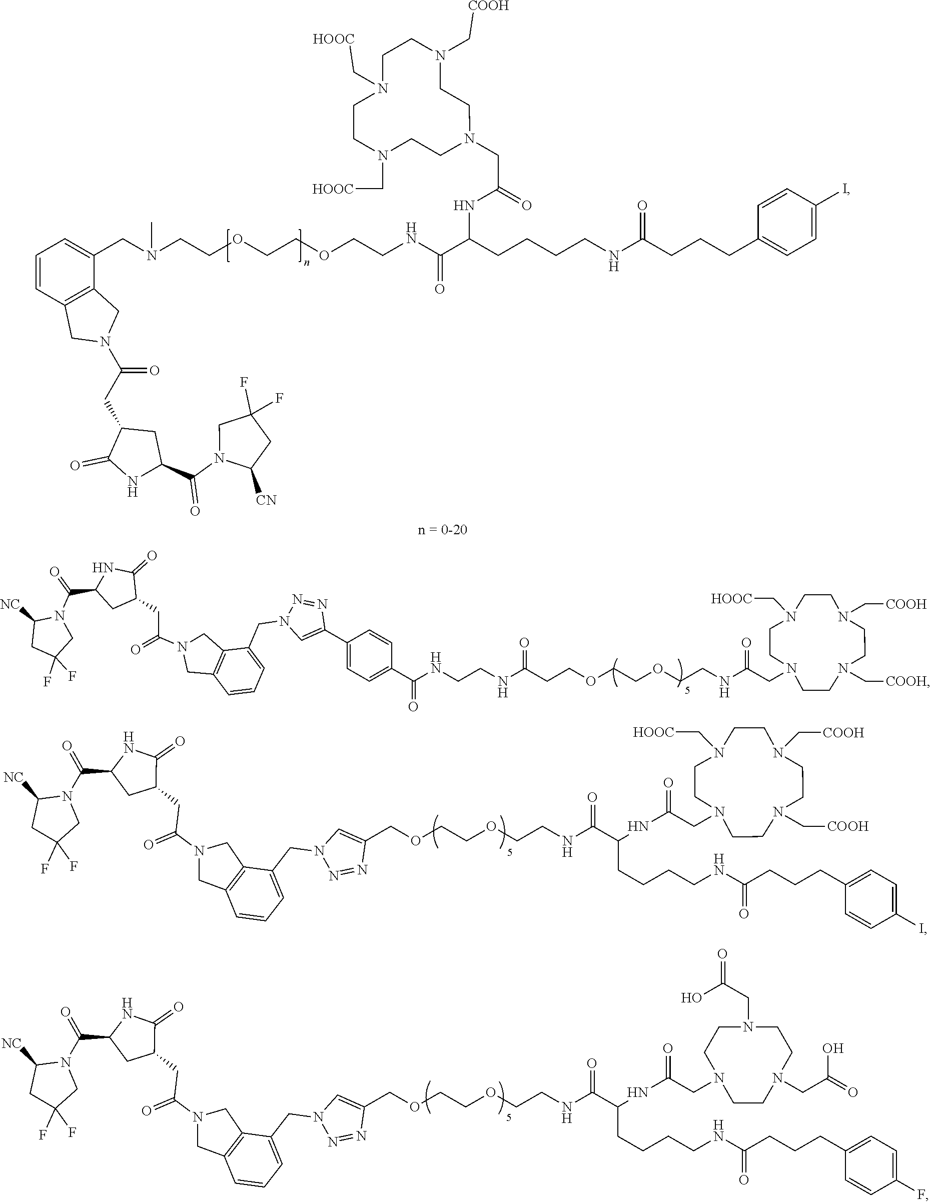 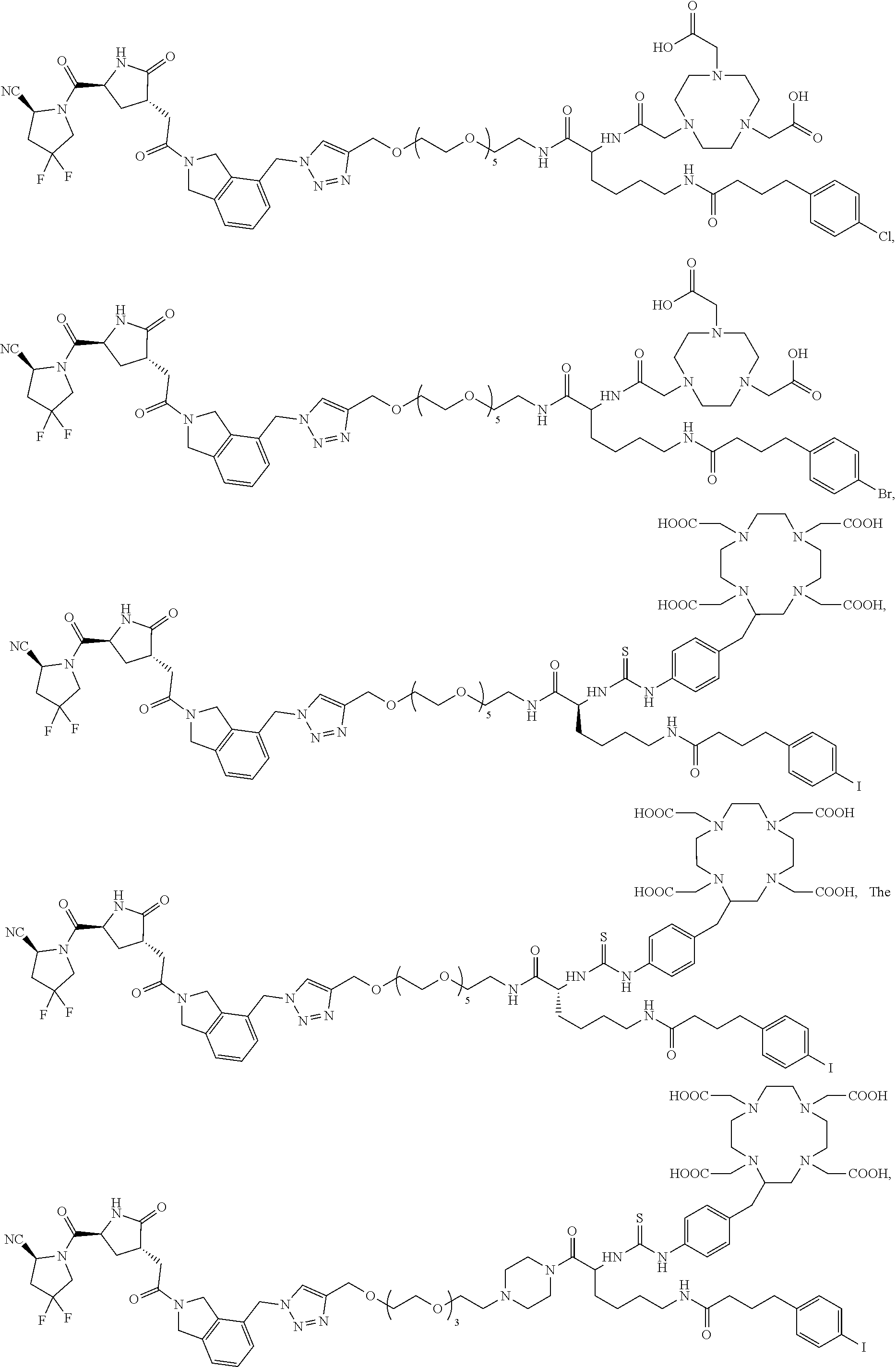 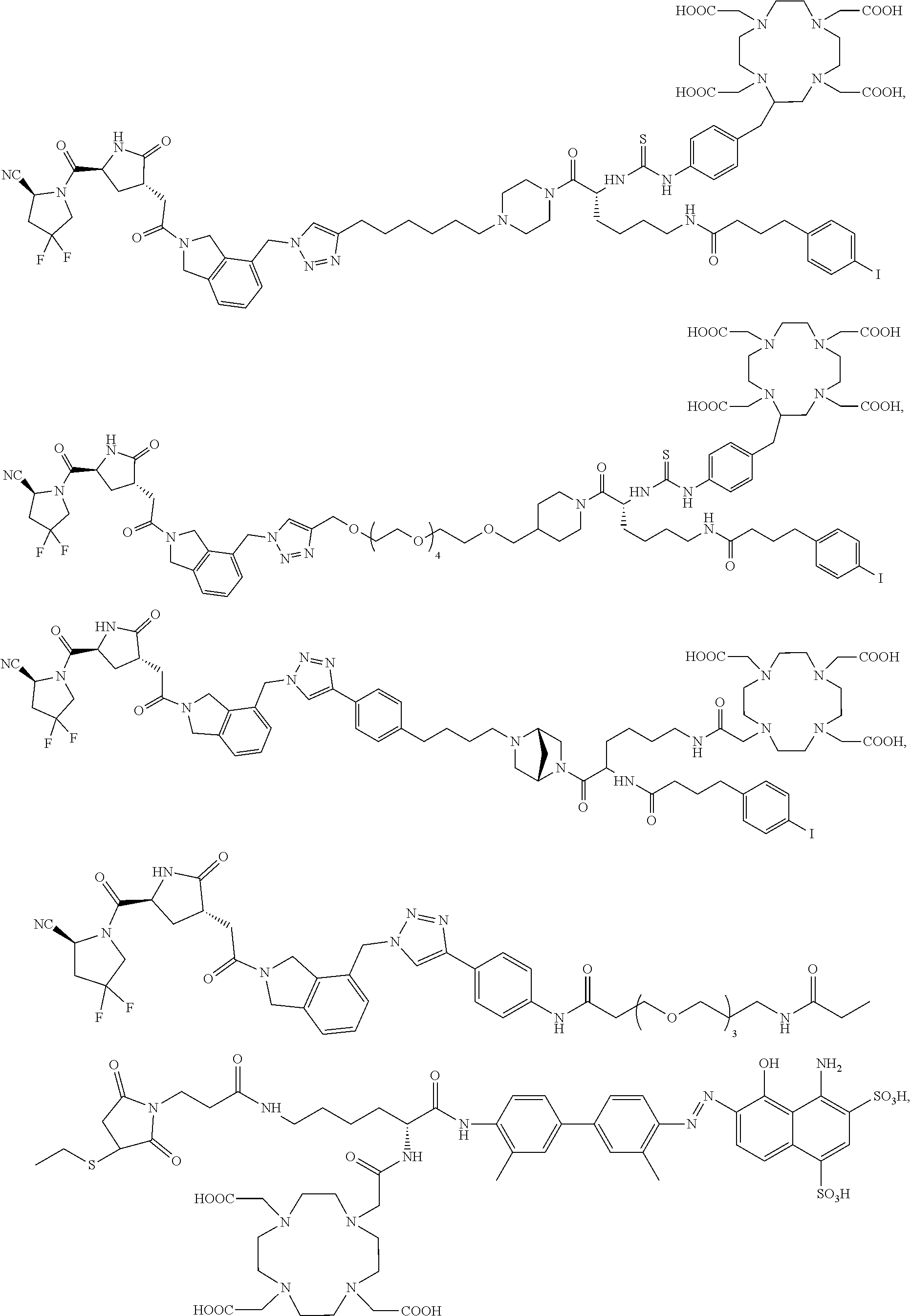 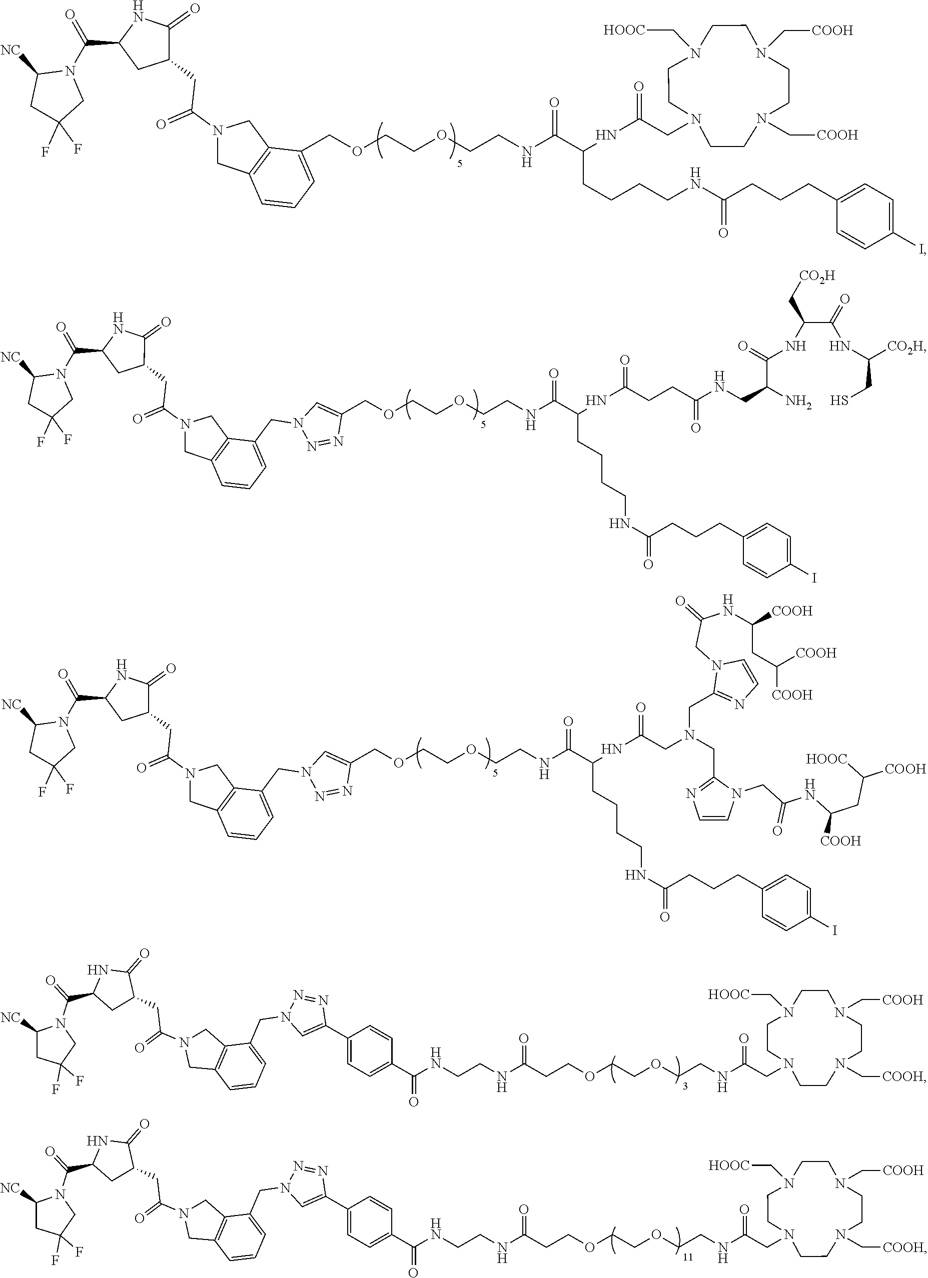 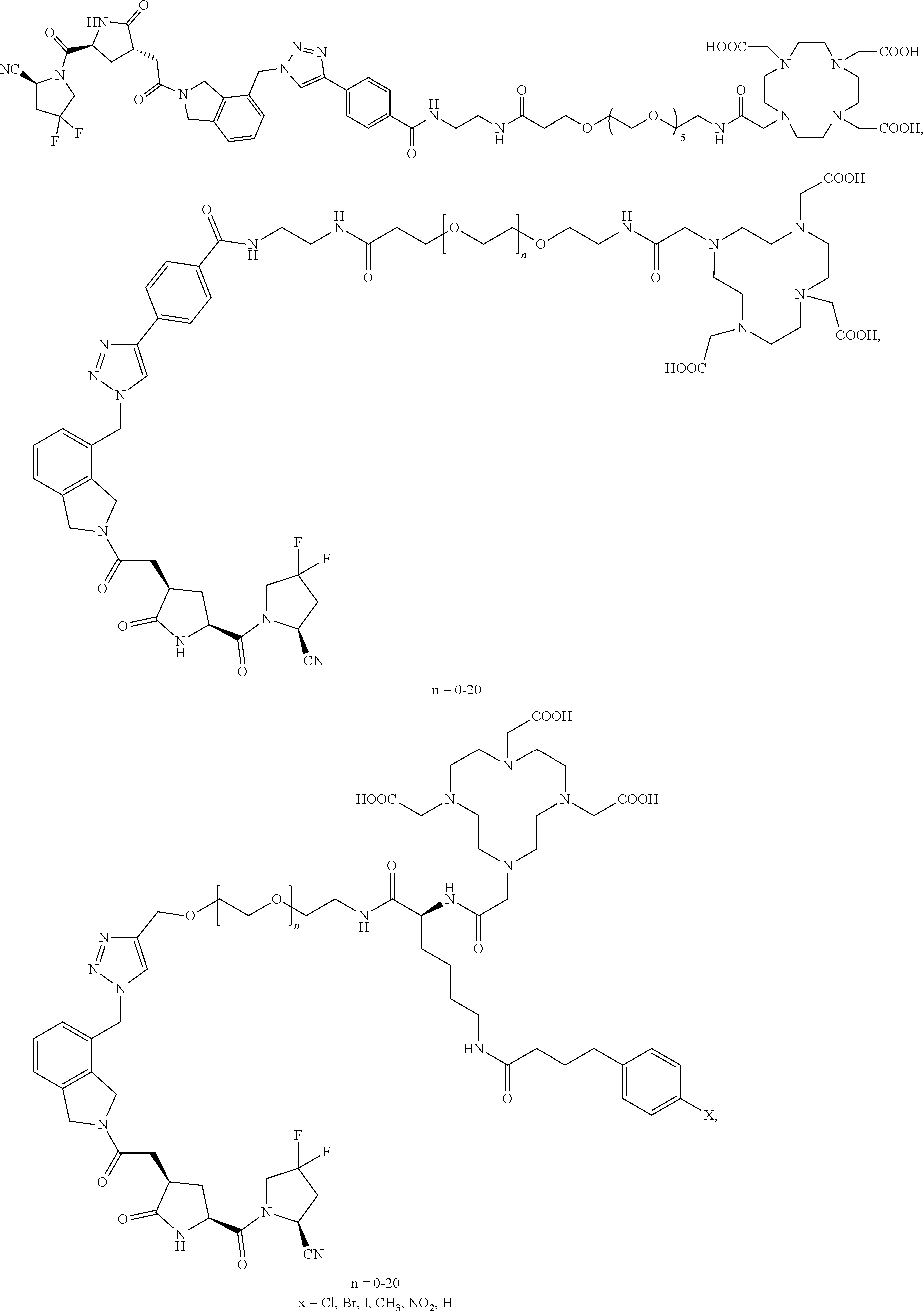 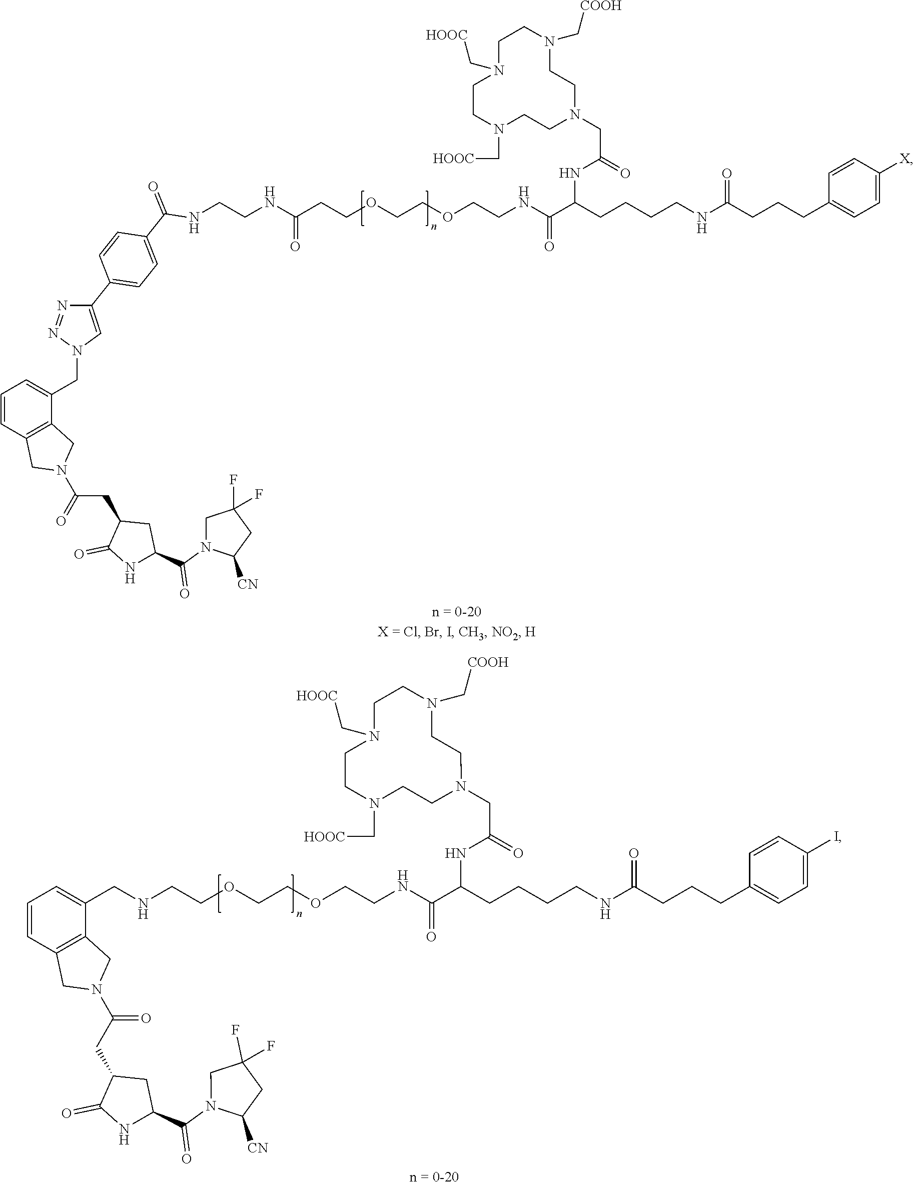 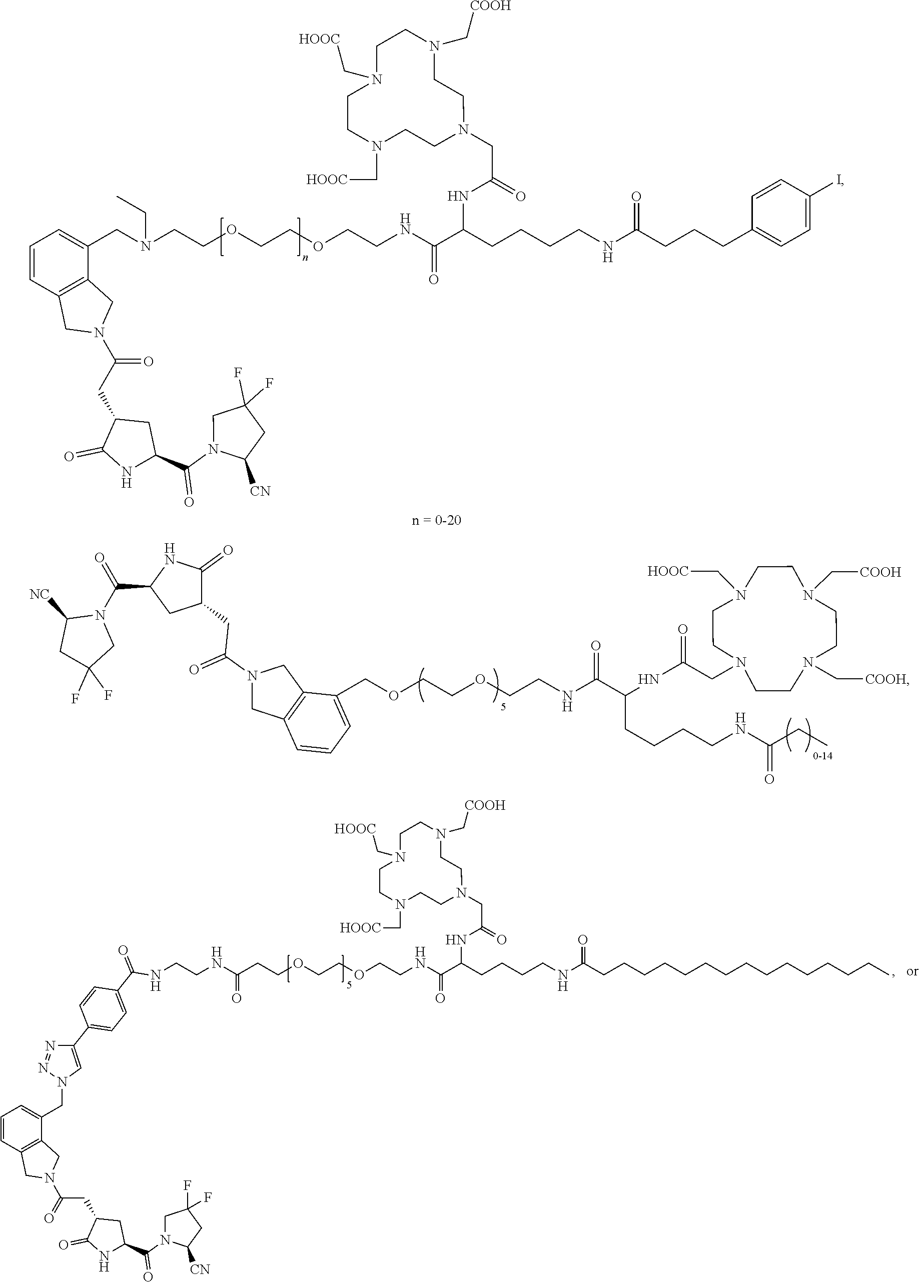 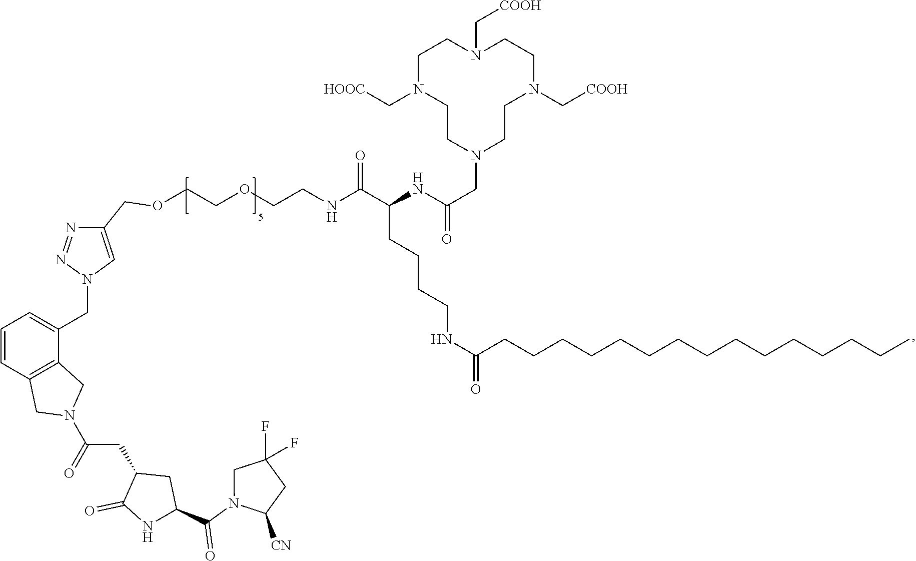
or any of the foregoing structures bound to an isotope suitable for radio-imaging, radiotherapy or magnetic resonance imaging.

67-103.

104. The compound of claim 1 having the structure:

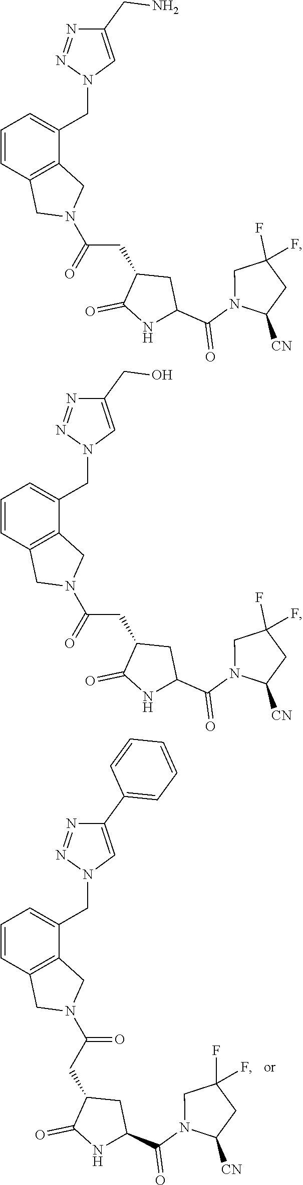

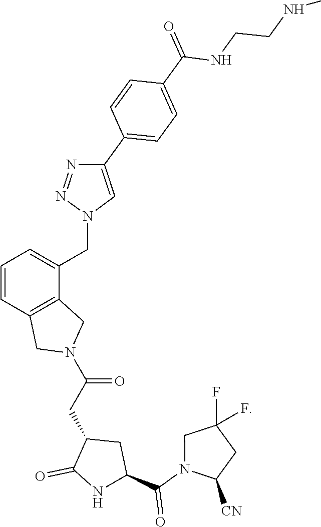 [SMILES: CNCCNC(=O)c1ccc(-c2cn(Cc3cccc4c3CN(C(=O)C[C@@H]3C[C@@H](C(=O)N5CC(F)(F)C[C@H]5C#N)NC3=O)C4)nn2)cc1]

105-107. (canceled)

108. A pharmaceutical composition comprising a compound of claim 1 and a pharmaceutically acceptable carrier.

109. A method for imaging cancer or fibrosis in a subject with the cancer or the fibrosis, the method comprising administering an effective amount of a compound of claim 1 or a pharmaceutical composition comprising such compound and a pharmaceutically acceptable carrier to the subject and imaging the subject.

110. The method of claim 109, further comprising generating an image of the cancer or the fibrosis in the subject.

111. The method of claim 109, wherein the fibrosis is selected from pulmonary fibrosis, renal fibrosis, and hepatic fibrosis.

112-114. (canceled)

115. A method for treating cancer, the method comprising administering a therapeutically effective amount of a compound of claim 1 or a pharmaceutical composition comprising such compound and a pharmaceutically acceptable carrier to a subject in need thereof.

116. The method of claim 115, wherein the cancer is selected from the group consisting of lung cancer, breast cancer, colorectal cancer, cervical cancer, and brain cancer (e.g., glioblastoma).

117. The method of claim 115, further comprising administering chemotherapy or radiotherapy to the subject.

118. A method of improving the affinity of a ligand for FAP, such ligand comprising an isoindoline scaffold, and the method comprising:
introducing a triazole moiety into the isoindoline scaffold of the ligand by molecular modeling to achieve a higher Schrodinger molecular docking score, whereupon the affinity of the ligand for FAP is improved.

119. A ligand for FAP, the ligand comprising an isoindoline scaffold into which a triazole moiety has been introduced and which has a Schrodinger molecular docking score of at least about −8.0 kcal/mol.
